SEQUENCE LISTING (892 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
atgcattctgcccccaagga

SEQ ID NO: 3 - DNA, 14121 bp, Homo sapiens
  CDS (129)..(13820)
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaacaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctcaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctgaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtagtggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttgacccaccaatacaagttgaaggagactattcagaagctaagcaatgtcctacaacaagttaagataaaagattactttgagaaattggttggatttattgatgatgctgtgaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtcgaaatgaaagtaaagatcatcagaaccattgaccagatgcagaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccacatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaaaaaccacaaaagccgaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattactcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacacttgcacaccgtgacttcagtgcagaatatgaagaagatggcaaatttgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaaaaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 4 - DNA, 21 bp, Artificial Sequence
  PCR Primer
tgctaaaggcacatatggcct

SEQ ID NO: 5 - DNA, 23 bp, Artificial Sequence
  PCR Primer
ctcaggttggactctccattgag

SEQ ID NO: 6 - DNA, 28 bp, Artificial Sequence
  PCR Probe
cttgtcagagggatcctaacactggccg

SEQ ID NO: 7 - DNA, 19 bp, Artificial Sequence
  PCR Primer
gaaggtgaaggtcggagtc

SEQ ID NO: 8 - DNA, 20 bp, Artificial Sequence
  PCR Primer
gaagatggtgatgggatttc

SEQ ID NO: 9 - DNA, 20 bp, Artificial Sequence
  PCR Probe
caagcttcccgttctcagcc

SEQ ID NO: 10 - DNA, 2354 bp, Mus musculus
gaattccaacttcctcacctctcacatacaattgaaatacctgcttttggcaaactgcatagcatccttaagatccaatctcctctctttatattagatgctaatgccaacatacagaatgtaacaacttcagggaacaaagcagagattgtggcttctgtcactgctaaaggagagtcccaatttgaagctctcaattttgattttcaagcacaagctcaattcctggagttaaatcctcatcctccagtcctgaaggaatccatgaacttctccagtaagcatgtgagaatggagcatgagggtgagatagtatttgatggaaaggccattgaggggaaatcagacacagtcgcaagtttacacacagagaaaaatgaagtagagtttaataatggtatgactgtcaaagtaaacaatcagctcacccttgacagtcacacaaagtacttccacaagttgagtgttcctaggctggacttctccagtaaggcttctcttaataatgaaatcaagacactattagaagctggacatgtggcattgacatcttcagggacagggtcatggaactgggcctgtcccaacttctcggatgaaggcatacattcgtcccaaattagctttactgtggatggtcccattgcttttgttggactatccaataacataaatggcaaacacttacgggtcatccaaaaactgacttatgaatctggcttcctcaactattctaagtttgaagttgagtcaaaagttgaatctcagcacgtgggctccagcattctaacagccaatggtcgggcactgctcaaggacgcaaaggcagaaatgactggtgagcacaatgccaacttaaatggaaaagttattggaactttgaaaaattctctcttcttttcagcacaaccatttgagattactgcatccacaaataatgaaggaaatttgaaagtgggttttccactaaagctgactgggaaaatagacttcctgaataactatgcattgtttctgagtccccgtgcccaacaagcaagctggcaagcgagtaccagattcaatcagtacaaatacaatcaaaacttttctgctataaacaatgaacacaacatagaagccagtataggaatgaatggagatgccaacctggatttcttaaacatacctttaacaattcctgaaattaacttgccttacacggagttcaaaactcccttactgaaggatttctccatatgggaagaaacaggcttgaaagaatttttgaagacaacaaagcaatcatttgatttgagtgtaaaggctcaatataaaaagaacagtgacaagcattccattgttgtccctctgggtatgttttatgaatttattctcaacaatgtcaattcgtgggacagaaaatttgagaaagtcagaaacaatgctttacattttcttaccacctcctataatgaagcaaaaattaaggttgataagtacaaaactgaaaattcccttaatcagccctctgggacctttcaaaatcatggctacactatcccagttgtcaacattgaagtatctccatttgctgtagagacactggcttccaggcatgtgatccccacagcaataagcaccccaagtgtcacaatccctggtcctaacatcatggtgccttcatacaagttagtgctgccacccctggagttgccagttttccatggtcctgggaatctattcaagtttttcctcccagatttcaagggattcaacactattgacaatatttatattccagccatgggcaactttacctatgacttttcttttaaatcaagtgtcatcacactgaataccaatgctggactttataaccaatcagatatcgttgcccatttcctttcttcctcttcatttgtcactgacgccctgcagtacaaattagagggaacatcacgtctgatgcgaaaaaggggattgaaactagccacagctgtctctctaactaacaaatttgtaaagggcagtcatgacagcaccattagtttaaccaagaaaaacatggaagcatcagtgagaacaactgccaacctccatgctcccatattctcaatgaacttcaagcaggaacttaatggaaataccaagtcaaaacccactgtttcatcatccattgaactaaactatgacttcaattcctcaaagctgcactctactgcaacaggaggcattgatcacaagttcagcttagaaagtctcacttcctacttttccattgagtcattcaccaaaggaaatatcaagagttccttcctttctcaggaatattcaggaagtgttgccaatgaagccaatgtatatctgaattc

SEQ ID NO: 11 - DNA, 19 bp, Artificial Sequence
  PCR Primer
cgtgggctccagcattcta

SEQ ID NO: 12 - DNA, 21 bp, Artificial Sequence
  PCR Primer
agtcatttctgcctttgcgtc

SEQ ID NO: 13 - DNA, 22 bp, Artificial Sequence
  PCR Probe
ccaatggtcgggcactgctcaa

SEQ ID NO: 14 - DNA, 20 bp, Artificial Sequence
  PCR Primer
ggcaaattcaacggcacagt

SEQ ID NO: 15 - DNA, 20 bp, Artificial Sequence
  PCR Primer
gggtctcgctcctggaagat

SEQ ID NO: 16 - DNA, 27 bp, Artificial Sequence
  PCR Probe
aaggccgagaatgggaagcttgtcatc

SEQ ID NO: 17 - DNA, 20 bp, Artificial Sequence
  Antisense Oligonucleotide
ccgcaggtcccggtgggaat

SEQ ID NO: 18 - DNA, 20 bp, Artificial Sequence
  Antisense Oligonucleotide
accgagaagggcactcagcc

SEQ ID NO: 19 - DNA, 20 bp, Artificial Sequence
  Antisense Oligonucleotide
gcctcggcctcgcggccctg

SEQ ID NO: 20 - DNA, 20 bp, Artificial Sequence
  Antisense Oligonucleotide
tccatcgccagctgcggtgg

SEQ ID NO: 21 - DNA, 20 bp, Artificial Sequence
  Antisense Oligonucleotide
cagcgccagcagcgccagca

SEQ ID NO: 22 - DNA, 20 bp, Artificial Sequence
  Antisense Oligonucleotide
gcccgccagcagcagcagca

SEQ ID NO: 23 - DNA, 20 bp, Artificial Sequence
  Antisense Oligonucleotide
cttgaatcagcagtcccagg

SEQ ID NO: 24 - DNA, 20 bp, Artificial Sequence
  Antisense Oligonucleotide
cttcagcaaggctttgccct

SEQ ID NO: 25 - DNA, 20 bp, Artificial Sequence
  Antisense Oligonucleotide
tttctgttgccacattgccc

SEQ ID NO: 26 - DNA, 20 bp, Artificial Sequence
  Antisense Oligonucleotide
ggaagaggtgttgctccttg

SEQ ID NO: 27 - DNA, 20 bp, Artificial Sequence
  Antisense Oligonucleotide
tgtgctaccatcccatactt

SEQ ID NO: 28 - DNA, 20 bp, Artificial Sequence
  Antisense Oligonucleotide
tcaaatgcgaggcccatctt

SEQ ID NO: 29 - DNA, 20 bp, Artificial Sequence
  Antisense Oligonucleotide
ggacacctcaatcagctgtg

SEQ ID NO: 30 - DNA, 20 bp, Artificial Sequence
  Antisense Oligonucleotide
tcagggccaccaggtaggtg

SEQ ID NO: 31 - DNA, 20 bp, Artificial Sequence
  Antisense Oligonucleotide
gtaatcttcatccccagtgc

SEQ ID NO: 32 - DNA, 20 bp, Artificial Sequence
  Antisense Oligonucleotide
tgctccatggtttggcccat

SEQ ID NO: 33 - DNA, 20 bp, Artificial Sequence
  Antisense Oligonucleotide
gcagccagtcgcttatctcc

SEQ ID NO: 34 - DNA, 20 bp, Artificial Sequence
  Antisense Oligonucleotide
gtatagccaaagtggtccac

SEQ ID NO: 35 - DNA, 20 bp, Artificial Sequence
  Antisense Oligonucleotide
cccaggagctggaggtcatg

SEQ ID NO: 36 - DNA, 20 bp, Artificial Sequence
  Antisense Oligonucleotide
ttgagcccttcctgatgacc

SEQ ID NO: 37 - DNA, 20 bp, Artificial Sequence
  Antisense Oligonucleotide
atctggaccccactcctagc

SEQ ID NO: 38 - DNA, 20 bp, Artificial Sequence
  Antisense Oligonucleotide
cagacccgactcgtggaaga

SEQ ID NO: 39 - DNA, 20 bp, Artificial Sequence
  Antisense Oligonucleotide
gccctcagtagattcatcat

SEQ ID NO: 40 - DNA, 20 bp, Artificial Sequence
  Antisense Oligonucleotide
gccatgccaccctcttggaa

SEQ ID NO: 41 - DNA, 20 bp, Artificial Sequence
  Antisense Oligonucleotide
aacccacgtgccggaaagtc

SEQ ID NO: 42 - DNA, 20 bp, Artificial Sequence
  Antisense Oligonucleotide
actcccagatgccttctgaa

SEQ ID NO: 43 - DNA, 20 bp, Artificial Sequence
  Antisense Oligonucleotide
atgtggtaacgagcccgaag

SEQ ID NO: 44 - DNA, 20 bp, Artificial Sequence
  Antisense Oligonucleotide
ggcgtagagacccatcacat

SEQ ID NO: 45 - DNA, 20 bp, Artificial Sequence
  Antisense Oligonucleotide
gtgttaggatccctctgaca

SEQ ID NO: 46 - DNA, 20 bp, Artificial Sequence
  Antisense Oligonucleotide
cccagtgatagctctgtgag

SEQ ID NO: 47 - DNA, 20 bp, Artificial Sequence
  Antisense Oligonucleotide
atttcagcatatgagcccat

SEQ ID NO: 48 - DNA, 20 bp, Artificial Sequence
  Antisense Oligonucleotide
ccctgaaccttagcaacagt

SEQ ID NO: 49 - DNA, 20 bp, Artificial Sequence
  Antisense Oligonucleotide
gctgaagccagcccagcgat

SEQ ID NO: 50 - DNA, 20 bp, Artificial Sequence
  Antisense Oligonucleotide
acagctgcccagtatgttct

SEQ ID NO: 51 - DNA, 20 bp, Artificial Sequence
  Antisense Oligonucleotide
cccaataagatttataacaa

SEQ ID NO: 52 - DNA, 20 bp, Artificial Sequence
  Antisense Oligonucleotide
tggcctaccagagacaggta

SEQ ID NO: 53 - DNA, 20 bp, Artificial Sequence
  Antisense Oligonucleotide
tcatacgtttagcccaatct

SEQ ID NO: 54 - DNA, 20 bp, Artificial Sequence
  Antisense Oligonucleotide
gcatggtcccaaggatggtc

SEQ ID NO: 55 - DNA, 20 bp, Artificial Sequence
  Antisense Oligonucleotide
agtgatggaagctgcgatac

SEQ ID NO: 56 - DNA, 20 bp, Artificial Sequence
  Antisense Oligonucleotide
atgagcatcatgcctcccag

SEQ ID NO: 57 - DNA, 20 bp, Artificial Sequence
  Antisense Oligonucleotide
gaacacatagccgaatgccg

SEQ ID NO: 58 - DNA, 20 bp, Artificial Sequence
  Antisense Oligonucleotide
gtggtgccctctaatttgta

SEQ ID NO: 59 - DNA, 20 bp, Artificial Sequence
  Antisense Oligonucleotide
cccgagaaagaaccgaaccc

SEQ ID NO: 60 - DNA, 20 bp, Artificial Sequence
  Antisense Oligonucleotide
tgccctgcagcttcactgaa

SEQ ID NO: 61 - DNA, 20 bp, Artificial Sequence
  Antisense Oligonucleotide
gaaatcccataagctcttgt

SEQ ID NO: 62 - DNA, 20 bp, Artificial Sequence
  Antisense Oligonucleotide
agaagctgcctcttcttccc

SEQ ID NO: 63 - DNA, 20 bp, Artificial Sequence
  Antisense Oligonucleotide
tcagggtgagccctgtgtgt

SEQ ID NO: 64 - DNA, 20 bp, Artificial Sequence
  Antisense Oligonucleotide
ctaatggccccttgataaac

SEQ ID NO: 65 - DNA, 20 bp, Artificial Sequence
  Antisense Oligonucleotide
acgttatccttgagtccctg

SEQ ID NO: 66 - DNA, 20 bp, Artificial Sequence
  Antisense Oligonucleotide
tatatcccaggtttccccgg

SEQ ID NO: 67 - DNA, 20 bp, Artificial Sequence
  Antisense Oligonucleotide
acctgggacagtaccgtccc

SEQ ID NO: 68 - DNA, 20 bp, Artificial Sequence
  Antisense Oligonucleotide
ctgcctactgcaaggctggc

SEQ ID NO: 69 - DNA, 20 bp, Artificial Sequence
  Antisense Oligonucleotide
agagaccttccgagccctgg

SEQ ID NO: 70 - DNA, 20 bp, Artificial Sequence
  Antisense Oligonucleotide
atgatacacaataaagactc

SEQ ID NO: 71 - DNA, 20 bp, Artificial Sequence
  Antisense Oligonucleotide
attgtatgtgagaggtgagg

SEQ ID NO: 72 - DNA, 20 bp, Artificial Sequence
  Antisense Oligonucleotide
gaggagattggatcttaagg

SEQ ID NO: 73 - DNA, 20 bp, Artificial Sequence
  Antisense Oligonucleotide
cttcaaattgggactctcct

SEQ ID NO: 74 - DNA, 20 bp, Artificial Sequence
  Antisense Oligonucleotide
tccaggaattgagcttgtgc

SEQ ID NO: 75 - DNA, 20 bp, Artificial Sequence
  Antisense Oligonucleotide
ttcaggactggaggatgagg

SEQ ID NO: 76 - DNA, 20 bp, Artificial Sequence
  Antisense Oligonucleotide
tctcaccctcatgctccatt

SEQ ID NO: 77 - DNA, 20 bp, Artificial Sequence
  Antisense Oligonucleotide
tgactgtcaagggtgagctg

SEQ ID NO: 78 - DNA, 20 bp, Artificial Sequence
  Antisense Oligonucleotide
gtccagcctaggaacactca

SEQ ID NO: 79 - DNA, 20 bp, Artificial Sequence
  Antisense Oligonucleotide
atgtcaatgccacatgtcca

SEQ ID NO: 80 - DNA, 20 bp, Artificial Sequence
  Antisense Oligonucleotide
ttcatccgagaagttgggac

SEQ ID NO: 81 - DNA, 20 bp, Artificial Sequence
  Antisense Oligonucleotide
atttgggacgaatgtatgcc

SEQ ID NO: 82 - DNA, 20 bp, Artificial Sequence
  Antisense Oligonucleotide
agttgaggaagccagattca

SEQ ID NO: 83 - DNA, 20 bp, Artificial Sequence
  Antisense Oligonucleotide
ttcccagtcagctttagtgg

SEQ ID NO: 84 - DNA, 20 bp, Artificial Sequence
  Antisense Oligonucleotide
agcttgcttgttgggcacgg

SEQ ID NO: 85 - DNA, 20 bp, Artificial Sequence
  Antisense Oligonucleotide
cctatactggcttctatgtt

SEQ ID NO: 86 - DNA, 20 bp, Artificial Sequence
  Antisense Oligonucleotide
tgaactccgtgtaaggcaag

SEQ ID NO: 87 - DNA, 20 bp, Artificial Sequence
  Antisense Oligonucleotide
gagaaatccttcagtaaggg

SEQ ID NO: 88 - DNA, 20 bp, Artificial Sequence
  Antisense Oligonucleotide
caatggaatgcttgtcactg

SEQ ID NO: 89 - DNA, 20 bp, Artificial Sequence
  Antisense Oligonucleotide
gcttcattataggaggtggt

SEQ ID NO: 90 - DNA, 20 bp, Artificial Sequence
  Antisense Oligonucleotide
acaactgggatagtgtagcc

SEQ ID NO: 91 - DNA, 20 bp, Artificial Sequence
  Antisense Oligonucleotide
gttaggaccagggattgtga

SEQ ID NO: 92 - DNA, 20 bp, Artificial Sequence
  Antisense Oligonucleotide
accatggaaaactggcaact

SEQ ID NO: 93 - DNA, 20 bp, Artificial Sequence
  Antisense Oligonucleotide
tgggaggaaaaacttgaata

SEQ ID NO: 94 - DNA, 20 bp, Artificial Sequence
  Antisense Oligonucleotide
tgggcaacgatatctgattg

SEQ ID NO: 95 - DNA, 20 bp, Artificial Sequence
  Antisense Oligonucleotide
ctgcagggcgtcagtgacaa

SEQ ID NO: 96 - DNA, 20 bp, Artificial Sequence
  Antisense Oligonucleotide
gcatcagacgtgatgttccc

SEQ ID NO: 97 - DNA, 20 bp, Artificial Sequence
  Antisense Oligonucleotide
cttggttaaactaatggtgc

SEQ ID NO: 98 - DNA, 20 bp, Artificial Sequence
  Antisense Oligonucleotide
atgggagcatggaggttggc

SEQ ID NO: 99 - DNA, 20 bp, Artificial Sequence
  Antisense Oligonucleotide
aatggatgatgaaacagtgg

SEQ ID NO: 100 - DNA, 20 bp, Artificial Sequence
  Antisense Oligonucleotide
atcaatgcctcctgttgcag

SEQ ID NO: 101 - DNA, 20 bp, Artificial Sequence
  Antisense Oligonucleotide
ggaagtgagactttctaagc

SEQ ID NO: 102 - DNA, 20 bp, Artificial Sequence
  Antisense Oligonucleotide
aggaaggaactcttgatatt

SEQ ID NO: 103 - DNA, 20 bp, Artificial Sequence
  Antisense Oligonucleotide
attggcttcattggcaacac

SEQ ID NO: 104 - DNA, 20 bp, Artificial Sequence
  Antisense Oligonucleotide
aggtgaggaagttggaattc

SEQ ID NO: 105 - DNA, 20 bp, Artificial Sequence
  Antisense Oligonucleotide
ttgttccctgaagttgttac

SEQ ID NO: 106 - DNA, 20 bp, Artificial Sequence
  Antisense Oligonucleotide
gttcatggattccttcagga

SEQ ID NO: 107 - DNA, 20 bp, Artificial Sequence
  Antisense Oligonucleotide
atgctccattctcacatgct

SEQ ID NO: 108 - DNA, 20 bp, Artificial Sequence
  Antisense Oligonucleotide
tgcgactgtgtctgatttcc

SEQ ID NO: 109 - DNA, 20 bp, Artificial Sequence
  Antisense Oligonucleotide
gtccctgaagatgtcaatgc

SEQ ID NO: 110 - DNA, 20 bp, Artificial Sequence
  Antisense Oligonucleotide
aggcccagttccatgaccct

SEQ ID NO: 111 - DNA, 20 bp, Artificial Sequence
  Antisense Oligonucleotide
ggagcccacgtgctgagatt

SEQ ID NO: 112 - DNA, 20 bp, Artificial Sequence
  Antisense Oligonucleotide
cgtccttgagcagtgcccga

SEQ ID NO: 113 - DNA, 20 bp, Artificial Sequence
  Antisense Oligonucleotide
cccatatggagaaatccttc

SEQ ID NO: 114 - DNA, 20 bp, Artificial Sequence
  Antisense Oligonucleotide
catgcctggaagccagtgtc

SEQ ID NO: 115 - DNA, 20 bp, Artificial Sequence
  Antisense Oligonucleotide
gtgttgaatcccttgaaatc

SEQ ID NO: 116 - DNA, 20 bp, Artificial Sequence
  Antisense Oligonucleotide
ggtaaagttgcccatggctg

SEQ ID NO: 117 - DNA, 20 bp, Artificial Sequence
  Antisense Oligonucleotide
gttataaagtccagcattgg

SEQ ID NO: 118 - DNA, 20 bp, Artificial Sequence
  Antisense Oligonucleotide
catcagacgtgatgttccct

SEQ ID NO: 119 - DNA, 20 bp, Artificial Sequence
  Antisense Oligonucleotide
tggctagtttcaatcccctt

SEQ ID NO: 120 - DNA, 20 bp, Artificial Sequence
  Antisense Oligonucleotide
ctgtcatgactgccctttac

SEQ ID NO: 121 - DNA, 20 bp, Artificial Sequence
  Antisense Oligonucleotide
gcttgaagttcattgagaat

SEQ ID NO: 122 - DNA, 20 bp, Artificial Sequence
  Antisense Oligonucleotide
ttcctgagaaaggaaggaac

SEQ ID NO: 123 - DNA, 20 bp, Artificial Sequence
  Antisense Oligonucleotide
tcagatatacattggcttca

SEQ ID NO: 124 - DNA, 20 bp, Artificial Sequence
  Antisense Oligonucleotide
ttcctcttcggccctggcgc

SEQ ID NO: 125 - DNA, 20 bp, Artificial Sequence
  Antisense Oligonucleotide
ctccactggaactctcagcc

SEQ ID NO: 126 - DNA, 20 bp, Artificial Sequence
  Antisense Oligonucleotide
cctccagctcaaccttgcag

SEQ ID NO: 127 - DNA, 20 bp, Artificial Sequence
  Antisense Oligonucleotide
gggttgaagccatacacctc

SEQ ID NO: 128 - DNA, 20 bp, Artificial Sequence
  Antisense Oligonucleotide
ccagcttgagctcatacctg

SEQ ID NO: 129 - DNA, 20 bp, Artificial Sequence
  Antisense Oligonucleotide
ccctcttgatgttcaggatg

SEQ ID NO: 130 - DNA, 20 bp, Artificial Sequence
  Antisense Oligonucleotide
gagcagtttccatacacggt

SEQ ID NO: 131 - DNA, 20 bp, Artificial Sequence
  Antisense Oligonucleotide
cccttcctcgtcttgacggt

SEQ ID NO: 132 - DNA, 20 bp, Artificial Sequence
  Antisense Oligonucleotide
ttgaagcgatcacactgccc

SEQ ID NO: 133 - DNA, 20 bp, Artificial Sequence
  Antisense Oligonucleotide
gcctttgatgagagcaagtg

SEQ ID NO: 134 - DNA, 20 bp, Artificial Sequence
  Antisense Oligonucleotide
tcctcttagcgtccagtgtg

SEQ ID NO: 135 - DNA, 20 bp, Artificial Sequence
  Antisense Oligonucleotide
cctctcagctcagtaaccag

SEQ ID NO: 136 - DNA, 20 bp, Artificial Sequence
  Antisense Oligonucleotide
gcactgaggctgtccacact

SEQ ID NO: 137 - DNA, 20 bp, Artificial Sequence
  Antisense Oligonucleotide
cgctgatccctcgccatgtt

SEQ ID NO: 138 - DNA, 20 bp, Artificial Sequence
  Antisense Oligonucleotide
gttgaccgcgtggctcagcg

SEQ ID NO: 139 - DNA, 20 bp, Artificial Sequence
  Antisense Oligonucleotide
gcagctcctgggtccctgta

SEQ ID NO: 140 - DNA, 20 bp, Artificial Sequence
  Antisense Oligonucleotide
cccatggtagaatttggaca

SEQ ID NO: 141 - DNA, 20 bp, Artificial Sequence
  Antisense Oligonucleotide
aatctcgatgaggtcagctg

SEQ ID NO: 142 - DNA, 20 bp, Artificial Sequence
  Antisense Oligonucleotide
gacaccatcaggaacttgac

SEQ ID NO: 143 - DNA, 20 bp, Artificial Sequence
  Antisense Oligonucleotide
gctcctctcccaagatgcgg

SEQ ID NO: 144 - DNA, 20 bp, Artificial Sequence
  Antisense Oligonucleotide
ggcacccatcagaagcagct

SEQ ID NO: 145 - DNA, 20 bp, Artificial Sequence
  Antisense Oligonucleotide
agtccggaatgatgatgccc

SEQ ID NO: 146 - DNA, 20 bp, Artificial Sequence
  Antisense Oligonucleotide
ctgagcagcttgactggtct

SEQ ID NO: 147 - DNA, 20 bp, Artificial Sequence
  Antisense Oligonucleotide
cccggtcagcggatagtagg

SEQ ID NO: 148 - DNA, 20 bp, Artificial Sequence
  Antisense Oligonucleotide
tgtcacaacttaggtggccc

SEQ ID NO: 149 - DNA, 20 bp, Artificial Sequence
  Antisense Oligonucleotide
gtctggcaatcccatgttct

SEQ ID NO: 150 - DNA, 20 bp, Artificial Sequence
  Antisense Oligonucleotide
cccacagacttgaagtggag

SEQ ID NO: 151 - DNA, 20 bp, Artificial Sequence
  Antisense Oligonucleotide
gaactgcccatcaatcttga

SEQ ID NO: 152 - DNA, 20 bp, Artificial Sequence
  Antisense Oligonucleotide
cccagagaggccaagctctg

SEQ ID NO: 153 - DNA, 20 bp, Artificial Sequence
  Antisense Oligonucleotide
tgtgttccctgaagcggcca

SEQ ID NO: 154 - DNA, 20 bp, Artificial Sequence
  Antisense Oligonucleotide
acccagaatcatggcctgat

SEQ ID NO: 155 - DNA, 20 bp, Artificial Sequence
  Antisense Oligonucleotide
ggtgcctgtctgctcagctg

SEQ ID NO: 156 - DNA, 20 bp, Artificial Sequence
  Antisense Oligonucleotide
atgtgaaacttgtctctccc

SEQ ID NO: 157 - DNA, 20 bp, Artificial Sequence
  Antisense Oligonucleotide
tatgtctgcagttgagatag

SEQ ID NO: 158 - DNA, 20 bp, Artificial Sequence
  Antisense Oligonucleotide
ttgaatccaggatgcagtac

SEQ ID NO: 159 - DNA, 20 bp, Artificial Sequence
  Antisense Oligonucleotide
gagtctctgagtcacctcac

SEQ ID NO: 160 - DNA, 20 bp, Artificial Sequence
  Antisense Oligonucleotide
gatagaatattgctctgcaa

SEQ ID NO: 161 - DNA, 20 bp, Artificial Sequence
  Antisense Oligonucleotide
cccttgctctaccaatgctt

SEQ ID NO: 162 - DNA, 20 bp, Artificial Sequence
  Antisense Oligonucleotide
tccattccctatgtcagcat

SEQ ID NO: 163 - DNA, 20 bp, Artificial Sequence
  Antisense Oligonucleotide
gactccttcagagccagcgg

SEQ ID NO: 164 - DNA, 20 bp, Artificial Sequence
  Antisense Oligonucleotide
cccatgctccgttctcaggt

SEQ ID NO: 165 - DNA, 20 bp, Artificial Sequence
  Antisense Oligonucleotide
cgcaggtcagcctgactaga

SEQ ID NO: 166 - DNA, 20 bp, Artificial Sequence
  Antisense Oligonucleotide
cagttagaacactgtggccc

SEQ ID NO: 167 - DNA, 20 bp, Artificial Sequence
  Antisense Oligonucleotide
cagtgtgatgacacttgatt

SEQ ID NO: 168 - DNA, 20 bp, Artificial Sequence
  Antisense Oligonucleotide
ctgtggctaacttcaatccc

SEQ ID NO: 169 - DNA, 20 bp, Artificial Sequence
  Antisense Oligonucleotide
cagtactgttatgactaccc

SEQ ID NO: 170 - DNA, 20 bp, Artificial Sequence
  Antisense Oligonucleotide
cactgaagaccgtgtgctct

SEQ ID NO: 171 - DNA, 20 bp, Artificial Sequence
  Antisense Oligonucleotide
tcgtactgtgctcccagagg

SEQ ID NO: 172 - DNA, 20 bp, Artificial Sequence
  Antisense Oligonucleotide
aagaggccctctagctgtaa

SEQ ID NO: 173 - DNA, 20 bp, Artificial Sequence
  Antisense Oligonucleotide
aagacccagaatgaatccgg

SEQ ID NO: 174 - DNA, 20 bp, Artificial Sequence
  Antisense Oligonucleotide
gtctacctcaaagcgtgcag

SEQ ID NO: 175 - DNA, 20 bp, Artificial Sequence
  Antisense Oligonucleotide
tagaggctaacgtaccatct

SEQ ID NO: 176 - DNA, 20 bp, Artificial Sequence
  Antisense Oligonucleotide
ccatatccatgcccacggtg

SEQ ID NO: 177 - DNA, 20 bp, Artificial Sequence
  Antisense Oligonucleotide
agtttcctcatcagattccc

SEQ ID NO: 178 - DNA, 20 bp, Artificial Sequence
  Antisense Oligonucleotide
cccagtggtacttgttgaca

SEQ ID NO: 179 - DNA, 20 bp, Artificial Sequence
  Antisense Oligonucleotide
cccagtggtgccactggctg

SEQ ID NO: 180 - DNA, 20 bp, Artificial Sequence
  Antisense Oligonucleotide
gtcaacagttcctggtacag

SEQ ID NO: 181 - DNA, 20 bp, Artificial Sequence
  Antisense Oligonucleotide
ccctagtgtatatcccaggt

SEQ ID NO: 182 - DNA, 20 bp, Artificial Sequence
  Antisense Oligonucleotide
ctgaagattacgtagcacct

SEQ ID NO: 183 - DNA, 20 bp, Artificial Sequence
  Antisense Oligonucleotide
gtccagccaactatacttgg

SEQ ID NO: 184 - DNA, 20 bp, Artificial Sequence
  Antisense Oligonucleotide
cctggagcaagcttcatgta

SEQ ID NO: 185 - DNA, 20 bp, Artificial Sequence
  Antisense Oligonucleotide
tggacagaccaggctgacat

SEQ ID NO: 186 - DNA, 20 bp, Artificial Sequence
  Antisense Oligonucleotide
atgtgtacttccggaggtgc

SEQ ID NO: 187 - DNA, 20 bp, Artificial Sequence
  Antisense Oligonucleotide
tcttcaggatgaagctgcag

SEQ ID NO: 188 - DNA, 20 bp, Artificial Sequence
  Antisense Oligonucleotide
tcagcaaggctttgccctca

SEQ ID NO: 189 - DNA, 20 bp, Artificial Sequence
  Antisense Oligonucleotide
ctgcttcccttctggaatgg

SEQ ID NO: 190 - DNA, 20 bp, Artificial Sequence
  Antisense Oligonucleotide
tgccacattgcccttcctcg

SEQ ID NO: 191 - DNA, 20 bp, Artificial Sequence
  Antisense Oligonucleotide
gctgatcagagttgacaagg

SEQ ID NO: 192 - DNA, 20 bp, Artificial Sequence
  Antisense Oligonucleotide
tactgacaggactggctgct

SEQ ID NO: 193 - DNA, 20 bp, Artificial Sequence
  Antisense Oligonucleotide
gatggcttctgccacatgct

SEQ ID NO: 194 - DNA, 20 bp, Artificial Sequence
  Antisense Oligonucleotide
gatgtggatttggtgctctc

SEQ ID NO: 195 - DNA, 20 bp, Artificial Sequence
  Antisense Oligonucleotide
tgactgcttcatcactgagg

SEQ ID NO: 196 - DNA, 20 bp, Artificial Sequence
  Antisense Oligonucleotide
ggtaggtgaccacatctatc

SEQ ID NO: 197 - DNA, 20 bp, Artificial Sequence
  Antisense Oligonucleotide
tcgcagctgctgtgctgagg

SEQ ID NO: 198 - DNA, 20 bp, Artificial Sequence
  Antisense Oligonucleotide
ttccaatgacccgcagaatc

SEQ ID NO: 199 - DNA, 20 bp, Artificial Sequence
  Antisense Oligonucleotide
gatcatcagtgatggctttg

SEQ ID NO: 200 - DNA, 20 bp, Artificial Sequence
  Antisense Oligonucleotide
agcctggatggcagctttct

SEQ ID NO: 201 - DNA, 20 bp, Artificial Sequence
  Antisense Oligonucleotide
gtctgaagaagaacctcctg

SEQ ID NO: 202 - DNA, 20 bp, Artificial Sequence
  Antisense Oligonucleotide
tatctgcctgtgaaggactc

SEQ ID NO: 203 - DNA, 20 bp, Artificial Sequence
  Antisense Oligonucleotide
ctgagttcaagatattggca

SEQ ID NO: 204 - DNA, 20 bp, Artificial Sequence
  Antisense Oligonucleotide
cttccaagccaatctcgatg

SEQ ID NO: 205 - DNA, 20 bp, Artificial Sequence
  Antisense Oligonucleotide
tgcaactgtaatccagctcc

SEQ ID NO: 206 - DNA, 20 bp, Artificial Sequence
  Antisense Oligonucleotide
ccagttcagcctgcatgttg

SEQ ID NO: 207 - DNA, 20 bp, Artificial Sequence
  Antisense Oligonucleotide
gtagagaccaaatgtaatgt

SEQ ID NO: 208 - DNA, 20 bp, Artificial Sequence
  Antisense Oligonucleotide
cgttggagtaagcgcctgag

SEQ ID NO: 209 - DNA, 20 bp, Artificial Sequence
  Antisense Oligonucleotide
cagctctaatctggtgtccc

SEQ ID NO: 210 - DNA, 20 bp, Artificial Sequence
  Antisense Oligonucleotide
ctgtcctctctctggagctc

SEQ ID NO: 211 - DNA, 20 bp, Artificial Sequence
  Antisense Oligonucleotide
caaggtcatactctgccgat

SEQ ID NO: 212 - DNA, 20 bp, Artificial Sequence
  Antisense Oligonucleotide
gtatggaaataacacccttg

SEQ ID NO: 213 - DNA, 20 bp, Artificial Sequence
  Antisense Oligonucleotide
taagctgtagcagatgagtc

SEQ ID NO: 214 - DNA, 20 bp, Artificial Sequence
  Antisense Oligonucleotide
tagatctctggaggatttgc

SEQ ID NO: 215 - DNA, 20 bp, Artificial Sequence
  Antisense Oligonucleotide
gtctagaacacccaggagag

SEQ ID NO: 216 - DNA, 20 bp, Artificial Sequence
  Antisense Oligonucleotide
accacagagtcagccttcat

SEQ ID NO: 217 - DNA, 20 bp, Artificial Sequence
  Antisense Oligonucleotide
aagcagacatctgtggtccc

SEQ ID NO: 218 - DNA, 20 bp, Artificial Sequence
  Antisense Oligonucleotide
ctctccattgagccggccag

SEQ ID NO: 219 - DNA, 20 bp, Artificial Sequence
  Antisense Oligonucleotide
cctgatattcagaacgcagc

SEQ ID NO: 220 - DNA, 20 bp, Artificial Sequence
  Antisense Oligonucleotide
cagtgcctaagatgtcagca

SEQ ID NO: 221 - DNA, 20 bp, Artificial Sequence
  Antisense Oligonucleotide
agcaccaggagactacactt

SEQ ID NO: 222 - DNA, 20 bp, Artificial Sequence
  Antisense Oligonucleotide
cccatccagactgaattttg

SEQ ID NO: 223 - DNA, 20 bp, Artificial Sequence
  Antisense Oligonucleotide
ggttctagccgtagtttccc

SEQ ID NO: 224 - DNA, 20 bp, Artificial Sequence
  Antisense Oligonucleotide
aggttaccagccacatgcag

SEQ ID NO: 225 - DNA, 20 bp, Artificial Sequence
  Antisense Oligonucleotide
atgtgcatcgatggtcatgg

SEQ ID NO: 226 - DNA, 20 bp, Artificial Sequence
  Antisense Oligonucleotide
ccagagagcgagtttcccat

SEQ ID NO: 227 - DNA, 20 bp, Artificial Sequence
  Antisense Oligonucleotide
ctagacacgagatgatgact

SEQ ID NO: 228 - DNA, 20 bp, Artificial Sequence
  Antisense Oligonucleotide
tccaagtcctggctgtattc

SEQ ID NO: 229 - DNA, 20 bp, Artificial Sequence
  Antisense Oligonucleotide
cgtccagtaagctccacgcc

SEQ ID NO: 230 - DNA, 20 bp, Artificial Sequence
  Antisense Oligonucleotide
tcaacggcatctctcatctc

SEQ ID NO: 231 - DNA, 20 bp, Artificial Sequence
  Antisense Oligonucleotide
tgatagtgctcatcaagact

SEQ ID NO: 232 - DNA, 20 bp, Artificial Sequence
  Antisense Oligonucleotide
gattctgatttggtacttag

SEQ ID NO: 233 - DNA, 20 bp, Artificial Sequence
  Antisense Oligonucleotide
ctctcgattaactcatggac

SEQ ID NO: 234 - DNA, 20 bp, Artificial Sequence
  Antisense Oligonucleotide
atacactgcaactgtggcct

SEQ ID NO: 235 - DNA, 20 bp, Artificial Sequence
  Antisense Oligonucleotide
gcaagagtccaccaatcaga

SEQ ID NO: 236 - DNA, 20 bp, Artificial Sequence
  Antisense Oligonucleotide
agagcctgaagactgacttc

SEQ ID NO: 237 - DNA, 20 bp, Artificial Sequence
  Antisense Oligonucleotide
tccctcatctgagaatctgg

SEQ ID NO: 238 - DNA, 20 bp, Artificial Sequence
  Antisense Oligonucleotide
cagtgcatcaatgacagatg

SEQ ID NO: 239 - DNA, 20 bp, Artificial Sequence
  Antisense Oligonucleotide
ccgaacccttgacatctcct

SEQ ID NO: 240 - DNA, 20 bp, Artificial Sequence
  Antisense Oligonucleotide
gcctcactagcaatagttcc

SEQ ID NO: 241 - DNA, 20 bp, Artificial Sequence
  Antisense Oligonucleotide
gacatttgccatggagagag

SEQ ID NO: 242 - DNA, 20 bp, Artificial Sequence
  Antisense Oligonucleotide
ctgtctcctaccaatgctgg

SEQ ID NO: 243 - DNA, 20 bp, Artificial Sequence
  Antisense Oligonucleotide
tctgcactgaagtcacggtg

SEQ ID NO: 244 - DNA, 20 bp, Artificial Sequence
  Antisense Oligonucleotide
tcccggaccctcaactcagt

SEQ ID NO: 245 - DNA, 20 bp, Artificial Sequence
  Antisense Oligonucleotide
gcaggtccagttcatatgtg

SEQ ID NO: 246 - DNA, 20 bp, Artificial Sequence
  Antisense Oligonucleotide
gccatccttctgagttcaga

SEQ ID NO: 247 - DNA, 20 bp, Artificial Sequence
  Antisense Oligonucleotide
gcctcagtctgcttcgcacc

SEQ ID NO: 248 - DNA, 20 bp, Artificial Sequence
  Antisense Oligonucleotide
ccccgcaggtcccggtggga

SEQ ID NO: 249 - DNA, 20 bp, Artificial Sequence
  Antisense Oligonucleotide
cagccccgcaggtcccggtg

SEQ ID NO: 250 - DNA, 20 bp, Artificial Sequence
  Antisense Oligonucleotide
caaccgagaagggcactcag

SEQ ID NO: 251 - DNA, 20 bp, Artificial Sequence
  Antisense Oligonucleotide
cctcagcggcagcaaccgag

SEQ ID NO: 252 - DNA, 20 bp, Artificial Sequence
  Antisense Oligonucleotide
tcctcagcggcagcaaccga

SEQ ID NO: 253 - DNA, 20 bp, Artificial Sequence
  Antisense Oligonucleotide
ctcctcagcggcagcaaccg

SEQ ID NO: 254 - DNA, 20 bp, Artificial Sequence
  Antisense Oligonucleotide
ggctcctcagcggcagcaac

SEQ ID NO: 255 - DNA, 20 bp, Artificial Sequence
  Antisense Oligonucleotide
ggcgggctcctcagcggcag

SEQ ID NO: 256 - DNA, 20 bp, Artificial Sequence
  Antisense Oligonucleotide
ggtccatcgccagctgcggt

SEQ ID NO: 257 - DNA, 20 bp, Artificial Sequence
  Antisense Oligonucleotide
ggcgggtccatcgccagctg

SEQ ID NO: 258 - DNA, 20 bp, Artificial Sequence
  Antisense Oligonucleotide
tagaggatgatagtaagttc

SEQ ID NO: 259 - DNA, 20 bp, Artificial Sequence
  Antisense Oligonucleotide
aaatgaagatttcttttaaa

SEQ ID NO: 260 - DNA, 20 bp, Artificial Sequence
  Antisense Oligonucleotide
tatgtgaaagttcaattgga

SEQ ID NO: 261 - DNA, 20 bp, Artificial Sequence
  Antisense Oligonucleotide
atataggcagtttgaatttt

SEQ ID NO: 262 - DNA, 20 bp, Artificial Sequence
  Antisense Oligonucleotide
gctcactgtatggttttatc

SEQ ID NO: 263 - DNA, 20 bp, Artificial Sequence
  Antisense Oligonucleotide
ggctcactgtatggttttat

SEQ ID NO: 264 - DNA, 20 bp, Artificial Sequence
  Antisense Oligonucleotide
ggctggctcactgtatggtt

SEQ ID NO: 265 - DNA, 20 bp, Artificial Sequence
  Antisense Oligonucleotide
aggctggctcactgtatggt

SEQ ID NO: 266 - DNA, 20 bp, Artificial Sequence
  Antisense Oligonucleotide
aaggctggctcactgtatgg

SEQ ID NO: 267 - DNA, 20 bp, Artificial Sequence
  Antisense Oligonucleotide
ctactgcaaggctggctcac

SEQ ID NO: 268 - DNA, 20 bp, Artificial Sequence
  Antisense Oligonucleotide
actgcctactgcaaggctgg

SEQ ID NO: 269 - DNA, 20 bp, Artificial Sequence
  Antisense Oligonucleotide
tgcttatagtctactgccta

SEQ ID NO: 270 - DNA, 20 bp, Artificial Sequence
  Antisense Oligonucleotide
ttctgcttatagtctactgc

SEQ ID NO: 271 - DNA, 20 bp, Artificial Sequence
  Antisense Oligonucleotide
tttggtgcaggtccagttca

SEQ ID NO: 272 - DNA, 20 bp, Artificial Sequence
  Antisense Oligonucleotide
cagctttggtgcaggtccag

SEQ ID NO: 273 - DNA, 20 bp, Artificial Sequence
  Antisense Oligonucleotide
gccagctttggtgcaggtcc

SEQ ID NO: 274 - DNA, 20 bp, Artificial Sequence
  Antisense Oligonucleotide
tggtgccagctttggtgcag

SEQ ID NO: 275 - DNA, 20 bp, Artificial Sequence
  Antisense Oligonucleotide
gccctggtgccagctttggt

SEQ ID NO: 276 - DNA, 20 bp, Artificial Sequence
  Antisense Oligonucleotide
gagttcagagaccttccgag

SEQ ID NO: 277 - DNA, 20 bp, Artificial Sequence
  Antisense Oligonucleotide
aaatgccatccttctgagtt

SEQ ID NO: 278 - DNA, 20 bp, Artificial Sequence
  Antisense Oligonucleotide
aaaaatgccatccttctgag

SEQ ID NO: 279 - DNA, 20 bp, Artificial Sequence
  Antisense Oligonucleotide
aaaataactcagatcctgat

SEQ ID NO: 280 - DNA, 20 bp, Artificial Sequence
  Antisense Oligonucleotide
agcaaaataactcagatcct

SEQ ID NO: 281 - DNA, 20 bp, Artificial Sequence
  Antisense Oligonucleotide
agtttagcaaaataactcag

SEQ ID NO: 282 - DNA, 20 bp, Artificial Sequence
  Antisense Oligonucleotide
tcccccaagtttagcaaaat

SEQ ID NO: 283 - DNA, 20 bp, Artificial Sequence
  Antisense Oligonucleotide
ttcctcctcccccaagttta

SEQ ID NO: 284 - DNA, 20 bp, Artificial Sequence
  Antisense Oligonucleotide
agactccatttatttgttcc

SEQ ID NO: 285 - DNA, 20 bp, Artificial Sequence
  Antisense Oligonucleotide
cttctgcttgagttacaaac

SEQ ID NO: 286 - DNA, 20 bp, Artificial Sequence
  Antisense Oligonucleotide
accttctgcttgagttacaa

SEQ ID NO: 287 - DNA, 20 bp, Artificial Sequence
  Antisense Oligonucleotide
gcaccttctgcttgagttac

SEQ ID NO: 288 - DNA, 20 bp, Artificial Sequence
  Antisense Oligonucleotide
tcgcaccttctgcttgagtt

SEQ ID NO: 289 - DNA, 20 bp, Artificial Sequence
  Antisense Oligonucleotide
cttcgcaccttctgcttgag

SEQ ID NO: 290 - DNA, 20 bp, Artificial Sequence
  Antisense Oligonucleotide
tgcttcgcaccttctgcttg

SEQ ID NO: 291 - DNA, 20 bp, Artificial Sequence
  Antisense Oligonucleotide
tctgcttcgcaccttctgct

SEQ ID NO: 292 - DNA, 20 bp, Artificial Sequence
  Antisense Oligonucleotide
agtctgcttcgcaccttctg

SEQ ID NO: 293 - DNA, 20 bp, Artificial Sequence
  Antisense Oligonucleotide
tcagtctgcttcgcaccttc

SEQ ID NO: 294 - DNA, 20 bp, Artificial Sequence
  Antisense Oligonucleotide
cctcagtctgcttcgcacct

SEQ ID NO: 295 - DNA, 20 bp, Artificial Sequence
  Antisense Oligonucleotide
agcctcagtctgcttcgcac

SEQ ID NO: 296 - DNA, 20 bp, Artificial Sequence
  Antisense Oligonucleotide
gtagcctcagtctgcttcgc

SEQ ID NO: 297 - DNA, 20 bp, Artificial Sequence
  Antisense Oligonucleotide
tggtagcctcagtctgcttc

SEQ ID NO: 298 - DNA, 20 bp, Artificial Sequence
  Antisense Oligonucleotide
catggtagcctcagtctgct

SEQ ID NO: 299 - DNA, 20 bp, Artificial Sequence
  Antisense Oligonucleotide
gtcatggtagcctcagtctg

SEQ ID NO: 300 - DNA, 20 bp, Artificial Sequence
  Antisense Oligonucleotide
atgtcatggtagcctcagtc

SEQ ID NO: 301 - DNA, 20 bp, Artificial Sequence
  Antisense Oligonucleotide
gaatgtcatggtagcctcag

SEQ ID NO: 302 - DNA, 20 bp, Artificial Sequence
  Antisense Oligonucleotide
ttgaatgtcatggtagcctc

SEQ ID NO: 303 - DNA, 20 bp, Artificial Sequence
  Antisense Oligonucleotide
atttgaatgtcatggtagcc

SEQ ID NO: 304 - DNA, 20 bp, Artificial Sequence
  Antisense Oligonucleotide
atatttgaatgtcatggtag

SEQ ID NO: 305 - DNA, 20 bp, Artificial Sequence
  Antisense Oligonucleotide
cagccacatgcagcttcagg

SEQ ID NO: 306 - DNA, 20 bp, Artificial Sequence
  Antisense Oligonucleotide
accagccacatgcagcttca

SEQ ID NO: 307 - DNA, 20 bp, Artificial Sequence
  Antisense Oligonucleotide
ttaccagccacatgcagctt

SEQ ID NO: 308 - DNA, 20 bp, Artificial Sequence
  Antisense Oligonucleotide
ggttaccagccacatgcagc

SEQ ID NO: 309 - DNA, 20 bp, Artificial Sequence
  Antisense Oligonucleotide
taggttaccagccacatgca

SEQ ID NO: 310 - DNA, 20 bp, Artificial Sequence
  Antisense Oligonucleotide
tttaggttaccagccacatg

SEQ ID NO: 311 - DNA, 20 bp, Artificial Sequence
  Antisense Oligonucleotide
cttttaggttaccagccaca

SEQ ID NO: 312 - DNA, 20 bp, Artificial Sequence
  Antisense Oligonucleotide
tccttttaggttaccagcca

SEQ ID NO: 313 - DNA, 20 bp, Artificial Sequence
  Antisense Oligonucleotide
gctccttttaggttaccagc

SEQ ID NO: 314 - DNA, 20 bp, Artificial Sequence
  Antisense Oligonucleotide
aggctccttttaggttacca

SEQ ID NO: 315 - DNA, 20 bp, Artificial Sequence
  Antisense Oligonucleotide
gtaggctccttttaggttac

SEQ ID NO: 316 - DNA, 20 bp, Artificial Sequence
  Antisense Oligonucleotide
tggtaggctccttttaggtt

SEQ ID NO: 317 - DNA, 20 bp, Artificial Sequence
  Antisense Oligonucleotide
tttggtaggctccttttagg

SEQ ID NO: 318 - DNA, 13993 bp, H. sapiens
  CDS (1).. (13692)
atggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaggctccttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccgactcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtcatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggcgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagactcccacatcccagaaaacctcttcttaaaaagcgatggccgcgtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatgggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcaatgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatacccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctcgcccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcataggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaaaaaccacaaaaccggaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtcaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaccctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacacttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaaaaattccatatgaaagtcaagaagctgattgactcactcattgattttctgaacttccccagattccagtttccggggaaacctgggatatacactagggaggaactttgcactatgttcatgagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaatttttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcat

SEQ ID NO: 319 - DNA, 20 bp, Artificial Sequence
  Antisense Oligonucleotide
gcctcagtctgcttcgcgcc

SEQ ID NO: 320 - DNA, 20 bp, Artificial Sequence
  Antisense Oligonucleotide
gctcactgttcagcatctgg

SEQ ID NO: 321 - DNA, 20 bp, Artificial Sequence
  Antisense Oligonucleotide
tgagaatctgggcgaggccc

SEQ ID NO: 322 - DNA, 20 bp, Artificial Sequence
  Antisense Oligonucleotide
gtccttcatatttgccatct

SEQ ID NO: 323 - DNA, 20 bp, Artificial Sequence
  Antisense Oligonucleotide
cctccctcatgaacatagtg

SEQ ID NO: 324 - DNA, 20 bp, Artificial Sequence
  Antisense Oligonucleotide
gacgtcagaacctatgatgg

SEQ ID NO: 325 - DNA, 20 bp, Artificial Sequence
  Antisense Oligonucleotide
tgagtgagtcaatcagcttc

SEQ ID NO: 326 - DNA, 20 bp, Artificial Sequence
  Antisense Oligonucleotide
gccttctgcttgagttacaa

SEQ ID NO: 327 - DNA, 20 bp, Artificial Sequence
  Antisense Oligonucleotide
gcgccttctgcttgagttac

SEQ ID NO: 328 - DNA, 20 bp, Artificial Sequence
  Antisense Oligonucleotide
tcgcgccttctgcttgagtt

SEQ ID NO: 329 - DNA, 20 bp, Artificial Sequence
  Antisense Oligonucleotide
cttcgcgccttctgcttgag

SEQ ID NO: 330 - DNA, 20 bp, Artificial Sequence
  Antisense Oligonucleotide
agtctgcttcgcgccttctg

SEQ ID NO: 331 - DNA, 20 bp, Artificial Sequence
  Antisense Oligonucleotide
tcagtctgcttcgcgccttc

SEQ ID NO: 332 - DNA, 20 bp, Artificial Sequence
  Antisense Oligonucleotide
cctcagtctgcttcgcgcct

SEQ ID NO: 333 - DNA, 20 bp, Artificial Sequence
  Antisense Oligonucleotide
agcctcagtctgcttcgcgc

SEQ ID NO: 334 - DNA, 43445 bp, H. sapiens
accaagacagcgctcaggactggttctcctcgtggctcccaattcagtccaggagaagcagagattttgtccccatggtgggtcatctgaagaaggcacccctggtcagggcaggcttctcagaccctgaggcgctggccatggccccactgagacacaggaagggccgcgccagagcactgaagacgcttggggaagggaacccacctgggacccagcccctggtggctgcggctgcatcccaggtgggccccctccccgaggctcttcaaggctcaaagagaagccagtgtagaaaagcaaacaggtcaggcccgggaggcgccctttggaccttttgcaatcctggcgctcttgcagcctgggcttcctataaatggggtgcgggcgccggccgcgcattcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccggtgagtgcgcggccgctctgcgggcgcagagggagcgggagggagccggcggcacgaggttggccggggcagcctgggcctaggccagagggagggcagccacagggtccagggcgagtggggggattggaccagctggcggcccctgcaggctcaggatggggggcgcgggatggaggggctgaggagggggtctccggagcctgcctccctcctgaaaggtgaaacctgtgccggtggtccccctgtcgggccctagcacccgctgggaagacgtgggaagctcacagatttctttctcctgtcttacagaagaggaaatgctggaaaatgtcagcctggtctgtccaagtaaggcatctgcgcatggggcgtggaagggcgcccagccccgtgcactctcctacacccgggtccctgagggcctcccactctacagggctgagatggcatcgtggtgtgccttgctctgaccccaggaagcaagttccctgagcctctgcccacacccaagggatgccaactctcttctacctggccttctgttctgtcccaaaagttcagcctgggggcgggggagggaagggattgtctctccgctggcctgtgcacactttgaagaaacatcactgtcctgtttatcagtgactagtcattgattcgaagcatgtgagggtgaggaaatactgactttaacctttgtgaagaaatcgaacctccacccccttcctatttacctgacccctgggggttaaaggaactggcctccaagcgcgaccctgtgtgctggagccgcggggcggacttctgatggggcagcaccgccatctagtggccgtctgtcatcactgcagctggactcaggacccagatgttctttttcttcaattgttcagaaaattcctctcaactacagtggaaacctccagaaattcttttctaggagtttgttaagttagttacgcttaatgcttaatgaactttgccttaagtatttggtagtcttagagtcacggaattacggcgtgttcaagctaaaaaagcattagagatagtactatttgcgtaatgttgtcatctcttaatttgccagagggtctctcatgcagattttctgagccccattacttgacacttgtcactcccttccctgtgcctcagatgagatattcaagacatgccagccaatttaaacattagcctcagcaaaaacataatggagaagtcaaatctataaaggaaaattaagtataaagtcaattaaaaaataatttgagttgaattaccatttttaattctctatgccactgcccctctctgcccagaattggctgtccttgggagagctatttctgctatgtggctgacgtatttctccccacgttagaagatgcgacccgattcaagcacctccggaagtacacatacaactatgaggctgagagttccagtggagtccctgggactgctgattcaagaagtgccaccaggatcaactgcaaggtatggaggatgcaggcaggagggacctagagcccacagctttcccccagccctgttccagcgggcgcccaacacgcgaccttcccggagggtgtgtactgagcaaacgcagaacatcccagaactgttgtaatctgatcaaagcactgggactttgcctctgtttgtaagtcagccacattgctgagatgtggtctgcccccaccaaatttcgcaagtcagaagtattttcccgttaacttcccagatgcaataggaatccatgatctagattagcagcagtgtgggtctgtagatttcagcgtgagagaggcccagtaggtgagctatgggaggcaggcaactcggaatcgcactgtgaaatgcagtttttataatttaagtcaaacagaatctgttgctgaaaaatgaatggaaagaagaaaaaaatataaacatacagtttgttctaaaataaaactttgcttattattgagactggttgtactcatgttacatacatgtggagcagatctacaggctgctattggggtttgggtggggaagagaagtcaagctgagcagtcacctttttttagagagtaccgtagctcttgtatgtgctgtccaatatggtagacatgagccacattgggctatttaaatggaatgaaattaaaaattcatattcgttgtcacattagctgcatttcaactgctcaacagccaccctggctactggctcccatattgaacagcacacatgtacaacatttctataaagttatttgaatagtgctggataataagtaggaatccgttgaaactccagctatatgcaaagctctaaataggccctaatagatataaccagttttttgggtgacattaaggagacatttgctgtggaaacgaaggatggccctcttcctgctttctgtttttcttcttcactttcactcctagtctgcagcgcttctatttaaccacagctctttataattaaagtgagtaactttagaaccaataaaaggacatcctccttcccatgcctaggggcaaacttaagaaatgtgttacccgggagggggaaaacgtcagcaataggactaagtctaggttggtgcacagagaacccaggaggcatgttgataaggcatgtggtgttgaggcgcaggcagtggtgttcccagcaccattccctttggtgctctgattagagattaagccctgggcttcaggggccacctctcattcttgatagacaacctcaatgctctgctaccctgaattctcaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtaagtcatgttgtacatgagcacacgcatgtgtgtgtgtccgctgaggtatgaacttgtgtgtttgcaccaggcacggatgtgactgtaagtatttgtattccgtatccatcgtggatcagggaattactgagttttcacaatcatcaaaaagagagaagcattagttaaccttccctagttaggttcctttaattatcattttcatgtgtttctaaaaatctcatgctttaaacttcttgagattataaaactgagatgctttgtttaaacaagtgaattcttatttaaagaactagtcaagactagtgcttggtggtctttggtgtggggtcccagaggcactggctgctgtggccggcacatggcggggcagggtctgttcaccgcagggcagaggagcaccaaggcttcggtggctccccctcctaggctggcattcagccactgcacgctgatcggccactgcagctgcatctctgctgactggtcagggcccatgtcgcacccattgtaaatattttcaacatcacccctgcctcatcctcaatcacagtttgtagggtcctaggtgtgtatgaatacaggcaggatagagttgttaacttggtagcatcagaaaactctgtctgtattagtctgttttcatgctgctgataaagacatacctgagactgggcaatttacaaaagaaaggtttattggactcacagttccacgtggctggggaggtctcacaatcatggcggaaggtgagggacagcaagtcacatcttatgtagatggtggctggcaaagagagcttgtgcagagaaactcctgtttttagaaccatcagatctcccgacacccatctgcaatcacgagaacagcacgggaaagacctgcccccatgattcaatcacctccccccgggtccctcccacaacacgtgggaattatgagagctacgagacgaaatttgggtggggacgcagagccaaaccatatcaccatccttgcccatttttcagttttgctaaacattagattcagatgccagtcctttcttgccaaaataggctgtgaggcttctttctttcctatgctttattttctccaagacttaactgtatatgagggagaggggtatggtggcaggaggaaagagtggtttattttttggtccttggtcttctccaaatacagaagagactcctgttcttgaaaaggagggctttccatgtttgcatcttcatgactttaactgtcttttttaaaaattgacatacaataattatacatatttattgagaacatagtgatattttgatacatgtaatgtatggtgatcagatcagagtaattagcatacccatcatctcaaacatttatcatttcttcgtgttgggaactttctgagagagtgtaggctgtgggagataagtccgtcaccttttcctcctgatgtaaccagagtggctgcagccaggtcctcagaaactcagagagtacccagtgggaaatccctaagaccaaagtcagcatgggcttcagccatggcctgacaccatacaaaagaatgactgtccaacaagtgtatgaaaataagctccaattcactggtagtcaagaaatgcgaattaatgtaacaacaagatatttatctgcttttacccatcatactgcaaaactggaaaacagtgatagcacctgttgctggcaggccagtgaggaaaagtgtgctgtcctgagctgctggtggaaacgagagccatcaggcaatatctactgtaatttaaaatacttaataccctttgacacagatattttagtctttgggactctagcccatgaaaataaaagcagtaatgtgtgaagataggcacataaggatgtttgttttggtattgtttgtgtggtttaaaaaaaatccagaaagagagagggcaaatgccatcaaatggggcaatgtgtgaataaattatatttagccatggaatggaatgttctgcatgcagcttttaaaaaaatctgttagagctgtaccaagtgactcagaaggatttttgtgaagtataattaagtgagaaaaacaagataaaagtatgcataatacaatgccacttgtataaaacaaacaatggcaaaatctttgtatgactctgtttgcactcacccatgtttacagaggattgtatgagtgtgcagaaacaaatggaacaaccactcgggtgtccgtatggggaggatgggcaaagagactgatatgggtggagaacagagcagggctggatgagccaagcaaaaaaagttaaaacacagctggacctggtggctcatgcctgtagtcccagcactttgggaggccgaggagggagaatcacctgaggtcaggagtttgagaccagcctggccaacatggtgaaaactgtctctactaaaaatacaaaaattagctgggtgtgatggcacatgccagtagtcctagctactccggaggctgaggcaggagaatcacttgatcccaggaggtggaggttgcagtgagctgaggttgcgccattgcactccagcccgggcgaccgagcgagactccatttcaaaaaaagaaaaagaaaaaagaaaaaaagaaaaaaaaagaatcaccaaaacttatgtatatgtgcatacttttttgaaaatgtatgtctatgtgtagctatattctatatttacaaataaatgatgtcagaagaacaattggttaaaaaaatatgagaaaagaaacttcagtgccacccagcttacttccagcaagttgtaatggagaaggacatttccgtgaccatcctctctctgggacaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggtgaggatttagaaagctgatagcagtggcccttgaaactcatcttcatgtgttagagaccagtcctaccatatacaaagcagatcactgagtcagctccatgactagttacataggaagccctggattggcgtgaaatactggtgcccgaggttcctcctgccccttaggctcactgacagatcatcccaagcaggcttatcaggttgggtctaattttaaaacagtcattgaggagtcctggccaccccacccctgcttttgtttgatgcttcacctgtgtttgctgggttatggtgtacacagtaaatcctgtgtgtattttaaacaccaaaaataatgggatctgttgctggtctcttttacgaatttcaggtttcactgtgagacagaattcatttcacctcagtcccatgagcacttttgtgtgttctaatttctctacgacaccataatgggagaagacaccgatgcaacctgcggaggcctttctgcagacccacctttaactggttttctctctcccaacttgggctggccaggcactagcaagaccacactctgcataggaagaaaaagaaagtccctcccaaagctagattccttctgctttttctttcacgatccccaccccatccctcccaagtacccaaggatgttgcccgtgttgaatacatgtggttgcatcttcttcctccataggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatggtaagtcccatgtcagcactgtcgtgcacagcaaggagcatcctcttattaatacaattccagaacttttgagctagtgggcacctttgaggacagcctgccctggctgttttttatacagactagagataggaccctgagcaggcacgggaaggtctgcccaggcttcacggcctgggatcagttgagccaaggcttgagtcaggctcctccctcccagcccagagctctgtctttcctcctgtccttctgtcactggcaccaaactgcctctaatctcatcacttgagagtaatgactactcacctctgagaaggttccggggatggatgtagggcagcaaaaccaccttctgttcttttctgcacaaggactccttgtgccagctccaagcctctggcctttgaagaagtcccaagacctgtgttctccccctctccctcatcccatgaagtggagtgacttagagtgctccagcttcttgtccttccacccccagtaccaccctgaccaaacatggccccactgccaccggcctggagcaccctctcctctctgttaactggggccatggagcaccatattacctgagcctgcctgacccctgcaacatcttccctgatatgagccccagcctgtctcagtgaacatgaataacttgggcaatcactgtcatgctgggcgctgttcctggtcattgtccttagggttgaaaacagggagtctgatgaccatgagtgccacagtcagaagaggataatgcactggcttaggggtcttttctgagcatctgctgtttgctcaaccccactctgggcagcaccaaggaagggacagtggcagatgaaccatggaccttcccctcaggatgcttccagtctaatgcaggagccaggtcaataaagtatacgtggtatactcaataaggtgataagctgaacagtgcagacaagaagtcctgggcctgaccaggaaggagaaagaattattcatgtagctcagcgggcaacatttcatggaagatgtggagcaggaacccaaaaaatgcaaagaatatgtaaatgaaagagacatgtaagaatgggcttttgggcaaagaaaagttactgagcaggtgtgtgaggggctatgtggtgggatgggcatgtggaggatacaaagtttagacattgtccagtgagggtggaaaaagaggagtctacagcttgactcagctttggggatgccgacttgttgcaccccctggtctaaatgtcaagtacccagttatcttctttctctgagtttatctagtggtacaggactcctgctcccttctaccttgaaggtaaatgcttttaacagaagatacagggactgatcaaaatgctcgtctccaatctctttcatagacccgccccttgtcaactctgatcagcagcagccagtcctgtcagtacacactggacgctaagaggaagcatgtggcagaagccatctgcaaggagcaacacctcttcctgcctttctcctacaagtaggtcatgtgatgcacccctgatttgtcatttaatgggtcagtgtgaactgaacacttctcaagtgctctgttccaggcaaacctgtgcctgggagggaggaatggagagggataaaatgccgcccctccctgtccccctttttaagcgaacaggccatttggcagaaaagtcctaggcatgcaaaacaatccaagaccaacaaaagatatctaagacccattctttaagggctgtagatccagaaaacctgaggatcactgcagggtaccctggttagaaaaggtttcatggaagatttgggatactgactggaaacttgtgtatccaaatccactttgaaaactgataatcaatgaatatatattgagtaactgccatattcttggctctatgttgtggaagatacgaaagaattttgagacattgcactagttcctacctctggccactccagactagtggagagtataaggcacgcatgtctttttgatgggaggataactagcgtgaccaggaagaggtggatgttattcattcagggccaacaatggctggatttacccatgctttgaaagatgggcaggacttgggtagatgcagagacagggaaaaccttcaacatggaaagaatagtatgttctggccatccgtgacatggtgtgcttccttggttaccaggaataagtatgggatggtagcacaagtgacacagactttgaaacttgaagacacaccaaagatcaacagccgcttctttggtgaaggtaagagtttctgtccacatagttgctggaaaatctactcaagatgtgcctatcatggcttagccacttgctgagccctgttaaatgtctgctgactaacaagtgatacagacactggtgttctggctacctctagtgagaaagcaaactcatttcatgatgtcaagttgcaatggcataaaggaaaagaagttcccaaagctacttaggcatttgtaaatagaaaactggaatcctaagtttaacatgacatatttgatagaactgacatcacccatcctgtgataagatccagagctgtcccagacgaggtggaccaagtgggagagaaccttcagagtctggccagatagtaacctcaggagtcagtctttagaggtagaaggaactctaacaatctcaagtccaacccttacccagtattgtattgtatttatatctgtccaaattccttcttgtacattacctcattgtcctttttgctcatagcaacctgtgatgtcaggtggtagagatgtgattttatacctattctacagaggagacagtgacacagagaggcttagagtttgatgtagtcaaggccgcagaatattagaggggggaaaataagtgccaggttgtaatctaagccaggactattctcattacaccacatttccatgatgacttttacctctcttcctggcataggtcacagtaggtggtggagaggatacaaaagtgtctcccctccccacaagctgctggtagacccaattagaagaaatggtgataagcacccatgtgcctggtcccagttgtaaccatgtcaacagtagcacctcctcaccaattatttcaagctaagggtaacctgatgatagactcagacaagtctggattccactttagctctacctcttagaccctgagagctcttgggaaacctaagttgctcatctctgggtcacacttcctcatctctgggtctcatctctttgtctcatctctgggactcagagctgagatccagggatgagcaatttacatggcccaaaaactctgtgggtctcagaagcagggctgaatttatcattaaattgaacaataatgccaccccacagggataggatgatgagtcagtgaaaacaagtcaatcacctatggcagagccagatctagcaggcattgaatacaggatagtttctttcccttttcccctgtgctgatactccacaatttccagcttccagtagacaaagatatggttgagatgaagaaagctagagttcctttgacactttccatcttccaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccaggtatctaatggttacagctcaactttttataaaactgatggtaactgactgaactttcaaaccttggccaaatggagaatctcagggaccatttggatatcaatccagttaatcaattagtcaatcagttcatgattgctggatagagaactatcagctgctgcgctgagttccatgaaacacacacgcgcatactgtgttcaaggcagctatgtatttgtgtgttaaaacagaaggagaatagttcccacattttgatgggtaacttttaattcctaggtctattgcaggtgctctccagaagcttataggctggtggagagagaactcagacgaaaaatataatatgatttctctacccttcaaggcactggctttaagtgctatgaaggtgagagaagggactgaggccaggaatgagacccagctaatgttggccaggcatattctgtgtgctggccaaaggactgtgataacagtcttcttgttgctacagatccacagtcccctcttggaacttttctcgattgggcttcttctgtgggtaatattcctaaggaaagcatcatggttctgagctccaagttgggttttgaagttagatttgaatagtgaatgaggtgattaagggctctcctggcagaggacacaccatgagcaatattttatgtgccctgaaggtggtctgtataactttatccatgtctttcttctcagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaagtgagtttccacactgtatttctcctcctaggagcagaggaacatcttgcacctctgtgcatctctgtattaaaactgaacccctccttccactttcaaactctgctccttactcttgtgttttttcttgatcatttttggggtaatgacttgaaataagaaatcagcaaacacaaattgaatttttaaaaatattttctctacattatattataaaagtttttgaacatagcaaagttgacagaatttcacagggaaaacccctagaaaaccagctatctcctactatttaagtgttattatatttgctttatcacatatacatccatccattaattcatcttattttctgaagcatttcaaagtaaattgcaaacatcaacacactttcccctaagtattacagcttgcatattattaacttcagttcaatattagttagcagttttttcctctgaatttttttgtttgtttgttttgtttttttttgttgttgttgtttttttgagatggtctcactgtgtcacccaggctggagtgcagtgatgcagtcacggctcactgaagcctcaaattcctgggctgaagtgatcctcccacctcagcctcctgagtagctgggaccacaggtgcatgctaccatgccctggctaatttttgtattcttggtagatacagggtttcaccatgttgctcaggctagcaggtttttcctttgatgaaattttttggctttttcttttttacatttttatataaatttatgtggaacaagtgtaattttgttacatgaatagattgtgcagtagttaagtcagggctttcagggtatccatcacccagacaacatatagtgtacccactaagtaatttctcaccatccatctccctccacttccacaccttctgagtctcaattgtctatcattccacacactatgtccttgtgtgcacattatttcactcccacttataaatgacaacacgcaatatttgtctttctgtgactgtcctgtttcacttaagacaatgacctccagttccatccatgttgctgcaaatgacatgattttattctttttatggccgaatagtattttattgcctatacatttcacatttttaatccaatcgtccattgatagacacttaggttgattccatgtctttgctattgtgaatagtgctgtgataaacatatgggtgcaggtttcctttggatataatgatttcttttcctttaggtatatacccagtaatgggattgttggatttattggtagttctatttttagttctttgagaaatctctgtattgttttccatagtggttgtacttatttacaatcccatcaacagtgattaactgtttccttttctctgtatcctcaccaacaactgttattttttgtcttttgaataatggccctcctgactcttgtaagatgttatctcattgtggttttaatttacatttctctaatgattagtaatgttatgcattttttcatatgcctattgccatttgtatgtcttcttttgaaaaaaatgtctattcatgtcctttgcctactttttaatgggattatttgggggatttttttgttgagttgtttgaattgcttgtacattccggatattagtaccccattggatgaatagtttgcaaatattttctcccattctgcaggttaccaccctgttgattatttgttttactgtgcagaaactttttactttaattaagttctatttgtctattttttgtttttgttgtctttgcctttgaggtcttattcacgaattctttgtctaggccaatgtccagagaagttttccctaggttttcttcttgcatttttatagtctcaggtcttatatttaagtctttgatccatcttgagttgatttttttatatggtgacagataggagtccagttttattcttctgcatatggcaatccatctttcccagcaccacttattgaaaagggtgtcctttccctagtgtatgtttttgtcaattttgtcaaagatccgttgactgtaagtatgtgactttatttctgggttcagtattctgttccattgatctatgtgtctatttttatgccagtaccatgctgtttagattactatagccttgttgtataatctgaagtcaggtaatgtgatgcctccagctatgttctttttgcttaaaattgcttcagctattcaggctctttttggattccatatgaattttataattattttttctaattcacaagtttgggttttaagacaaacctaactggggttaccaagtcctgactctcttctcttattctgtagctatcataagacaaaccctacagggacccaggagctgctggacattgctaattacctgatggaacagattcaagatgactgcactggggatgaagattacacctatttgattctgcgggtaatctcagtcttttatatgacatacatcatttcagaagcacttttcctggacaccttttacttccctctcctgcaccctgatgggttcttgtttcttttcttcaatgcaggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggtaaagtccacaagaagaggtctgaaagtgaaagtttattaacaaggatttggaaggtactaggggaatgagactctagatttcatctactgactttattctgctgtttctttcctttccttccttccttccttccttccttcctccctccctccctttcttctttccttccttccttccttctttcgagatggaatctcactctattgcccaggctggagtgcagtggcatgatctcggctcactgcaacttctgcctcctgggttcaagcaattctctctgcctcagcctcctgagtaactgggattacaggcatgtgccattacacccagctaatttttgtattttttagtagagatggagttttgccatgttggccaggctggtcttgagctcctgacctcaggtgatccgcctgcctcagccttgcaaagtgctgggattacaggcgtgagccactgcacctggcctctactgttttctaattgcaaatttcaacaagcctattgacttgactgcctagcagtatgtgacgtgagagaaatacttgactttgctgctatgtcaacatgcagaacgtgagatgtttttgcttcctaccgtccacctaccagattgaccatccctctcatcatggaaaaacatgcttaattttcccccaataagcttaggctaggatagccaacttggccccctcttaggtgcaaagactccagaactttggaaactaccctatttattagccccaaactcttactaccccttctcatctttatcctcacattaaaataacttacgttaaaacaacttgattttcacttagtggtggatctccaaacaaatcacaacttggccataatttatgtgttttaatggaattgaattcaacaggcattccacaggctttttctgggaacccttacttgatagtgctctaggaaacactggcaagaagattcaataccagcatttgaagaacgattacagagaaattagacctgtgcttaagaaagagctagcagacaatgccagtgtttgccaggcatgttctgtgttctgaccacaggacagtgataaccatctcctcttttgactgcaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagagtaagtaagagctattcaccccatataccactgagggccctgagctggaattccaaccctaggttttggcatagccactgtctgcccttgcttctgaaacaaacacttgtgcaaatgtgtagcagatctagacccaaagacttagggtcaatgaaatcaagacattttggtagtgattggaaatccatatttacttggggtgcaagagtcaaaggataataacatggtgtgtcagctcaaaatatacttcttcttatctagtctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgaggtaagtgtgaagagtttgaggttctctagcccattttgtacagcatcataaacagagagtccctgggagccaggagctacccagaggaaaactaagaaccaccaggcacttcctaccatgattctgaggctttcttctttccctccttccccgccttcctctctccccgctaggggtcacctgaagcatgacttcttaacattaatagaaatgcaggcctggcgaggtggctcactcctgtaatcccagcactttgggaggccgaggcgggtggatcatgaggtcaggatatcgacaccatcctggctaacacggtgaaagcccatctctactaaaaatacaaaaaattagccgggcgtggtggcaggcacctgtagtcccagctacttgggaggatgaggcaggagaatggcgtgaacccaggaggctgagcttgcagtgagccgagagattgcgccactgcgctccagcctgggcgacagagcaagactccatctcaaaaaaaaaaaaaaaaaaaaaaattgaaatgcaaatgtctcgtctttaagtcccaaagccaaggaagcatatgtgctgcctagtcagatctgcttcaaatctcaaatcactcccaactctgaatcctttgttgaattatttgtcctatctgaaccttagctgcctcttctagaaaaaagcaagtaataaggtcaagattctagtgagattttaataaagcagctcctgtgaaatgctaaggtcagctcctggcctgtggtattcaaatacttgtttagataaatggacatcaagagtggggactactaggctggcatacaacaaagaaacctgatgccattttcttgtctgattttctttctcagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggtgtgtatttgtgaagtatcttcttaaggaaagctttgggtctcaatgcaaaaacaattcttttctaagcatggaagtcctcaaaatactatctaactgaagggataactatggtttttatcaaccagacctgctggggtaagggccagtatcctctgcagttaaagatctcctgaattcagtgtgcccagaaaccagactcacaataagtactctaggataacaagagtatgaactctgggctgggtgtggtggttcatgcctgtaatcccagcactttgggaggccaaggtgggcagatcacaaggtcaggaatttgagaccagcctggccaacatactgaaaccccgtctctactaaaaatacaaaaaaactagctgggcatggtagtgggtgcctgtaatcctagctactcgggaggctgagacaggagaattgcttgaacccgggaggtggaggttgcagtgagccgagatcacgccgttacactccagcccgggtgacagtgtgagactgtatcttaaaaaaaaaaaaagtatgaactctgggcatagatttaattctaacttccctgtcttgaagctgtgcgcacttggggaagttggttgatattatgtgtatctgtttctgtctgtatcccagactactaataacagtccaaacctcacaaggttatttaaagacaatgaaataaggcatctaaaatgccaagcacagtgcctgatgctggcattggttgttcaataagcagacactattacgagttctaaattaatattttcattattattaactgctgtctttggctctcactcccatcagtgcactagcaaatgagaccaaacttccactttgaagctagcaatgagcccccatttaaggagggaaataggttgtatgatctggagcttattcttgaattttttgctacccaaagtgtggtctggtcagaaatacagcttctcatgcttcacccacaatctactgaatcagaagcgcattttagcaagacctcatgtgacttgtatgcacattcaactttgcagagcaaggcagtaatttacccctccaggctcactgttgagcacgagctccatcttctaatttcctgacccccacttgaggccgaggatctttgatctgctttgagtctgtcagtttcacattttttttttcccaatgcctgggcatccatctctgagattcttcttctctctgagaagaacttgtctaggatcaagtgtttttcaaacttctggtgaatttatataacagctacattttcttaagaaacaccttgtagtcttcactggtcaaagaagagaaggctaagcagggaacgggtgggggatagaggatcttctaatcttgaggatcctggcatactggagaatagggacccctcctctcatcccaccacatcttactatgtctacagattttttaattaagaatagctttaggagtgccactatccctgacaagaccttagttctttaatctctgcttagaggaattagcctggacttcagtgtctccctgttcctcacctggagcattttttaggcccatcctggctgcatcagacaggtcccacattgggaactgaaaggtgtttgacattgctgacatctcactggccattttattactaaactctcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatggtaagtcagcaggccccactgggggcccatgagaccagacgttggtttttttttagatcgcccagactcccttacgatcccagctgcacaagcccgaaaagatgcttgtactttcttcagagatggaggtttgccttgaatttcactgaagatgactcttggatcacatggaaatgttaacatttagaaattaagctattcataatgttagctgtatttttaagagcattaatttattcatctggaaaacaatgttcggtataccttcctctacctttgctgaaggtccttttatttttatttttatttttttaattttttgagatggagtcttgctcccaggctggagtgcagtgatacaatctcggctcactgcaactctgccttccgggttcaagcaattctcctgcctcagcctcccaagtagctgggactgtggacgtgcaccagcatgcccggctaatttgtgtatctttagtagagacaagcctgttgacaaccatgtcaggctggtttcgaactcctgacctcaagtgatcctccagcctgggcctcccacagtgctggaataacaggtgtgagccactgcacctgacctgaaggtccttttaagattgaaatgatacaatgattataaaagaaagtatttggcaaactataattcactatctaaatatgctataatttttattattaattcataaaaggaaatatataaatgtactcctatggcttgattaaaaaaatgttgactttaagaaaacaggtctcaagctattttattgaaatattatttaaaaaataaaacccaatgcaaattgatatgtacatcatctcaataggcctttggtttcaaaaaattgattttatcataatataatacatttcaagtacaccttcacttacagtcagactccagaacaccagaattaagccatggcatatatgatacttaaagtccataaagctctgaggcccagcaatattcttaagagccttctgagtccacttgaaaatgacatgatatctatctagtgaaatttcttatatcctgattcactgaaaacggtaaaaacatcagtttgatctttatttatcaaactattcagctcatcaaaatatgctagtccttcctttccagataaagaggaattactctccaatgtatgggaggttgtaattaacaaaaccgactttaaaaagacttacttttatttgctctcccttgttgggtctacagattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacgtaagattctgtttgccttttgatttcttaggttattactttcttccagggtgcatttcttgttaaaacatatttaaaaatgtgtttccacttcaagacaaaatgcttcatcattgtaatcacctcattatttttttatgaaaaacttcaagcttccaccagaatgcactacctcactagctccagtagtggtatggccataagacaagaactcagttctctcaacaaatgagtattcctatcatctttttaatctggttttgcctcacgttaactcaggtgctttctagttctgggtagtatactccaactctagagaactgagaactcgctttccttcttccaaacaaatcccagtaatgtttccaaaggtctgagttatccaggaaatctttgcccggaggtgagaaagggtggttgatctgactgacaggggactgaagtatttaatgaatctgaataggttgttttctgacttatagatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggagggtaattctttcagccaagtctgcctagccagtttgaaagagagaacagagaatgtacctgcagaattttgccaggctaaacagttgattgagatcattcaggtcctgaggaagcaggagaggagtagaaaggaaagattccgggttacctattttaattctagcctagacttactacataactacataattacctttcttctacttttcacattttactaaactgtcctttatctttctgctttgagacttattaagacctactgcttaattagtttttattaagttgtgattttttgttatctatttgttttgagaatgaagaaacaatagctctggagagatcatctttggaaaattaatattttcccccccaaaaaatacctaagaacatattgatttgaggtagctaggtaggtaaagcatgaaactcctaacctcgtgataatggaatacagcctcttttggagagttccattttaagtggcaccctcaaccattgatttgccttagttttcatattttagacacattcatgtgttcattcaaaaataatatttaattggccagccacggtggttcatgcctgtaatcctagcactttgggagccccaggtggatggatcgcttgagccctggtgtttggataccagcctgggcaacatggcaaaaccccatctctacaaaaaaaattaaataaataacaaaattagccagtcgtggtggcacatgcctgtagctccagctactcagaaggctgagatgggaggatcaactgagcccaagagttcaagccttcagtgaaccatgcttgcaccactgcactccagcctgggagacagagcaagatcctgtctcacaaaaaacaaaaaatagtatatttaattgcctaatatataccacgtatgttgagtgagacacacaaggtccctgacctttgaacgcttacattttataagggagacacacaattaagcaagcagtaatcatagagtaagggctaagttatagaaagtattagagtaccatgaaattttatatcatgtagcctgtgctagtcagggaatgcattctgaagcaagtgtacttgacctgataactgaggactgtgtcagagtcatttaggcaaaggagaaaggagtgagtgttccaggcaaaaggaaaagcatgtaatggcctgaaggtaaaggaatatggttcaaggaactggaagaagtgcagaatggtaaggggctcagagatgatggggagaggtaggcaggggagagagcatgcccagctgcgaaagccatcctaaggagtttggactcttttgaaggcacaggagttgaaaaggggagcagaaataagataggggtgatgttttagaagaaatactctgactctagtgtggaagatgggtgagaaggaggcacagctggacacgaagagaccattggacatctcttacgatcctatgtggctaagagctgataatggcctgcagtggagaaaagccaggtatagaaaggagtgagcagattctacaactttctaagaggcagaatcataagtactgggtgattaactgggtatggggacaaggcaaaagaaagaagaaaagaggaaggaggcgcccttcattttaataagaactacagtgggagagcttctggtttcaaggaaagtgacaaattcagttttggatgtgctgtatttgatgtcctcctatgaaacaaccagtttagaaatctagctgtcaaatagacctatggatctgagcccagtaaagaggcttgggctccacatatggatttgggaatcattagtatacagaggttgttgtggttaaacagcaactggtatagagtgagacatgagagatgaggacagaaatatggagaagacaaacatataaaggaagaaggggaataaccagcaatgagttagaagaagtgaccagagaagcagaaggagaaccaaagccataaaaggtcacagaagccaaagagcagccacaggggagatcaccccatgggtaggcgaaagctggcattaggactccagcacatcagcaaagcttggtcttgtggcacccccaacttggagaaacaatacttggaggaaaatgtgctatttcaaagaaagcatccttagaaaaaaccaggccaatgttgaactttcttacatgtactaagtttttaagtacacacttggaaggaaggtgccatcatctcttcagatgtgagaggctccagcgtcttagtctggtcatgagtgcgcaactctatggaaggcttctgggaggtcaaggaagatgaaacctaaatatgcccattggatgtaggagcaaggagggcattagagacattgatgaaagcattttcaggagatggagtgagcagtcagagcacattgggaggaagtagagactgcaaaggcagacaactcttgatggtgaggaagatgagaaagcaagaaaagaaagaaaggagcataggggaggggcacaggggaagagacttgagcgtgcttaatgcaggtggaaggaagcaggtagagagtaggagatttcatatgaaagagacagtttctcttgccctgcattgtaggaaggaaggggcacactgaagttcagccccagtgatcagctatttaacatctctgagcctctgcttctgtaaaatgagaaccataagcctactgttgtggggattacaggtaacagatggaaagaactcagccagaagcttcagagtcactctcatggcttgtcatgttgatgttctttctaatattatttgtttctcagtaaattaaatagttagagataggtgtggactgagggaagacaggaggataagggggtatttgcaccctgagaatttgtgatgtccattttgattcatgacttggcaataactcaggtatttttgttcttcaccagcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccaggttagagatgctcagtgcctgacccagcattttctcaccttccacatcatggccacctagcatggcacaggaaaaaatactctgtgttgtaagaccctgtcactagccttctgggtttgcaccatctttgggtatttaaagcagggtcctctggccaacacattgggtgtcaccttttgcttccttgtgcatgggatgggatcacagcacagatcccaatttgctcctaattcagtgtccatgtttctgagcctccagacccatcgctatgagcttcctggagcccaccaatgtgcttgaagccttcaccgtacttaggtggctccctgtcttcagcccccaagttccagtgcttgttctcagctttgctgaaacaaccagccaactcctgctctgcttgtccaaagtcttgggaatcctggtgtctgcccttgccttgggttcttgtaggactgagggatcaaaaagatcatcttagttaagggcaagagacaatgttaaaataaggaccatatttttgttgcatttgaggctgaattgttttgggaacataatcaccatccttgaaagctctaacattatgcactgtcttcattgtaatgtctttagattagagctggaactgaggcctacaggagagattgagcagtattctgtcagcgcaacctatgagctccagagagaggacagagccttggtggataccctgaagtttgtaactcaagcagaaggtgagtattcaaaacacagctgcctcatctctgctcgcagtctcaggttcagaattcatgaggagaagacatgtaatttaacctatttaacaaataggttaactgagtacccactaagcggcaggcctattctaagacctgggttaactgagtacccaataagcggcaggcctattctaagacctggggctagaacagtgaacaatggagtctctgccttcatggaagttacagtgaacaaccaaacaagttaatatttggaatatcagataagtactgaggaggaaaacagagcgtagactggtctatggagggctaggagtaggagggaggaagaagggcagggaaagcagtgcatttggaataataagggaaagtctccctggtaaagtgagcataaggagacctatcagaaataagaggagaagccgtgtggtaagactgttaacaggcagagggaccagcaagtgcaaaggccctgaggctgacacactactaccatgtttcaaggaaaggaaggaagacagtatggctggagcagaaagaccagggagaaaagaggtagaagatgaggacagagagatatggagaggtgaaggaaggataatctcataggccatggtaagaactttggctttttctatgaattaaacgaaagccattggggagtcctcatgatttgatttatgtttatgttgagaaaagactatgggcagacaagggcagagaaactaatatgtaggttatcacaataatccaggcaggaatcagtgttgttttggatcagggcaatggcagaagagatatgagaaggggatggattctggccatattttgaagattaggctgacaagatttgctgatacagtggatgttgagtgtaagaggaaaaggggaatgaagacaaacctaaggtttttggcccgggcaactgaaaaatggaacttccatttattgagatggaaagggctactggaggagcaggttttagggaatgggagaaatttaggtgttcactttggaaaaaaaattatatagggatagcgaggagcaggttttagggaatggggcacatttaggtgttcactttggaaaaatttttatatagggatagcatatcacagaattaaactaggaagaaaatcccatgatagaaagcactggaggagcagggcacgctggggaaatagtgtttggtaaacattgttttacgaaggatataaaatggaccagcctatggattgaaggacgcccgggaatcttgttacaaagaaagggggagttggggagatggagcccagggcaagggcagcaaggaaccaggacaggcatcttgggtagaaagtaatatagagatgtcgtgtcttcctggcccagaagggctgcgagcctttgctgttccacaaacaagctaagtgctccccatttcagggcctttgcattcctgaccttctgcctggaatgtgctcctcccagaactcagcgtggctccaacctcttttcattctggtctctgcccacatgtgcccttatcagagagaatttctctgaccaccaagtatgaaataacacttcttctatccctttcttttatccttgtatccagttttactcttcttcataacattcattaccatctgacatgagcaagttacttgtttattgcctgtacacctcccccactagaaggtaagccccatgaaagcaaggattccccagtaccaagagcagtgcccagcacacaataggctcataacaggcaatccataaagacttgcatacatgaacacaactgagtttaaaattatcagtaaatgagacccattaaaaaattttaatgagaaaaaaaaaattcagtaaaatcctgaactgtgtttttgtttaagcacattgattccttggagtttctctaccttttcctctctttccttccaaaacatagcttctttatttatttatttatttatttgtttgtttatttatttatttatttatttatttattttttgagatggagtctcgctcttttgcccaggctgcagtgcagtggtgccatctcggctcactgcaagccccgcctcccgggttcatgccattctcctgcctcagcctcctgagtagctgggactacaggcacccaccaacgcgcccggctaattttttgtatttttagtagagacggggtttcaccatgttagccagaatggtcttgatctcctgacctcatgatctgcccgccttggcctcccaaagtgctgggattacaggtgtgagccaccgcacccggcccaaaacatagcttcttaccacacatctcttgattctcttatacactcgtccaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaaggtaaagaaggccgagggtcatctgacctgcactgcaggcctgggtggttcttttcattattcctcttccacttcatacctgaccaagccatgttctcccctagtctacaatcagagtggcagagagagccctcaacaattttttttttttttgagatggagtctcactctgtcaccaggctggagtgcagtggcacaatctcggctcactgcaacctccgcctcccgagttcaagtgattctcctgcttaagcctcccaaggagctggaactataggtgcatgccaccacacccagctaatttttatatttttagtagagacagggtttcaccatattgaccaggatggtctcgatctcctgacctcgtgatccacctgccttggcctcccaaagtgctgggattacaggtgtaagccactgcacccggccaagctctcaacattttaaccctctgcgcatgtccagttggattttcctaccatttatcaggcacttactattcatgtatcaagcacagtgctgggtgctttaaagaaattatctcggtcctcacaataaactgcgaggtcactgtgagttttcctgtttcatggataaggaaatggtagctcagaggggttaaatcatttggtcaaaatcacagagctagtaaatagcagagcaggattcaaacagttttcaaaaaacttctctttctcctaaacctgtttgcaaagtccttaatttgtgctgaatgttggctttagaagttgatgagtttgatctgtggctgtttctctgaaccatccttgtatctggttttgatcaccacaaatggaacttctgtttaatcctgcatatctccattgaaaggacaaaatcattggtgccaactgattttctttaccatagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatggtatgtgtctcttcccctgtgtgagcacttccaaagtaatgcaggtgttgagacctgtggttacaggctgaactagtaccattcacaactatttcctacgtattttcagatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgtaagtatgagtctgccagtcaataaatacatggatataagtgctaattacatcctcaactctgagctaggtgcaggaaggtttccaaagatgtataaggcatgcttccttccccccagggaattcttggggagaaaaaaaaactttcacaagtgtgtagttacccagttacacaaagctgaatgtgatacatatcaaagagatgctactaagtagaacagttctttgcctagtggtatcaaaggaagcttcaggacaccagctaggaggctgactatgttagacattccttttataaatatggacagtgatcagtgactggcaacgaagattcataattttctgttatttatttttaactttcagtgcattgtccagcttaataattaacttgtcaaatcggtatttttgcctaatgttcattgctctttgaggctcatccaagcccattaccttaaaaatctcctgtcattttgtaggcaatgagctcatggcttcagaaggcatctgggagtcttccttatacccagactttgcaagaccacctcaatagcctgaaggagttcaacctccagaacatgggattgccagacttccacatcccagaaaacctcttcttaaaaaggtaaaagaagaaagcagcaaggcttcttgaaccatgcaaagtaaatgaaagattttacatagcatgatttagacatttttttaaatttttaaaggaaataatttaagcattttaaggagattaataactatagcacaaacactgtggcatctttgcattagtaaacatgagaacaccaaccctgtcaggaagaatctaagaaagtcattagaggattctggtactttcaccctaagatattttattcagtacaacctgttataagcaaattctccctctgactgtgaagaattcagaatggctagaggcgttattgactacaggcttgctgttaagctagagagagtcagaacagccattgagcactaaatggaggcagcattctgagaaaatactttaacccaggcttactgacttccatacctatgttctttccacaaatcaagttgtctcaattcagtttagcaaatttgtatcaagtatcccctatgtgcaaaatgctagactaggtacagtgagaagatagaaactgggtaaggtatagccttttctttcaagaagataccatggagacatcaacaaatgagaaataattaattatataagcaaaattatgacatgctctttgagaaaggtgcaagggactatgtaactgtaagaatgagacaaattggctatgacttaggtgggatggtaatgataaggagtggcccttagaagagctttgtcaggatttgagtgtttgacaggtggaggtaaaagcaaaggggtccaggcataggagtagcacaaagaaaagtgcagagtggctttgggaatggggcaagtacaatattgttgtgaaggtcagaggcagagaactttgaatgactgatgtctgactgtggggatgttatctttgttgttcatttcagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggtgagctatgctcaggtaaagggtgcaccgggctagttcatggcaggctctaagaggagagcctcctccagggaggaaaggactttggctttctagcagataatcttccttgctacttggaagtcttttattttattcaacaaatagaaatatttattaaacatatcacgtgtattaaatattctagtaggcagtaacagaaagtagacagataagccagcaattataattcagtgtgagaggtgctatgataaagtgtagtatataagtataaggtagagtggaagcactcaacaagggaacctaaacaaagcctgtggtggtcaggcaaggcttcctggaggaatgccttttgctatcagattttatctttgcattacagatggaggagtctattgcacaattggcccagaaaaatggggctttattattgaaagactttcaacatagagattgctctggaaatgtactgcttaatttaaccaatgtcttttcatttttatgttaggatctggagaaacaacatatgaccacaagaatacgttcacactatcatatgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacattcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatggtaagaaatatcctgcctcctctcctagatactgtatattttcaatgagagttatgagtaaataattatgtatttagttgtgagtagatgtacaattactcaatgtcacaaaattttaagtaagaaaagagatacatgtataccctacacgtaaaaaccaaactgtagaaaatctagtgtcattcaagacaaacagctttaaagaaaatggatttttctgtaattattttaggactaacaatgtcttttaactatttattttaaaataagtgtgagctgtacattgcatattttaaacacaagtgaaatatctggttaggatagaattctcccagttttcacaatgaaaacatcaacgtcctactgttatgaatctaataaaatacaaaatctctcctatacagttttgggaacacacaaaatcgaagatggtacgttagcctctaagactaaaggaacatttgcacaccgtgacttcagtgcagaatatgaagaagatggcaaatatgaaggacttcagtatggagcttttattgaattgaaaccttataccttttgaaaactcattgtgattttcttcatctccatacccctttcgtgatagctcatctgtttttctgctttcagggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcaggtaaataccacctaatgagtgacacgcccccaagagcgagtggagaattggggcagatacatttaattcaggaccaaatattcagagattccccaaactaggtgaaagacaggcggtaagcaacttcttctctgaggaaatattctctagaaagtattacaatgagtccttgattgattttaatgtttagatgcacacatgacatcccatcagcactattatttattaattctgggcaaatccaggaagatgagggttatacctcatcatctaaatcataggcaagctcagccataggcagggtatatttttcagagaggactggtttctgtagtatttaaaactttaaaattcttccccacaatagaattgctagatgagatacatcaaattcctctcatgtcatttacaagctctgccagggccaaatcaagggtgacattaccagaggagaagaccaaacatggttctatgactgttactaaaagtttgtcatgggcttggagaatgcgtactgatgttgggattctgggtctctgcagggtgggctccaacttgccttttttgctatttcttcttttcctatctgtcatttcctgactcttcttctctctcctcttctttctcttccccccactcctcttccagttttcagtcctaggaaggctttaattttaagtgtcacaatgtaaatgacaaacagcaagcgtttttgttaaatcctttctggggcatgtgataaagagaaattaacaacagtagacttatttaaccataaaacaaacacatgaactgacatatgaaagataaatccctttcagtatatgaaagattctctgatctttatttttaactgctaatgaagttttagtgtactatattgtgtaattggagtaattgaaaacatgttatttttttttttctctctgtttagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcataccactgaatgtggctcatttgtattgaaagacagtgaaacgagggcattgataaaatgttctggcacagcaaaacctctagaacacatagtgtgatttaagtaacagaataaaaatggaaacggagaaattatggagggaaatattttgcaaaaatatttaaaaagatgaggtaattgtgtttttataattaaatattttataattaaaatatttataattaaaatatttataattaaatattttataattaaaatatttataattaaatattttataattaaagtatttataattaaatattttataattaaaatatttataattaaatattttataattaaaatatttataattaaatattttataattaaaatatttataattaaatattttataat

SEQ ID NO: 335 - DNA, 20 bp, Artificial Sequence
  Antisense Oligonucleotide
tctgtaagacaggagaaaga

SEQ ID NO: 336 - DNA, 20 bp, Artificial Sequence
  Antisense Oligonucleotide
atttcctcttctgtaagaca

SEQ ID NO: 337 - DNA, 20 bp, Artificial Sequence
  Antisense Oligonucleotide
gatgccttacttggacagac

SEQ ID NO: 338 - DNA, 20 bp, Artificial Sequence
  Antisense Oligonucleotide
agaaatagctctcccaagga

SEQ ID NO: 339 - DNA, 20 bp, Artificial Sequence
  Antisense Oligonucleotide
gtcgcatcttctaacgtggg

SEQ ID NO: 340 - DNA, 20 bp, Artificial Sequence
  Antisense Oligonucleotide
tcctccataccttgcagttg

SEQ ID NO: 341 - DNA, 20 bp, Artificial Sequence
  Antisense Oligonucleotide
tggctcatgtctaccatatt

SEQ ID NO: 342 - DNA, 20 bp, Artificial Sequence
  Antisense Oligonucleotide
cagttgaaatgcagctaatg

SEQ ID NO: 343 - DNA, 20 bp, Artificial Sequence
  Antisense Oligonucleotide
tgcagactaggagtgaaagt

SEQ ID NO: 344 - DNA, 20 bp, Artificial Sequence
  Antisense Oligonucleotide
aggaggatgtccttttattg

SEQ ID NO: 345 - DNA, 20 bp, Artificial Sequence
  Antisense Oligonucleotide
atcagagcaccaaagggaat

SEQ ID NO: 346 - DNA, 20 bp, Artificial Sequence
  Antisense Oligonucleotide
ccagctcaacctgagaattc

SEQ ID NO: 347 - DNA, 20 bp, Artificial Sequence
  Antisense Oligonucleotide
catgacttacctggacatgg

SEQ ID NO: 348 - DNA, 20 bp, Artificial Sequence
  Antisense Oligonucleotide
cctcagcggacacacacaca

SEQ ID NO: 349 - DNA, 20 bp, Artificial Sequence
  Antisense Oligonucleotide
gtcacatccgtgcctggtgc

SEQ ID NO: 350 - DNA, 20 bp, Artificial Sequence
  Antisense Oligonucleotide
cagtgcctctgggaccccac

SEQ ID NO: 351 - DNA, 20 bp, Artificial Sequence
  Antisense Oligonucleotide
agctgcagtggccgatcagc

SEQ ID NO: 352 - DNA, 20 bp, Artificial Sequence
  Antisense Oligonucleotide
gacctccccagccacgtgga

SEQ ID NO: 353 - DNA, 20 bp, Artificial Sequence
  Antisense Oligonucleotide
tctgatcaccatacattaca

SEQ ID NO: 354 - DNA, 20 bp, Artificial Sequence
  Antisense Oligonucleotide
atttcccactgggtactctc

SEQ ID NO: 355 - DNA, 20 bp, Artificial Sequence
  Antisense Oligonucleotide
ggctgaagcccatgctgact

SEQ ID NO: 356 - DNA, 20 bp, Artificial Sequence
  Antisense Oligonucleotide
gttggacagtcattcttttg

SEQ ID NO: 357 - DNA, 20 bp, Artificial Sequence
  Antisense Oligonucleotide
cacttgttggacagtcattc

SEQ ID NO: 358 - DNA, 20 bp, Artificial Sequence
  Antisense Oligonucleotide
attttaaattacagtagata

SEQ ID NO: 359 - DNA, 20 bp, Artificial Sequence
  Antisense Oligonucleotide
ctgttctccacccatatcag

SEQ ID NO: 360 - DNA, 20 bp, Artificial Sequence
  Antisense Oligonucleotide
gagctcatacctgtcccaga

SEQ ID NO: 361 - DNA, 20 bp, Artificial Sequence
  Antisense Oligonucleotide
ttcaagggccactgctatca

SEQ ID NO: 362 - DNA, 20 bp, Artificial Sequence
  Antisense Oligonucleotide
ccagtatttcacgccaatcc

SEQ ID NO: 363 - DNA, 20 bp, Artificial Sequence
  Antisense Oligonucleotide
ggcaggaggaacctcgggca

SEQ ID NO: 364 - DNA, 20 bp, Artificial Sequence
  Antisense Oligonucleotide
ttttaaaattagacccaacc

SEQ ID NO: 365 - DNA, 20 bp, Artificial Sequence
  Antisense Oligonucleotide
tgactgttttaaaattagac

SEQ ID NO: 366 - DNA, 20 bp, Artificial Sequence
  Antisense Oligonucleotide
cccagcaaacacaggtgaag

SEQ ID NO: 367 - DNA, 20 bp, Artificial Sequence
  Antisense Oligonucleotide
gagtgtggtcttgctagtgc

SEQ ID NO: 368 - DNA, 20 bp, Artificial Sequence
  Antisense Oligonucleotide
ctatgcagagtgtggtcttg

SEQ ID NO: 369 - DNA, 20 bp, Artificial Sequence
  Antisense Oligonucleotide
agaagatgcaaccacatgta

SEQ ID NO: 370 - DNA, 20 bp, Artificial Sequence
  Antisense Oligonucleotide
acacggtatcctatggagga

SEQ ID NO: 371 - DNA, 20 bp, Artificial Sequence
  Antisense Oligonucleotide
tgggacttaccatgcctttg

SEQ ID NO: 372 - DNA, 20 bp, Artificial Sequence
  Antisense Oligonucleotide
ggttttgctgccctacatcc

SEQ ID NO: 373 - DNA, 20 bp, Artificial Sequence
  Antisense Oligonucleotide
acaaggagtccttgtgcaga

SEQ ID NO: 374 - DNA, 20 bp, Artificial Sequence
  Antisense Oligonucleotide
atgttcactgagacaggctg

SEQ ID NO: 375 - DNA, 20 bp, Artificial Sequence
  Antisense Oligonucleotide
gaaggtccatggttcatctg

SEQ ID NO: 376 - DNA, 20 bp, Artificial Sequence
  Antisense Oligonucleotide
attagactggaagcatcctg

SEQ ID NO: 377 - DNA, 20 bp, Artificial Sequence
  Antisense Oligonucleotide
gagattggagacgagcattt

SEQ ID NO: 378 - DNA, 20 bp, Artificial Sequence
  Antisense Oligonucleotide
catgacctacttgtaggaga

SEQ ID NO: 379 - DNA, 20 bp, Artificial Sequence
  Antisense Oligonucleotide
tggatttggatacacaagtt

SEQ ID NO: 380 - DNA, 20 bp, Artificial Sequence
  Antisense Oligonucleotide
actcaatatatattcattga

SEQ ID NO: 381 - DNA, 20 bp, Artificial Sequence
  Antisense Oligonucleotide
caaggaagcacaccatgtca

SEQ ID NO: 382 - DNA, 20 bp, Artificial Sequence
  Antisense Oligonucleotide
atacttattcctggtaacca

SEQ ID NO: 383 - DNA, 20 bp, Artificial Sequence
  Antisense Oligonucleotide
ggtagccagaacaccagtgt

SEQ ID NO: 384 - DNA, 20 bp, Artificial Sequence
  Antisense Oligonucleotide
actagaggtagccagaacac

SEQ ID NO: 385 - DNA, 20 bp, Artificial Sequence
  Antisense Oligonucleotide
accacctgacatcacaggtt

SEQ ID NO: 386 - DNA, 20 bp, Artificial Sequence
  Antisense Oligonucleotide
tactgtgacctatgccagga

SEQ ID NO: 387 - DNA, 20 bp, Artificial Sequence
  Antisense Oligonucleotide
ggaggtgctactgttgacat

SEQ ID NO: 388 - DNA, 20 bp, Artificial Sequence
  Antisense Oligonucleotide
tccagacttgtctgagtcta

SEQ ID NO: 389 - DNA, 20 bp, Artificial Sequence
  Antisense Oligonucleotide
tctaagaggtagagctaaag

SEQ ID NO: 390 - DNA, 20 bp, Artificial Sequence
  Antisense Oligonucleotide
ccagagatgagcaacttagg

SEQ ID NO: 391 - DNA, 20 bp, Artificial Sequence
  Antisense Oligonucleotide
ggccatgtaaattgctcatc

SEQ ID NO: 392 - DNA, 20 bp, Artificial Sequence
  Antisense Oligonucleotide
aaagaaactatcctgtattc

SEQ ID NO: 393 - DNA, 20 bp, Artificial Sequence
  Antisense Oligonucleotide
ttcttagtacctggaagatg

SEQ ID NO: 394 - DNA, 20 bp, Artificial Sequence
  Antisense Oligonucleotide
cattagatacctggacacct

SEQ ID NO: 395 - DNA, 20 bp, Artificial Sequence
  Antisense Oligonucleotide
gtttcatggaactcagcgca

SEQ ID NO: 396 - DNA, 20 bp, Artificial Sequence
  Antisense Oligonucleotide
ctggagagcacctgcaatag

SEQ ID NO: 397 - DNA, 20 bp, Artificial Sequence
  Antisense Oligonucleotide
tgaagggtagagaaatcata

SEQ ID NO: 398 - DNA, 20 bp, Artificial Sequence
  Antisense Oligonucleotide
ggaaactcacttgttgaccg

SEQ ID NO: 399 - DNA, 20 bp, Artificial Sequence
  Antisense Oligonucleotide
aggtgcaagatgttcctctg

SEQ ID NO: 400 - DNA, 20 bp, Artificial Sequence
  Antisense Oligonucleotide
tgcacagaggtgcaagatgt

SEQ ID NO: 401 - DNA, 20 bp, Artificial Sequence
  Antisense Oligonucleotide
cacaagagtaaggagcagag

SEQ ID NO: 402 - DNA, 20 bp, Artificial Sequence
  Antisense Oligonucleotide
gatggatggtgagaaattac

SEQ ID NO: 403 - DNA, 20 bp, Artificial Sequence
  Antisense Oligonucleotide
tagacaattgagactcagaa

SEQ ID NO: 404 - DNA, 20 bp, Artificial Sequence
  Antisense Oligonucleotide
atgtgcacacaaggacatag

SEQ ID NO: 405 - DNA, 20 bp, Artificial Sequence
  Antisense Oligonucleotide
acatacaaatggcaataggc

SEQ ID NO: 406 - DNA, 20 bp, Artificial Sequence
  Antisense Oligonucleotide
taggcaaaggacatgaatag

SEQ ID NO: 407 - DNA, 20 bp, Artificial Sequence
  Antisense Oligonucleotide
ttatgatagctacagaataa

SEQ ID NO: 408 - DNA, 20 bp, Artificial Sequence
  Antisense Oligonucleotide
ctgagattacccgcagaatc

SEQ ID NO: 409 - DNA, 20 bp, Artificial Sequence
  Antisense Oligonucleotide
gatgtatgtcatataaaaga

SEQ ID NO: 410 - DNA, 20 bp, Artificial Sequence
  Antisense Oligonucleotide
tttccaatgacctgcattga

SEQ ID NO: 411 - DNA, 20 bp, Artificial Sequence
  Antisense Oligonucleotide
agggatggtcaatctggtag

SEQ ID NO: 412 - DNA, 20 bp, Artificial Sequence
  Antisense Oligonucleotide
ggctaataaatagggtagtt

SEQ ID NO: 413 - DNA, 20 bp, Artificial Sequence
  Antisense Oligonucleotide
tcctagagcactatcaagta

SEQ ID NO: 414 - DNA, 20 bp, Artificial Sequence
  Antisense Oligonucleotide
cctcctggtcctgcagtcaa

SEQ ID NO: 415 - DNA, 20 bp, Artificial Sequence
  Antisense Oligonucleotide
catttgcacaagtgtttgtt

SEQ ID NO: 416 - DNA, 20 bp, Artificial Sequence
  Antisense Oligonucleotide
ctgacacaccatgttattat

SEQ ID NO: 417 - DNA, 20 bp, Artificial Sequence
  Antisense Oligonucleotide
ctttttcagactagataaga

SEQ ID NO: 418 - DNA, 20 bp, Artificial Sequence
  Antisense Oligonucleotide
tcacacttacctcgatgagg

SEQ ID NO: 419 - DNA, 20 bp, Artificial Sequence
  Antisense Oligonucleotide
aagaaaatggcatcaggttt

SEQ ID NO: 420 - DNA, 20 bp, Artificial Sequence
  Antisense Oligonucleotide
ccaagccaatctgagaaaga

SEQ ID NO: 421 - DNA, 20 bp, Artificial Sequence
  Antisense Oligonucleotide
aaatacacacctgctcatgt

SEQ ID NO: 422 - DNA, 20 bp, Artificial Sequence
  Antisense Oligonucleotide
cttcacaaatacacacctgc

SEQ ID NO: 423 - DNA, 20 bp, Artificial Sequence
  Antisense Oligonucleotide
agtggaagtttggtctcatt

SEQ ID NO: 424 - DNA, 20 bp, Artificial Sequence
  Antisense Oligonucleotide
ttgctagcttcaaagtggaa

SEQ ID NO: 425 - DNA, 20 bp, Artificial Sequence
  Antisense Oligonucleotide
tcaagaataagctccagatc

SEQ ID NO: 426 - DNA, 20 bp, Artificial Sequence
  Antisense Oligonucleotide
gcatacaagtcacatgaggt

SEQ ID NO: 427 - DNA, 20 bp, Artificial Sequence
  Antisense Oligonucleotide
tacaaggtgtttcttaagaa

SEQ ID NO: 428 - DNA, 20 bp, Artificial Sequence
  Antisense Oligonucleotide
atgcagccaggatgggccta

SEQ ID NO: 429 - DNA, 20 bp, Artificial Sequence
  Antisense Oligonucleotide
ttaccatatcctgagagttt

SEQ ID NO: 430 - DNA, 20 bp, Artificial Sequence
  Antisense Oligonucleotide
gcaaaggtagaggaaggtat

SEQ ID NO: 431 - DNA, 20 bp, Artificial Sequence
  Antisense Oligonucleotide
aaggaccttcagcaaaggta

SEQ ID NO: 432 - DNA, 20 bp, Artificial Sequence
  Antisense Oligonucleotide
cataggagtacatttatata

SEQ ID NO: 433 - DNA, 20 bp, Artificial Sequence
  Antisense Oligonucleotide
attatgataaaatcaatttt

SEQ ID NO: 434 - DNA, 20 bp, Artificial Sequence
  Antisense Oligonucleotide
agaaatttcactagatagat

SEQ ID NO: 435 - DNA, 20 bp, Artificial Sequence
  Antisense Oligonucleotide
agcatattttgatgagctga

SEQ ID NO: 436 - DNA, 20 bp, Artificial Sequence
  Antisense Oligonucleotide
gaaaggaaggactagcatat

SEQ ID NO: 437 - DNA, 20 bp, Artificial Sequence
  Antisense Oligonucleotide
cctctccaatctgtagaccc

SEQ ID NO: 438 - DNA, 20 bp, Artificial Sequence
  Antisense Oligonucleotide
ctggataactcagacctttg

SEQ ID NO: 439 - DNA, 20 bp, Artificial Sequence
  Antisense Oligonucleotide
agtcagaaaacaacctattc

SEQ ID NO: 440 - DNA, 20 bp, Artificial Sequence
  Antisense Oligonucleotide
cagcctgcatctataagtca

SEQ ID NO: 441 - DNA, 20 bp, Artificial Sequence
  Antisense Oligonucleotide
aaagaattaccctccactga

SEQ ID NO: 442 - DNA, 20 bp, Artificial Sequence
  Antisense Oligonucleotide
tctttcaaactggctaggca

SEQ ID NO: 443 - DNA, 20 bp, Artificial Sequence
  Antisense Oligonucleotide
gcctggcaaaattctgcagg

SEQ ID NO: 444 - DNA, 20 bp, Artificial Sequence
  Antisense Oligonucleotide
ctacctcaaatcaatatgtt

SEQ ID NO: 445 - DNA, 20 bp, Artificial Sequence
  Antisense Oligonucleotide
tgctttacctacctagctac

SEQ ID NO: 446 - DNA, 20 bp, Artificial Sequence
  Antisense Oligonucleotide
accttgtgtgtctcactcaa

SEQ ID NO: 447 - DNA, 20 bp, Artificial Sequence
  Antisense Oligonucleotide
atgcattccctgactagcac

SEQ ID NO: 448 - DNA, 20 bp, Artificial Sequence
  Antisense Oligonucleotide
catctctgagccccttacca

SEQ ID NO: 449 - DNA, 20 bp, Artificial Sequence
  Antisense Oligonucleotide
gctgggcatgctctctcccc

SEQ ID NO: 450 - DNA, 20 bp, Artificial Sequence
  Antisense Oligonucleotide
gctttcgcagctgggcatgc

SEQ ID NO: 451 - DNA, 20 bp, Artificial Sequence
  Antisense Oligonucleotide
actcctttctatacctggct

SEQ ID NO: 452 - DNA, 20 bp, Artificial Sequence
  Antisense Oligonucleotide
attctgcctcttagaaagtt

SEQ ID NO: 453 - DNA, 20 bp, Artificial Sequence
  Antisense Oligonucleotide
ccaagcctctttactgggct

SEQ ID NO: 454 - DNA, 20 bp, Artificial Sequence
  Antisense Oligonucleotide
cactcatgaccagactaaga

SEQ ID NO: 455 - DNA, 20 bp, Artificial Sequence
  Antisense Oligonucleotide
acctcccagaagccttccat

SEQ ID NO: 456 - DNA, 20 bp, Artificial Sequence
  Antisense Oligonucleotide
ttcatatgaaatctcctact

SEQ ID NO: 457 - DNA, 20 bp, Artificial Sequence
  Antisense Oligonucleotide
tatttaatttactgagaaac

SEQ ID NO: 458 - DNA, 20 bp, Artificial Sequence
  Antisense Oligonucleotide
taatgtgttgctggtgaaga

SEQ ID NO: 459 - DNA, 20 bp, Artificial Sequence
  Antisense Oligonucleotide
catctctaacctggtgtccc

SEQ ID NO: 460 - DNA, 20 bp, Artificial Sequence
  Antisense Oligonucleotide
gtgccatgctaggtggccat

SEQ ID NO: 461 - DNA, 20 bp, Artificial Sequence
  Antisense Oligonucleotide
agcaaattgggatctgtgct

SEQ ID NO: 462 - DNA, 20 bp, Artificial Sequence
  Antisense Oligonucleotide
tctggaggctcagaaacatg

SEQ ID NO: 463 - DNA, 20 bp, Artificial Sequence
  Antisense Oligonucleotide
tgaagacagggagccaccta

SEQ ID NO: 464 - DNA, 20 bp, Artificial Sequence
  Antisense Oligonucleotide
aggattcccaagactttgga

SEQ ID NO: 465 - DNA, 20 bp, Artificial Sequence
  Antisense Oligonucleotide
cagctctaatctaaagacat

SEQ ID NO: 466 - DNA, 20 bp, Artificial Sequence
  Antisense Oligonucleotide
gaatactcaccttctgcttg

SEQ ID NO: 467 - DNA, 20 bp, Artificial Sequence
  Antisense Oligonucleotide
atctctctgtcctcatcttc

SEQ ID NO: 468 - DNA, 20 bp, Artificial Sequence
  Antisense Oligonucleotide
ccaactccccctttctttgt

SEQ ID NO: 469 - DNA, 20 bp, Artificial Sequence
  Antisense Oligonucleotide
tctgggccaggaagacacga

SEQ ID NO: 470 - DNA, 20 bp, Artificial Sequence
  Antisense Oligonucleotide
tattgtgtgctgggcactgc

SEQ ID NO: 471 - DNA, 20 bp, Artificial Sequence
  Antisense Oligonucleotide
tgcttcgcacctggacgagt

SEQ ID NO: 472 - DNA, 20 bp, Artificial Sequence
  Antisense Oligonucleotide
ccttctttaccttaggtggc

SEQ ID NO: 473 - DNA, 20 bp, Artificial Sequence
  Antisense Oligonucleotide
gctctctctgccactctgat

SEQ ID NO: 474 - DNA, 20 bp, Artificial Sequence
  Antisense Oligonucleotide
aacttctaaagccaacattc

SEQ ID NO: 475 - DNA, 20 bp, Artificial Sequence
  Antisense Oligonucleotide
tgtgtcacaactatggtaaa

SEQ ID NO: 476 - DNA, 20 bp, Artificial Sequence
  Antisense Oligonucleotide
agacacataccataatgcca

SEQ ID NO: 477 - DNA, 20 bp, Artificial Sequence
  Antisense Oligonucleotide
ttctcttcatctgaaaatac

SEQ ID NO: 478 - DNA, 20 bp, Artificial Sequence
  Antisense Oligonucleotide
tgaggatgtaattagcactt

SEQ ID NO: 479 - DNA, 20 bp, Artificial Sequence
  Antisense Oligonucleotide
agctcattgcctacaaaatg

SEQ ID NO: 480 - DNA, 20 bp, Artificial Sequence
  Antisense Oligonucleotide
gttctcatgtttactaatgc

SEQ ID NO: 481 - DNA, 20 bp, Artificial Sequence
  Antisense Oligonucleotide
gaattgagacaacttgattt

SEQ ID NO: 482 - DNA, 20 bp, Artificial Sequence
  Antisense Oligonucleotide
ccggccatcgctgaaatgaa

SEQ ID NO: 483 - DNA, 20 bp, Artificial Sequence
  Antisense Oligonucleotide
catagctcaccttgcacatt

SEQ ID NO: 484 - DNA, 20 bp, Artificial Sequence
  Antisense Oligonucleotide
cggtgcaccctttacctgag

SEQ ID NO: 485 - DNA, 20 bp, Artificial Sequence
  Antisense Oligonucleotide
tctccagatcctaacataaa

SEQ ID NO: 486 - DNA, 20 bp, Artificial Sequence
  Antisense Oligonucleotide
ttgaatgacactagattttc

SEQ ID NO: 487 - DNA, 20 bp, Artificial Sequence
  Antisense Oligonucleotide
aaaatccattttctttaaag

SEQ ID NO: 488 - DNA, 20 bp, Artificial Sequence
  Antisense Oligonucleotide
cagctcacacttattttaaa

SEQ ID NO: 489 - DNA, 20 bp, Artificial Sequence
  Antisense Oligonucleotide
gttcccaaaactgtatagga

SEQ ID NO: 490 - DNA, 20 bp, Artificial Sequence
  Antisense Oligonucleotide
agctccatactgaagtcctt

SEQ ID NO: 491 - DNA, 20 bp, Artificial Sequence
  Antisense Oligonucleotide
caattcaataaaagctccat

SEQ ID NO: 492 - DNA, 20 bp, Artificial Sequence
  Antisense Oligonucleotide
gttttcaaaaggtataaggt

SEQ ID NO: 493 - DNA, 20 bp, Artificial Sequence
  Antisense Oligonucleotide
ttcccattccctgaaagcag

SEQ ID NO: 494 - DNA, 20 bp, Artificial Sequence
  Antisense Oligonucleotide
tggtatttacctgagggctg

SEQ ID NO: 495 - DNA, 20 bp, Artificial Sequence
  Antisense Oligonucleotide
ataaataatagtgctgatgg

SEQ ID NO: 496 - DNA, 20 bp, Artificial Sequence
  Antisense Oligonucleotide
ctatggctgagcttgcctat

SEQ ID NO: 497 - DNA, 20 bp, Artificial Sequence
  Antisense Oligonucleotide
ctctctgaaaaatataccct

SEQ ID NO: 498 - DNA, 20 bp, Artificial Sequence
  Antisense Oligonucleotide
ttgatgtatctcatctagca

SEQ ID NO: 499 - DNA, 20 bp, Artificial Sequence
  Antisense Oligonucleotide
tagaaccatgtttggtcttc

SEQ ID NO: 500 - DNA, 20 bp, Artificial Sequence
  Antisense Oligonucleotide
tttctctttatcacatgccc

SEQ ID NO: 501 - DNA, 20 bp, Artificial Sequence
  Antisense Oligonucleotide
tatagtacactaaaacttca

SEQ ID NO: 502 - DNA, 20 bp, Artificial Sequence
  Antisense Oligonucleotide
ctggagaggactaaacagag

SEQ ID NO: 503 - DNA, 568 bp, H. sapiens
  misc_feature 44, 99, 156, 468: n = A,T,C or G
ccaaaagattgattgactgtccattcaaagctacacgcaatttntgatatacatcacgtagttactgaaaaagctgcaatcaacacagttcatggaccnctaccatgaagcttgctccaggagaacttctatcattcctctaattttttaaaaganatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcataccactgaatgtggctcatttgtantaaaagacagtgaaacgagggcattgataaaatgttctggcacagcaaaacctctagaacacatagtgtgatttaagtaacagaataaaaatggaaacgg

SEQ ID NO: 504 - DNA, 20 bp, Artificial Sequence
  Antisense Oligonucleotide
acattttatcaatgccctcg

SEQ ID NO: 505 - DNA, 20 bp, Artificial Sequence
  Antisense Oligonucleotide
gccagaacattttatcaatg

SEQ ID NO: 506 - DNA, 20 bp, Artificial Sequence
  Antisense Oligonucleotide
agaggttttgctgtgccaga

SEQ ID NO: 507 - DNA, 20 bp, Artificial Sequence
  Antisense Oligonucleotide
ctagaggttttgctgtgcca

SEQ ID NO: 508 - DNA, 20 bp, Artificial Sequence
  Antisense Oligonucleotide
tctagaggttttgctgtgcc

SEQ ID NO: 509 - DNA, 20 bp, Artificial Sequence
  Antisense Oligonucleotide
aatcacactatgtgttctag

SEQ ID NO: 510 - DNA, 20 bp, Artificial Sequence
  Antisense Oligonucleotide
aaatcacactatgtgttcta

SEQ ID NO: 511 - DNA, 20 bp, Artificial Sequence
  Antisense Oligonucleotide
taaatcacactatgtgttct

SEQ ID NO: 512 - DNA, 20 bp, Artificial Sequence
  Antisense Oligonucleotide
cttaaatcacactatgtgtt

SEQ ID NO: 513 - DNA, 20 bp, Artificial Sequence
  Antisense Oligonucleotide
tattctgttacttaaatcac

SEQ ID NO: 514 - DNA, 20 bp, Artificial Sequence
  Antisense Oligonucleotide
tggtagcctcagtctgcttc

SEQ ID NO: 515 - DNA, 20 bp, Artificial Sequence
  Antisense Oligonucleotide
agtctgcttcgcgccttctg

SEQ ID NO: 516 - DNA, 20 bp, H. sapiens
gcgccagggccgaagaggaa

SEQ ID NO: 517 - DNA, 20 bp, H. sapiens
caggtatgagctcaagctgg

SEQ ID NO: 518 - DNA, 20 bp, H. sapiens
catcctgaacatcaagaggg

SEQ ID NO: 519 - DNA, 20 bp, H. sapiens
gggcagtgtgatcgcttcaa

SEQ ID NO: 520 - DNA, 20 bp, H. sapiens
cacttgctctcatcaaaggc

SEQ ID NO: 521 - DNA, 20 bp, H. sapiens
cacactggacgctaagagga

SEQ ID NO: 522 - DNA, 20 bp, H. sapiens
cgctgagccacgcggtcaac

SEQ ID NO: 523 - DNA, 20 bp, H. sapiens
tgtccaaattctaccatggg

SEQ ID NO: 524 - DNA, 20 bp, H. sapiens
cagctgacctcatcgagatt

SEQ ID NO: 525 - DNA, 20 bp, H. sapiens
gtcaagttcctgatggtgtc

SEQ ID NO: 526 - DNA, 20 bp, H. sapiens
agctgcttctgatgggtgcc

SEQ ID NO: 527 - DNA, 20 bp, H. sapiens
gggcatcatcattccggact

SEQ ID NO: 528 - DNA, 20 bp, H. sapiens
cctactatccgctgaccggg

SEQ ID NO: 529 - DNA, 20 bp, H. sapiens
gggccacctaagttgtgaca

SEQ ID NO: 530 - DNA, 20 bp, H. sapiens
agaacatgggattgccagac

SEQ ID NO: 531 - DNA, 20 bp, H. sapiens
ctccacttcaagtctgtggg

SEQ ID NO: 532 - DNA, 20 bp, H. sapiens
cagagcttggcctctctggg

SEQ ID NO: 533 - DNA, 20 bp, H. sapiens
tggccgcttcagggaacaca

SEQ ID NO: 534 - DNA, 20 bp, H. sapiens
cagctgagcagacaggcacc

SEQ ID NO: 535 - DNA, 20 bp, H. sapiens
gggagagacaagtttcacat

SEQ ID NO: 536 - DNA, 20 bp, H. sapiens
gtactgcatcctggattcaa

SEQ ID NO: 537 - DNA, 20 bp, H. sapiens
gtgaggtgactcagagactc

SEQ ID NO: 538 - DNA, 20 bp, H. sapiens
ttgcagagcaatattctatc

SEQ ID NO: 539 - DNA, 20 bp, H. sapiens
aagcattggtagagcaaggg

SEQ ID NO: 540 - DNA, 20 bp, H. sapiens
ccgctggctctgaaggagtc

SEQ ID NO: 541 - DNA, 20 bp, H. sapiens
tctagtcaggctgacctgcg

SEQ ID NO: 542 - DNA, 20 bp, H. sapiens
gggccacagtgttctaactg

SEQ ID NO: 543 - DNA, 20 bp, H. sapiens
aatcaagtgtcatcacactg

SEQ ID NO: 544 - DNA, 20 bp, H. sapiens
gggtagtcataacagtactg

SEQ ID NO: 545 - DNA, 20 bp, H. sapiens
agagcacacggtcttcagtg

SEQ ID NO: 546 - DNA, 20 bp, H. sapiens
ttacagctagagggcctctt

SEQ ID NO: 547 - DNA, 20 bp, H. sapiens
caccgtgggcatggatatgg

SEQ ID NO: 548 - DNA, 20 bp, H. sapiens
gggaatctgatgaggaaact

SEQ ID NO: 549 - DNA, 20 bp, H. sapiens
tgtcaacaagtaccactggg

SEQ ID NO: 550 - DNA, 20 bp, H. sapiens
acctgggatatacactaggg

SEQ ID NO: 551 - DNA, 20 bp, H. sapiens
ccaagtatagttggctggac

SEQ ID NO: 552 - DNA, 20 bp, H. sapiens
tacatgaagcttgctccagg

SEQ ID NO: 553 - DNA, 20 bp, H. sapiens
atgtcagcctggtctgtcca

SEQ ID NO: 554 - DNA, 20 bp, H. sapiens
gcacctccggaagtacacat

SEQ ID NO: 555 - DNA, 20 bp, H. sapiens
ctgcagcttcatcctgaaga

SEQ ID NO: 556 - DNA, 20 bp, H. sapiens
tgagggcaaagccttgctga

SEQ ID NO: 557 - DNA, 20 bp, H. sapiens
ccattccagaagggaagcag

SEQ ID NO: 558 - DNA, 20 bp, H. sapiens
cgaggaagggcaatgtggca

SEQ ID NO: 559 - DNA, 20 bp, H. sapiens
ccttgtcaactctgatcagc

SEQ ID NO: 560 - DNA, 20 bp, H. sapiens
agcagccagtcctgtcagta

SEQ ID NO: 561 - DNA, 20 bp, H. sapiens
agcatgtggcagaagccatc

SEQ ID NO: 562 - DNA, 20 bp, H. sapiens
gagagcaccaaatccacatc

SEQ ID NO: 563 - DNA, 20 bp, H. sapiens
cctcagtgatgaagcagtca

SEQ ID NO: 564 - DNA, 20 bp, H. sapiens
gatagatgtggtcacctacc

SEQ ID NO: 565 - DNA, 20 bp, H. sapiens
cctcagcacagcagctgcga

SEQ ID NO: 566 - DNA, 20 bp, H. sapiens
gattctgcgggtcattggaa

SEQ ID NO: 567 - DNA, 20 bp, H. sapiens
caaagccatcactgatgatc

SEQ ID NO: 568 - DNA, 20 bp, H. sapiens
agaaagctgccatccaggct

SEQ ID NO: 569 - DNA, 20 bp, H. sapiens
caggaggttcttcttcagac

SEQ ID NO: 570 - DNA, 20 bp, H. sapiens
gagtccttcacaggcagata

SEQ ID NO: 571 - DNA, 20 bp, H. sapiens
tgccaatatcttgaactcag

SEQ ID NO: 572 - DNA, 20 bp, H. sapiens
catcgagattggcttggaag

SEQ ID NO: 573 - DNA, 20 bp, H. sapiens
ggagctggattacagttgca

SEQ ID NO: 574 - DNA, 20 bp, H. sapiens
caacatgcaggctgaactgg

SEQ ID NO: 575 - DNA, 20 bp, H. sapiens
acattacatttggtctctac

SEQ ID NO: 576 - DNA, 20 bp, H. sapiens
ctcaggcgcttactccaacg

SEQ ID NO: 577 - DNA, 20 bp, H. sapiens
gggacaccagattagagctg

SEQ ID NO: 578 - DNA, 20 bp, H. sapiens
gagctccagagagaggacag

SEQ ID NO: 579 - DNA, 20 bp, H. sapiens
atcggcagagtatgaccttg

SEQ ID NO: 580 - DNA, 20 bp, H. sapiens
caagggtgttatttccatac

SEQ ID NO: 581 - DNA, 20 bp, H. sapiens
gactcatctgctacagctta

SEQ ID NO: 582 - DNA, 20 bp, H. sapiens
gcaaatcctccagagatcta

SEQ ID NO: 583 - DNA, 20 bp, H. sapiens
ctctcctgggtgttctagac

SEQ ID NO: 584 - DNA, 20 bp, H. sapiens
atgaaggctgactctgtggt

SEQ ID NO: 585 - DNA, 20 bp, H. sapiens
gggaccacagatgtctgctt

SEQ ID NO: 586 - DNA, 20 bp, H. sapiens
ctggccggctcaatggagag

SEQ ID NO: 587 - DNA, 20 bp, H. sapiens
gctgcgttctgaatatcagg

SEQ ID NO: 588 - DNA, 20 bp, H. sapiens
tgctgacatcttaggcactg

SEQ ID NO: 589 - DNA, 20 bp, H. sapiens
aagtgtagtctcctggtgct

SEQ ID NO: 590 - DNA, 20 bp, H. sapiens
caaaattcagtctggatggg

SEQ ID NO: 591 - DNA, 20 bp, H. sapiens
gggaaactacggctagaacc

SEQ ID NO: 592 - DNA, 20 bp, H. sapiens
ctgcatgtggctggtaacct

SEQ ID NO: 593 - DNA, 20 bp, H. sapiens
ccatgaccatcgatgcacat

SEQ ID NO: 594 - DNA, 20 bp, H. sapiens
atgggaaactcgctctctgg

SEQ ID NO: 595 - DNA, 20 bp, H. sapiens
agtcatcatctcgtgtctag

SEQ ID NO: 596 - DNA, 20 bp, H. sapiens
gaatacagccaggacttgga

SEQ ID NO: 597 - DNA, 20 bp, H. sapiens
ggcgtggagcttactggacg

SEQ ID NO: 598 - DNA, 20 bp, H. sapiens
gagatgagagatgccgttga

SEQ ID NO: 599 - DNA, 20 bp, H. sapiens
agtcttgatgagcactatca

SEQ ID NO: 600 - DNA, 20 bp, H. sapiens
ctaagtaccaaatcagaatc

SEQ ID NO: 601 - DNA, 20 bp, H. sapiens
gtccatgagttaatcgagag

SEQ ID NO: 602 - DNA, 20 bp, H. sapiens
aggccacagttgcagtgtat

SEQ ID NO: 603 - DNA, 20 bp, H. sapiens
tctgattggtggactcttgc

SEQ ID NO: 604 - DNA, 20 bp, H. sapiens
gaagtcagtcttcaggctct

SEQ ID NO: 605 - DNA, 20 bp, H. sapiens
ccagattctcagatgaggga

SEQ ID NO: 606 - DNA, 20 bp, H. sapiens
catctgtcattgatgcactg

SEQ ID NO: 607 - DNA, 20 bp, H. sapiens
aggagatgtcaagggttcgg

SEQ ID NO: 608 - DNA, 20 bp, H. sapiens
ggaactattgctagtgaggc

SEQ ID NO: 609 - DNA, 20 bp, H. sapiens
ctctctccatggcaaatgtc

SEQ ID NO: 610 - DNA, 20 bp, H. sapiens
caccgtgacttcagtgcaga

SEQ ID NO: 611 - DNA, 20 bp, H. sapiens
actgagttgagggtccggga

SEQ ID NO: 612 - DNA, 20 bp, H. sapiens
cacatatgaactggacctgc

SEQ ID NO: 613 - DNA, 20 bp, H. sapiens
tctgaactcagaaggatggc

SEQ ID NO: 614 - DNA, 20 bp, H. sapiens
ggtgcgaagcagactgaggc

SEQ ID NO: 615 - DNA, 20 bp, H. sapiens
tcccaccgggacctgcgggg

SEQ ID NO: 616 - DNA, 20 bp, H. sapiens
caccgggacctgcggggctg

SEQ ID NO: 617 - DNA, 20 bp, H. sapiens
ctgagtgcccttctcggttg

SEQ ID NO: 618 - DNA, 20 bp, H. sapiens
ctcggttgctgccgctgagg

SEQ ID NO: 619 - DNA, 20 bp, H. sapiens
tcggttgctgccgctgagga

SEQ ID NO: 620 - DNA, 20 bp, H. sapiens
cggttgctgccgctgaggag

SEQ ID NO: 621 - DNA, 20 bp, H. sapiens
gttgctgccgctgaggagcc

SEQ ID NO: 622 - DNA, 20 bp, H. sapiens
ctgccgctgaggagcccgcc

SEQ ID NO: 623 - DNA, 20 bp, H. sapiens
accgcagctggcgatggacc

SEQ ID NO: 624 - DNA, 20 bp, H. sapiens
cagctggcgatggacccgcc

SEQ ID NO: 625 - DNA, 20 bp, H. sapiens
gaacttactatcatcctcta

SEQ ID NO: 626 - DNA, 20 bp, H. sapiens
tccaattgaactttcacata

SEQ ID NO: 627 - DNA, 20 bp, H. sapiens
aaaattcaaactgcctatat

SEQ ID NO: 628 - DNA, 20 bp, H. sapiens
gataaaaccatacagtgagc

SEQ ID NO: 629 - DNA, 20 bp, H. sapiens
ataaaaccatacagtgagcc

SEQ ID NO: 630 - DNA, 20 bp, H. sapiens
aaccatacagtgagccagcc

SEQ ID NO: 631 - DNA, 20 bp, H. sapiens
accatacagtgagccagcct

SEQ ID NO: 632 - DNA, 20 bp, H. sapiens
ccatacagtgagccagcctt

SEQ ID NO: 633 - DNA, 20 bp, H. sapiens
gtgagccagccttgcagtag

SEQ ID NO: 634 - DNA, 20 bp, H. sapiens
ccagccttgcagtaggcagt

SEQ ID NO: 635 - DNA, 20 bp, H. sapiens
taggcagtagactataagca

SEQ ID NO: 636 - DNA, 20 bp, H. sapiens
gcagtagactataagcagaa

SEQ ID NO: 637 - DNA, 20 bp, H. sapiens
tgaactggacctgcaccaaa

SEQ ID NO: 638 - DNA, 20 bp, H. sapiens
ctggacctgcaccaaagctg

SEQ ID NO: 639 - DNA, 20 bp, H. sapiens
ggacctgcaccaaagctggc

SEQ ID NO: 640 - DNA, 20 bp, H. sapiens
ctgcaccaaagctggcacca

SEQ ID NO: 641 - DNA, 20 bp, H. sapiens
accaaagctggcaccagggc

SEQ ID NO: 642 - DNA, 20 bp, H. sapiens
ctcggaaggtctctgaactc

SEQ ID NO: 643 - DNA, 20 bp, H. sapiens
aactcagaaggatggcattt

SEQ ID NO: 644 - DNA, 20 bp, H. sapiens
ctcagaaggatggcattttt

SEQ ID NO: 645 - DNA, 20 bp, H. sapiens
atcaggatctgagttatttt

SEQ ID NO: 646 - DNA, 20 bp, H. sapiens
aggatctgagttattttgct

SEQ ID NO: 647 - DNA, 20 bp, H. sapiens
ctgagttattttgctaaact

SEQ ID NO: 648 - DNA, 20 bp, H. sapiens
attttgctaaacttggggga

SEQ ID NO: 649 - DNA, 20 bp, H. sapiens
taaacttgggggaggaggaa

SEQ ID NO: 650 - DNA, 20 bp, H. sapiens
ggaacaaataaatggagtct

SEQ ID NO: 651 - DNA, 20 bp, H. sapiens
gtttgtaactcaagcagaag

SEQ ID NO: 652 - DNA, 20 bp, H. sapiens
ttgtaactcaagcagaaggt

SEQ ID NO: 653 - DNA, 20 bp, H. sapiens
gtaactcaagcagaaggtgc

SEQ ID NO: 654 - DNA, 20 bp, H. sapiens
aactcaagcagaaggtgcga

SEQ ID NO: 655 - DNA, 20 bp, H. sapiens
ctcaagcagaaggtgcgaag

SEQ ID NO: 656 - DNA, 20 bp, H. sapiens
caagcagaaggtgcgaagca

SEQ ID NO: 657 - DNA, 20 bp, H. sapiens
agcagaaggtgcgaagcaga

SEQ ID NO: 658 - DNA, 20 bp, H. sapiens
cagaaggtgcgaagcagact

SEQ ID NO: 659 - DNA, 20 bp, H. sapiens
gaaggtgcgaagcagactga

SEQ ID NO: 660 - DNA, 20 bp, H. sapiens
aggtgcgaagcagactgagg

SEQ ID NO: 661 - DNA, 20 bp, H. sapiens
gtgcgaagcagactgaggct

SEQ ID NO: 662 - DNA, 20 bp, H. sapiens
gcgaagcagactgaggctac

SEQ ID NO: 663 - DNA, 20 bp, H. sapiens
gaagcagactgaggctacca

SEQ ID NO: 664 - DNA, 20 bp, H. sapiens
agcagactgaggctaccatg

SEQ ID NO: 665 - DNA, 20 bp, H. sapiens
cagactgaggctaccatgac

SEQ ID NO: 666 - DNA, 20 bp, H. sapiens
gactgaggctaccatgacat

SEQ ID NO: 667 - DNA, 20 bp, H. sapiens
ctgaggctaccatgacattc

SEQ ID NO: 668 - DNA, 20 bp, H. sapiens
gaggctaccatgacattcaa

SEQ ID NO: 669 - DNA, 20 bp, H. sapiens
ggctaccatgacattcaaat

SEQ ID NO: 670 - DNA, 20 bp, H. sapiens
ctaccatgacattcaaatat

SEQ ID NO: 671 - DNA, 20 bp, H. sapiens
cctgaagctgcatgtggctg

SEQ ID NO: 672 - DNA, 20 bp, H. sapiens
tgaagctgcatgtggctggt

SEQ ID NO: 673 - DNA, 20 bp, H. sapiens
aagctgcatgtggctggtaa

SEQ ID NO: 674 - DNA, 20 bp, H. sapiens
gctgcatgtggctggtaacc

SEQ ID NO: 675 - DNA, 20 bp, H. sapiens
tgcatgtggctggtaaccta

SEQ ID NO: 676 - DNA, 20 bp, H. sapiens
catgtggctggtaacctaaa

SEQ ID NO: 677 - DNA, 20 bp, H. sapiens
tgtggctggtaacctaaaag

SEQ ID NO: 678 - DNA, 20 bp, H. sapiens
tggctggtaacctaaaagga

SEQ ID NO: 679 - DNA, 20 bp, H. sapiens
gctggtaacctaaaaggagc

SEQ ID NO: 680 - DNA, 20 bp, H. sapiens
tggtaacctaaaaggagcct

SEQ ID NO: 681 - DNA, 20 bp, H. sapiens
gtaacctaaaaggagcctac

SEQ ID NO: 682 - DNA, 20 bp, H. sapiens
aacctaaaaggagcctacca

SEQ ID NO: 683 - DNA, 20 bp, H. sapiens
cctaaaaggagcctaccaaa

SEQ ID NO: 684 - DNA, 20 bp, H. sapiens
ggcgcgaagcagactgaggc

SEQ ID NO: 685 - DNA, 20 bp, H. sapiens
cactatgttcatgagggagg

SEQ ID NO: 686 - DNA, 20 bp, H. sapiens
ccatcataggttctgacgtc

SEQ ID NO: 687 - DNA, 20 bp, H. sapiens
gaagctgattgactcactca

SEQ ID NO: 688 - DNA, 20 bp, H. sapiens
ttgtaactcaagcagaaggc

SEQ ID NO: 689 - DNA, 20 bp, H. sapiens
gtaactcaagcagaaggcgc

SEQ ID NO: 690 - DNA, 20 bp, H. sapiens
aactcaagcagaaggcgcga

SEQ ID NO: 691 - DNA, 20 bp, H. sapiens
ctcaagcagaaggcgcgaag

SEQ ID NO: 692 - DNA, 20 bp, H. sapiens
cagaaggcgcgaagcagact

SEQ ID NO: 693 - DNA, 20 bp, H. sapiens
gaaggcgcgaagcagactga

SEQ ID NO: 694 - DNA, 20 bp, H. sapiens
aggcgcgaagcagactgagg

SEQ ID NO: 695 - DNA, 20 bp, H. sapiens
gcgcgaagcagactgaggct

SEQ ID NO: 696 - DNA, 20 bp, H. sapiens
gaagcagactgaggctacca

SEQ ID NO: 697 - DNA, 20 bp, H. sapiens
cagaaggcgcgaagcagact

SEQ ID NO: 698 - DNA, 20 bp, H. sapiens
tctttctcctgtcttacaga

SEQ ID NO: 699 - DNA, 20 bp, H. sapiens
cccacgttagaagatgcgac

SEQ ID NO: 700 - DNA, 20 bp, H. sapiens
aatatggtagacatgagcca

SEQ ID NO: 701 - DNA, 20 bp, H. sapiens
cattagctgcatttcaactg

SEQ ID NO: 702 - DNA, 20 bp, H. sapiens
actttcactcctagtctgca

SEQ ID NO: 703 - DNA, 20 bp, H. sapiens
ccatgtccaggtaagtcatg

SEQ ID NO: 704 - DNA, 20 bp, H. sapiens
gcaccaggcacggatgtgac

SEQ ID NO: 705 - DNA, 20 bp, H. sapiens
gtggggtcccagaggcactg

SEQ ID NO: 706 - DNA, 20 bp, H. sapiens
gctgatcggccactgcagct

SEQ ID NO: 707 - DNA, 20 bp, H. sapiens
tccacgtggctggggaggtc

SEQ ID NO: 708 - DNA, 20 bp, H. sapiens
tgtaatgtatggtgatcaga

SEQ ID NO: 709 - DNA, 20 bp, H. sapiens
gagagtacccagtgggaaat

SEQ ID NO: 710 - DNA, 20 bp, H. sapiens
agtcagcatgggcttcagcc

SEQ ID NO: 711 - DNA, 20 bp, H. sapiens
caaaagaatgactgtccaac

SEQ ID NO: 712 - DNA, 20 bp, H. sapiens
gaatgactgtccaacaagtg

SEQ ID NO: 713 - DNA, 20 bp, H. sapiens
tatctactgtaatttaaaat

SEQ ID NO: 714 - DNA, 20 bp, H. sapiens
ctgatatgggtggagaacag

SEQ ID NO: 715 - DNA, 20 bp, H. sapiens
tctgggacaggtatgagctc

SEQ ID NO: 716 - DNA, 20 bp, H. sapiens
tgatagcagtggcccttgaa

SEQ ID NO: 717 - DNA, 20 bp, H. sapiens
ggattggcgtgaaatactgg

SEQ ID NO: 718 - DNA, 20 bp, H. sapiens
tgcccgaggttcctcctgcc

SEQ ID NO: 719 - DNA, 20 bp, H. sapiens
gcactagcaagaccacactc

SEQ ID NO: 720 - DNA, 20 bp, H. sapiens
caagaccacactctgcatag

SEQ ID NO: 721 - DNA, 20 bp, H. sapiens
tcctccataggataccgtgt

SEQ ID NO: 722 - DNA, 20 bp, H. sapiens
ggatgtagggcagcaaaacc

SEQ ID NO: 723 - DNA, 20 bp, H. sapiens
tctgcacaaggactccttgt

SEQ ID NO: 724 - DNA, 20 bp, H. sapiens
cagcctgtctcagtgaacat

SEQ ID NO: 725 - DNA, 20 bp, H. sapiens
caggatgcttccagtctaat

SEQ ID NO: 726 - DNA, 20 bp, H. sapiens
aaatgctcgtctccaatctc

SEQ ID NO: 727 - DNA, 20 bp, H. sapiens
aacttgtgtatccaaatcca

SEQ ID NO: 728 - DNA, 20 bp, H. sapiens
tgacatggtgtgcttccttg

SEQ ID NO: 729 - DNA, 20 bp, H. sapiens
acactggtgttctggctacc

SEQ ID NO: 730 - DNA, 20 bp, H. sapiens
gtgttctggctacctctagt

SEQ ID NO: 731 - DNA, 20 bp, H. sapiens
tcctggcataggtcacagta

SEQ ID NO: 732 - DNA, 20 bp, H. sapiens
atgtcaacagtagcacctcc

SEQ ID NO: 733 - DNA, 20 bp, H. sapiens
tagactcagacaagtctgga

SEQ ID NO: 734 - DNA, 20 bp, H. sapiens
cctaagttgctcatctctgg

SEQ ID NO: 735 - DNA, 20 bp, H. sapiens
tgcgctgagttccatgaaac

SEQ ID NO: 736 - DNA, 20 bp, H. sapiens
ctattgcaggtgctctccag

SEQ ID NO: 737 - DNA, 20 bp, H. sapiens
cagaggaacatcttgcacct

SEQ ID NO: 738 - DNA, 20 bp, H. sapiens
ctctgctccttactcttgtg

SEQ ID NO: 739 - DNA, 20 bp, H. sapiens
gtaatttctcaccatccatc

SEQ ID NO: 740 - DNA, 20 bp, H. sapiens
ttctgagtctcaattgtcta

SEQ ID NO: 741 - DNA, 20 bp, H. sapiens
ctatgtccttgtgtgcacat

SEQ ID NO: 742 - DNA, 20 bp, H. sapiens
gcctattgccatttgtatgt

SEQ ID NO: 743 - DNA, 20 bp, H. sapiens
ctattcatgtcctttgccta

SEQ ID NO: 744 - DNA, 20 bp, H. sapiens
gattctgcgggtaatctcag

SEQ ID NO: 745 - DNA, 20 bp, H. sapiens
tcaatgcaggtcattggaaa

SEQ ID NO: 746 - DNA, 20 bp, H. sapiens
ctaccagattgaccatccct

SEQ ID NO: 747 - DNA, 20 bp, H. sapiens
tacttgatagtgctctagga

SEQ ID NO: 748 - DNA, 20 bp, H. sapiens
ttgactgcaggaccaggagg

SEQ ID NO: 749 - DNA, 20 bp, H. sapiens
aacaaacacttgtgcaaatg

SEQ ID NO: 750 - DNA, 20 bp, H. sapiens
aatgagaccaaacttccact

SEQ ID NO: 751 - DNA, 20 bp, H. sapiens
ttccactttgaagctagcaa

SEQ ID NO: 752 - DNA, 20 bp, H. sapiens
gatctggagcttattcttga

SEQ ID NO: 753 - DNA, 20 bp, H. sapiens
acctcatgtgacttgtatgc

SEQ ID NO: 754 - DNA, 20 bp, H. sapiens
ttcttaagaaacaccttgta

SEQ ID NO: 755 - DNA, 20 bp, H. sapiens
taggcccatcctggctgcat

SEQ ID NO: 756 - DNA, 20 bp, H. sapiens
aaactctcaggatatggtaa

SEQ ID NO: 757 - DNA, 20 bp, H. sapiens
ataccttcctctacctttgc

SEQ ID NO: 758 - DNA, 20 bp, H. sapiens
tacctttgctgaaggtcctt

SEQ ID NO: 759 - DNA, 20 bp, H. sapiens
atctatctagtgaaatttct

SEQ ID NO: 760 - DNA, 20 bp, H. sapiens
tcagctcatcaaaatatgct

SEQ ID NO: 761 - DNA, 20 bp, H. sapiens
atatgctagtccttcctttc

SEQ ID NO: 762 - DNA, 20 bp, H. sapiens
caaaggtctgagttatccag

SEQ ID NO: 763 - DNA, 20 bp, H. sapiens
tgacttatagatgcaggctg

SEQ ID NO: 764 - DNA, 20 bp, H. sapiens
tcagtggagggtaattcttt

SEQ ID NO: 765 - DNA, 20 bp, H. sapiens
tgcctagccagtttgaaaga

SEQ ID NO: 766 - DNA, 20 bp, H. sapiens
cctgcagaattttgccaggc

SEQ ID NO: 767 - DNA, 20 bp, H. sapiens
gtagctaggtaggtaaagca

SEQ ID NO: 768 - DNA, 20 bp, H. sapiens
ttgagtgagacacacaaggt

SEQ ID NO: 769 - DNA, 20 bp, H. sapiens
gtgctagtcagggaatgcat

SEQ ID NO: 770 - DNA, 20 bp, H. sapiens
ggggagagagcatgcccagc

SEQ ID NO: 771 - DNA, 20 bp, H. sapiens
gcatgcccagctgcgaaagc

SEQ ID NO: 772 - DNA, 20 bp, H. sapiens
agccaggtatagaaaggagt

SEQ ID NO: 773 - DNA, 20 bp, H. sapiens
aactttctaagaggcagaat

SEQ ID NO: 774 - DNA, 20 bp, H. sapiens
tcttagtctggtcatgagtg

SEQ ID NO: 775 - DNA, 20 bp, H. sapiens
agtaggagatttcatatgaa

SEQ ID NO: 776 - DNA, 20 bp, H. sapiens
tcttcaccagcaacacatta

SEQ ID NO: 777 - DNA, 20 bp, H. sapiens
atggccacctagcatggcac

SEQ ID NO: 778 - DNA, 20 bp, H. sapiens
catgtttctgagcctccaga

SEQ ID NO: 779 - DNA, 20 bp, H. sapiens
taggtggctccctgtcttca

SEQ ID NO: 780 - DNA, 20 bp, H. sapiens
tccaaagtcttgggaatcct

SEQ ID NO: 781 - DNA, 20 bp, H. sapiens
acaaagaaagggggagttgg

SEQ ID NO: 782 - DNA, 20 bp, H. sapiens
tcgtgtcttcctggcccaga

SEQ ID NO: 783 - DNA, 20 bp, H. sapiens
gcagtgcccagcacacaata

SEQ ID NO: 784 - DNA, 20 bp, H. sapiens
actcgtccaggtgcgaagca

SEQ ID NO: 785 - DNA, 20 bp, H. sapiens
gccacctaaggtaaagaagg

SEQ ID NO: 786 - DNA, 20 bp, H. sapiens
atcagagtggcagagagagc

SEQ ID NO: 787 - DNA, 20 bp, H. sapiens
tttaccatagttgtgacaca

SEQ ID NO: 788 - DNA, 20 bp, H. sapiens
cattttgtaggcaatgagct

SEQ ID NO: 789 - DNA, 20 bp, H. sapiens
gcattagtaaacatgagaac

SEQ ID NO: 790 - DNA, 20 bp, H. sapiens
ttcatttcagcgatggccgg

SEQ ID NO: 791 - DNA, 20 bp, H. sapiens
gaaaatctagtgtcattcaa

SEQ ID NO: 792 - DNA, 20 bp, H. sapiens
tcctatacagttttgggaac

SEQ ID NO: 793 - DNA, 20 bp, H. sapiens
aaggacttcagtatggagct

SEQ ID NO: 794 - DNA, 20 bp, H. sapiens
atggagcttttattgaattg

SEQ ID NO: 795 - DNA, 20 bp, H. sapiens
ccatcagcactattatttat

SEQ ID NO: 796 - DNA, 20 bp, H. sapiens
ataggcaagctcagccatag

SEQ ID NO: 797 - DNA, 20 bp, H. sapiens
tgctagatgagatacatcaa

SEQ ID NO: 798 - DNA, 20 bp, H. sapiens
gaagaccaaacatggttcta

SEQ ID NO: 799 - DNA, 20 bp, H. sapiens
ctctgtttagtcctctccag

SEQ ID NO: 800 - DNA, 20 bp, H. sapiens
cattgataaaatgttctggc

SEQ ID NO: 801 - DNA, 20 bp, H. sapiens
tctggcacagcaaaacctct

SEQ ID NO: 802 - DNA, 20 bp, H. sapiens
tggcacagcaaaacctctag

SEQ ID NO: 803 - DNA, 20 bp, H. sapiens
tagaacacatagtgtgattt

SEQ ID NO: 804 - DNA, 20 bp, H. sapiens
aacacatagtgtgatttaag

SEQ ID NO: 805 - DNA, 20 bp, Artificial Sequence
  Antisense Oligonucleotide
ctttccgttggacccctggg

SEQ ID NO: 806 - DNA, 20 bp, Artificial Sequence
  Antisense Oligonucleotide
tcccgcctgtgacatgcatt

SEQ ID NO: 807 - DNA, 20 bp, Artificial Sequence
  Antisense Oligonucleotide
ttctacctcgcgcgatttac

SEQ ID NO: 808 - DNA, 432 bp, O. cuniculus
gatcttaccttctccaagcaaaatgcattgctacgtgctgagtatcaggctgattacaagtcactgaggttcttcaccctgctttctgggttgttgaatacccatggtcttgaattaaatgctgacatcttgggcactgacaaaatgaatactgctgctcacaaggcaactctaagaattggccaaaatggagtatctaccagtgcaacaaccagcttgaggtacagtcccctgatgctggagaatgagctgaacgcagagcttgccctttctggggcatctatgaaattagcaacaaatggccgcttcaaggaacacaatgcaaaattcagcctagatgggaaagctaccctcacagagttatccctgggaagcgcttaccaggccatgattctgggtgctgacagcaagaacattttcaacttcaagatc

SEQ ID NO: 809 - DNA, 660 bp, O. cuniculus
ctgggaaaactcccacagcaagttaatgattatctgagtacattcaattgggagagacaagtttccagtgccaaggagaaactaactactttcacaaaaaattataaaattacagagaatgatatacaaactgcattggataatgccaaaatcaacttaaatgaaaaactgtctcaacttcagacatatgtgatataatttgatcagtatattaaagataattttgatctacatgattttaaaatagctatagctagtattatagatcaaatcatggaaaaattaaaaattcttgatgaacgttatcatatccgtgcacatttaattaaatcaatccataatttatatttgtttattgaagctattgattttaacaaaattggaagtagtactgcatcttggattcaaaatgtggataccaagtatcaagtcagaatctggatacaagaaatattgcaacagtttaagacacagattcagaatacaaacatcccatacctggctgaaaaactgaaacaacagattgaggctattgatgtcagagtgcttttagatcaattgagaactacaattccatttcgtataataaaggacattattgaacatttcaaatactttgttataaatattattgaaaattttgaagtaattgacaaaatc

SEQ ID NO: 810 - DNA, 543 bp, O. cuniculus
  misc_feature (45): n = a, c, g, or t
  misc_feature (118): n = a, c, g, or t
  misc_feature (148): n = a, c, g, or t
  misc_feature (173): n = a, c, g, or t
  misc_feature (180): n = a, c, g, or t
cagaacatcggagacaacgcattggattttctcactaaatcttanaatgaagcaaaaattaagtttgataagtacaaagttgaaaaatcgctcaacaggctccccaggacctttcagnctcctggatacattattccaattttcaatnttgaagtatctccactcacaatagnagacgtnagcattcagtcatgtgatcccaaaatcaataagcacccccaatgtcaccatcctggattcaagcttctatgtgccttcatatacattggctctgccatccctagagctgccagtcttccatgtccccaggaatctactcaaggtctctcttccagatttcaaggaattgaaaaccattaacaatatttttattccagccatgggcaacattacctatgaattttccttcaaatcaacgatcattacactgaataccaatgctggactttataaccaatcagacattgttgcccatatcctttcttcctcttcatctgtcattgatgcactacagtacaaattagagggcacgctcaagtttga

SEQ ID NO: 811 - DNA, 19 bp, Artificial Sequence
  Primer
aagcacccccaatgtcacc

SEQ ID NO: 812 - DNA, 20 bp, Artificial Sequence
  Primer
gggatggcagagccaatgta

SEQ ID NO: 813 - DNA, 29 bp, Artificial Sequence
  Probe
tcctggattcaagcttctatgtgccttca

SEQ ID NO: 814 - DNA, 20 bp, Artificial Sequence
  Antisense Oligonucleotide
tgcttggagaaggtaagatc

SEQ ID NO: 815 - DNA, 20 bp, Artificial Sequence
  Antisense Oligonucleotide
gcgttgtctccgatgttctg

SEQ ID NO: 816 - DNA, 20 bp, Artificial Sequence
  Antisense Oligonucleotide
taatcattaacttgctgtgg

SEQ ID NO: 817 - DNA, 20 bp, Artificial Sequence
  Antisense Oligonucleotide
tcagcacgtagcaatgcatt

SEQ ID NO: 818 - DNA, 20 bp, Artificial Sequence
  Antisense Oligonucleotide
gcctgatactcagcacgtag

SEQ ID NO: 819 - DNA, 20 bp, Artificial Sequence
  Antisense Oligonucleotide
caattgaatgtactcagata

SEQ ID NO: 820 - DNA, 20 bp, Artificial Sequence
  Antisense Oligonucleotide
acctcagtgacttgtaatca

SEQ ID NO: 821 - DNA, 20 bp, Artificial Sequence
  Antisense Oligonucleotide
cactggaaacttgtctctcc

SEQ ID NO: 822 - DNA, 20 bp, Artificial Sequence
  Antisense Oligonucleotide
agtagttagtttctccttgg

SEQ ID NO: 823 - DNA, 20 bp, Artificial Sequence
  Antisense Oligonucleotide
tcagtgcccaagatgtcagc

SEQ ID NO: 824 - DNA, 20 bp, Artificial Sequence
  Antisense Oligonucleotide
attggaataatgtatccagg

SEQ ID NO: 825 - DNA, 20 bp, Artificial Sequence
  Antisense Oligonucleotide
ttggcattatccaatgcagt

SEQ ID NO: 826 - DNA, 20 bp, Artificial Sequence
  Antisense Oligonucleotide
gttgccttgtgagcagcagt

SEQ ID NO: 827 - DNA, 20 bp, Artificial Sequence
  Antisense Oligonucleotide
attgtgagtggagatacttc

SEQ ID NO: 828 - DNA, 20 bp, Artificial Sequence
  Antisense Oligonucleotide
catatgtctgaagttgagac

SEQ ID NO: 829 - DNA, 20 bp, Artificial Sequence
  Antisense Oligonucleotide
gtagatactccattttggcc

SEQ ID NO: 830 - DNA, 20 bp, Artificial Sequence
  Antisense Oligonucleotide
ggatcacatgactgaatgct

SEQ ID NO: 831 - DNA, 20 bp, Artificial Sequence
  Antisense Oligonucleotide
tcaagctggttgttgcactg

SEQ ID NO: 832 - DNA, 20 bp, Artificial Sequence
  Antisense Oligonucleotide
ggactgtacctcaagctggt

SEQ ID NO: 833 - DNA, 20 bp, Artificial Sequence
  Antisense Oligonucleotide
gctcattctccagcatcagg

SEQ ID NO: 834 - DNA, 20 bp, Artificial Sequence
  Antisense Oligonucleotide
ttgatctataatactagcta

SEQ ID NO: 835 - DNA, 20 bp, Artificial Sequence
  Antisense Oligonucleotide
atggaagactggcagctcta

SEQ ID NO: 836 - DNA, 20 bp, Artificial Sequence
  Antisense Oligonucleotide
ttgtgttccttgaagcggcc

SEQ ID NO: 837 - DNA, 20 bp, Artificial Sequence
  Antisense Oligonucleotide
tgtgcacggatatgataacg

SEQ ID NO: 838 - DNA, 20 bp, Artificial Sequence
  Antisense Oligonucleotide
gaccttgagtagattcctgg

SEQ ID NO: 839 - DNA, 20 bp, Artificial Sequence
  Antisense Oligonucleotide
gaaatctggaagagagacct

SEQ ID NO: 840 - DNA, 20 bp, Artificial Sequence
  Antisense Oligonucleotide
gtagctttcccatctaggct

SEQ ID NO: 841 - DNA, 20 bp, Artificial Sequence
  Antisense Oligonucleotide
gataactctgtgagggtagc

SEQ ID NO: 842 - DNA, 20 bp, Artificial Sequence
  Antisense Oligonucleotide
atgttgcccatggctggaat

SEQ ID NO: 843 - DNA, 20 bp, Artificial Sequence
  Antisense Oligonucleotide
aagatgcagtactacttcca

SEQ ID NO: 844 - DNA, 20 bp, Artificial Sequence
  Antisense Oligonucleotide
gcacccagaatcatggcctg

SEQ ID NO: 845 - DNA, 20 bp, Artificial Sequence
  Antisense Oligonucleotide
cttgatacttggtatccaca

SEQ ID NO: 846 - DNA, 20 bp, Artificial Sequence
  Antisense Oligonucleotide
cagtgtaatgatcgttgatt

SEQ ID NO: 847 - DNA, 20 bp, Artificial Sequence
  Antisense Oligonucleotide
taaagtccagcattggtatt

SEQ ID NO: 848 - DNA, 20 bp, Artificial Sequence
  Antisense Oligonucleotide
caacaatgtctgattggtta

SEQ ID NO: 849 - DNA, 20 bp, Artificial Sequence
  Antisense Oligonucleotide
gaagaggaagaaaggatatg

SEQ ID NO: 850 - DNA, 20 bp, Artificial Sequence
  Antisense Oligonucleotide
tgacagatgaagaggaagaa

SEQ ID NO: 851 - DNA, 20 bp, Artificial Sequence
  Antisense Oligonucleotide
ttgtactgtagtgcatcaat

SEQ ID NO: 852 - DNA, 20 bp, Artificial Sequence
  Antisense Oligonucleotide
gcctcaatctgttgtttcag

SEQ ID NO: 853 - DNA, 20 bp, Artificial Sequence
  Antisense Oligonucleotide
acttgagcgtgccctctaat

SEQ ID NO: 854 - DNA, 20 bp, Artificial Sequence
  Antisense Oligonucleotide
gaaatggaattgtagttctc

SEQ ID NO: 855 - DNA, 479 bp, M. fascicularis
  misc_feature (7): n = A,T,C or G
  misc_feature (469)..(474): n = A,T,C or G
  misc_feature (476)..( 479): n = A,T,C or G
tgcgtcnagactccgccccctactatccgctgaccggggacaccagattagagctggaactgaggcctacaggagaagttgagcagtattctgtcagtgcaacctatgagctccagagagaggacagagccttggtggacaccctgaagtttgtaactcaagcagaaggtgtaaagcagactgaggctaccatgacattcaaatataatcggcagagtatgaccttgtccagtgaagtccaaattccggattttgaggttgaccttggaacaatcctcagagttaatgatgaatctactgagggcagaaagtcttacagactcaccctggacattcagaaccagaaaattactgaggtcaccctcatgggccacctaagttgtgacacaaaggaagaaggaaaaatcaaaggtgttatttccgtaccccgtttgcaagcagaagccagaagtgagatcctcgcccacannnnnnannnn

SEQ ID NO: 856 - DNA, 20 bp, Artificial Sequence
  Antisense Oligonucleotide
gtccctcaacatctgaatgc

SEQ ID NO: 857 - DNA, 20 bp, Artificial Sequence
  Antisense Oligonucleotide
ctgctagcctctggatttga

SEQ ID NO: 858 - DNA, 20 bp, Artificial Sequence
  Antisense Oligonucleotide
ccttccctgaaggttcctcc

SEQ ID NO: 859 - DNA, 20 bp, Artificial Sequence
  Antisense Oligonucleotide
ctcttactgtgctgtggaca

SEQ ID NO: 860 - DNA, 13938 bp, H. sapiens
ctgggattgggacacactttctggacactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 861 - DNA, 25 bp, Artificial Sequence
  PCR Primer
cagctccttattgttatacgaggga

SEQ ID NO: 862 - DNA, 18 bp, Artificial Sequence
  PCR Primer
tgcgtctgagcattgcgt

SEQ ID NO: 863 - DNA, 24 bp, Artificial Sequence
  PCR Probe
cccggtgtcaggtgggagtactgc

SEQ ID NO: 864 - DNA, 20 bp, Artificial Sequence
  Antisense Oligonucleotide
gcctcagtcttcttcgcacc

SEQ ID NO: 865 - DNA, 20 bp, Artificial Sequence
  Antisense Oligonucleotide
gcctcagtcttattcgcacc

SEQ ID NO: 866 - DNA, 20 bp, Artificial Sequence
  Antisense Oligonucleotide
gcctcagtattattcgcacc

SEQ ID NO: 867 - DNA, 20 bp, Artificial Sequence
  Antisense Oligonucleotide
gcctcattattattcgcacc

SEQ ID NO: 868 - DNA, 20 bp, Artificial Sequence
  Antisense Oligonucleotide
gcctcattattattagcacc

SEQ ID NO: 869 - DNA, 20 bp, Artificial Sequence
  Antisense Oligonucleotide
gcctcattattattatcacc

SEQ ID NO: 870 - DNA, 20 bp, Artificial Sequence
  Antisense Oligonucleotide
gcctaattattattatcacc

SEQ ID NO: 871 - DNA, 19 bp, Artificial Sequence
  Antisense Oligonucleotide
gcctcagtctgcttcgcac

SEQ ID NO: 872 - DNA, 18 bp, Artificial Sequence
  Antisense Oligonucleotide
gcctcagtctgcttcgca

SEQ ID NO: 873 - DNA, 15 bp, Artificial Sequence
  Antisense Oligonucleotide
gcctcagtctgcttc

SEQ ID NO: 874 - DNA, 18 bp, Artificial Sequence
  Antisense Oligonucleotide
cctcagtctgcttcgcac

SEQ ID NO: 875 - DNA, 16 bp, Artificial Sequence
  Antisense Oligonucleotide
ctcagtctgcttcgca

SEQ ID NO: 876 - DNA, 14 bp, Artificial Sequence
  Antisense Oligonucleotide
tcagtctgcttcgc

SEQ ID NO: 877 - DNA, 10 bp, Artificial Sequence
  Antisense Oligonucleotide
gcctcagtct

SEQ ID NO: 878 - DNA, 10 bp, Artificial Sequence
  Antisense Oligonucleotide
gcttcgcacc

SEQ ID NO: 879 - RNA, 20 nt, Artificial Sequence
  Antisense Oligonucleotide
uugaagccauacaccucuuu

SEQ ID NO: 880 - RNA, 20 nt, Artificial Sequence
  Antisense Oligonucleotide
ugaccaggacugccuguucu

SEQ ID NO: 881 - RNA, 20 nt, Artificial Sequence
  Antisense Oligonucleotide
gaauagggcuguagcuguaa

SEQ ID NO: 882 - RNA, 20 nt, Artificial Sequence
  Antisense Oligonucleotide
uauacugaucaaauuguauc

SEQ ID NO: 883 - RNA, 20 nt, Artificial Sequence
  Antisense Oligonucleotide
uggaauucugguaugugaag

SEQ ID NO: 884 - RNA, 20 nt, Artificial Sequence
  Antisense Oligonucleotide
aaaucaaaugauugcuuugu

SEQ ID NO: 885 - RNA, 20 nt, Artificial Sequence
  Antisense Oligonucleotide
gugaugacacuugauuuaaa

SEQ ID NO: 886 - RNA, 20 nt, Artificial Sequence
  Antisense Oligonucleotide
gaagcugccucuucuuccca

SEQ ID NO: 887 - RNA, 20 nt, Artificial Sequence
  Antisense Oligonucleotide
gagaguuggucugaaaaauc

SEQ ID NO: 888 - DNA, 20 bp, Artificial Sequence
  Antisense Oligonucleotide
gtgcgcgcgagcccgaaatc

SEQ ID NO: 889 - DNA, 21 bp, Artificial Sequence
  Antisense Oligonucleotide
cuucuggcauccgguuuagtt

SEQ ID NO: 890 - DNA, 466 bp, M. fascicularis
  misc_feature 9: n = A,T,C or G
ggatcggcngaccctgagctgcatatggctggtaatctaaaaggagcctaccaaaataatgaaataaaacacatctataccatctcttctgctgccttatcagcaagctacaaagcagacactgttgctaaggttcagggtgtggagtttagccatcggctcaacacagacatcgctgggctggcttcagccattgacattagcacaaactataattcagactcattgcatttcagcaatgtcttccattctgtaatggctccatttaccatgaccattgatacacatacaaatggcaacgggaaacttgttctctggggagaacatactgggcagctgtatagcaaattcctgttgaaagcagaacctctggcattcactttctctcatgattacaaaggctccacgagtcatcatctcatgtctaggaaaagcatcagtgcagctcttgaacacaaagtcagta

SEQ ID NO: 891 - DNA, 20 bp, Artificial Sequence
  Antisense Oligonucleotide
gcctcagtctgctttacacc

SEQ ID NO: 892 - DNA, 20 bp, Artificial Sequence
  Antisense Oligonucleotide
agattaccagccatatgcag

(19) United States
(12) Patent Application Publication
(10) Pub. No.: US 2018/0094260 A1
(43) Pub. Date: Apr. 5, 2018

(54) EFFECTS OF APOLIPOPROTEIN B INHIBITION ON GENE EXPRESSION PROFILES IN ANIMALS

(71) Applicant: Kastle Therapeutics, LLC, Chicago, IL (US)

(72) Inventors: Rosanne M. Crooke, Carlsbad, CA (US); Mark J. Graham, San Clemente, CA (US); Susan M. Freier, San Diego, CA (US)

(73) Assignee: Kastle Therapeutics, LLC, Chicago, IL (US)

(21) Appl. No.: 15/589,965

(22) Filed: May 8, 2017

Related U.S. Application Data
(63) Continuation of application No. 14/500,729, filed on Sep. 29, 2014, now abandoned.
Application No. 14/500,729 is a continuation of application No. 12/720,581, filed on Mar. 9, 2010, now abandoned.
Application No. 12/720,581 is a continuation of application No. 11/123,656, filed on May 5, 2005, now abandoned.
Application No. 11/123,656 is a continuation-in-part of application No. 10/712,795, filed on Nov. 13, 2003, now Pat. No. 7,511,131.
(60) Provisional application No. 60/568,825, filed on May 5, 2004.
(60) Provisional application No. 60/426,234, filed on Nov. 13, 2002.

(51) Int. Cl. C12N 15/113; A61K 38/00

(52) U.S. Cl. CPC C12N 15/113; C12N 2310/3525; C12N 2310/346; C12N 2310/341; C12N 2310/3341; C12N 2310/321; C12N 2310/315; C12N 2310/14; C12N 2310/13; A61K 38/00; Y02P 20/582

(57) ABSTRACT

Antisense compounds, compositions and methods are provided for modulating the expression of apolipoprotein B. The compositions comprise antisense compounds, particularly antisense oligonucleotides, targeted to nucleic acids encoding apolipoprotein B. Methods of using these compounds for modulation of apolipoprotein B expression and for treatment of diseases associated with expression of apolipoprotein B are provided. Methods are provided for modulating the expression of genes involved in lipid metabolism, useful in the treatment of conditions associated with cardiovascular risk. Antisense oligonucleotides targeted to apolipoprotein B reduce the level of apolipoprotein B mRNA, lower serum cholesterol and shift liver gene expression profiles from those of an obese animal towards those of a lean animal. Further provided are methods for improving the cardiovascular risk of a subject through antisense inhibition of apolipoprotein B. Also provided are methods for employing antisense oligonucleotides targeted to apolipoprotein B to modulate a cellular pathway or metabolic process.

### CROSS REFERENCE TO RELATED APPLICATIONS

This application is a continuation of U.S. application Ser. No. 12/720,581 filed Mar. 9, 2010, which is a continuation of U.S. application Ser. No. 11/123,656, filed May 5, 2005, which is a continuation-in-part of U.S. application Ser. No. 10/712,795, filed Nov. 13, 2003 (now U.S. Pat. No. 7,511,131), each of which is incorporated by reference herein in its entirety. U.S. application Ser. No. 11/123,656 also claims the benefit of priority of U.S. provisional application No. 60/568,825, filed May 5, 2004, which is incorporated by reference herein in its entirety.

### SEQUENCE LISTING

The present specification is being filed with a computer readable form (CRF) copy of the Sequence Listing. The CRF entitled 10103-062-999_SEQ LIST.txt, which was created on Sep. 29, 2014 and is 343,116 bytes in size, is identical to the paper copy of the Sequence Listing and is incorporated herein by reference in its entirety.

### FIELD OF THE INVENTION

The present invention provides compositions and methods for modulating the expression of apolipoprotein B. In particular, this invention relates to compounds, particularly oligonucleotides, specifically hybridizable with nucleic acids encoding apolipoprotein B. Such compounds have been shown to modulate the expression of apolipoprotein B. The present invention provides methods for modulating the expression of genes involved in lipid metabolism. In particular, this invention relates to the modulation of such genes following the antisense inhibition of apolipoprotein B, which has been shown to improve lipid profiles in animals. The invention also provides methods lowering the cardiovascular risk profile of an animal.

### BACKGROUND OF THE INVENTION

Lipoproteins are globular, micelle-like particles that consist of a non-polar core of acylglycerols and cholesteryl esters surrounded by an amphiphilic coating of protein, phospholipid and cholesterol. Lipoproteins have been classified into five broad categories on the basis of their functional and physical properties: chylomicrons, which transport dietary lipids from intestine to tissues; very low density lipoproteins (VLDL); intermediate density lipoproteins (IDL); low density lipoproteins (LDL); all of which transport triacylglycerols and cholesterol from the liver to tissues; and high density lipoproteins (HDL), which transport endogenous cholesterol from tissues to the liver.

Lipoprotein particles undergo continuous metabolic processing and have variable properties and compositions. Lipoprotein densities increase without decreasing particle diameter because the density of their outer coatings is less than that of the inner core. The protein components of lipoproteins are known as apoliproteins. At least nine apolipoproteins are distributed in significant amounts among the various human lipoproteins.

Apolipoprotein B (also known as ApoB, apolipoprotein B-100; ApoB-100, apolipoprotein B-48; ApoB-48 and Ag(x) antigen), is a large glycoprotein that serves an indispensable role in the assembly and secretion of lipids and in the transport and receptor-mediated uptake and delivery of distinct classes of lipoproteins. The importance of apolipoprotein B spans a variety of functions, from the absorption and processing of dietary lipids to the regulation of circulating lipoprotein levels (Davidson and Shelness, Annu. Rev. Nutr., 2000, 20, 169-193). This latter property underlies its relevance in terms of atherosclerosis susceptibility, which is highly correlated with the ambient concentration of apolipoprotein B-containing lipoproteins (Davidson and Shelness, Annu. Rev. Nutr., 2000, 20, 169-193).

Two forms of apolipoprotein B exist in mammals. ApoB-100 represents the full-length protein containing 4536 amino acid residues synthesized exclusively in the human liver (Davidson and Shelness, Annu. Rev. Nutr., 2000, 20, 169-193). A truncated form known as ApoB-48 is collinear with the amino terminal 2152 residues and is synthesized in the small intestine of all mammals (Davidson and Shelness, Annu. Rev. Nutr., 2000, 20, 169-193).

ApoB-100 is the major protein component of LDL and contains the domain required for interaction of this lipoprotein species with the LDL receptor. In addition, ApoB-100 contains an unpaired cysteine residue which mediates an interaction with apolipoprotein(a) and generates another distinct atherogenic lipoprotein called Lp(a) (Davidson and Shelness, Annu. Rev. Nutr., 2000, 20, 169-193).

In humans, ApoB-48 circulates in association with chylomicrons and chylomicron remnants and these particles are cleared by a distinct receptor known as the LDL-receptor-related protein (Davidson and Shelness, Annu. Rev. Nutr., 2000, 20, 169-193). ApoB-48 can be viewed as a crucial adaptation by which dietary lipid is delivered from the small intestine to the liver, while ApoB-100 participates in the transport and delivery of endogenous plasma cholesterol (Davidson and Shelness, Annu. Rev. Nutr., 2000, 20, 169-193).

The basis by which the common structural gene for apolipoprotein B produces two distinct protein isoforms is a process known as RNA editing. A site specific cytosine-to-uracil editing reaction produces a UAA stop codon and translational termination of apolipoprotein B to produce ApoB-48 (Davidson and Shelness, Annu. Rev. Nutr., 2000, 20, 169-193).

Apolipoprotein B was cloned in 1985 (Law et al., Proc. Natl. Acad. Sci. U.S.A., 1985, 82, 8340-8344) and mapped to chromosome 2p23-2p24 in 1986 (Deeb et al., Proc. Natl. Acad. Sci. U.S.A., 1986, 83, 419-422).

Disclosed and claimed in U.S. Pat. No. 5,786,206 are methods and compositions for determining the level of low density lipoproteins (LDL) in plasma which include isolated DNA sequences encoding epitope regions of apolipoprotein B-100 (Smith et al., 1998).

Transgenic mice expressing human apolipoprotein B and fed a high-fat diet were found to develop high plasma cholesterol levels and displayed an 11-fold increase in atherosclerotic lesions over non-transgenic littermates (Kim and Young, J. Lipid Res., 1998, 39, 703-723; Nishina et al., J. Lipid Res., 1990, 31, 859-869).

In addition, transgenic mice expressing truncated forms of human apolipoprotein B have been employed to identify the carboxyl-terminal structural features of ApoB-100 that are required for interactions with apolipoprotein(a) to generate the Lp(a) lipoprotein particle and to investigate structural features of the LDL receptor-binding region of ApoB-100 (Kim and Young, J. Lipid Res., 1998, 39, 703-723; McCormick et al., J. Biol. Chem., 1997, 272, 23616-23622).

Apolipoprotein B knockout mice (bearing disruptions of both ApoB-100 and ApoB-48) have been generated which are protected from developing hypercholesterolemia when fed a high-fat diet (Farese et al., Proc. Natl. Acad. Sci. U.S.A., 1995, 92, 1774-1778; Kim and Young, J. Lipid Res., 1998, 39, 703-723). The incidence of atherosclerosis has been investigated in mice expressing exclusively ApoB-100 or ApoB-48 and susceptibility to atherosclerosis was found to be dependent on total cholesterol levels. Whether the mice synthesized ApoB-100 or ApoB-48 did not affect the extent of the atherosclerosis, indicating that there is probably no major difference in the intrinsic atherogenicity of ApoB-100 versus ApoB-48 (Kim and Young, J. Lipid Res., 1998, 39, 703-723; Veniant et al., J. Clin. Invest., 1997, 100, 180-188).

Elevated plasma levels of the ApoB-100-containing lipoprotein Lp(a) are associated with increased risk for atherosclerosis and its manifestations, which may include hypercholesterolemia (Seed et al., N. Engl. J. Med., 1990, 322, 1494-1499), myocardial infarction (Sandkamp et al., Clin. Chem., 1990, 36, 20-23), and thrombosis (Nowak-Gottl et al., Pediatrics, 1997, 99, E11).

The plasma concentration of Lp(a) is strongly influenced by heritable factors and is refractory to most drug and dietary manipulation (Katan and Beynen, Am. J. Epidemiol., 1987, 125, 387-399; Vessby et al., Atherosclerosis, 1982, 44, 61-71). Pharmacologic therapy of elevated Lp(a) levels has been only modestly successful and apheresis remains the most effective therapeutic modality (Hajjar and Nachman, Annu. Rev. Med., 1996, 47, 423-442).

Disclosed and claimed in U.S. Pat. No. 6,156,315 and the corresponding PCT publication WO 99/18986 is a method for inhibiting the binding of LDL to blood vessel matrix in a subject, comprising administering to the subject an effective amount of an antibody or a fragment thereof, which is capable of binding to the amino-terminal region of apolipoprotein B, thereby inhibiting the binding of low density lipoprotein to blood vessel matrix (Goldberg and Pillarisetti, 2000; Goldberg and Pillarisetti, 1999).

Disclosed and claimed in U.S. Pat. No. 6,096,516 are vectors containing cDNA encoding murine recombinant antibodies which bind to human ApoB-100 for the purpose of for diagnosis and treatment of cardiovascular diseases (Kwak et al., 2000).

Disclosed and claimed in European patent application EP 911344 published Apr. 28, 1999 (and corresponding to U.S. Pat. No. 6,309,844) is a monoclonal antibody which specifically binds to ApoB-48 and does not specifically bind to ApoB-100, which is useful for diagnosis and therapy of hyperlipidemia and arterial sclerosis (Uchida and Kurano, 1998).

Disclosed and claimed in PCT publication WO 01/30354 are methods of treating a patient with a cardiovascular disorder, comprising administering a therapeutically effective amount of a compound to said patient, wherein said compound acts for a period of time to lower plasma concentrations of apolipoprotein B or apolipoprotein B-containing lipoproteins by stimulating a pathway for apolipoprotein B degradation (Fisher and Williams, 2001).

Disclosed and claimed in U.S. Pat. No. 5,220,006 is a cloned cis-acting DNA sequence that mediates the suppression of atherogenic apolipoprotein B (Ross et al., 1993).

Disclosed and claimed in PCT publication WO 01/12789 is a ribozyme which cleaves ApoB-100 mRNA specifically at position 6679 (Chan et al., 2001).

To date, strategies aimed at inhibiting apolipoprotein B function have been limited to Lp(a) apheresis, antibodies, antibody fragments and ribozymes. However, with the exception of Lp(a) apheresis, these investigative strategies are untested as therapeutic protocols. Consequently, there remains a long felt need for additional agents capable of effectively inhibiting apolipoprotein B function.

Antisense technology is an effective means of reducing the expression of specific gene products and may therefore prove to be uniquely useful in a number of therapeutic, diagnostic and research applications involving modulation of apolipoprotein B expression.

The present invention provides compositions and methods for modulating apolipoprotein B expression, including inhibition of the alternative isoform of apolipoprotein B, ApoB-48.

### SUMMARY OF THE INVENTION

The present invention is directed to compounds, particularly antisense oligonucleotides, which are targeted to a nucleic acid encoding apolipoprotein B, and which modulate the expression of apolipoprotein B. Pharmaceutical and other compositions comprising the compounds of the invention are also provided. Further provided are methods of modulating the expression of apolipoprotein B in cells or tissues comprising contacting said cells or tissues with one or more of the antisense compounds or compositions of the invention. Further provided are methods of treating an animal, particularly a human, suspected of having or being prone to a disease or condition associated with expression of apolipoprotein B by administering a therapeutically or prophylactically effective amount of one or more of the antisense compounds or compositions of the invention.

In particular, the invention provides a compound 8 to 50 nucleobases in length targeted to a nucleic acid molecule encoding apolipoprotein B, wherein said compound specifically hybridizes with and inhibits the expression of a nucleic acid molecule encoding apolipoprotein B, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854.

The invention further provides compound 8 to 50 nucleobases in length which specifically hybridizes with at least an 8-nucleobase portion of an active site on a nucleic acid molecule encoding apolipoprotein B, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854, said active site being a region in said nucleic acid wherein binding of said compound to said site significantly inhibits apolipoprotein B expression as compared to a control.

The invention also provides a compound 8 to 50 nucleobases in length targeted to a nucleic acid molecule encoding apolipoprotein B, wherein said compound specifically hybridizes with said nucleic acid and inhibits expression of apolipoprotein B, wherein the apolipoprotein B is encoded by a polynucleotide selected from the group consisting of: (a) SEQ ID NO: 3 and (b) a naturally occurring variant apolipoprotein B-encoding polynucleotide that hybridizes to the complement of the polynucleotide of (a) under stringent conditions, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854.

In another aspect the invention provides a compound 8 to 50 nucleobases in length targeted to a nucleic acid molecule encoding apolipoprotein B, wherein said compound specifically hybridizes with said nucleic acid and inhibits expression of apolipoprotein B, wherein the apolipoprotein B is encoded by a polynucleotide selected from the group consisting of SEQ ID NO: 3 and SEQ ID NO: 17, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854.

The invention also provides a compound 8 to 50 nucleobases in length targeted to a nucleic acid molecule encoding apolipoprotein B, wherein said compound specifically hybridizes with an active site in said nucleic acid and inhibits expression of apolipoprotein B, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854, said active site being a region in said nucleic acid wherein binding of said compound to said site significantly inhibits apolipoprotein B expression as compared to a control.

In another aspect the invention provides an oligonucleotide mimetic compound 8 to 50 nucleobases in length targeted to a nucleic acid molecule encoding apolipoprotein B, wherein said compound specifically hybridizes with said nucleic acid and inhibits expression of apolipoprotein B, said compound comprising at least 8 contiguous nucleobases of any one of SEQ ID NOs: 127-134, 136, 138-174, 176-317, 319-321, 323-333, 335-339, 341-374, 376-416, 418-500, 502-510, 512-804, 815, 816, 819-821, 824, 825, 827, 828, 830, 831, 833-835, 837-839, 842, 843, and 845-854.

In another aspect, the invention provides an antisense compound 8 to 50 nucleobases in length, wherein said compound specifically hybridizes with nucleotides 2920-3420 as set forth in SEQ ID NO:3 and inhibits expression of mRNA encoding human apolipoprotein B after 16 to 24 hours by at least 30% in 80% confluent HepG2 cells in culture at a concentration of 150 nM. In preferred embodiments, the antisense compound 8 to 50 nucleobases in length specifically hybridizes with nucleotides 3230-3288 as set forth in SEQ ID NO:3 and inhibits expression of mRNA encoding human apolipoprotein B after 16 to 24 hours by at least 30% in 80% confluent HepG2 cells in culture at a concentration of 150 nM. In another aspect, the compounds inhibits expression of mRNA encoding apolipoprotein B by at least 50%, after 16 to 24 hours in 80% confluent HepG2 cells in culture at a concentration of 150 nM.

In one aspect, the compounds of the invention are targeted to a nucleic acid molecule encoding apolipoprotein B, wherein said compound specifically hybridizes with and inhibits expression of the long form of apolipoprotein B, ApoB-100. In another aspect, the compounds specifically hybridizes with said nucleic acid and inhibits expression of mRNA encoding apolipoprotein B by at least 5% in 80% confluent HepG2 cells in culture at an optimum concentration. In yet another aspect, the compounds inhibits expression of mRNA encoding apolipoprotein B by at least 10%, at least 15%, at least 20%, at least 25%, at least 30%, at least 35%, at least 40%, or at least 50%.

In one aspect, the compounds are antisense oligonucleotides, and in one embodiment the compound has a sequence comprising SEQ ID NO: 224, the antisense oligonucleotide hybridizes with a region complementary to SEQ ID NO: 224, the compound comprises SEQ ID NO: 224, the compound consists essentially of SEQ ID NO: 224 or the compound consists of SEQ ID NO: 224.

In another aspect, the compound has a sequence comprising SEQ ID NO: 247, the antisense oligonucleotide hybridizes with a region complementary to SEQ ID NO: 247, the compound comprises SEQ ID NO: 247, the compound consists essentially of SEQ ID NO: 247 or the compound consists of SEQ ID NO: 247.

In another aspect, the compound has a sequence comprising SEQ ID NO: 319, the antisense oligonucleotide hybridizes with a region complementary to SEQ ID NO: 319, the compound comprises SEQ ID NO: 319, the compound consists essentially of SEQ ID NO: 319 or the compound consists of SEQ ID NO: 319.

In one embodiment, the compounds comprise at least one modified internucleoside linkage, and in another embodiment, the modified internucleoside linkage is a phosphorothioate linkage.

In another aspect, the compounds comprise at least one modified sugar moiety, and in one aspect, the modified sugar moiety is a 2′-O-methoxyethyl sugar moiety.

In another embodiment, the compounds comprise at least one modified nucleobase, and in one aspect, the modified nucleobase is a 5-methylcytosine.

In yet another aspect, the compounds are chimeric oligonucleotides. Preferred chimeric compounds include those having one or more phosphorothioate linkages and further comprising 2′-methoxyethoxyl nucleotide wings and a ten nucleobase 2′-deoxynucleotide gap.

In another aspect, the compounds specifically hybridizes with and inhibits the expression of a nucleic acid molecule encoding an alternatively spliced form of apolipoprotein B.

The invention also provide compositions comprising a compound of the invention and a pharmaceutically acceptable carrier or diluent. In one aspect, the composition further comprises a colloidal dispersion system, and in another aspect, the compound in the composition is an antisense oligonucleotide. In certain embodiments, the composition comprises an antisense compound of the invention hybridized to a complementary strand. Hybridization of the antisense strand can form one or more blunt ends or one or more overhanging ends. In some embodiments, the overhanging end comprises a modified base.

The invention further provides methods of inhibiting the expression of apolipoprotein B in cells or tissues comprising contacting said cells or tissues with a compound of the invention so that expression of apolipoprotein B is inhibited. Methods are also provided for treating an animal having a disease or condition associated with apolipoprotein B comprising administering to said animal a therapeutically or prophylactically effective amount of a compound of the invention so that expression of apolipoprotein B is inhibited. In various aspects, the condition is associated with abnormal lipid metabolism, the condition is associated with abnormal cholesterol metabolism, the condition is atherosclerosis, the condition is an abnormal metabolic condition, the abnormal metabolic condition is hyperlipidemia, the disease is diabetes, the diabetes is Type 2 diabetes, the condition is obesity, and/or the disease is cardiovascular disease.

The invention also provide methods of modulating glucose levels in an animal comprising administering to said animal a compound of the invention, and in one aspect, the animal is a human. In various embodiments, the glucose levels are plasma glucose levels, the glucose levels are serum glucose levels, and/or the animal is a diabetic animal.

The invention also provides methods of preventing or delaying the onset of a disease or condition associated with apolipoprotein B in an animal comprising administering to said animal a therapeutically or prophylactically effective amount of a compound of the invention. In one aspect, the animal is a human. In other aspects, the condition is an abnormal metabolic condition, the abnormal metabolic condition is hyperlipidemia, the disease is diabetes, the diabetes is Type 2 diabetes, the condition is obesity, the condition is atherosclerosis, the condition involves abnormal lipid metabolism, and/or the condition involves abnormal cholesterol metabolism.

The invention also provides methods of preventing or delaying the onset of an increase in glucose levels in an animal comprising administering to said animal a therapeutically or prophylactically effective amount of a compound of the invention. In one aspect, the animal is a human. In other aspects, the glucose levels are serum glucose levels, and/or the glucose levels are plasma glucose levels.

The invention also provides methods of modulating serum cholesterol levels in an animal comprising administering to said animal a therapeutically or prophylactically effective amount of a compound of the invention. In one aspect, the animal is a human.

The invention also provides methods of modulating lipoprotein levels in an animal comprising administering to said animal a therapeutically or prophylactically effective amount of a compound of the invention. In one aspect, the animal is a human. In other aspects, the lipoprotein is VLDL, the lipoprotein is HDL, and/or the lipoprotein is LDL.

The invention also provides methods of modulating serum triglyceride levels in an animal comprising administering to said animal a therapeutically or prophylactically effective amount of a compound of the invention. In one aspect, the animal is a human.

The invention also proves use of a compound of the invention for the manufacture of a medicament for the treatment of a disease or condition associated with apolipoprotein B expression, a medicament for the treatment of a condition associated with abnormal lipid metabolism, a medicament for the treatment of a condition associated with abnormal cholesterol metabolism, a medicament for the treatment of atherosclerosis, a medicament for the treatment of hyperlipidemia, a medicament for the treatment of diabetes, a medicament for the treatment of Type 2 diabetes, a medicament for the treatment of obesity, a medicament for the treatment of cardiovascular disease, a medicament for preventing or delaying the onset of increased glucose levels, a medicament for preventing or delaying the onset of increased serum glucose levels, a medicament for preventing or delaying the onset of increased plasma glucose levels, a medicament for the modulation of serum cholesterol levels, a medicament for the modulation of serum lipoprotein levels, a medicament for the modulation of serum VLDL levels, a medicament for the modulation of serum HDL levels, and/or a medicament for the modulation of serum LDL levels, a medicament for the modulation of serum triglyceride levels.

In another aspect, the invention provides methods of decreasing circulating lipoprotein levels comprising the step of administering to an individual an amount of a compound of the invention sufficient to reduce apolipoprotein B expression. In another aspect, the invention provides methods of reducing lipoprotein transport comprising the step of administering to an individual an amount of a compound of the invention sufficient to reduce apolipoprotein B expression. The invention also provides methods of reducing lipoprotein absorption/adsorption comprising the step of administering to an individual an amount of a compound of the invention sufficient to reduce apolipoprotein B expression.

In another aspect, the invention contemplates methods of decreasing circulating triglyceride levels comprising the step of administering to an individual an amount of a compound of the invention sufficient to reduce apolipoprotein B expression. Also provided are methods of reducing triglyceride transport comprising the step of administering to an individual an amount of a compound of the invention sufficient to reduce apolipoprotein B expression. The invention further provides methods of reducing triglyceride absorption/adsorption comprising the step of administering to an individual an amount of a compound of the invention sufficient to reduce apolipoprotein B expression.

In another aspect, the invention provides methods of decreasing circulating cholesterol levels, including cholesteryl esters and/or unesterified cholesterol, comprising the step of administering to an individual an amount of a compound of the invention sufficient to reduce apolipoprotein B expression. Also contemplated are methods of reducing cholesterol transport, including cholesteryl esters and/or unesterified cholesterol, comprising the step of administering to an individual an amount of a compound of the invention sufficient to reduce apolipoprotein B expression. The invention also provides methods of reducing cholesterol absorption/adsorption, including cholesteryl esters and/or unesterified cholesterol, comprising the step of administering to an individual an amount of a compound of the invention sufficient to reduce apolipoprotein B expression.

In another aspect, the invention provides methods of decreasing circulating lipid levels comprising the step of administering to an individual an amount of a compound of the invention sufficient to reduce apolipoprotein B expression. The invention also provides methods of reducing lipid transport in plasma comprising the step of administering to an individual an amount of a compound of the invention sufficient to reduce apolipoprotein B expression. In addition, the invention provides methods of reducing lipid absorption/adsorption comprising the step of administering to an individual an amount of a compound of the invention sufficient to reduce apolipoprotein B expression.

The invention further contemplates methods of decreasing circulating dietary lipid levels comprising the step of administering to an individual an amount of a compound of the invention sufficient to reduce apolipoprotein B expression. Also provided are methods of reducing dietary lipid transport comprising the step of administering to an individual an amount of a compound of the invention sufficient to reduce apolipoprotein B expression, as well as methods of reducing dietary lipid absorption/adsorption comprising the step of administering to an individual an amount of a compound of the invention sufficient to reduce apolipoprotein B expression.

In another aspect, the invention provides methods of decreasing circulating fatty acid levels comprising the step of administering to an individual an amount of a compound of the invention sufficient to reduce apolipoprotein B expression. The invention also provides methods of reducing fatty acid transport comprising the step of administering to an individual an amount of a compound of the invention sufficient to reduce apolipoprotein B expression. Also contemplated are methods of reducing fatty acid absorption comprising the step of administering to an individual an amount of a compound of the invention sufficient to reduce apolipoprotein B expression.

The invention also provides methods of decreasing circulating acute phase reactants comprising the step of administering to an individual an amount of a compound of the invention sufficient to reduce apolipoprotein B expression. In another aspect, the invention provides methods of reducing acute phase reactants transport comprising the step of administering to an individual an amount of a compound of the invention sufficient to reduce apolipoprotein B expression, as well as methods of reducing acute phase reactants absorption comprising the step of administering to an individual an amount of a compound of the invention sufficient to reduce apolipoprotein B expression.

In another aspect, the invention provides methods of decreasing circulating chylomicrons comprising the step of administering to an individual an amount of a compound of the invention sufficient to reduce apolipoprotein B expression, methods of reducing chylomicron transport comprising the step of administering to an individual an amount of a compound of the invention sufficient to reduce apolipoprotein B expression, and methods of reducing chylomicron absorption comprising the step of administering to an individual an amount of a compound of the invention sufficient to reduce apolipoprotein B expression.

The invention further provides methods of decreasing circulating chylomicron remnant particles comprising the step of administering to an individual an amount of a compound of the invention sufficient to reduce apolipoprotein B expression, methods of reducing chylomicron remnant transport comprising the step of administering to an individual an amount of a compound of the invention sufficient to reduce apolipoprotein B expression, and methods of reducing chylomicron remnant absorption comprising the step of administering to an individual an amount of a compound of the invention sufficient to reduce apolipoprotein B expression.

The invention further contemplates methods of decreasing circulating VLDL, IDL, LDL, and/or HDL comprising the step of administering to an individual an amount of a compound of the invention sufficient to reduce apolipoprotein B expression. Likewise, the invention provides methods of reducing VLDL, IDL, LDL, and/or HDL transport comprising the step of administering to an individual an amount of a compound of the invention sufficient to reduce apolipoprotein B expression, in addition to methods of reducing VLDL, IDL, LDL, and/or HDL absorption comprising the step of administering to an individual an amount of a compound of the invention sufficient to reduce apolipoprotein B expression.

In still another aspect, the invention provides methods of treating a condition associated with apolipoprotein B expression comprising the step of administering to an individual an amount of a compound of the invention sufficient to inhibit apolipoprotein B expression, said condition selected from hyperlipoproteinemia, familial type 3 hyperlipoprotienemia (familial dysbetalipoproteinemia), and familial hyperalphalipoprotienemia; hyperlipidemia, mixed hyperlipidemias, multiple lipoprotein-type hyperlipidemia, and familial combined hyperlipidemia; hypertriglyceridemia, familial hypertriglyceridemia, and familial lipoprotein lipase; hypercholesterolemia, familial hypercholesterolemia, polygenic hypercholesterolemia, and familial defective apolipoprotein B; cardiovascular disorders including atherosclerosis and coronary artery disease; peripheral vascular disease; von Gierke's disease (glycogen storage disease, type I); lipodystrophies (congenital and acquired forms); Cushing's syndrome; sexual ateloitic dwarfism (isolated growth hormone deficiency); diabetes mellitus; hyperthyroidism; hypertension; anorexia nervosa; Werner's syndrome; acute intermittent porphyria; primary biliary cirrhosis; extrahepatic biliary obstruction; acute hepatitis; hepatoma; systemic lupus erythematosis; monoclonal gammopathies (including myeloma, multiple myeloma, macroglobulinemia, and lymphoma); endocrinopathies; obesity; nephrotic syndrome; metabolic syndrome; inflammation; hypothyroidism; uremia (hyperurecemia); impotence; obstructive liver disease; idiopathic hypercalcemia; dysglobulinemia; elevated insulin levels; Syndrome X; Dupuytren's contracture; and Alzheimer's disease and dementia.

The invention also provides methods of reducing the risk of a condition comprising the step of administering to an individual an amount of a compound of the invention sufficient to inhibit apolipoprotein B expression, said condition selected from pregnancy; intermittent claudication; gout; and mercury toxicity and amalgam illness.

The invention further provides methods of inhibiting cholesterol particle binding to vascular endothelium comprising the step of administering to an individual an amount of a compound of the invention sufficient to inhibit apolipoprotein B expression, and as a result, the invention also provides methods of reducing the risk of: (i) cholesterol particle oxidization; (ii) monocyte binding to vascular endothelium; (iii) monocyte differentiation into macrophage; (iv) macrophage ingestion of oxidized lipid particles and release of cytokines (including, but limited to IL-1,TNF-alpha, TGF-beta); (v) platelet formation of fibrous fibrofatty lesions and inflammation; (vi) endothelium lesions leading to clots; and (vii) clots leading to myocardial infarction or stroke, also comprising the step of administering to an individual an amount of a compound of the invention sufficient to inhibit apolipoprotein B expression.

The invention also provides methods of reducing hyperlipidemia associated with alcoholism, smoking, use of oral contraceptives, use of glucocorticoids, use of beta-adrenergic blocking agents, or use of isotretinion (13-cis-retinoic acid) comprising the step of administering to an individual an amount of a compound of the invention sufficient to inhibit apolipoprotein B expression.

In certain aspects, the invention provides an antisense oligonucleotide compound 8 to 50 nucleobases in length comprising at least 8 contiguous nucleotides of SEQ ID NO:247 and having a length from at least 12 or at least 14 to 30 nucleobases.

In a further aspect, the invention provides an antisense oligonucleotide compound 20 nucleobases in length having a sequence of nucleobases as set forth in SEQ ID NO:247 and comprising 5-methylcytidine at nucleobases 2, 3, 5, 9, 12, 15, 17, 19, and 20, wherein every internucleoside linkage is a phosphothioate linkage, nucleobases 1-5 and 16-20 comprise a 2′-methoxyethoxyl modification, and nucleobases 6-15 are deoxynucleotides.

In another aspect, the invention provides a compound comprising a first nucleobase strand, 8 to 50 nucleobases in length and comprising a sequence of at least 8 contiguous nucleobases of the sequence set forth in SEQ ID NO:3, hybridized to a second nucleobase strand, 8 to 50 nucleobases in length and comprising a sequence sufficiently complementary to the first strand so as to permit stable hybridization, said compound inhibiting expression of mRNA encoding human apolipoprotein B after 16 to 24 hours by at least 30% or by at least 50% in 80% confluent HepG2 cells in culture at a concentration of 100 nM.

Further provided is a vesicle, such as a liposome, comprising a compound or composition of the invention

Preferred methods of administration of the compounds or compositions of the invention to an animal are intravenously, subcutaneously, or orally. Administrations can be repeated.

In another aspect, the invention provides a method of reducing lipoprotein(a) secretion by hepatocytes comprising (a) contacting hepatocytes with an amount of a composition comprising a non-catalytic compound 8 to 50 nucleobases in length that specifically hybridizes with mRNA encoding human apolipoprotein B and inhibits expression of the mRNA after 16 to 24 hours by at least 30% or at least 50% in 80% confluent HepG2 cells in culture at a concentration of 150 nM, wherein said amount is effective to inhibit expression of apolipoprotein B in the hepatocytes; and (b) measuring lipoprotein(a) secretion by the hepatocytes.

The invention further provides a method of a treating a condition associated with apolipoprotein B expression in a primate, such as a human, comprising administering to the primate a therapeutically or prophylactically effective amount of a non-catalytic compound 8 to 50 nucleobases in length that specifically hybridizes with mRNA encoding human apolipoprotein B and inhibits expression of the mRNA after 16 to 24 hours by at least 30% or by at least 50% in 80% confluent HepG2 cells in culture at a concentration of 150 nM.

The invention provides a method of reducing apolipoprotein B expression in the liver of an animal, comprising administering to the animal between 2 mg/kg and 20 mg/kg of a non-catalytic compound 8 to 50 nucleobases in length that specifically hybridizes with mRNA encoding human apolipoprotein B by at least 30% or by at least 50% in 80% confluent HepG2 cells in culture at a concentration of 150 nM.

Also provided is a method of making a compound of the invention comprising specifically hybridizing in vitro a first nucleobase strand comprising a sequence of at least 8 contiguous nucleobases of the sequence set forth in SEQ ID NO:3 to a second nucleobase strand comprising a sequence sufficiently complementary to said first strand so as to permit stable hybridization.

The invention further provides use of a compound of the invention in the manufacture of a medicament for the treatment of any and all conditions disclosed herein.

The present invention is directed to compounds, particularly antisense oligonucleotides, which are targeted to a nucleic acid encoding apolipoprotein B. Such compounds modulate the expression of apolipoprotein B and result in a lean animal gene expression profile. Pharmaceutical and other compositions comprising the compounds of the invention are also provided. Further provided are methods of modulating the expression of apolipoprotein B and effecting a lean animal expression profile in cells or tissues comprising contacting said cells or tissues with one or more of the antisense compounds or compositions of the invention. Further provided are methods of treating an animal, particularly a human, suspected of having or being prone to a disease or condition associated with cardiovascular disease by administering a therapeutically or prophylactically effective amount of one or more of the antisense compounds or compositions of the invention.

The present invention provides methods comprising contacting an animal with an antisense oligonucleotide 15-30 nucleobases in length and modulating the level of a target gene mRNA, wherein the antisense oligonucleotide reduces the level of apolipoprotein B mRNA, and wherein the target gene is selected from the group consisting of Lcat, Lip1, Lipc, Ppara, Pparg, Pcx, Apoa4, Apoc1, Apoc2, Apoc4, Mttp, Prkaa1, Prkaa2, Prkab1, Prkag1, Srebp-1, Scd2, Scd1, Acadl, Acadm, Acads, Acox1, Cpt1a, Cpt2, Crat, Elov12, Elov13, Acadsb, Fads2, Fasn, Fac12, Fac14, Abcd2, Dbi, Fabp1, Fabp2, Fabp7, Acat-1, Acca-1, Cyp7a1, Cyp7b1, Soat2, Ldlr, Hmgcs1, Hmgcs2, Car5a, Gck, Gck and G6pc. In some aspects, the target gene mRNA is reduced, and this reduction occurs in a time-dependent manner or in a dose-dependent manner. Alternatively, the target gene mRNA is increased in a time-dependent manner or in a dose-dependent manner. In further aspects, the modulation of the target gene mRNA levels occurs in both a time- and dose-dependent manner.

Further provided are methods that result in a shift of a gene expression profile of an obese animal to that of a lean animal. Such methods comprise contacting an animal with an antisense oligonucleotide 15 to 30 nucleobases in length targeted to apolipoprotein B, resulting in the shift of a gene expression profile of an obese animal to that of a lean animal. In one aspect, the gene expression profile is a liver gene expression profile.

The invention also provides methods of reducing the risk of cardiovascular disease in an individual comprising the step of administering to an individual an amount of a compound of the invention sufficient to inhibit apolipoprotein B expression and modulate a gene expression profile. Risk factors for cardiovascular disease that are recognized by the Adult Treatment Panel III of the National Cholesterol Education Program include: previous coronary events, a family history of cardiovascular disease, elevated LDL-cholesterol, low HDL-cholesterol, elevated serum triglyceride, obesity, and physical inactivity, and metabolic syndrome.

The invention further provides methods of inhibiting the expression of apolipoprotein B and modulating a gene expression profile in cells or tissues comprising contacting said cells or tissues with a compound of the invention so that expression of apolipoprotein B is inhibited. Methods are also provided for treating an animal having a cardiovascular disease or condition comprising administering to said animal a therapeutically or prophylactically effective amount of a compound of the invention so that expression of apolipoprotein B is inhibited and gene expression profiles are altered. In various aspects, the condition is associated with abnormal lipid metabolism, the condition is associated with abnormal cholesterol metabolism, the condition is atherosclerosis, the condition is an abnormal metabolic condition, the abnormal metabolic condition is hyperlipidemia, the disease is diabetes, the diabetes is Type 2 diabetes, the condition is obesity, and/or the disease is cardiovascular disease.

The invention also provides methods of preventing or delaying the onset of a disease or condition associated with cardiovascular disease in an animal comprising administering to said animal a therapeutically or prophylactically effective amount of a compound of the invention. In one aspect, the animal is a human. In other aspects, the condition is an abnormal metabolic condition, the abnormal metabolic condition is hyperlipidemia, the disease is diabetes, the diabetes is Type 2 diabetes, the condition is obesity, the condition is atherosclerosis, the condition involves abnormal lipid metabolism, and/or the condition involves abnormal cholesterol metabolism.

Preferred methods of administration of the compounds or compositions of the invention to an animal are intravenously, subcutaneously, or orally. Administrations can be repeated.

Further provided are methods for altering a cellular pathway or metabolic process comprising contacting a cell with an antisense oligonucleotide that specifically hybridizes to and inhibits the expression of a nucleic acid molecule encoding apolipoprotein B. Cellular pathways and metabolic processes include apoptosis, angiogenesis, leptic secretion and T-cell co-stimulation. In some aspects, the antisense oligonucleotide comprises SEQ ID NO: 20. In one embodiment, apoptosis is induced in cancer cells, for example, breast cancer cells. In a further embodiment, angiogenesis, leptin secretion and T-cell co-stimulation are inhibited.

### DETAILED DESCRIPTION OF THE INVENTION

The present invention employs oligomeric compounds, particularly antisense oligonucleotides, for use in modulating the function of nucleic acid molecules encoding apolipoprotein B, ultimately modulating the amount of apolipoprotein B produced. This is accomplished by providing antisense compounds which specifically hybridize with one or more nucleic acids encoding apolipoprotein B.

The present invention employs oligomeric compounds, particularly antisense oligonucleotides, for use in modulating the level of nucleic acid molecules encoding apolipoprotein B, ultimately resulting in the modulation of the mRNA levels of genes whose expression patterns are characteristic of an obese animal. Such modulation of gene expression patterns shifts a gene profile of an obese animal to that of a lean animal. This is accomplished by providing antisense compounds which specifically hybridize with one or more nucleic acids encoding apolipoprotein B.

As used herein, the terms “target nucleic acid” and “nucleic acid encoding apolipoprotein B” encompass DNA encoding apolipoprotein B, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of apolipoprotein B. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding apolipoprotein B. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding apolipoprotein B, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are hereinbelow identified as preferred embodiments of the invention. The target sites to which these preferred sequences are complementary are hereinbelow referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For use in kits and diagnostics, the antisense compounds of the present invention, either alone or in combination with other antisense compounds or therapeutics, can be used as tools in differential and/or combinatorial analyses to elucidate expression patterns of a portion or the entire complement of genes expressed within cells and tissues.

Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. Thus, this term includes oligonucleotides composed of naturally-occurring nucleobases, sugars and covalent internucleoside (backbone) linkages (RNA and DNA) as well as oligonucleotides having non-naturally-occurring portions which function similarly (oligonucleotide mimetics). Oligonucleotide mimetics are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12, about 14, about 20 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. In preferred embodiments, the antisense compound is non-catalytic oligonucleotide, i.e., is not dependent on a catalytic property of the oligonucleotide for its modulating activity. Antisense compounds of the invention can include double-stranded molecules wherein a first strand is stably hybridized to a second strand.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,750,692; 5,763,588; 6,005,096; and 5,681,941, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthra-quinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Chimeric antisense compounds can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

Both gapmer and hemimer compounds have also been referred to in the art as hybrids. In a gapmer that is 20 nucleotides in length, a gap or wing can be 1, 2, 3, 4, 5, 6, 7, 8, 9, 10, 11, 12, 13, 14, 15, 16, 17 or 18 nucleotides in length. In one embodiment, a 20-nucleotide gapmer is comprised of a gap 8 nucleotides in length, flanked on both the 5′ and 3′ sides by wings 6 nucleotides in length. In another embodiment, a 20-nucleotide gapmer is comprised of a gap 10 nucleotides in length, flanked on both the 5′ and 3′ sides by wings 5 nucleotides in length. In another embodiment, a 20-nucleotide gapmer is comprised of a gap 12 nucleotides in length flanked on both the 5′ and 3′ sides by wings 4 nucleotides in length. In a further embodiment, a 20-nucleotide gapmer is comprised of a gap 14 nucleotides in length flanked on both the 5′ and 3′ sides by wings 3 nucleotides in length. In another embodiment, a 20-nucleotide gapmer is comprised of a gap 16 nucleotides in length flanked on both the 5′ and 3′ sides by wings 2 nucleotides in length. In a further embodiment, a 20-nucleotide gapmer is comprised of a gap 18 nucleotides in length flanked on both the 5′ and 3′ ends by wings 1 nucleotide in length. Alternatively, the wings are of different lengths, for example, a 20-nucleotide gapmer may be comprised of a gap 10 nucleotides in length, flanked by a 6-nucleotide wing on one side (5′ or 3′) and a 4-nucleotide wing on the other side (5′ or 3′). In a hemimer, an “open end” chimeric antisense compound, 20 nucleotides in length, a gap segment, located at either the 5′ or 3′ terminus of the oligomeric compound, can be 1, 2, 3, 4, 5, 6, 7, 8, 9, 10, 11, 12, 13, 14, 15, 16, 17, 18 or 19 nucleotides in length. For example, a 20-nucleotide hemimer can have a gap segment of 10 nucleotides at the 5′ end and a second segment of 10 nucleotides at the 3′ end. Alternatively, a 20-nucleotide hemimer can have a gap segment of 10 nucleotides at the 3′ end and a second segment of 10 nucleotides at the 5′ end.

Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of apolipoprotein B is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding apolipoprotein B, enabling sandwich and other assays to easily be constructed to exploit this fact. Hybridization of the antisense oligonucleotides of the invention with a nucleic acid encoding apolipoprotein B can be detected by means known in the art. Such means may include conjugation of an enzyme to the oligonucleotide, radiolabelling of the oligonucleotide or any other suitable detection means. Kits using such detection means for detecting the level of apolipoprotein B in a sample may also be prepared.

The primers and probes disclosed herein are useful for research and diagnostics, because these compounds hybridize to nucleic acids encoding apolipoprotein B, enabling sandwich and other assays to easily be constructed to exploit this fact. Hybridization of the disclosed primers and probes with a nucleic acid encoding apolipoprotein B can be detected by means known in the art. Such means may include conjugation of an enzyme to the oligonucleotide, radiolabelling of the oligonucleotide or any other suitable detection means. Kits using such detection means for detecting the level of apolipoprotein B in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999) each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.1 μm in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome® I (glyceryl dilaurate/cholesterol/polyoxyethylene-10-stearyl ether) and Novasome® II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15 G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Pulsatile Delivery

The compounds of the present invention may also be administered by pulsatile delivery. “Pulsatile delivery” refers to a pharmaceutical formulations that delivers a first pulse of drug combined with a penetration enhancer and a second pulse of penetration enhancer to promote absorption of drug which is not absorbed upon release with the first pulse of penetration enhancer.

One embodiment of the present invention is a delayed release oral formulation for enhanced intestinal drug absorption, comprising:
(a) a first population of carrier particles comprising said drug and a penetration enhancer, wherein said drug and said penetration enhancer are released at a first location in the intestine; and
(b) a second population of carrier particles comprising a penetration enhancer and a delayed release coating or matrix, wherein the penetration enhancer is released at a second location in the intestine downstream from the first location, whereby absorption of the drug is enhanced when the drug reaches the second location.

Alternatively, the penetration enhancer in (a) and (b) is different.

This enhancement is obtained by encapsulating at least two populations of carrier particles. The first population of carrier particles comprises a biologically active substance and a penetration enhancer, and the second (and optionally additional) population of carrier particles comprises a penetration enhancer and a delayed release coating or matrix.

A “first pass effect” that applies to orally administered drugs is degradation due to the action of gastric acid and various digestive enzymes. One means of ameliorating first pass clearance effects is to increase the dose of administered drug, thereby compensating for proportion of drug lost to first pass clearance. Although this may be readily achieved with i.v. administration by, for example, simply providing more of the drug to an animal, other factors influence the bioavailability of drugs administered via non-parenteral means. For example, a drug may be enzymatically or chemically degraded in the alimentary canal or blood stream and/or may be impermeable or semipermeable to various mucosal membranes.

It is also contemplated that these pharmaceutical compositions are capable of enhancing absorption of biologically active substances when administered via the rectal, vaginal, nasal or pulmonary routes. It is also contemplated that release of the biologically active substance can be achieved in any part of the gastrointestinal tract.

Liquid pharmaceutical compositions of oligonucleotide can be prepared by combining the oligonucleotide with a suitable vehicle, for example sterile pyrogen free water, or saline solution. Other therapeutic compounds may optionally be included.

The present invention also contemplates the use of solid particulate compositions. Such compositions preferably comprise particles of oligonucleotide that are of respirable size. Such particles can be prepared by, for example, grinding dry oligonucleotide by conventional means, fore example with a mortar and pestle, and then passing the resulting powder composition through a 400 mesh screen to segregate large particles and agglomerates. A solid particulate composition comprised of an active oligonucleotide can optionally contain a dispersant which serves to facilitate the formation of an aerosol, for example lactose.

In accordance with the present invention, oligonucleotide compositions can be aerosolized. Aerosolization of liquid particles can be produced by any suitable means, such as with a nebulizer. See, for example, U.S. Pat. No. 4,501,729. Nebulizers are commercially available devices which transform solutions or suspensions into a therapeutic aerosol mist either by means of acceleration of a compressed gas, typically air or oxygen, through a narrow venturi orifice or by means of ultrasonic agitation. Suitable nebulizers include those sold by Blairex® under the name PARI LC PLUS, PARI DURA-NEB 2000, PARI-BABY Size, PARI PRONEB Compressor with LC PLUS, PARI WALKHALER Compressor/Nebulizer System, PARI LC PLUS Reusable Nebulizer, and PARI LC Jet+® Nebulizer.

Exemplary formulations for use in nebulizers consist of an oligonucleotide in a liquid, such as sterile, pyragen free water, or saline solution, wherein the oligonucleotide comprises up to about 40% w/w of the formulation. Preferably, the oligonucleotide comprises less than 20% w/w. If desired, further additives such as preservatives (for example, methyl hydroxybenzoate) antioxidants, and flavoring agents can be added to the composition.

Solid particles comprising an oligonucleotide can also be aerosolized using any solid particulate medicament aerosol generator known in the art. Such aerosol generators produce respirable particles, as described above, and further produce reproducible metered dose per unit volume of aerosol. Suitable solid particulate aerosol generators include insufflators and metered dose inhalers. Metered dose inhalers are used in the art and are useful in the present invention.

Preferably, liquid or solid aerosols are produced at a rate of from about 10 to 150 liters per minute, more preferably from about 30 to 150 liters per minute, and most preferably about 60 liters per minute.

Enhanced bioavailability of biologically active substances is also achieved via the oral administration of the compositions and methods of the present invention. The term “bioavailability” refers to a measurement of what portion of an administered drug reaches the circulatory system when a non-parenteral mode of administration is used to introduce the drug into an animal.

Penetration enhancers include, but are not limited to, members of molecular classes such as surfactants, fatty acids, bile salts, chelating agents, and non-chelating non-surfactant molecules. (Lee et al., Critical Reviews in Therapeutic Drug Carrier Systems, 1991, p. 92). Carriers are inert molecules that may be included in the compositions of the present invention to interfere with processes that lead to reduction in the levels of bioavailable drug.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

The effects of treatments with therapeutic compositions can be assessed following collection of tissues or fluids from a patient or subject receiving said treatments. It is known in the art that a biopsy sample can be procured from certain tissues without resulting in detrimental effects to a patient or subject. In certain embodiments, a tissue and its constituent cells comprise, but are not limited to, blood (e.g., hematopoietic cells, such as human hematopoietic progenitor cells, human hematopoietic stem cells, CD34⁺ cells CD4⁺ cells), lymphocytes and other blood lineage cells, bone marrow, breast, cervix, colon, esophagus, lymph node, muscle, peripheral blood, oral mucosa and skin. In other embodiments, a fluid and its constituent cells comprise, but are not limited to, blood, urine, semen, synovial fluid, lymphatic fluid and cerebro-spinal fluid. Tissues or fluids procured from patients can be evaluated for expression levels of the target mRNA or protein. Additionally, the mRNA or protein expression levels of other genes known or suspected to be associated with the specific disease state, condition or phenotype can be assessed. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the disease or condition in the aforementioned tissues and fluids, collected from a patient or subject receiving treatment, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

Combination Therapy

The invention also provides methods of combination therapy, wherein one or more compounds of the invention and one or more other therapeutic/prophylactic compounds are administered treat a condition and/or disease state as described herein. In various aspects, the compound(s) of the invention and the therapeutic/prophylactic compound(s) are co-administered as a mixture or administered individually. In one aspect, the route of administration is the same for the compound(s) of the invention and the therapeutic/prophylactic compound(s), while in other aspects, the compound(s) of the invention and the therapeutic/prophylactic compound(s) are administered by a different routes. In one embodiment, the dosages of the compound(s) of the invention and the therapeutic/prophylactic compound(s) are amounts that are therapeutically or prophylactically effective for each compound when administered individually. Alternatively, the combined administration permits use of lower dosages than would be required to achieve a therapeutic or prophylactic effect if administered individually, and such methods are useful in decreasing one or more side effects of the reduced-dose compound.

In one aspect, a compound of the present invention and one or more other therapeutic/prophylactic compound(s) effective at treating a condition are administered wherein both compounds act through the same or different mechanisms. Therapeutic/prophylactic compound(s) include, but are not limited to, bile salt sequestering resins (e.g., cholestyramine, colestipol, and colesevelam hydrochloride), HMGCoA-redectase inhibitors (e.g., lovastatin, cerivastatin, prevastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), implitapide, inhibitors of bile acid transporters (apical sodium-dependent bile acid transporters), regulators of hepatic CYP7a, estrogen replacement therapeutics (e.g., tamoxigen), and anti-inflammatories (e.g., glucocorticoids).

Accordingly, the invention further provides use of a compound of the invention and one or more other therapeutic/prophylactic compound(s) as described herein in the manufacture of a medicament for the treatment and/or prevention of a disease or condition as described herein.

Targeted Delivery

In another aspect, methods are provided to target a compound of the invention to a specific tissue, organ or location in the body. Exemplary targets include liver, lung, kidney, heart, and atherosclerotic plaques within a blood vessel. Methods of targeting compounds are well known in the art.

In one embodiment, the compound is targeted by direct or local administration. For example, when targeting a blood vessel, the compound is administered directly to the relevant portion of the vessel from inside the lumen of the vessel, e.g., single balloon or double balloon catheter, or through the adventitia with material aiding slow release of the compound, e.g., a pluronic gel system as described by Simons et al., Nature 359: 67-70 (1992). Other slow release techniques for local delivery of the compound to a vessel include coating a stent with the compound. Methods of delivery of antisense compounds to a blood vessel are disclosed in U.S. Pat. No. 6,159,946, which is incorporated by reference in its entirety.

When targeting a particular tissue or organ, the compound may be administered in or around that tissue or organ. For example, U.S. Pat. No. 6,547,787, incorporated herein by reference in its entirety, discloses methods and devices for targeting therapeutic agents to the heart. In one aspect, administration occurs by direct injection or by injection into a blood vessel associated with the tissue or organ. For example, when targeting the liver, the compound may be administered by injection or infusion through the portal vein.

In another aspect, methods of targeting a compound are provided which include associating the compound with an agent that directs uptake of the compound by one or more cell types. Exemplary agents include lipids and lipid-based structures such as liposomes generally in combination with an organ- or tissue-specific targeting moiety such as, for example, an antibody, a cell surface receptor, a ligand for a cell surface receptor, a polysaccharide, a drug, a hormone, a hapten, a special lipid and a nucleic acid as described in U.S. Pat. No. 6,495,532, the disclosure of which is incorporated herein by reference in its entirety. U.S. Pat. No. 5,399,331, the disclosure of which is incorporated herein by reference in its entirety, describes the coupling of proteins to liposomes through use of a crosslinking agent having at least one maleimido group and an amine reactive function; U.S. Pat. Nos. 4,885,172, 5,059,421 and 5,171,578, the disclosures of which are incorporated herein by reference in their entirety, describe linking proteins to liposomes through use of the glycoprotein streptavidin and coating targeting liposomes with polysaccharides. Other lipid based targeting agents include, for example, micelle and crystalline products as described in U.S. Pat. No. 6,217,886, the disclosure of which is incorporated herein by reference in its entirety.

In another aspect, targeting agents include porous polymeric microspheres which are derived from copolymeric and homopolymeric polyesters containing hydrolyzable ester linkages which are biodegradable, as described in U.S. Pat. No. 4,818,542, the disclosure of which is incorporated herein by reference in its entirety. Typical polyesters include polyglycolic (PGA) and polylactic (PLA) acids, and copolymers of glycolide and L(-lactide) (PGL), which are particularly suited for the methods and compositions of the present invention in that they exhibit low human toxicity and are biodegradable. The particular polyester or other polymer, oligomer, or copolymer utilized as the microspheric polymer matrix is not critical and a variety of polymers may be utilized depending on desired porosity, consistency, shape and size distribution. Other biodegradable or bioerodible polymers or copolymers include, for example, gelatin, agar, starch, arabinogalactan, albumin, collagen, natural and synthetic materials or polymers, such as, poly(ε-caprolactone), poly(ε-caprolactone-CO-lactic acid), poly(ε-caprolactone-CO-glycolic acid), poly(β-hydroxy butyric acid), polyethylene oxide, polyethylene, poly(alkyl-2-cyanoacrylate), (e.g., methyl, ethyl, butyl), hydrogels such as poly(hydroxyethyl methacrylate), polyamides (e.g., polyacrylamide), poly(amino acids) (i.e., L-leucine, L-aspartic acid, β-methyl-L-aspartate, β-benzyl-L-aspartate, glutamic acid), poly(2-hydroxyethyl DL-aspartamide), poly(ester urea), poly(L-phenylalanine/ethylene glycol/1,6-diisocyanatohexane) and poly(methyl methacrylate). The exemplary natural and synthetic polymers suitable for targeted delivery are either readily available commercially or are obtainable by condensation polymerization reactions from the suitable monomers or, comonomers or oligomers.

In still another embodiment, U.S. Pat. No. 6,562,864, the disclosure of which is incorporated herein by reference in its entirety, describes catechins, including epi and other carbo-cationic isomers and derivatives thereof, which as monomers, dimers and higher multimers can form complexes with nucleophilic and cationic bioactive agents for use as delivery agents. Catechin multimers have a strong affinity for polar proteins, such as those residing in the vascular endothelium, and on cell/organelle membranes and are particularly useful for targeted delivery of bioactive agents to select sites in vivo. In treatment of vascular diseases and disorders, such as atherosclerosis and coronary artery disease, delivery agents include substituted catechin multimers, including amidated catechin multimers which are formed from reaction between catechin and nitrogen containing moities such as ammonia.

Other targeting strategies of the invention include ADEPT (antibody-directed enzyme prodrug therapy), GDEPT (gene-directed EPT) and VDEPT (virus-directed EPT) as described in U.S. Pat. No. 6,433,012, the disclosure of which is incorporated herein by reference in its entirety.

The present invention further provides medical devices and kits for targeted delivery, wherein the device is, for example, a syringe, stent, or catheter. Kits include a device for administering a compound and a container comprising a compound of the invention. In one aspect, the compound is preloaded into the device. In other embodiments, the kit provides instructions for methods of administering the compound and dosages. U.S. patents describing medical devices and kits for delivering antisense compounds include U.S. Pat. Nos. 6,368,356; 6,344,035; 6,344,028; 6,287,285; 6,200,304; 5,824,049; 5,749,915; 5,674,242; 5,670,161; 5,609,629; 5,593,974; and 5,470,307 (all incorporated herein by reference in their entirety).

The present invention further provides methods for shifting a gene expression profile of an animal from that of an obese animal to that of a lean animal. A “lean animal” is an animal on a standard diet that is not considered to have a hyperlipidemic condition. An “obese animal” is obese and/or consumes a high-fat diet, and exhibits one or more indicators of hyperlipidemia, for example, elevated serum LDL-cholesterol, lowered serum HDL-cholesterol, or elevated serum triglycerides. Expression profiles are identified by the comparison of mRNA levels in a lean animal (“lean animal profile” or “lean profile”) with mRNA levels of selected genes in a high-fat fed or obese animal (“obese animal profile” or “obese profile”). A lean animal gene expression profile is characterized by the reduction of mRNA levels of about 5-10 genes, selected from the group consisting of Lip1, Ppara, Pparg, Pcx, Apoa4, Apoc1, Apoc2, Apoc4, Mttp, Srebp-1, Scd1, Acadl, Acadm, Acads, Acox1, Cpt1a, Cpt2, Crat, Elovl2, Elovl3, Acadsb, Fads2, Facl2, Dbi, Fabp1, Fabp2, Acat-1, Acca-1, Hmgcs1, Hmgcs2, Gck, and G6pc. In addition, a lean animal gene expression profile is characterized by the increase of mRNA levels of at least 2 genes selected from the group consisting of Prkaa2, Prkab1, Scd2, and Soat2. Methods for shifting a gene expression profile from that of an obese animal to that of a lean animal include contacting an animal with an antisense oligonucleotide targeted to apolipoprotein B, which results in a gene expression profile characteristic of a lean animal. Also provided are methods for differentiating a lean animal profile from a high-fat, apolipoprotein B oligonucleotide-treated animal profile. Such differentiating genes are Prkag1, Facl4, Fabp7, and Cyp7b1, 2 or more of which are lowered in lean animals, but are raised in high-fat fed, apolipoprotein B oligonucleotide-treated animals. Additional differentiating genes are Lip1, Lipc, Scd1, Cpt1a, Fasn, Abcd2, Dbi, Cyp7a1, Ldlr, Hmgcs1, and Car5a, 2 or more of which are raised in lean animals, but are lowered in high-fat fed, apolipoprotein B oligonucleotide-treated animals.

While the present invention has been described with specificity in accordance with certain embodiments, the following examples serve only to illustrate the invention and are not intended to limit the same. Each of the references, GENBANK® accession numbers, and the like recited in the present application is incorporated herein by reference in its entirety.

### EXAMPLES

### Example 1

Nucleoside Phosphoramidites for Oligonucleotide Synthesis Deoxy and 2′-alkoxy Amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides was utilized, except the wait step after pulse delivery of tetrazole and base was increased to 360 seconds.

Oligonucleotides containing 5-methyl-2′-deoxycytidine (5-Me-C) nucleotides were synthesized according to published methods [Sanghvi, et. al., Nucleic Acids Research, 1993, 21, 3197-3203] using commercially available phosphoramidites (Glen Research, Sterling Va. or ChemGenes, Needham Mass.).

### 2′-Fluoro Amidites

### 2′-Fluorodeoxyadenosine Amidites

2′-fluoro oligonucleotides were synthesized as described previously [Kawasaki, et. al., J. Med. Chem., 1993, 36, 831-841] and U.S. Pat. No. 5,670,633, herein incorporated by reference. Briefly, the protected nucleoside N6-benzoyl-2′-deoxy-2′-fluoroadenosine was synthesized utilizing commercially available 9-beta-D-arabinofuranosyladenine as starting material and by modifying literature procedures whereby the 2′-alpha-fluoro atom is introduced by a S_{N}2-displacement of a 2′-beta-trityl group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies and standard methods were used to obtain the 5′-dimethoxytrityl-(DMT) and 5′-DMT-3′-phosphoramidite intermediates.

### 2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate diisobutyrylarabinofuranosylguanosine. Deprotection of the TPDS group was followed by protection of the hydroxyl group with THP to give diisobutyryl di-THP protected arabinofuranosylguanine. Selective 0-deacylation and triflation was followed by treatment of the crude product with fluoride, then deprotection of the THP groups.

Standard methodologies were used to obtain the 5′-DMT- and 5′-DMT-3′-phosphoramidites.

### 2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′phosphoramidites.

### 2′-Fluorodeoxycytidine

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′phosphoramidites.

### 2′-O-(2-Methoxyethyl) Modified Amidites

2′-O-Methoxyethyl-substituted nucleoside amidites are prepared as follows, or alternatively, as per the methods of Martin, P., Helvetica Chimica Acta, 1995, 78, 486-504.

### 2,2′-Anhydro[1-(beta-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid that was crushed to a light tan powder (57 g, 85% crude yield). The NMR spectrum was consistent with the structure, contaminated with phenol as its sodium salt (ca. 5%). The material was used as is for further reactions (or it can be purified further by column chromatography using a gradient of methanol in ethyl acetate (10-25%) to give a white solid, mp 222-4° C.).

### 2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product. Additional material was obtained by reworking impure fractions.

### 2′-O-Methoxyethyl-5′-O-dimethoxytrityl-5-methyluridine

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/hexane/acetone (5:5:1) containing 0.5% Et₃NH. The pure fractions were evaporated to give 164 g of product. Approximately 20 g additional was obtained from the impure fractions to give a total yield of 183 g (57%).

### 3′-O-Acetyl-2′-O-methoxyethyl-5′-O-dimethoxytrityl-5-methyluridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by TLC by first quenching the TLC sample with the addition of MeOH. Upon completion of the reaction, as judged by TLC, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/hexane (4:1). Pure product fractions were evaporated to yield 96 g (84%). An additional 1.5 g was recovered from later fractions.

### 3′-O-Acetyl-2′-O-methoxyethyl-5′-O-dimethoxytrityl-5-methyl-4-triazoleuridine

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 h using an overhead stirrer. POCl₃ was added dropwise, over a 30 minute period, to the stirred solution maintained at 0-10° C., and the resulting mixture stirred for an additional 2 hours. The first solution was added dropwise, over a 45 minute period, to the latter solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

### 2′-O-Methoxyethyl-5′-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (TLC showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

### N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, TLC showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/hexane (1:1) containing 0.5% Et₃NH as the eluting solvent. The pure product fractions were evaporated to give 90 g (90%) of the title compound.

### N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) was dissolved in CH₂Cl₂ (1 L). Tetrazole diisopropylamine (7.1 g) and 2-cyanoethoxy-tetra(isopropyl)phosphite (40.5 mL, 0.123 M) were added with stirring, under a nitrogen atmosphere. The resulting mixture was stirred for 20 hours at room temperature (TLC showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAc/hexane (3:1) as the eluting solvent. The pure fractions were combined to give 90.6 g (87%) of the title compound.

### 2′-O-(Aminooxyethyl) Nucleoside Amidites and 2′-O-(dimethylamino-oxyethyl) Nucleoside Amidites

### 2′-(Dimethylaminooxyethoxy) Nucleoside Amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

### 5′-O-tert-Butyldiphenylsilyl-O²-2′-anhydro-5-methyluridine

O²-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 h at ambient temperature. TLC (Rf 0.22, ethyl acetate) indicated a complete reaction. The solution was concentrated under reduced pressure to a thick oil. This was partitioned between dichloromethane (1 L) and saturated sodium bicarbonate (2×1 L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 h) to 149 g (74.8%) of white solid. TLC and NMR were consistent with pure product.

### 5′-O-tert-Butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure <100 psig). The reaction vessel was cooled to ambient and opened. TLC (Rf 0.67 for desired product and Rf 0.82 for ara-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. Alternatively, once the low boiling solvent is gone, the remaining solution can be partitioned between ethyl acetate and water; the product will be in the organic phase. The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

### 2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P₂O₅ under high vacuum for two days at 40° C. The reaction mixture was flushed with argon and dry THF (369.8 mL, Aldrich, sure seal bottle) was added to get a clear solution. Diethyl-azodicarboxylate (6.98 mL, 44.36 mmol) was added dropwise to the reaction mixture. The rate of addition is maintained such that resulting deep red coloration is just discharged before adding the next drop. After the addition was complete, the reaction was stirred for 4 hrs. By that time TLC showed the completion of the reaction (ethylacetate:hexane, 60:40). The solvent was evaporated in vacuum. Residue obtained was placed on a flash column and eluted with ethyl acetate:hexane (60:40), to get 2′-O-([2-phthalimidoxy)ethyl]-5′-t-butyldiphenylsilyl-5-methyluridine as white foam (21.819 g, 86%).

### 5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate was washed with ice cold CH₂Cl₂ and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eq.) was added and the resulting mixture was stirred for 1 h. Solvent was removed under vacuum; residue chromatographed to get 5′-O-tert-butyldiphenylsilyl-2′-O-[(2-formadoximinooxy) ethyl]-5-methyluridine as white foam (1.95 g, 78%).

### 5′-O-tert-Butyldiphenylsilyl-2′-O—[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL). Sodium cyanoborohydride (0.39 g, 6.13 mmol) was added to this solution at 10° C. under inert atmosphere. The reaction mixture was stirred for 10 minutes at 10° C. After that the reaction vessel was removed from the ice bath and stirred at room temperature for 2 h, the reaction monitored by TLC (5% MeOH in CH₂Cl₂). Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mmol) was added and the reaction mixture was stirred at room temperature for 10 minutes. Reaction mixture cooled to 10° C. in an ice bath, sodium cyanoborohydride (0.39 g, 6.13 mmol) was added and reaction mixture stirred at 10° C. for 10 minutes. After 10 minutes, the reaction mixture was removed from the ice bath and stirred at room temperature for 2 hrs. To the reaction mixture 5% NaHCO₃ (25 mL) solution was added and extracted with ethyl acetate (2×25 mL). Ethyl acetate layer was dried over anhydrous Na₂SO₄ and evaporated to dryness. The residue obtained was purified by flash column chromatography and eluted with 5% MeOH in CH₂Cl₂ to get 5′-O-tert-butyldiphenylsilyl-2′-O—[N,N-dimethylaminooxyethyl]-5-methyluridine as a white foam (14.6 g, 80%).

### 2′-O-(dimethylaminooxyethyl)-5-methyluridine

Triethylamine trihydrofluoride (3.91 mL, 24.0 mmol) was dissolved in dry THF and triethylamine (1.67 mL, 12 mmol, dry, kept over KOH). This mixture of triethylamine-2HF was then added to 5′-O-tert-butyldiphenylsilyl-2′-O—[N,N-dimethylaminooxyethyl]-5-methyluridine (1.40 g, 2.4 mmol) and stirred at room temperature for 24 hrs. Reaction was monitored by TLC (5% MeOH in CH₂Cl₂). Solvent was removed under vacuum and the residue placed on a flash column and eluted with 10% MeOH in CH₂Cl₂ to get 2′-O-(dimethylaminooxyethyl)-5-methyluridine (766 mg, 92.5%).

### 5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C. It was then co-evaporated with anhydrous pyridine (20 mL). The residue obtained was dissolved in pyridine (11 mL) under argon atmosphere. 4-dimethylaminopyridine (26.5 mg, 2.60 mmol), 4,4′-dimethoxytrityl chloride (880 mg, 2.60 mmol) was added to the mixture and the reaction mixture was stirred at room temperature until all of the starting material disappeared. Pyridine was removed under vacuum and the residue chromatographed and eluted with 10% MeOH in CH₂Cl₂ (containing a few drops of pyridine) to get 5′-O-DMT-2′-O-(dimethylamino-oxyethyl)-5-methyluridine (1.13 g, 80%).

### 5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL). To the residue N,N-diisopropylamine tetrazonide (0.29 g, 1.67 mmol) was added and dried over P₂O₅ under high vacuum overnight at 40° C. Then the reaction mixture was dissolved in anhydrous acetonitrile (8.4 mL) and 2-cyanoethyl-N,N,N¹,N¹-tetraisopropylphosphoramidite (2.12 mL, 6.08 mmol) was added. The reaction mixture was stirred at ambient temperature for 4 hrs under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO₃ (40 mL). Ethyl acetate layer was dried over anhydrous Na₂SO₄ and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

### 2′-(Aminooxyethoxy) Nucleoside Amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

### N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-([2-phthalmidoxy]ethyl)-5′-O-(4,4′-dimethoxytrityl)guanosine-3′-[(2-cyanoethyl)-N,N-diisopropylphosphoramidite].

### 2′-dimethylaminoethoxyethoxy (2′-DMAEOE) Nucleoside Amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

### 2′-O-[2(2-N,N-dimethylaminoethoxy)ethyl]-5-methyl uridine

2[2-(Dimethylamino)ethoxy]ethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O²-,2′-anhydro-5-methyluridine (1.2 g, 5 mmol), and sodium bicarbonate (2.5 mg) are added and the bomb is sealed, placed in an oil bath and heated to 155° C. for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate and concentrated. The residue is columned on silica gel using methanol/methylene chloride 1:20 (which has 2% triethylamine) as the eluent. As the column fractions are concentrated a colorless solid forms which is collected to give the title compound as a white solid.

### 5′-O-dimethoxytrityl-2′-O-[2(2-N,N-dimethylaminoethoxy)ethyl)]-5-methyl uridine

To 0.5 g (1.3 mmol) of 2′-O-[2(2-N,N-dimethylaminoethoxy)ethyl)]-5-methyl uridine in anhydrous pyridine (8 mL), triethylamine (0.36 mL) and dimethoxytrityl chloride (DMT-C1, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO₃ solution, followed by saturated NaCl solution and dried over anhydrous sodium sulfate. Evaporation of the solvent followed by silica gel chromatography using MeOH:CH₂Cl₂:Et₃N (20:1, v/v, with 1% triethylamine) gives the title compound.

### 5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) are added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

### Example 2

Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 h), the oligonucleotides were purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

### Example 5

Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample was again lyophilized to dryness. The pellet is resuspended in 1M TBAF in THF for 24 hrs at room temperature to deprotect the 2′ positions. The reaction is then quenched with 1M TEAA and the sample is then reduced to ½ volume by rotovac before being desalted on a G25 size exclusion column. The oligo recovered is then analyzed spectrophotometrically for yield and for purity by capillary electrophoresis and by mass spectrometry.

### [2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]-[2′-deoxy Phosphorothioate]-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 6

Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis—96 Well Plate Format

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (BECKMAN P/ACE® MDQ) or, for individually prepared samples, on a commercial CE apparatus (e.g., BECKMAN P/ACE® 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

A. Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following 7 cell types are provided for illustrative purposes, but other cell types can be routinely used, provided that the target is expressed in the cell type chosen. This can be readily determined by methods routine in the art, for example Northern blot analysis, Ribonuclease protection assays, or RT PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence.

NHDF Cells:

Human neonatal dermal fibroblast (NHDF) were obtained from the Clonetics Corporation (Walkersville Md.). NHDFs were routinely maintained in Fibroblast Growth Medium (Clonetics Corporation, Walkersville Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville Md.). HEKs were routinely maintained in Keratinocyte Growth Medium (Clonetics Corporation, Walkersville Md.) formulated as recommended by the supplier. Cells were routinely maintained for up to 10 passages as recommended by the supplier.

HepG2 Cells:

The human hepatoblastoma cell line HepG2 was obtained from the American Type Culture Collection (Manassas, Va.). HepG2 cells were routinely cultured in Eagle's MEM supplemented with 10% fetal calf serum, non-essential amino acids, and 1 mM sodium pyruvate (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

AML12 Cells:

The AML12 (alpha mouse liver 12) cell line was established from hepatocytes from a mouse (CD1 strain, line MT42) transgenic for human TGF alpha. Cells are cultured in a 1:1 mixture of Dulbecco's modified Eagle's medium and Ham's F12 medium with 0.005 mg/ml insulin, 0.005 mg/ml transferrin, 5 ng/ml selenium, and 40 ng/ml dexamethasone, and 90%; 10% fetal bovine serum. For subculturing, spent medium is removed and fresh media of 0.25% trypsin, 0.03% EDTA solution is added. Fresh trypsin solution (1 to 2 ml) is added and the culture is left to sit at room temperature until the cells detach.

Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

Primary Mouse Hepatocytes:

Primary mouse hepatocytes were prepared from CD-1 mice purchased from Charles River Labs (Wilmington, Mass.) and were routinely cultured in Hepatoyte Attachment Media (Gibco) supplemented with 10% Fetal Bovine Serum (Gibco/Life Technologies, Gaithersburg, Md.), 250 nM dexamethasone (Sigma), and 10 nM bovine insulin (Sigma). Cells were seeded into 96-well plates (Falcon-Primaria #3872) at a density of 10000 cells/well for use in RT-PCR analysis.

For Northern blotting or other analyses, cells are plated onto 100 mm or other standard tissue culture plates coated with rat tail collagen (200 ug/mL) (Becton Dickinson) and treated similarly using appropriate volumes of medium and oligonucleotide.

Hep3B Cells:

The human hepatocellular carcinoma cell line Hep3B was obtained from the American Type Culture Collection (Manassas, Va.). Hep3B cells were routinely cultured in Dulbeccos's MEM high glucose supplemented with 10% fetal calf serum, L-glutamine and pyridoxine hydrochloride (Gibco/Life Technologies, Gaithersburg, Md.). Cells were routinely passaged by trypsinization and dilution when they reached 90% confluence. Cells were seeded into 24-well plates (Falcon-Primaria #3846) at a density of 50,000 cells/well for use in RT-PCR analysis.

For Northern blotting or other analyses, cells may be seeded onto 100 mm or other standard tissue culture plates and treated similarly, using appropriate volumes of medium and oligonucleotide.

Rabbit Primary Hepatocytes:

Primary rabbit hepatocytes were purchased from Invitro Technologies (Gaithersburg, Md.) and maintained in Dulbecco's modified Eagle's medium (Gibco). When purchased, the cells had been seeded into 96-well plates for use in RT-PCR analysis and were confluent.

For Northern blotting or other analyses, cells may be seeded onto 100 mm or other standard tissue culture plates and treated similarly using appropriate volumes of medium and oligonucleotide.

Hela Cells:

The human epitheloid carcinoma cell line HeLa was obtained from the American Tissue Type Culture Collection (Manassas, Va.). HeLa cells were routinely cultured in DMEM, high glucose (Invitrogen Corporation, Carlsbad, Calif.) supplemented with 10% fetal bovine serum (Invitrogen Corporation, Carlsbad, Calif.). Cells were seeded into 24-well plates (Falcon-Primaria #3846) at a density of 50,000 cells/well for use in RT-PCR analysis. Cells were routinely passaged by trypsinization and dilution when they reached 90% confluence. Cells 96-well plates (Falcon-Primaria #3872) at a density of 5,000 cells/well for use in RT-PCR analysis. For Northern blotting or other analyses, cells may be seeded onto 100 mm or other standard tissue culture plates and treated similarly, using appropriate volumes of medium and oligonucleotide.

Human Mammary Epithelial Cells:

Normal human mammary epithelial cells (HMECs) were obtained from the American Type Culture Collection (Manassas Va.). HMECs were routinely cultured in DMEM low glucose (Gibco/Life Technologies, Gaithersburg, Md.) supplemented with 10% fetal calf serum (Gibco/Life Technologies, Gaithersburg, Md.). Cells were routinely passaged by trypsinization and dilution when they reached 90% confluence. Cells were seeded into 96-well plates (Falcon-Primaria #353872, BD Biosciences, Bedford, Mass.) at a density of 7000 cells/well for use in RT-PCR analysis. For Northern blotting or other analyses, cells may be seeded onto 100 mm or other standard tissue culture plates and treated similarly, using appropriate volumes of medium and oligonucleotide.

Treatment with Antisense Compounds:

When cells reached 80% confluency, they were treated with oligonucleotide. For cells grown in 96-well plates, wells were washed once with 200 μL OPTI-MEM™-1 reduced-serum medium (Gibco BRL) and then treated with 130 μL of OPTI-MEM™-1 containing 3.75 μg/mL LIPOFECTIN™ (Gibco BRL) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920, TCCGTCATCGCTCCTCAGGG, SEQ ID NO: 1, a 2′-O-methoxyethyl gapmer (2′-O-methoxyethyls shown in bold) with a phosphorothioate backbone which is targeted to human H-ras. For mouse or rat cells the positive control oligonucleotide is ISIS 15770, ATGCATTCTGCCCCCAAGGA, SEQ ID NO: 2, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-Ha-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 5 nM to 300 nM.

B. Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, Ribonuclease protection assays, or real-time PCR.

HepG2 Cells:

The human hepatoblastoma cell line HepG2 was obtained from the American Type Culture Collection (Manassas, Va.). HepG2 cells were routinely cultured in Eagle's MEM supplemented with 10% fetal bovine serum, non-essential amino acids, and 1 mM sodium pyruvate (Invitrogen Life Technologies, Carlsbad, Calif.). Cells were routinely passaged by trypsinization and dilution when they reached 90% confluence. Cells were seeded into 96-well plates (Falcon-Primaria #3872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

AmL12 Cells:

The AML12 (alpha mouse liver 12) cell line was established from hepatocytes from a mouse (CD1 strain, line MT42) transgenic for human TGF alpha. Cells are cultured in a 1:1 mixture of Dulbecco's modified Eagle's medium and Ham's F12 medium with 0.005 mg/ml insulin, 0.005 mg/ml transferrin, 5 ng/ml selenium, and 40 ng/ml dexamethasone, and 90%:10% fetal bovine serum (medium and additives from Invitrogen Life Technologies, Carlsbad Calif. and Sigma-Aldrich, St. Louis, Mo.). For subculturing, spent medium is removed and fresh media of 0.25% trypsin, 0.03% EDTA solution is added. Fresh trypsin solution (1 to 2 ml) is added and the culture is left to sit at room temperature until the cells detach. Cells were routinely passaged by trypsinization and dilution when they reached approximately 90% confluence. Cells were seeded into 96-well plates (Falcon-Primaria #3872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

Primary Mouse Hepatocytes:

Primary mouse hepatocytes were prepared from CD-1 mice purchased from Charles River Labs (Wilmington, Mass.) and were routinely cultured in Hepatoyte Attachment Media (Invitrogen Life Technologies, Carlsbad, Calif.) supplemented with 10% Fetal Bovine Serum (Invitrogen Life Technologies, Carlsbad, Calif.), 250 nM dexamethasone (Sigma), and 10 nM bovine insulin (both from Sigma-Aldrich, St. Louis, Mo.). Cells were seeded into 96-well plates (Falcon-Primaria #3872, BD Biosciences, Bedford, Mass.) at a density of approximately 10,000 cells/well for use in antisense oligonucleotide transfection experiments. For Northern blotting or other analyses, cells are plated onto 100 mm or other standard tissue culture plates coated with rat tail collagen (200 ug/mL) (BD Biosciences, Bedford, Mass.) and treated similarly using appropriate volumes of medium and oligonucleotide.

Hep3B Cells:

The human hepatocellular carcinoma cell line Hep3B was obtained from the American Type Culture Collection (Manassas, Va.). Hep3B cells were routinely cultured in Dulbeccos's MEM high glucose supplemented with 10% fetal bovine serum, L-glutamine and pyridoxine hydrochloride (Invitrogen Life Technologies, Carlsbad, Calif.). Cells were routinely passaged by trypsinization and dilution when they reached approximately 90% confluence. Cells were seeded into 24-well plates (Falcon-Primaria #3846, BD Biosciences, Bedford, Mass.) at a density of approximately 50,000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

HeLa Cells:

The human epitheloid carcinoma cell line HeLa was obtained from the American Tissue Type Culture Collection (Manassas, Va.). HeLa cells were routinely cultured in DMEM, high glucose (Invitrogen Corporation, Carlsbad, Calif.) supplemented with 10% fetal bovine serum (Invitrogen Corporation, Carlsbad, Calif.). Cells were routinely passaged by trypsinization and dilution when they reached approximately 90% confluence. Cells were seeded onto 96-well plates (Falcon-Primaria #3872, BD Biosciences, Bedford, Mass.) at a density of approximately 5,000 cells/well for use in antisense oligonucleotide transfection experiments. Alternatively, cells were seeded into 24-well plates (Falcon-Primaria #3846, BD Biosciences, Bedford, Mass.) at a density of approximately 50,000 cells/well for use in RT-PCR analysis. For Northern blotting or other analyses, cells may be seeded onto 100 mm or other standard tissue culture plates and treated similarly, using appropriate volumes of medium and oligonucleotide.

Human Mammary Epithelial Cells:

Normal human mammary epithelial cells (HMECs) were obtained from the American Type Culture Collection (Manassas Va.). HMECs were routinely cultured in DMEM low glucose supplemented with 10% fetal bovine serum (Invitrogen Corporation, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

Treatment with Antisense Compounds:

When cells reached 65-75% confluency, they were treated with oligonucleotide. Oligonucleotide was mixed with LIPOFECTIN® Invitrogen Life Technologies, Carlsbad, Calif.) in OPTI-MEM® 1 reduced serum medium (Invitrogen Life Technologies, Carlsbad, Calif.) to achieve the desired concentration of oligonucleotide and a LIPOFECTIN® concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEM® 1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920 (TCCGTCATCGCTCCTCAGGG, SEQ ID NO: 1; targeted to human H-ras), a chimeric oligonucleotide having a 9 nucleotide gap segment composed of 2′-deoxynucleotides, which is flanked on the 5′ side and 3′ sides by 3 nucleotide and 8 nucleotide wing segments, respectively. The wings are composed of 2′-O-methoxyethyl nucleotides. For mouse or rat cells the positive control oligonucleotide is ISIS 15770 (ATGCATTCTGCCCCCAAGGA, SEQ ID NO: 2; targeted to rodent c-raf), a chimeric oligonucleotide having a 10 nucleotide gap segment composed of 2′-deoxynucleotides, which is flanked on the 5′ side and 3′ sides by 5 nucleotide wing segments. The wings are composed of 2′-O-methoxyethyl nucleotides. Both compounds have phosphorothioate internucleoside (backbone) linkages and cytidines in the wing segments are 5-methylcytidines. The concentration of positive control oligonucleotide that results in 80% inhibition of H-ras (for ISIS 13920) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 5 nM to 300 nM.

### Example 10

Analysis of Oligonucleotide Inhibition of Apolipoprotein B Expression

Antisense modulation of apolipoprotein B expression can be assayed in a variety of ways known in the art. For example, apolipoprotein B mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM® 7700 Sequence Detection System, available from PE-Applied Biosystems, Foster City, Calif. and used according to manufacturer's instructions.

Protein levels of apolipoprotein B can be quantitated in a variety of ways well known in the art, such as immunoprecipitation, Western blot analysis (immunoblotting), ELISA or fluorescence-activated cell sorting (FACS). Antibodies directed to apolipoprotein B can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 11

Poly(A)+ mRNA Isolation

Poly(A)+ mRNA was isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C. was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 12

Total RNA Isolation

Total RNA was isolated using an RNEASY® 96 kit and buffers purchased from Qiagen Inc. (Valencia Calif.) following the manufacturer's recommended procedures. Briefly, for cells grown on 96-well plates, growth medium was removed from the cells and each well was washed with 200 μL cold PBS. 100 μL Buffer RLT was added to each well and the plate vigorously agitated for 20 seconds. 100 μL of 70% ethanol was then added to each well and the contents mixed by pipetting three times up and down. The samples were then transferred to the RNEASY® 96 well plate attached to a QIAvac® manifold fitted with a waste collection tray and attached to a vacuum source. Vacuum was applied for 15 seconds. 1 mL of Buffer RW1 was added to each well of the RNEASY® 96 plate and the vacuum again applied for 15 seconds. 1 mL of Buffer RPE was then added to each well of the RNEASY® 96 plate and the vacuum applied for a period of 15 seconds. The Buffer RPE wash was then repeated and the vacuum was applied for an additional 10 minutes. The plate was then removed from the QIAvac® manifold and blotted dry on paper towels. The plate was then re-attached to the QIAvac® manifold fitted with a collection tube rack containing 1.2 mL collection tubes. RNA was then eluted by pipetting 60 μL water into each well, incubating 1 minute, and then applying the vacuum for 30 seconds. The elution step was repeated with an additional 60 μL water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

A. Real-Time Quantitative PCR Analysis of Apolipoprotein B mRNA Levels

Quantitation of apolipoprotein B mRNA levels was determined by real-time quantitative PCR using the ABI PRISM® 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE™, FAM™, or VIC™, obtained from either Operon Technologies Inc., Alameda, Calif. or PE-Applied Biosystems, Foster City, Calif.) is attached to the 5′ end of the probe and a quencher dye (e.g., TAMRA™, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM® 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from PE-Applied Biosystems, Foster City, Calif. RT-PCR reactions were carried out by adding 25 μL PCR cocktail (1× TAQMAN™ buffer A, 5.5 mM MgCl₂, 300 μM each of dATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL total RNA solution. The RT reaction was carried out by incubation for 30 minutes at 48° C. Following a 10 minute incubation at 95° C. to activate the AMPLITAQ GOLD™, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ are taught in Jones, L. J., et al, Analytical Biochemistry, 1998, 265, 368-374.

In this assay, 175 μL of RiboGreen™ working reagent (RiboGreen™ reagent diluted 1:2865 in 10 mM Tris-HCl, 1 mM EDTA, pH 7.5) is pipetted into a 96-well plate containing 25 uL purified, cellular RNA. The plate is read in a CytoFluor 4000 (PE Applied Biosystems) with excitation at 480 nm and emission at 520 nm.

Probes and primers to human apolipoprotein B were designed to hybridize to a human apolipoprotein B sequence, using published sequence information (GenBank accession number NM_000384.1, incorporated herein as SEQ ID NO: 3). For human apolipoprotein B the PCR primers were:
forward primer: TGCTAAAGGCACATATGGCCT (SEQ ID NO: 4)
reverse primer: CTCAGGTTGGACTCTCCATTGAG (SEQ ID NO: 5) and the PCR probe was: FAM-CTTGTCAGAGGGATCCTAACACTGGCCG-TAMRA (SEQ ID NO: 6) where FAM (PE-Applied Biosystems, Foster City, Calif.) is the fluorescent reporter dye) and TAMRA (PE-Applied Biosystems, Foster City, Calif.) is the quencher dye.

For human GAPDH the PCR primers were:
forward primer: GAAGGTGAAGGTCGGAGTC (SEQ ID NO: 7)
reverse primer: GAAGATGGTGATGGGATTTC (SEQ ID NO: 8) and the PCR probe was: 5′ JOE-CAAGCTTCCCGTTCTCAGCC-TAMRA 3′ (SEQ ID NO: 9) where JOE (PE-Applied Biosystems, Foster City, Calif.) is the fluorescent reporter dye) and TAMRA (PE-Applied Biosystems, Foster City, Calif.) is the quencher dye.

Probes and primers to mouse apolipoprotein B were designed to hybridize to a mouse apolipoprotein B sequence, using published sequence information (GenBank accession number M35186, incorporated herein as SEQ ID NO: 10). For mouse apolipoprotein B the PCR primers were:
forward primer: CGTGGGCTCCAGCATTCTA (SEQ ID NO: 11)
reverse primer: AGTCATTTCTGCCTTTGCGTC (SEQ ID NO: 12) and the PCR probe was: FAM-CCAATGGTCGGGCACTGCTCAA-TAMRA (SEQ ID NO: 13) where FAM (PE-Applied Biosystems, Foster City, Calif.) is the fluorescent reporter dye) and TAMRA (PE-Applied Biosystems, Foster City, Calif.) is the quencher dye. For mouse GAPDH the PCR primers were:
forward primer: GGCAAATTCAACGGCACAGT (SEQ ID NO: 14)
reverse primer: GGGTCTCGCTCCTGGAAGAT (SEQ ID NO:15) and the PCR probe was: 5′ JOE-AAGGCCGAGAATGGGAAGCTTGTCATC-TAMRA 3′ (SEQ ID NO: 16) where JOE (PE-Applied Biosystems, Foster City, Calif.) is the fluorescent reporter dye) and TAMRA (PE-Applied Biosystems, Foster City, Calif.) is the quencher dye.

B. Real-Time Quantitative PCR Analysis of Apolipoprotein B mRNA Levels

Quantitation of apolipoprotein B mRNA levels was determined by real-time quantitative PCR using the ABI PRISM® 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE™, FAM™, or VIC™, obtained from either Operon Technologies Inc., Alameda, Calif. or PE-Applied Biosystems, Foster City, Calif.) is attached to the 5′ end of the probe and a quencher dye (e.g., TAMRA™, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM® 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time PCR are normalized using either the expression level of GAPDH, a gene whose expression is constant, or by quantifying total RNA using RIBOGREEN® (Molecular Probes, Inc. Eugene, Oreg.). GAPDH expression is quantified by real time PCR, by being run simultaneously with the target, multiplexing, or separately. Total RNA is quantified using RIBOGREEN® RNA quantification reagent from Molecular Probes. Methods of RNA quantification by RIBOGREEN® are taught in Jones, L. J., et al, Analytical Biochemistry, 1998, 265, 368-374.

In this assay, 175 μL of RIBOGREEN® working reagent (RIBOGREEN® reagent diluted 1:2865 in 10 mM Tris-HCl, 1 mM EDTA, pH 7.5) is pipetted into a 96-well plate containing 25 uL purified, cellular RNA. The plate is read in a CytoFluor 4000 (PE Applied Biosystems) with excitation at 480 nm and emission at 520 nm.

Probes and primers to human apolipoprotein B were designed to hybridize to a human apolipoprotein B sequence, using published sequence information (GENBANK® accession number NM_000384.1, incorporated herein as SEQ ID NO: 3). For human apolipoprotein B the PCR primers are:
forward primer: TGCTAAAGGCACATATGGCCT (SEQ ID NO: 4)
reverse primer: CTCAGGTTGGACTCTCCATTGAG (SEQ ID NO: 5) and the PCR probe is: FAM-CTTGTCAGAGGGATCCTAACACTGGCCG-TAMRA (SEQ ID NO: 6) where FAM™ (PE-Applied Biosystems, Foster City, Calif.) is the fluorescent reporter dye) and TAMRA™ (PE-Applied Biosystems, Foster City, Calif.) is the quencher dye.

For human GAPDH the PCR primers are:
forward primer: GAAGGTGAAGGTCGGAGTC (SEQ ID NO: 7)
reverse primer: GAAGATGGTGATGGGATTTC (SEQ ID NO: 8) and the PCR probe is: 5′ JOE-CAAGCTTCCCGTTCTCAGCC-TAMRA 3′ (SEQ ID NO: 9) where JOE™ (PE-Applied Biosystems, Foster City, Calif.) is the fluorescent reporter dye) and TAMRA™ (PE-Applied Biosystems, Foster City, Calif.) is the quencher dye.

Probes and primers to mouse apolipoprotein B were designed to hybridize to a mouse apolipoprotein B sequence, using published sequence information (GENBANK® accession number M35186, incorporated herein as SEQ ID NO: 10). For mouse apolipoprotein B the PCR primers are:
forward primer: CGTGGGCTCCAGCATTCTA (SEQ ID NO: 11)
reverse primer: AGTCATTTCTGCCTTTGCGTC (SEQ ID NO: 12) and the PCR probe is: FAM-CCAATGGTCGGGCACTGCTCAA-TAMRA (SEQ ID NO: 13) where FAM™ (PE-Applied Biosystems, Foster City, Calif.) is the fluorescent reporter dye) and TAMRA™ (PE-Applied Biosystems, Foster City, Calif.) is the quencher dye. For mouse GAPDH the PCR primers are:
forward primer: GGCAAATTCAACGGCACAGT (SEQ ID NO: 14)
reverse primer: GGGTCTCGCTCCTGGAAGAT (SEQ ID NO:15) and the PCR probe is: 5′ JOE-AAGGCCGAGAATGGGAAGCTTGTCATC-TAMRA 3′ (SEQ ID NO: 16) where JOE™ (PE-Applied Biosystems, Foster City, Calif.) is the fluorescent reporter dye) and TAMRA™ (PE-Applied Biosystems, Foster City, Calif.) is the quencher dye.

### Example 14

Northern Blot Analysis of Apolipoprotein B mRNA Levels

Eighteen hours after antisense treatment, cell monolayers were washed twice with cold PBS and lysed in 1 mL RNAZOL® (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND®-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER® UV Crosslinker 2400 (Stratagene, Inc, La Jolla, Calif.) and then robed using QUICKHYB® hybridization solution (Stratagene, La Jolla, Calif.) using manufacturer's recommendations for stringent conditions.

To detect human apolipoprotein B, a human apolipoprotein B specific probe was prepared by PCR using the forward primer TGCTAAAGGCACATATGGCCT (SEQ ID NO: 4) and the reverse primer CTCAGGTTGGACTCTCCATTGAG (SEQ ID NO: 5). To normalize for variations in loading and transfer efficiency membranes were stripped and probed for human glyceraldehyde-3-phosphate dehydrogenase (GAPDH) RNA (Clontech, Palo Alto, Calif.).

To detect mouse apolipoprotein B, a human apolipoprotein B specific probe was prepared by PCR using the forward primer CGTGGGCTCCAGCATTCTA (SEQ ID NO: 11) and the reverse primer AGTCATTTCTGCCTTTGCGTC (SEQ ID NO: 12). To normalize for variations in loading and transfer efficiency membranes were stripped and probed for mouse glyceraldehyde-3-phosphate dehydrogenase (GAPDH) RNA (Clontech, Palo Alto, Calif.).

Hybridized membranes were visualized and quantitated using a PHOSPHORIMAGER® and IMAGEQUANT® Software V3.3 (Molecular Dynamics, Sunnyvale, Calif.). Data was normalized to GAPDH levels in untreated controls.

### Example 15

Antisense Inhibition of Human Apolipoprotein B Expression by Chimeric Phosphorothioate Oligonucleotides Having 2′-MOE Wings and a Deoxy Gap

In accordance with the present invention, a series of oligonucleotides was designed to target different regions of the human apolipoprotein B RNA, using published sequence (GenBank accession number NM_000384.1, incorporated herein as SEQ ID NO: 3). The oligonucleotides are shown in Table 1. “Target site” indicates the first (5′-most) nucleotide number on the particular target sequence to which the oligonucleotide binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Data are averages from two experiments in which HepG2 cells were treated with 150 nM of the compounds in Table 1. If present, “N.D.” indicates “no data”.

TABLE 1
Inhibition of human apolipoprotein B mRNA levels by
chimeric phosphorothioate oligonucleotides having
2′-MOE wings and a deoxy gap

| | | TARGET | TARGET | | | SEQ ID |
|---|---|---|---|---|---|---|
| ISIS # | REGION | SEQ ID NO | SITE | SEQUENCE | % INHIB | NO |
| 147780 | 5′UTR | 3 | 1 | CCGCAGGTCCCGGTGGGAAT | 40 | 17 |
| 147781 | 5′UTR | 3 | 21 | ACCGAGAAGGGCACTCAGCC | 35 | 18 |
| 147782 | 5′UTR | 3 | 71 | GCCTCGGCCTCGCGGCCCTG | 67 | 19 |
| 147783 | Start | 3 | 114 | TCCATCGCCAGCTGCGGTGG | N.D. | 20 |
| | Codon | | | | | |
| 147784 | Coding | 3 | 151 | CAGCGCCAGCAGCGCCAGCA | 70 | 21 |
| 147785 | Coding | 3 | 181 | GCCCGCCAGCAGCAGCAGCA | 29 | 22 |
| 147786 | Coding | 3 | 321 | CTTGAATCAGCAGTCCCAGG | 34 | 23 |
| 147787 | Coding | 3 | 451 | CTTCAGCAAGGCTTTGCCCT | N.D. | 24 |
| 147788 | Coding | 3 | 716 | TTTCTGTTGCCACATTGCCC | 95 | 25 |
| 147789 | Coding | 3 | 911 | GGAAGAGGTGTTGCTCCTTG | 24 | 26 |
| 147790 | Coding | 3 | 951 | TGTGCTACCATCCCATACTT | 33 | 27 |
| 147791 | Coding | 3 | 1041 | TCAAATGCGAGGCCCATCTT | N.D. | 28 |
| 147792 | Coding | 3 | 1231 | GGACACCTCAATCAGCTGTG | 26 | 29 |
| 147793 | Coding | 3 | 1361 | TCAGGGCCACCAGGTAGGTG | N.D. | 30 |
| 147794 | Coding | 3 | 1561 | GTAATCTTCATCCCCAGTGC | 47 | 31 |
| 147795 | Coding | 3 | 1611 | TGCTCCATGGTTTGGCCCAT | N.D. | 32 |
| 147796 | Coding | 3 | 1791 | GCAGCCAGTCGCTTATCTCC | 8 | 33 |
| 147797 | Coding | 3 | 2331 | GTATAGCCAAAGTGGTCCAC | N.D. | 34 |
| 147798 | Coding | 3 | 2496 | CCCAGGAGCTGGAGGTCATG | N.D. | 35 |
| 147799 | Coding | 3 | 2573 | TTGAGCCCTTCCTGATGACC | N.D. | 36 |
| 147800 | Coding | 3 | 2811 | ATCTGGACCCCACTCCTAGC | N.D. | 37 |
| 147801 | Coding | 3 | 2842 | CAGACCCGACTCGTGGAAGA | 38 | 38 |
| 147802 | Coding | 3 | 3367 | GCCCTCAGTAGATTCATCAT | N.D. | 39 |
| 147803 | Coding | 3 | 3611 | GCCATGCCACCCTCTTGGAA | N.D. | 40 |
| 147804 | Coding | 3 | 3791 | AACCCACGTGCCGGAAAGTC | N.D. | 41 |
| 147805 | Coding | 3 | 3841 | ACTCCCAGATGCCTTCTGAA | N.D. | 42 |
| 147806 | Coding | 3 | 4281 | ATGTGGTAACGAGCCCGAAG | 100 | 43 |
| 147807 | Coding | 3 | 4391 | GGCGTAGAGACCCATCACAT | 25 | 44 |
| 147808 | Coding | 3 | 4641 | GTGTTAGGATCCCTCTGACA | N.D. | 45 |
| 147809 | Coding | 3 | 5241 | CCCAGTGATAGCTCTGTGAG | 60 | 46 |
| 147810 | Coding | 3 | 5355 | ATTTCAGCATATGAGCCCAT | 0 | 47 |
| 147811 | Coding | 3 | 5691 | CCCTGAACCTTAGCAACAGT | N.D. | 48 |
| 147812 | Coding | 3 | 5742 | GCTGAAGCCAGCCCAGCGAT | N.D. | 49 |
| 147813 | Coding | 3 | 5891 | ACAGCTGCCCAGTATGTTCT | N.D. | 50 |
| 147814 | Coding | 3 | 7087 | CCCAATAAGATTTATAACAA | 34 | 51 |
| 147815 | Coding | 3 | 7731 | TGGCCTACCAGAGACAGGTA | 45 | 52 |
| 147816 | Coding | 3 | 7841 | TCATACGTTTAGCCCAATCT | 100 | 53 |
| 147817 | Coding | 3 | 7901 | GCATGGTCCCAAGGATGGTC | 0 | 54 |
| 147818 | Coding | 3 | 8491 | AGTGATGGAAGCTGCGATAC | 30 | 55 |
| 147819 | Coding | 3 | 9181 | ATGAGCATCATGCCTCCCAG | N.D. | 56 |
| 147820 | Coding | 3 | 9931 | GAACACATAGCCGAATGCCG | 100 | 57 |
| 147821 | Coding | 3 | 10263 | GTGGTGCCCTCTAATTTGTA | N.D. | 58 |
| 147822 | Coding | 3 | 10631 | CCCGAGAAAGAACCGAACCC | N.D. | 59 |
| 147823 | Coding | 3 | 10712 | TGCCCTGCAGCTTCACTGAA | 19 | 60 |
| 147824 | Coding | 3 | 11170 | GAAATCCCATAAGCTCTTGT | N.D. | 61 |
| 147825 | Coding | 3 | 12301 | AGAAGCTGCCTCTTCTTCCC | 72 | 62 |
| 147826 | Coding | 3 | 12401 | TCAGGGTGAGCCCTGTGTGT | 80 | 63 |
| 147827 | Coding | 3 | 12471 | CTAATGGCCCCTTGATAAAC | 13 | 64 |
| 147828 | Coding | 3 | 12621 | ACGTTATCCTTGAGTCCCTG | 12 | 65 |
| 147829 | Coding | 3 | 12741 | TATATCCCAGGTTTCCCCGG | 64 | 66 |
| 147830 | Coding | 3 | 12801 | ACCTGGGACAGTACCGTCCC | N.D. | 67 |
| 147831 | 3′UTR | 3 | 13921 | CTGCCTACTGCAAGGCTGGC | 0 | 68 |
| 147832 | 3′UTR | 3 | 13991 | AGAGACCTTCCGAGCCCTGG | N.D. | 69 |
| 147833 | 3′UTR | 3 | 14101 | ATGATACACAATAAAGACTC | 25 | 70 |

As shown in Table 1, SEQ ID NOs 17, 18, 19, 21, 23, 25, 27, 31, 38, 43, 46, 51, 52, 53, 55, 57, 62, 63 and 66 demonstrated at least 30% inhibition of human apolipoprotein B expression in this assay and are therefore preferred. The target sites to which these preferred sequences are complementary are herein referred to as “active sites” and are therefore preferred sites for targeting by compounds of the present invention. As apolipoprotein B exists in two forms in mammals (ApoB-48 and ApoB-100) which are collinear at the amino terminus, antisense oligonucleotides targeting nucleotides 1-6530 hybridize to both forms, while those targeting nucleotides 6531-14121 are specific to the long form of apolipoprotein B.

### Example 16

Antisense Inhibition of Human Apolipoprotein B Expression by Chimeric Phosphorothioate Oligonucleotides Having 2′-MOE Wings and a Deoxy Gap-Dose Response Study

In accordance with the present invention, a subset of the antisense oligonuclotides in Example 15 were further investigated in dose-response studies. Treatment doses were 50, 150 and 250 nM. The compounds were analyzed for their effect on human apolipoprotein B mRNA levels in HepG2 cells by quantitative real-time PCR as described in other examples herein. Data are averages from two experiments and are shown in Table 2.

TABLE 2
Inhibition of human apolipoprotein B mRNA levels
by chimeric phosphorothioate oligonucleotides
having 2′-MOE wings and a deoxy gap

| | Percent Inhibition | | |
|---|---|---|---|
| ISIS # | 50 nM | 150 nM | 250 nM |
| 147788 | 54 | 63 | 72 |
| 147806 | 23 | 45 | 28 |
| 147816 | 25 | 81 | 65 |
| 147820 | 10 | 0 | 73 |

### Example 17

Antisense Inhibition of Mouse Apolipoprotein B Expression by Chimeric Phosphorothioate Oligonucleotides Having 2′-MOE Wings and a Deoxy Gap

In accordance with the present invention, a series of oligonucleotides was designed to target different regions of the mouse apolipoprotein B RNA, using published sequence (GenBank accession number M35186, incorporated herein as SEQ ID NO: 10). The oligonucleotides are shown in Table 3. “Target site” indicates the first (5′-most) nucleotide number on the particular target sequence to which the oligonucleotide binds. All compounds in Table 3 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on mouse apolipoprotein B mRNA levels in primary mouse hepatocytes by quantitative real-time PCR as described in other examples herein. Primary mouse hepatocytes were treated with 150 nM of the compounds in Table 3. Data are averages from two experiments. If present, “N.D.” indicates “no data”.

TABLE 3
Inhibition of mouse apolipoprotein B mRNA levels by
chimeric phosphorothioate oligonucleotides having
2′-MOE wings and a deoxy gap

| | | TARGET | TARGET | | | SEQ ID |
|---|---|---|---|---|---|---|
| ISIS # | REGION | SEQ ID NO | SITE | SEQUENCE | % INHIB | NO |
| 147475 | Coding | 10 | 13 | ATTGTATGTGAGAGGTGAGG | 79 | 71 |
| 147476 | Coding | 10 | 66 | GAGGAGATTGGATCTTAAGG | 13 | 72 |
| 147477 | Coding | 10 | 171 | CTTCAAATTGGGACTCTCCT | N.D | 73 |
| 147478 | Coding | 10 | 211 | TCCAGGAATTGAGCTTGTGC | 78 | 74 |
| 147479 | Coding | 10 | 238 | TTCAGGACTGGAGGATGAGG | N.D | 75 |
| 147480 | Coding | 10 | 291 | TCTCACCCTCATGCTCCATT | 54 | 76 |
| 147481 | Coding | 10 | 421 | TGACTGTCAAGGGTGAGCTG | 24 | 77 |
| 147482 | Coding | 10 | 461 | GTCCAGCCTAGGAACACTCA | 59 | 78 |
| 147483 | Coding | 10 | 531 | ATGTCAATGCCACATGTCCA | N.D | 79 |
| 147484 | Coding | 10 | 581 | TTCATCCGAGAAGTTGGGAC | 49 | 80 |
| 147485 | Coding | 10 | 601 | ATTTGGGACGAATGTATGCC | 64 | 81 |
| 147486 | Coding | 10 | 711 | AGTTGAGGAAGCCAGATTCA | N.D | 82 |
| 147487 | Coding | 10 | 964 | TTCCCAGTCAGCTTTAGTGG | 73 | 83 |
| 147488 | Coding | 10 | 1023 | AGCTTGCTTGTTGGGCACGG | 72 | 84 |
| 147489 | Coding | 10 | 1111 | CCTATACTGGCTTCTATGTT | 5 | 85 |
| 147490 | Coding | 10 | 1191 | TGAACTCCGTGTAAGGCAAG | N.D | 86 |
| 147491 | Coding | 10 | 1216 | GAGAAATCCTTCAGTAAGGG | 71 | 87 |
| 147492 | Coding | 10 | 1323 | CAATGGAATGCTTGTCACTG | 68 | 88 |
| 147493 | Coding | 10 | 1441 | GCTTCATTATAGGAGGTGGT | 41 | 89 |
| 147494 | Coding | 10 | 1531 | ACAACTGGGATAGTGTAGCC | 84 | 90 |
| 147495 | Coding | 10 | 1631 | GTTAGGACCAGGGATTGTGA | 0 | 91 |
| 147496 | Coding | 10 | 1691 | ACCATGGAAAACTGGCAACT | 19 | 92 |
| 147497 | Coding | 10 | 1721 | TGGGAGGAAAAACTTGAATA | N.D | 93 |
| 147498 | Coding | 10 | 1861 | TGGGCAACGATATCTGATTG | 0 | 94 |
| 147499 | Coding | 10 | 1901 | CTGCAGGGCGTCAGTGACAA | 29 | 95 |
| 147500 | Coding | 10 | 1932 | GCATCAGACGTGATGTTCCC | N.D | 96 |
| 147501 | Coding | 10 | 2021 | CTTGGTTAAACTAATGGTGC | 18 | 97 |
| 147502 | Coding | 10 | 2071 | ATGGGAGCATGGAGGTTGGC | 16 | 98 |
| 147503 | Coding | 10 | 2141 | AATGGATGATGAAACAGTGG | 26 | 99 |
| 147504 | Coding | 10 | 2201 | ATCAATGCCTCCTGTTGCAG | N.D | 100 |
| 147505 | Coding | 10 | 2231 | GGAAGTGAGACTTTCTAAGC | 76 | 101 |
| 147506 | Coding | 10 | 2281 | AGGAAGGAACTCTTGATATT | 58 | 102 |
| 147507 | Coding | 10 | 2321 | ATTGGCTTCATTGGCAACAC | 81 | 103 |
| 147759 | Coding | 10 | 1 | AGGTGAGGAAGTTGGAATTC | 19 | 104 |
| 147760 | Coding | 10 | 121 | TTGTTCCCTGAAGTTGTTAC | N.D | 105 |
| 147761 | Coding | 10 | 251 | GTTCATGGATTCCTTCAGGA | 45 | 106 |
| 147762 | Coding | 10 | 281 | ATGCTCCATTCTCACATGCT | 46 | 107 |
| 147763 | Coding | 10 | 338 | TGCGACTGTGTCTGATTTCC | 34 | 108 |
| 147764 | Coding | 10 | 541 | GTCCCTGAAGATGTCAATGC | 97 | 109 |
| 147765 | Coding | 10 | 561 | AGGCCCAGTTCCATGACCCT | 59 | 110 |
| 147766 | Coding | 10 | 761 | GGAGCCCACGTGCTGAGATT | 59 | 111 |
| 147767 | Coding | 10 | 801 | CGTCCTTGAGCAGTGCCCGA | 5 | 112 |
| 147768 | Coding | 10 | 1224 | CCCATATGGAGAAATCCTTC | 24 | 113 |
| 147769 | Coding | 10 | 1581 | CATGCCTGGAAGCCAGTGTC | 89 | 114 |
| 147770 | Coding | 10 | 1741 | GTGTTGAATCCCTTGAAATC | 67 | 115 |
| 147771 | Coding | 10 | 1781 | GGTAAAGTTGCCCATGGCTG | 68 | 116 |
| 147772 | Coding | 10 | 1841 | GTTATAAAGTCCAGCATTGG | 78 | 117 |
| 147773 | Coding | 10 | 1931 | CATCAGACGTGATGTTCCCT | 85 | 118 |
| 147774 | Coding | 10 | 1956 | TGGCTAGTTTCAATCCCCTT | 84 | 119 |
| 147775 | Coding | 10 | 2002 | CTGTCATGACTGCCCTTTAC | 52 | 120 |
| 147776 | Coding | 10 | 2091 | GCTTGAAGTTCATTGAGAAT | 92 | 121 |
| 147777 | Coding | 10 | 2291 | TTCCTGAGAAAGGAAGGAAC | N.D | 122 |
| 147778 | Coding | 10 | 2331 | TCAGATATACATTGGCTTCA | 14 | 123 |

As shown in Table 3, SEQ ID Nos 71, 74, 76, 78, 81, 83, 84, 87, 88, 90, 101, 102, 103, 109, 111, 111, 114, 115, 116, 117, 118, 119, 120 and 121 demonstrated at least 50% inhibition of mouse apolipoprotein B expression in this assay and are therefore preferred. The target sites to which these preferred sequences are complementary are herein referred to as “active sites” and are therefore preferred sites for targeting by compounds of the present invention.

### Example 18

Antisense Inhibition Mouse Apolipoprotein B Expression by Chimeric Phosphorothioate Oligonucleotides Having 2′-MOE Wings and a Deoxy Gap—Dose Response Study

In accordance with the present invention, a subset of the antisense oligonuclotides in Example 17 were further investigated in dose-response studies. Treatment doses were 50, 150 and 300 nM. The compounds were analyzed for their effect on mouse apolipoprotein B mRNA levels in primary hepatocytes cells by quantitative real-time PCR as described in other examples herein. Data are averages from two experiments and are shown in Table 4.

TABLE 4
Inhibition of mouse apolipoprotein B mRNA levels
by chimeric phosphorothioate oligonucleotides
having 2′-MOE wings and a deoxy gap

| | Percent Inhibition | | |
|---|---|---|---|
| ISIS # | 50 nM | 150 nM | 300 nM |
| 147483 | 56 | 88 | 89 |
| 147764 | 48 | 84 | 90 |
| 147769 | 3 | 14 | 28 |
| 147776 | 0 | 17 | 44 |

### Example 19

Western Blot Analysis of Apolipoprotein B Protein Levels

Western blot analysis (immunoblot analysis) was carried out using standard methods. Cells were harvested 16-20 h after oligonucleotide treatment, washed once with PBS, suspended in Laemmli buffer (100 ul/well), boiled for 5 minutes and loaded on a 16% SDS-PAGE gel. Gels were run for 1.5 hours at 150 V, and transferred to membrane for western blotting. Appropriate primary antibody directed to apolipoprotein B was used, with a radiolabelled or fluorescently labeled secondary antibody directed against the primary antibody species. Bands were visualized using a PHOSPHORIMAGER™ (Molecular Dynamics, Sunnyvale Calif.) or the ECL+ chemiluminescent detection system (Amersham Biosciences, Piscataway, N.J.).

### Example 20

Effects of Antisense Inhibition of Apolipoprotein B (ISIS 147764) in C57BL/6 Mice: Lean Animals Vs. High Fat Fed Animals.

C57BL/6 mice, a strain reported to be susceptible to hyperlipidemia-induced atherosclerotic plaque formation were used in the following studies to evaluate antisense oligonucleotides as potential lipid lowering compounds in lean versus high fat fed mice.

Male C57BL/6 mice were divided into two matched groups; (1) wild-type control animals (lean animals) and (2) animals receiving a high fat diet (60% kcal fat). Control animals received saline treatment and were maintained on a normal rodent diet. After overnight fasting, mice from each group were dosed intraperitoneally every three days with saline or 50 mg/kg ISIS 147764 (SEQ ID No: 109) for six weeks. At study termination and forty eight hours after the final injections, animals were sacrificed and evaluated for target mRNA levels in liver, cholesterol and triglyceride levels, liver enzyme levels and serum glucose levels.

The results of the comparative studies are shown in Table 5.

TABLE 5
Effects of ISIS 147764 treatment on apolipoprotein B mRNA, cholesterol, lipid,
triglyceride, liver enzyme and glucose levels in lean and high fat mice.

| | Percent Change | | | | | | | | |
|---|---|---|---|---|---|---|---|---|---|
| Treatment | | | Lipoproteins | | | | Liver Enzymes | | |
| Group | mRNA | CHOL | VLDL | LDL | HDL | TRIG | AST | ALT | GLUC |
| Lean- | −73 | −63 | No | −64 | −44 | −34 | Slight | No | No |
| control | | | change | | | | decrease | change | change |
| High Fat | −87 | −67 | No | −87 | −65 | No | Slight | Slight | −28 |
| Group | | | change | | | change | decrease | increase | |

It is evident from these data that treatment with ISIS 147764 lowered cholesterol as well as LDL and HDL lipoproteins and serum glucose in both lean and high fat mice and that the effects demonstrated are, in fact, due to the inhibition of apolipoprotein B expression as supported by the decrease in mRNA levels. No significant changes in liver enzyme levels were observed, indicating that the antisense oligonucleotide was not toxic to either treatment group.

### Example 21

Effects of Antisense Inhibition of Apolipoprotein B (ISIS 147764) on High Fat Fed Mice; 6 Week Timecourse Study

In accordance with the present invention, a 6-week timecourse study was performed to further investigate the effects of ISIS 147764 on lipid and glucose metabolism in high fat fed mice.

Male C57BL/6 mice (n=8) receiving a high fat diet (60% kcal fat) were evaluated over the course of 6 weeks for the effects of treatment with the antisense oligonucleotide, ISIS 147764. Control animals received saline treatment (50 mg/kg). A subset of animals received a daily oral dose (20 mg/kg) atorvastatin calcium (Lipitor®, Pfizer Inc.). All mice, except atorvastatin-treated animals, were dosed intraperitoneally every three days (twice a week), after fasting overnight, with 5, 25, 50 mg/kg ISIS 147764 (SEQ ID No: 109) or saline (50 mg/kg) for six weeks. Serum cholesterol and lipoproteins were analyzed at 0, 2 and 6 week interim timepoints. At study termination, animals were sacrificed 48 hours after the final injections and evaluated for levels of target mRNA levels in liver, cholesterol, lipoprotein, triglyceride, liver enzyme (AST and ALT) and serum glucose levels as well as body, liver, spleen and fat pad weights.

### Example 22

Effects of Antisense Inhibition of Apolipoprotein B (ISIS 147764) in High Fat Fed Mice—mRNA Expression in Liver

Male C57BL/6 mice (n=8) receiving a high fat diet (60% kcal fat) were evaluated over the course of 6 weeks for the effects of ISIS 147764 on mRNA expression. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks. At study termination, animals were sacrificed 48 hours after the final injections and evaluated for levels of target mRNA levels in liver. ISIS 147764 showed a dose-response effect, reducing mRNA levels by 15, 75 and 88% at doses of 5, 25 and 50 mg/kg, respectively.

Liver protein samples collected at the end of the treatment period were subjected to immunoblot analysis using an antibody directed to mouse apolipoprotein B protein (Gladstone Institute, San Francisco, Calif.). These data demonstrate that treatment with ISIS 147764 decreases apolipoprotein B protein expression in liver in a dose-dependent manner, in addition to reducing mRNA levels.

### Example 23

Effects of Antisense Inhibition of Apolipoprotein B (ISIS 147764) on Serum Cholesterol and Triglyceride Levels

Male C57BL/6 mice (n=8) receiving a high fat diet (60% kcal fat) were evaluated over the course of 6 weeks for the effects of ISIS 147764 on serum cholesterol and triglyceride levels. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks.

Serum cholesterol levels were measured at 0, 2 and 6 weeks and this data is shown in Table 6. Values in the table are expressed as percent inhibition and are normalized to the saline control.

In addition to serum cholesterol, at study termination, animals were sacrificed 48 hours after the final injections and evaluated for triglyceride levels.

Mice treated with ISIS 147764 showed a reduction in both serum cholesterol (240 mg/dL for control animals and 225, 125 and 110 mg/dL for doses of 5, 25, and 50 mg/kg, respectively) and triglycerides (115 mg/dL for control animals and 125, 150 and 85 mg/dL for doses of 5, 25, and 50 mg/kg, respectively) to normal levels by study end. These data were also compared to the effects of atorvastatin calcium at an oral dose of 20 mg/kg which showed only a minimal decrease in serum cholesterol of 20 percent at study termination.

TABLE 6
Percent Inhibition of mouse apolipoprotein
B cholesterol levels by ISIS 147764

Percent Inhibition

| time | Saline | 5 mg/kg | 25 mg/kg | 50 mg/kg |
|---|---|---|---|---|
| 0 weeks | 0 | 0 | 0 | 0 |
| 2 weeks | 0 | 5 | 12 | 20 |
| 6 weeks | 0 | 10 | 45 | 55 |

### Example 24

Effects of Antisense Inhibition of Apolipoprotein B (ISIS 147764) on Lipoprotein Levels

Male C57BL/6 mice (n=8) receiving a high fat diet (60% kcal fat) were evaluated over the course of 6 weeks for the effects of ISIS 147764 on lipoprotein (VLDL, LDL and HDL) levels. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks.

Lipoprotein levels were measured at 0, 2 and 6 weeks and this data is shown in Table 7. Values in the table are expressed as percent inhibition and are normalized to the saline control. Negative values indicate an observed increase in lipoprotein levels.

These data were also compared to the effects of atorvastatin calcium at a daily oral dose of 20 mg/kg at 0, 2 and 6 weeks.

These data demonstrate that at a dose of 50 mg/kg, ISIS 147764 is capable of lowering all categories of serum lipoproteins investigated to a greater extent than atorvastatin.

TABLE 7
Percent Inhibition of mouse apolipoprotein B lipoprotein
levels by ISIS 147764 as compared to atorvastatin

| | | Percent Inhibition | | | | |
|---|---|---|---|---|---|---|
| | | Dose | | | | |
| Lipo- | Time | | 5 | 25 | 50 | atorvastatin |
| protein | (weeks) | Saline | mg/kg | mg/kg | mg/kg | (20 mg/kg) |
| VLDL | 0 | 0 | 0 | 0 | 0 | 0 |
| | 2 | 0 | 25 | 30 | 40 | 15 |
| | 6 | 0 | 10 | −30 | 15 | −5 |
| LDL | 0 | 0 | 0 | 0 | 0 | 0 |
| | 2 | 0 | −30 | 10 | 40 | 10 |
| | 6 | 0 | −10 | 55 | 90 | −10 |
| HDL | 0 | 0 | 0 | 0 | 0 | 0 |
| | 2 | 0 | 5 | 10 | 10 | 15 |
| | 6 | 0 | 10 | 45 | 50 | 20 |

### Example 25

Effects of Antisense Inhibition of Apolipoprotein B (ISIS 147764) on Serum AST and ALT Levels

Male C57BL/6 mice (n=8) receiving a high fat diet (60% kcal fat) were evaluated over the course of 6 weeks for the effects of ISIS 147764 on liver enzyme (AST and ALT) levels. Increased levels of the liver enzymes ALT and AST indicate toxicity and liver damage. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks. AST and ALT levels were measured at 6 weeks.

Mice treated with ISIS 147764 showed no significant change in AST levels over the duration of the study compared to saline controls (105, 70 and 80 IU/L for doses of 5, 25 and 50 mg/kg, respectively compared to 65 IU/L for saline control). Mice treated with atorvastatin at a daily oral dose of 20 mg/kg had AST levels of 85 IU/L.

ALT levels were increased by all treatments with ISIS 147764 over the duration of the study compared to saline controls (50, 70 and 100 IU/L for doses of 5, 25 and 50 mg/kg, respectively compared to 25 IU/L for saline control). Mice treated with atorvastatin at a daily oral dose of 20 mg/kg had AST levels of 40 IU/L.

### Example 26

Effects of Antisense Inhibition of Apolipoprotein B (ISIS 147764) on Serum Glucose Levels

Male C57BL/6 mice (n=8) receiving a high fat diet (60% kcal fat) were evaluated over the course of 6 weeks for the effects of ISIS 147764 on serum glucose levels. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks.

At study termination, animals were sacrificed 48 hours after the final injections and evaluated for serum glucose levels. ISIS 147764 showed a dose-response effect, reducing serum glucose levels to 225, 190 and 180 mg/dL at doses of 5, 25 and 50 mg/kg, respectively compared to the saline control of 300 mg/dL. Mice treated with atorvastatin at a daily oral dose of 20 mg/kg had serum glucose levels of 215 mg/dL. These data demonstrate that ISIS 147764 is capable of reducing serum glucose levels in high fat fed mice.

### Example 27

Effects of Antisense Inhibition of Apolipoprotein B (ISIS 147764) on Body, Spleen, Liver and Fat Pad Weight

Male C57BL/6 mice (n=8) receiving a high fat diet (60% kcal fat) were evaluated over the course of 6 weeks for the effects of ISIS 147764 on body, spleen, liver and fat pad weight. Control animals received saline treatment (50 mg/kg). Mice were dosed intraperitoneally every three days (twice a week), after fasting overnight, with 5, 25, 50 mg/kg ISIS 147764 (SEQ ID No: 109) or saline (50 mg/kg) for six weeks.

At study termination, animals were sacrificed 48 hours after the final injections and body, spleen, liver and fat pad weights were measured. These data are shown in Table 8. Values are expressed as percent change in body weight or ogan weight compared to the saline-treated control animals. Data from mice treated with atorvastatin at a daily dose of 20 mg/kg are also shown in the table. Negative values indicated a decrease in weight.

TABLE 8
Effects of antisense inhibition of mouse
apolipoprotein B on body and organ weight

Percent Change

| | Dose | | | Atorvastatin |
|---|---|---|---|---|
| Tissue | 5 mg/kg | 25 mg/kg | 50 mg/kg | 20 mg/kg |
| Total | 5 | 5 | −4 | 1 |
| Body Wt. | | | | |
| Spleen | 10 | 10 | 46 | 10 |
| Liver | 18 | 70 | 80 | 15 |
| Fat | 10 | 6 | −47 | 7 |

These data show a decrease in fat over the dosage range of ISIS 147764 counterbalanced by an increase in both spleen and liver weight with increased dose to give an overall decrease in total body weight.

### Example 28

Effects of Antisense Inhibition of Apolipoprotein B (ISIS 147764) in B6.129P-Apoe^{tm1Unc} knockout mice: Lean animals vs. High Fat Fed animals. B6.129P-ApoE^{tm1Unc} knockout mice (herein referred to as ApoE knockout mice) obtained from The Jackson Laboratory (Bar Harbor, Me.), are homozygous for the Apoe^{tm1Unc} mutation and show a marked increase in total plasma cholesterol levels that are unaffected by age or sex. These animals present with fatty streaks in the proximal aorta at 3 months of age. These lesions increase with age and progress to lesions with less lipid but more elongated cells, typical of a more advanced stage of pre-atherosclerotic lesion.

The mutation in these mice resides in the apolipoprotein E (ApoE) gene. The primary role of the ApoE protein is to transport cholesterol and triglycerides throughout the body. It stabilizes lipoprotein structure, binds to the low density lipoprotein receptor (LDLR) and related proteins, and is present in a subclass of HDLs, providing them the ability to bind to LDLR. ApoE is expressed most abundantly in the liver and brain. Female B6.129P-Apoetm1Unc knockout mice (ApoE knockout mice) were used in the following studies to evaluate antisense oligonucleotides as potential lipid lowering compounds.

Female ApoE knockout mice ranged in age from 5 to 7 weeks and were placed on a normal diet for 2 weeks before study initiation. ApoE knockout mice were then fed ad libitum a 60% fat diet, with 0.15% added cholesterol to induce dyslipidemia and obesity. Control animals were maintained on a high-fat diet with no added cholesterol. After overnight fasting, mice from each group were dosed intraperitoneally every three days with saline, 50 mg/kg of a control antisense oligonucleotide (ISIS 29837; TCGATCTCCTTTTATGCCCG; SEQ ID NO. 124) or 5, 25 or 50 mg/kg ISIS 147764 (SEQ ID No: 109) for six weeks.

The control oligonucleotide is a chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

At study termination and forty eight hours after the final injections, animals were sacrificed and evaluated for target mRNA levels in liver by RT-PCR methods verified by Northern Blot analysis, glucose levels, cholesterol and lipid levels by HPLC separation methods and triglyceride and liver enzyme levels (performed by LabCorp Preclinical Services; San Diego, Calif.). Data from ApoE knockout mice treated with atorvastatin at a daily dose of 20 mg/kg are also shown in the table for comparison.

The results of the comparative studies are shown in Table 9. Data are normalized to saline controls.

TABLE 9
Effects of ISIS 147764 treatment on apolipoprotein B mRNA, cholesterol, glucose,
lipid, triglyceride and liver enzyme levels in ApoE knockout mice.

| | | Percent Inhibition | | | | |
|---|---|---|---|---|---|---|
| | | Dose | | | | |
| | | | 5 | 25 | 50 | atorvastatin |
| | | Control | mg/kg | mg/kg | mg/kg | (20 mg/kg) |
| mRNA | | 0 | 2 | 42 | 70 | 10 |
| | Glucose Levels (mg/dL) | | | | | |
| Glucose | | 225 | 195 | 209 | 191 | 162 |
| | Cholesterol Levels (mg/dL) | | | | | |
| Cholesterol | | 1750 | 1630 | 1750 | 1490 | 938 |
| | | Lipoprotein Levels (mg/dL) | | | | |
| Lipoprotein | HDL | 51 | 49 | 62 | 61 | 42 |
| | LDL | 525 | 475 | 500 | 325 | 250 |
| | VLDL | 1190 | 1111 | 1194 | 1113 | 653 |
| | Liver Enzyme Levels (IU/L) | | | | | |
| Liver | AST | 55 | 50 | 60 | 85 | 75 |
| Enzymes | ALT | 56 | 48 | 59 | 87 | 76 |

It is evident from these data that treatment with ISIS 147764 lowered glucose and cholesterol as well as all lipoproteins investigated (HDL, LDL and VLDL) in ApoE knockout mice. Further, these decreases correlated with a decrease in both protein and RNA levels of apolipoprotein B, demonstrating an antisense mechanism of action. No significant changes in liver enzyme levels were observed, indicating that the antisense oligonucleotide was not toxic to either treatment group.

### Example 29

Antisense Inhibition of Human Apolipoprotein B Expression by Chimeric Phosphorothioate Oligonucleotides Having 2′-MOE Wings and a Deoxy Gap: Additional Oligonucleotides

In accordance with the present invention, another series of oligonucleotides was designed to target different regions of the human apolipoprotein B RNA, using published sequence (GenBank accession number NM_000384.1, incorporated herein as SEQ ID NO: 3). The oligonucleotides are shown in Table 10. “Target site” indicates the first (5′-most) nucleotide number on the particular target sequence to which the oligonucleotide binds. All compounds in Table 10 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Data are averages from two experiments in which HepG2 cells were treated with 150 nM of the compounds in Table 10. If present, “N.D.” indicates “no data”.

TABLE 10
Inhibition of human apolipoprotein B mRNA levels by
chimeric phosphorothioate oligonucleotides having
2′-MOE wings and a deoxy gap

| | | TARGET | | | | |
|---|---|---|---|---|---|---|
| | | SEQ ID | TARGET | | | SEQ ID |
| ISIS # | REGION | NO | SITE | SEQUENCE | % INHIB | NO |
| 270985 | 5′UTR | 3 | 199 | TTCCTCTTCGGCCCTGGCGC | 75 | 124 |
| 270986 | coding | 3 | 299 | CTCCACTGGAACTCTCAGCC | 0 | 125 |
| 270987 | exon: | 3 | 359 | CCTCCAGCTCAACCTTGCAG | 0 | 126 |
| | exon | | | | | |
| | junction | | | | | |
| 270988 | coding | 3 | 429 | GGGTTGAAGCCATACACCTC | 6 | 127 |
| 270989 | exon: | 3 | 509 | CCAGCTTGAGCTCATACCTG | 64 | 128 |
| | exon | | | | | |
| | junction | | | | | |
| 270990 | coding | 3 | 584 | CCCTCTTGATGTTCAGGATG | 42 | 129 |
| 270991 | coding | 3 | 669 | GAGCAGTTTCCATACACGGT | 21 | 130 |
| 270992 | coding | 3 | 699 | CCCTTCCTCGTCTTGACGGT | 8 | 131 |
| 270993 | coding | 3 | 756 | TTGAAGCGATCACACTGCCC | 69 | 132 |
| 270994 | coding | 3 | 799 | GCCTTTGATGAGAGCAAGTG | 51 | 133 |
| 270995 | coding | 3 | 869 | TCCTCTTAGCGTCCAGTGTG | 40 | 134 |
| 270996 | coding | 3 | 1179 | CCTCTCAGCTCAGTAACCAG | 0 | 135 |
| 270997 | coding | 3 | 1279 | GCACTGAGGCTGTCCACACT | 24 | 136 |
| 270998 | coding | 3 | 1419 | CGCTGATCCCTCGCCATGTT | 1 | 137 |
| 270999 | coding | 3 | 1459 | GTTGACCGCGTGGCTCAGCG | 76 | 138 |
| 271000 | coding | 3 | 1499 | GCAGCTCCTGGGTCCCTGTA | 22 | 139 |
| 271001 | coding | 3 | 1859 | CCCATGGTAGAATTTGGACA | 53 | 140 |
| 271002 | exon: | 3 | 2179 | AATCTCGATGAGGTCAGCTG | 48 | 141 |
| | exon | | | | | |
| | junction | | | | | |
| 271003 | coding | 3 | 2299 | GACACCATCAGGAACTTGAC | 46 | 142 |
| 271004 | coding | 3 | 2459 | GCTCCTCTCCCAAGATGCGG | 10 | 143 |
| 271005 | coding | 3 | 2518 | GGCACCCATCAGAAGCAGCT | 32 | 144 |
| 271006 | coding | 3 | 2789 | AGTCCGGAATGATGATGCCC | 42 | 145 |
| 271007 | coding | 3 | 2919 | CTGAGCAGCTTGACTGGTCT | 26 | 146 |
| 271008 | coding | 3 | 3100 | CCCGGTCAGCGGATAGTAGG | 37 | 147 |
| 271010 | exon: | 3 | 3449 | TGTCACAACTTAGGTGGCCC | 57 | 148 |
| | exon | | | | | |
| | junction | | | | | |
| 271011 | coding | 3 | 3919 | GTCTGGCAATCCCATGTTCT | 51 | 149 |
| 271012 | coding | 3 | 4089 | CCCACAGACTTGAAGTGGAG | 55 | 150 |
| 271013 | coding | 3 | 4579 | GAACTGCCCATCAATCTTGA | 19 | 151 |
| 271014 | coding | 3 | 5146 | CCCAGAGAGGCCAAGCTCTG | 54 | 152 |
| 271015 | coding | 3 | 5189 | TGTGTTCCCTGAAGCGGCCA | 43 | 153 |
| 271016 | coding | 3 | 5269 | ACCCAGAATCATGGCCTGAT | 19 | 154 |
| 271017 | coding | 3 | 6049 | GGTGCCTGTCTGCTCAGCTG | 30 | 155 |
| 271018 | coding | 3 | 6520 | ATGTGAAACTTGTCTCTCCC | 44 | 156 |
| 271019 | coding | 3 | 6639 | TATGTCTGCAGTTGAGATAG | 15 | 157 |
| 271020 | coding | 3 | 6859 | TTGAATCCAGGATGCAGTAC | 35 | 158 |
| 271021 | coding | 3 | 7459 | GAGTCTCTGAGTCACCTCAC | 38 | 159 |
| 271022 | coding | 3 | 7819 | GATAGAATATTGCTCTGCAA | 100 | 160 |
| 271023 | coding | 3 | 7861 | CCCTTGCTCTACCAATGCTT | 44 | 161 |
| 271025 | coding | 3 | 8449 | TCCATTCCCTATGTCAGCAT | 16 | 162 |
| 271026 | coding | 3 | 8589 | GACTCCTTCAGAGCCAGCGG | 39 | 163 |
| 271027 | coding | 3 | 8629 | CCCATGCTCCGTTCTCAGGT | 26 | 164 |
| 271028 | coding | 3 | 8829 | CGCAGGTCAGCCTGACTAGA | 98 | 165 |
| 271030 | coding | 3 | 9119 | CAGTTAGAACACTGTGGCCC | 52 | 166 |
| 271031 | coding | 3 | 10159 | CAGTGTGATGACACTTGATT | 49 | 167 |
| 271032 | coding | 3 | 10301 | CTGTGGCTAACTTCAATCCC | 22 | 168 |
| 271033 | coding | 3 | 10349 | CAGTACTGTTATGACTACCC | 34 | 169 |
| 271034 | coding | 3 | 10699 | CACTGAAGACCGTGTGCTCT | 35 | 170 |
| 271035 | coding | 3 | 10811 | TCGTACTGTGCTCCCAGAGG | 23 | 171 |
| 271036 | coding | 3 | 10839 | AAGAGGCCCTCTAGCTGTAA | 95 | 172 |
| 271037 | coding | 3 | 11039 | AAGACCCAGAATGAATCCGG | 23 | 173 |
| 271038 | coding | 3 | 11779 | GTCTACCTCAAAGCGTGCAG | 29 | 174 |
| 271039 | coding | 3 | 11939 | TAGAGGCTAACGTACCATCT | 4 | 175 |
| 271041 | coding | 3 | 12149 | CCATATCCATGCCCACGGTG | 37 | 176 |
| 271042 | coding | 3 | 12265 | AGTTTCCTCATCAGATTCCC | 57 | 177 |
| 271043 | coding | 3 | 12380 | CCCAGTGGTACTTGTTGACA | 68 | 178 |
| 271044 | coding | 3 | 12526 | CCCAGTGGTGCCACTGGCTG | 22 | 179 |
| 271045 | coding | 3 | 12579 | GTCAACAGTTCCTGGTACAG | 19 | 180 |
| 271046 | coding | 3 | 12749 | CCCTAGTGTATATCCCAGGT | 61 | 181 |
| 271048 | coding | 3 | 13009 | CTGAAGATTACGTAGCACCT | 7 | 182 |
| 271049 | coding | 3 | 13299 | GTCCAGCCAACTATACTTGG | 54 | 183 |
| 271050 | coding | 3 | 13779 | CCTGGAGCAAGCTTCATGTA | 42 | 184 |
| 281586 | exon: | 3 | 229 | TGGACAGACCAGGCTGACAT | 80 | 185 |
| | exon | | | | | |
| | junction | | | | | |
| 281587 | coding | 3 | 269 | ATGTGTACTTCCGGAGGTGC | 77 | 186 |
| 281588 | coding | 3 | 389 | TCTTCAGGATGAAGCTGCAG | 80 | 187 |
| 281589 | coding | 3 | 449 | TCAGCAAGGCTTTGCCCTCA | 90 | 188 |
| 281590 | coding | 3 | 529 | CTGCTTCCCTTCTGGAATGG | 84 | 189 |
| 281591 | coding | 3 | 709 | TGCCACATTGCCCTTCCTCG | 90 | 190 |
| 281592 | coding | 3 | 829 | GCTGATCAGAGTTGACAAGG | 56 | 191 |
| 281593 | coding | 3 | 849 | TACTGACAGGACTGGCTGCT | 93 | 192 |
| 281594 | coding | 3 | 889 | GATGGCTTCTGCCACATGCT | 74 | 193 |
| 281595 | coding | 3 | 1059 | GATGTGGATTTGGTGCTCTC | 76 | 194 |
| 281596 | coding | 3 | 1199 | TGACTGCTTCATCACTGAGG | 77 | 195 |
| 281597 | coding | 3 | 1349 | GGTAGGTGACCACATCTATC | 36 | 196 |
| 281598 | coding | 3 | 1390 | TCGCAGCTGCTGTGCTGAGG | 70 | 197 |
| 281599 | exon: | 3 | 1589 | TTCCAATGACCCGCAGAATC | 74 | 198 |
| | exon | | | | | |
| | junction | | | | | |
| 281600 | coding | 3 | 1678 | GATCATCAGTGATGGCTTTG | 52 | 199 |
| 281601 | coding | 3 | 1699 | AGCCTGGATGGCAGCTTTCT | 83 | 200 |
| 281602 | coding | 3 | 1749 | GTCTGAAGAAGAACCTCCTG | 84 | 201 |
| 281603 | coding | 3 | 1829 | TATCTGCCTGTGAAGGACTC | 82 | 202 |
| 281604 | coding | 3 | 1919 | CTGAGTTCAAGATATTGGCA | 78 | 203 |
| 281605 | exon: | 3 | 2189 | CTTCCAAGCCAATCTCGATG | 82 | 204 |
| | exon | | | | | |
| | junction | | | | | |
| 281606 | coding | 3 | 2649 | TGCAACTGTAATCCAGCTCC | 86 | 205 |
| 281607 | exon: | 3 | 2729 | CCAGTTCAGCCTGCATGTTG | 84 | 206 |
| | exon | | | | | |
| | junction | | | | | |
| 281608 | coding | 3 | 2949 | GTAGAGACCAAATGTAATGT | 62 | 207 |
| 281609 | coding | 3 | 3059 | CGTTGGAGTAAGCGCCTGAG | 70 | 208 |
| 281610 | exon: | 3 | 3118 | CAGCTCTAATCTGGTGTCCC | 69 | 209 |
| | exon | | | | | |
| | junction | | | | | |
| 281611 | coding | 3 | 3189 | CTGTCCTCTCTCTGGAGCTC | 93 | 210 |
| 281612 | coding | 3 | 3289 | CAAGGTCATACTCTGCCGAT | 83 | 211 |
| 281613 | coding | 3 | 3488 | GTATGGAAATAACACCCTTG | 70 | 212 |
| 281614 | coding | 3 | 3579 | TAAGCTGTAGCAGATGAGTC | 63 | 213 |
| 281615 | coding | 3 | 4039 | TAGATCTCTGGAGGATTTGC | 81 | 214 |
| 281616 | coding | 3 | 4180 | GTCTAGAACACCCAGGAGAG | 66 | 215 |
| 281617 | coding | 3 | 4299 | ACCACAGAGTCAGCCTTCAT | 89 | 216 |
| 281618 | coding | 3 | 4511 | AAGCAGACATCTGTGGTCCC | 90 | 217 |
| 281619 | coding | 3 | 4660 | CTCTCCATTGAGCCGGCCAG | 96 | 218 |
| 281620 | coding | 3 | 4919 | CCTGATATTCAGAACGCAGC | 89 | 219 |
| 281621 | coding | 3 | 5009 | CAGTGCCTAAGATGTCAGCA | 53 | 220 |
| 281622 | coding | 3 | 5109 | AGCACCAGGAGACTACACTT | 88 | 221 |
| 281623 | coding | 3 | 5212 | CCCATCCAGACTGAATTTTG | 59 | 222 |
| 281624 | coding | 3 | 5562 | GGTTCTAGCCGTAGTTTCCC | 75 | 223 |
| 281625 | coding | 3 | 5589 | AGGTTACCAGCCACATGCAG | 94 | 224 |
| 281626 | coding | 3 | 5839 | ATGTGCATCGATGGTCATGG | 88 | 225 |
| 281627 | coding | 3 | 5869 | CCAGAGAGCGAGTTTCCCAT | 82 | 226 |
| 281628 | coding | 3 | 5979 | CTAGACACGAGATGATGACT | 81 | 227 |
| 281629 | coding | 3 | 6099 | TCCAAGTCCTGGCTGTATTC | 83 | 228 |
| 281630 | coding | 3 | 6144 | CGTCCAGTAAGCTCCACGCC | 82 | 229 |
| 281631 | coding | 3 | 6249 | TCAACGGCATCTCTCATCTC | 88 | 230 |
| 281632 | coding | 3 | 6759 | TGATAGTGCTCATCAAGACT | 75 | 231 |
| 281633 | coding | 3 | 6889 | GATTCTGATTTGGTACTTAG | 73 | 232 |
| 281634 | coding | 3 | 7149 | CTCTCGATTAACTCATGGAC | 81 | 233 |
| 281635 | coding | 3 | 7549 | ATACACTGCAACTGTGGCCT | 89 | 234 |
| 281636 | coding | 3 | 7779 | GCAAGAGTCCACCAATCAGA | 68 | 235 |
| 281637 | coding | 3 | 7929 | AGAGCCTGAAGACTGACTTC | 74 | 236 |
| 281638 | coding | 3 | 8929 | TCCCTCATCTGAGAATCTGG | 66 | 237 |
| 281640 | coding | 3 | 10240 | CAGTGCATCAATGACAGATG | 87 | 238 |
| 281641 | coding | 3 | 10619 | CCGAACCCTTGACATCTCCT | 72 | 239 |
| 281642 | coding | 3 | 10659 | GCCTCACTAGCAATAGTTCC | 59 | 240 |
| 281643 | coding | 3 | 10899 | GACATTTGCCATGGAGAGAG | 61 | 241 |
| 281644 | coding | 3 | 11209 | CTGTCTCCTACCAATGCTGG | 26 | 242 |
| 281645 | exon: | 3 | 11979 | TCTGCACTGAAGTCACGGTG | 78 | 243 |
| | exon | | | | | |
| | junction | | | | | |
| 281646 | coding | 3 | 12249 | TCCCGGACCCTCAACTCAGT | 76 | 244 |
| 281648 | 3′UTR | 3 | 13958 | GCAGGTCCAGTTCATATGTG | 81 | 245 |
| 281649 | 3′UTR | 3 | 14008 | GCCATCCTTCTGAGTTCAGA | 76 | 246 |
| 301012 | exon: | 3 | 3249 | GCCTCAGTCTGCTTCGCACC | 87 | 247 |
| | exon | | | | | |
| | junction | | | | | |
| 301013 | 5′UTR | 3 | 3 | CCCCGCAGGTCCCGGTGGGA | 82 | 248 |
| 301014 | 5′UTR | 3 | 6 | CAGCCCCGCAGGTCCCGGTG | 88 | 249 |
| 301015 | 5′UTR | 3 | 23 | CAACCGAGAAGGGCACTCAG | 53 | 250 |
| 301016 | 5′UTR | 3 | 35 | CCTCAGCGGCAGCAACCGAG | 62 | 251 |
| 301017 | 5′UTR | 3 | 36 | TCCTCAGCGGCAGCAACCGA | 47 | 252 |
| 301018 | 5′UTR | 3 | 37 | CTCCTCAGCGGCAGCAACCG | 45 | 253 |
| 301019 | 5′UTR | 3 | 39 | GGCTCCTCAGCGGCAGCAAC | 70 | 254 |
| 301020 | 5′UTR | 3 | 43 | GGCGGGCTCCTCAGCGGCAG | 85 | 255 |
| 301021 | 5′UTR | 3 | 116 | GGTCCATCGCCAGCTGCGGT | 89 | 256 |
| 301022 | Start | 3 | 120 | GGCGGGTCCATCGCCAGCTG | 69 | 257 |
| | Codon | | | | | |
| 301023 | Stop | 3 | 13800 | TAGAGGATGATAGTAAGTTC | 69 | 258 |
| | Codon | | | | | |
| 301024 | 3′UTR | 3 | 13824 | AAATGAAGATTTCTTTTAAA | 5 | 259 |
| 301025 | 3′UTR | 3 | 13854 | TATGTGAAAGTTCAATTGGA | 76 | 260 |
| 301026 | 3′UTR | 3 | 13882 | ATATAGGCAGTTTGAATTTT | 57 | 261 |
| 301027 | 3′UTR | 3 | 13903 | GCTCACTGTATGGTTTTATC | 89 | 262 |
| 301028 | 3′UTR | 3 | 13904 | GGCTCACTGTATGGTTTTAT | 93 | 263 |
| 301029 | 3′UTR | 3 | 13908 | GGCTGGCTCACTGTATGGTT | 90 | 264 |
| 301030 | 3′UTR | 3 | 13909 | AGGCTGGCTCACTGTATGGT | 90 | 265 |
| 301031 | 3′UTR | 3 | 13910 | AAGGCTGGCTCACTGTATGG | 90 | 266 |
| 301032 | 3′UTR | 3 | 13917 | CTACTGCAAGGCTGGCTCAC | 63 | 267 |
| 301033 | 3′UTR | 3 | 13922 | ACTGCCTACTGCAAGGCTGG | 77 | 268 |
| 301034 | 3′UTR | 3 | 13934 | TGCTTATAGTCTACTGCCTA | 88 | 269 |
| 301035 | 3′UTR | 3 | 13937 | TTCTGCTTATAGTCTACTGC | 82 | 270 |
| 301036 | 3′UTR | 3 | 13964 | TTTGGTGCAGGTCCAGTTCA | 88 | 271 |
| 301037 | 3′UTR | 3 | 13968 | CAGCTTTGGTGCAGGTCCAG | 90 | 272 |
| 301038 | 3′UTR | 3 | 13970 | GCCAGCTTTGGTGCAGGTCC | 86 | 273 |
| 301039 | 3′UTR | 3 | 13974 | TGGTGCCAGCTTTGGTGCAG | 73 | 274 |
| 301040 | 3′UTR | 3 | 13978 | GCCCTGGTGCCAGCTTTGGT | 74 | 275 |
| 301041 | 3′UTR | 3 | 13997 | GAGTTCAGAGACCTTCCGAG | 85 | 276 |
| 301042 | 3′UTR | 3 | 14012 | AAATGCCATCCTTCTGAGTT | 81 | 277 |
| 301043 | 3′UTR | 3 | 14014 | AAAAATGCCATCCTTCTGAG | 81 | 278 |
| 301044 | 3′UTR | 3 | 14049 | AAAATAACTCAGATCCTGAT | 76 | 279 |
| 301045 | 3′UTR | 3 | 14052 | AGCAAAATAACTCAGATCCT | 90 | 280 |
| 301046 | 3′UTR | 3 | 14057 | AGTTTAGCAAAATAACTCAG | 80 | 281 |
| 301047 | 3′UTR | 3 | 14064 | TCCCCCAAGTTTAGCAAAAT | 56 | 282 |
| 301048 | 3′UTR | 3 | 14071 | TTCCTCCTCCCCCAAGTTTA | 67 | 283 |
| 301217 | 3′UTR | 3 | 14087 | AGACTCCATTTATTTGTTCC | 81 | 284 |

### Example 30

Antisense Inhibition of Apolipoprotein B—Gene Walk

In accordance with the present invention, a “gene walk” was conducted in which another series of oligonucleotides was designed to target the regions of the human apolipoprotein B RNA (GenBank accession number NM_000384.1, incorporated herein as SEQ ID NO: 3) which are near the target site of SEQ ID Nos 224 or 247. The oligonucleotides are shown in Table 11. “Target site” indicates the first (5′-most) nucleotide number on the particular target sequence to which the oligonucleotide binds. All compounds in Table 11 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Treatment doses were 50 nM and 150 nM and are indicated in Table 11. Data are averages from two experiments. If present, “N.D.” indicates “no data”.

TABLE 11
Inhibition of human apolipoprotein B mRNA levels
by chimeric phosphorothioate oligonucleotides having
2′-MOE wings and a deoxy gap - Gene walk

| | | TARGET | | | | | |
|---|---|---|---|---|---|---|---|
| | | SEQ ID | TARGET | | % INHIB | % INHIB | SEQ ID |
| ISIS # | REGION | NO | SITE | SEQUENCE | 150 nM | 50 nM | NO |
| 308589 | exon: | 3 | 3230 | CTTCTGCTTGAGTTACAAAC | 94 | 20 | 285 |
| | exon | | | | | | |
| | junction | | | | | | |
| 308590 | exon: | 3 | 3232 | ACCTTCTGCTTGAGTTACAA | 98 | 26 | 286 |
| | exon | | | | | | |
| | junction | | | | | | |
| 308591 | exon: | 3 | 3234 | GCACCTTCTGCTTGAGTTAC | 92 | 76 | 287 |
| | exon | | | | | | |
| | junction | | | | | | |
| 308592 | exon: | 3 | 3236 | TCGCACCTTCTGCTTGAGTT | 96 | 49 | 288 |
| | exon | | | | | | |
| | junction | | | | | | |
| 308593 | exon: | 3 | 3238 | CTTCGCACCTTCTGCTTGAG | 80 | 41 | 289 |
| | exon | | | | | | |
| | junction | | | | | | |
| 308594 | exon: | 3 | 3240 | TGCTTCGCACCTTCTGCTTG | 88 | 57 | 290 |
| | exon | | | | | | |
| | junction | | | | | | |
| 308595 | exon: | 3 | 3242 | TCTGCTTCGCACCTTCTGCT | 82 | 60 | 291 |
| | exon | | | | | | |
| | junction | | | | | | |
| 308596 | exon: | 3 | 3244 | AGTCTGCTTCGCACCTTCTG | 94 | 81 | 292 |
| | exon | | | | | | |
| | junction | | | | | | |
| 308597 | exon: | 3 | 3246 | TCAGTCTGCTTCGCACCTTC | 91 | 66 | 293 |
| | exon | | | | | | |
| | junction | | | | | | |
| 308598 | exon: | 3 | 3248 | CCTCAGTCTGCTTCGCACCT | 85 | 59 | 294 |
| | exon | | | | | | |
| | junction | | | | | | |
| 308599 | exon: | 3 | 3250 | AGCCTCAGTCTGCTTCGCAC | 94 | 79 | 295 |
| | exon | | | | | | |
| | junction | | | | | | |
| 308600 | coding | 3 | 3252 | GTAGCCTCAGTCTGCTTCGC | 89 | 72 | 296 |
| 308601 | coding | 3 | 3254 | TGGTAGCCTCAGTCTGCTTC | 91 | 63 | 297 |
| 308602 | coding | 3 | 3256 | CATGGTAGCCTCAGTCTGCT | 92 | 83 | 298 |
| 308603 | coding | 3 | 3258 | GTCATGGTAGCCTCAGTCTG | 97 | 56 | 299 |
| 308604 | coding | 3 | 3260 | ATGTCATGGTAGCCTCAGTC | 90 | 73 | 300 |
| 308605 | coding | 3 | 3262 | GAATGTCATGGTAGCCTCAG | 81 | 50 | 301 |
| 308606 | coding | 3 | 3264 | TTGAATGTCATGGTAGCCTC | 97 | 54 | 302 |
| 308607 | coding | 3 | 3266 | ATTTGAATGTCATGGTAGCC | 77 | 9 | 303 |
| 308608 | coding | 3 | 3268 | ATATTTGAATGTCATGGTAG | 85 | 70 | 304 |
| 308609 | coding | 3 | 5582 | CAGCCACATGCAGCTTCAGG | 96 | 78 | 305 |
| 308610 | coding | 3 | 5584 | ACCAGCCACATGCAGCTTCA | 90 | 40 | 306 |
| 308611 | coding | 3 | 5586 | TTACCAGCCACATGCAGCTT | 95 | 59 | 307 |
| 308612 | coding | 3 | 5588 | GGTTACCAGCCACATGCAGC | 90 | 75 | 308 |
| 308613 | coding | 3 | 5590 | TAGGTTACCAGCCACATGCA | 87 | 43 | 309 |
| 308614 | coding | 3 | 5592 | TTTAGGTTACCAGCCACATG | 92 | 74 | 310 |
| 308615 | coding | 3 | 5594 | CTTTTAGGTTACCAGCCACA | 85 | 45 | 311 |
| 308616 | coding | 3 | 5596 | TCCTTTTAGGTTACCAGCCA | 81 | 39 | 312 |
| 308617 | coding | 3 | 5598 | GCTCCTTTTAGGTTACCAGC | 87 | 77 | 313 |
| 308618 | coding | 3 | 5600 | AGGCTCCTTTTAGGTTACCA | 77 | 61 | 314 |
| 308619 | coding | 3 | 5602 | GTAGGCTCCTTTTAGGTTAC | 74 | 69 | 315 |
| 308620 | coding | 3 | 5604 | TGGTAGGCTCCTTTTAGGTT | 88 | 69 | 316 |
| 308621 | coding | 3 | 5606 | TTTGGTAGGCTCCTTTTAGG | 91 | 56 | 317 |

As shown in Tables 10 and 11, SEQ ID Nos 124, 128, 129, 132, 133, 134, 138, 140, 141, 142, 144, 145, 147, 148, 149, 150, 152, 153, 155, 156, 158, 159, 160, 161, 163, 165, 166, 167, 169, 170, 172, 176, 177, 178, 181, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 243, 244, 245, 246, 247, 248, 249, 250, 251, 252, 253, 254, 255, 256, 257, 258, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, and 317 demonstrated at least 30% inhibition of human apolipoprotein B expression in this assay and are therefore preferred. More preferred are SEQ ID Nos 224, 247, and 262. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Tables 10 and 11. “Target site” indicates the first (5′-most) nucleotide number on the particular target nucleic acid to which the oligonucleotide binds. Also shown in Table 18 is the species in which each of the preferred target segments was found.

### Example 31

Antisense Inhibition of Human Apolipoprotein B Expression by Chimeric Phosphorothioate Oligonucleotides Having 2′-MOE Wings and a Deoxy Gap: Targeting GenBank Accession Number M14162.1

In accordance with the present invention, another series of oligonucleotides was designed to target different regions of the human apolipoprotein B RNA, using published sequence (GenBank accession number M14162.1, incorporated herein as SEQ ID NO: 318). The oligonucleotides are shown in Table 12. “Target site” indicates the first (5′-most) nucleotide number on the particular target sequence to which the oligonucleotide binds. All compounds in Table 12 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Data are averages from two experiments in which HepG2 cells were treated with 150 nM of the compounds in Table 12. If present, “N.D.” indicates “no data”.

TABLE 12
Inhibition of human apolipoprotein B mRNA levels by
chimeric phosphorothioate oligonucleotides having
2′-MOE wings and a deoxy gap

| | | TARGET SEQ | TARGET | | | SEQ |
|---|---|---|---|---|---|---|
| ISIS # | REGION | ID NO | SITE | SEQUENCE | % INHIB | ID NO |
| 271009 | coding | 318 | 3121 | GCCTCAGTCTGCTTCGCGCC | 75 | 319 |
| 271024 | coding | 318 | 8031 | GCTCACTGTTCAGCATCTGG | 27 | 320 |
| 271029 | coding | 318 | 8792 | TGAGAATCTGGGCGAGGCCC | N.D. | 321 |
| 271040 | coding | 318 | 11880 | GTCCTTCATATTTGCCATCT | 0 | 322 |
| 271047 | coding | 318 | 12651 | CCTCCCTCATGAACATAGTG | 32 | 323 |
| 281639 | coding | 318 | 9851 | GACGTCAGAACCTATGATGG | 38 | 324 |
| 281647 | coding | 318 | 12561 | TGAGTGAGTCAATCAGCTTC | 73 | 325 |

### Example 32

Antisense Inhibition of Human Apolipoprotein B—Gene Walk Targeting GenBank Accession Number M14162.1

In accordance with the present invention, a “gene walk” was conducted in which another series of oligonucleotides was designed to target the regions of the human apolipoprotein B RNA (GenBank accession number M14162.1, incorporated herein as SEQ ID NO: 318) which are near the target site of SEQ ID NO: 319. The oligonucleotides are shown in Table 13. “Target site” indicates the first (5′-most) nucleotide number on the particular target sequence to which the oligonucleotide binds. All compounds in Table 13 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Treatment doses were 50 nM and 150 nM and are indicated in Table 13. Data are averages from two experiments. If present, “N.D.” indicates “no data”.

TABLE 13
Inhibition of human apolipoprotein B mRNA levels
by chimeric phosphorothioate oligonucleotides having
2′-MOE wings and a deoxy gap

| | | TARGET | | | % | % | |
|---|---|---|---|---|---|---|---|
| | | SEQ ID | TARGET | | INHIB | INHIB | SEQ ID |
| ISIS # | REGION | NO | SITE | SEQUENCE | 150 nM | 50 nM | NO |
| 308622 | coding | 318 | 3104 | GCCTTCTGCTTGAGTTACAA | 87 | 25 | 326 |
| 308623 | coding | 318 | 3106 | GCGCCTTCTGCTTGAGTTAC | 71 | 62 | 327 |
| 308624 | coding | 318 | 3108 | TCGCGCCTTCTGCTTGAGTT | 89 | 69 | 328 |
| 308625 | coding | 318 | 3110 | CTTCGCGCCTTCTGCTTGAG | 83 | 64 | 329 |
| 308626 | coding | 318 | 3116 | AGTCTGCTTCGCGCCTTCTG | 94 | 38 | 330 |
| 308627 | coding | 318 | 3118 | TCAGTCTGCTTCGCGCCTTC | 89 | 67 | 331 |
| 308628 | coding | 318 | 3120 | CCTCAGTCTGCTTCGCGCCT | 92 | 61 | 332 |
| 308629 | coding | 318 | 3122 | AGCCTCAGTCTGCTTCGCGC | 95 | 77 | 333 |

As shown in Tables 12 and 13, SEQ ID Nos 319, 323, 324, 325, 326, 327, 328, 329, 330, 331, 332, and 333 demonstrated at least 30% inhibition of human apolipoprotein B expression in this assay and are therefore preferred. More preferred is SEQ ID NO: 319. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Tables 12 and 13. “Target site” indicates the first (5′-most) nucleotide number on the particular target nucleic acid to which the oligonucleotide binds. Also shown in Table 18 is the species in which each of the preferred target segments was found.

### Example 33

Antisense Inhibition of Human Apolipoprotein B Expression by Chimeric Phosphorothioate Oligonucleotides Having 2′-MOE Wings and a Deoxy Gap—Targeting the Genomic Sequence

In accordance with the present invention, another series of oligonucleotides was designed to target different regions of the human apolipoprotein B RNA, using published sequence (the complement of nucleotides 39835 to 83279 of the sequence with GenBank accession number NT_022227.9, representing a genomic sequence, incorporated herein as SEQ ID NO: 334). The oligonucleotides are shown in Table 14. “Target site” indicates the first (5′-most) nucleotide number on the particular target sequence to which the oligonucleotide binds. All compounds in Table 14 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Data are averages from two experiments in which HepG2 cells were treated with 150 nM of the oligonucleotides in Table 14. If present, “N.D.” indicates “no data”.

TABLE 14
Inhibition of human apolipoprotein B mRNA levels by
chimeric phosphorothioate oligonucleotides having
2′-MOE wings and a deoxy gap

| | | TARGET | | | | |
|---|---|---|---|---|---|---|
| | | SEQ ID | TARGET | | % | SEQ ID |
| ISIS # | REGION | NO | SITE | SEQUENCE | INHIB | NO |
| 301049 | intron: | 334 | 904 | TCTGTAAGACAGGAGAAAGA | 41 | 335 |
| | exon | | | | | |
| | junction | | | | | |
| 301050 | intron: | 334 | 913 | ATTTCCTCTTCTGTAAGACA | 22 | 336 |
| | exon | | | | | |
| | junction | | | | | |
| 301051 | exon: | 334 | 952 | GATGCCTTACTTGGACAGAC | 27 | 337 |
| | intron | | | | | |
| | junction | | | | | |
| 301052 | intron | 334 | 1945 | AGAAATAGCTCTCCCAAGGA | 13 | 338 |
| 301053 | intron: | 334 | 1988 | GTCGCATCTTCTAACGTGGG | 45 | 339 |
| | exon | | | | | |
| | junction | | | | | |
| 301054 | exon: | 334 | 2104 | TCCTCCATACCTTGCAGTTG | 0 | 340 |
| | intron | | | | | |
| | junction | | | | | |
| 301055 | intron | 334 | 2722 | TGGCTCATGTCTACCATATT | 49 | 341 |
| 301056 | intron | 334 | 2791 | CAGTTGAAATGCAGCTAATG | 35 | 342 |
| 301057 | intron | 334 | 3045 | TGCAGACTAGGAGTGAAAGT | 30 | 343 |
| 301058 | intron | 334 | 3117 | AGGAGGATGTCCTTTTATTG | 27 | 344 |
| 301059 | intron | 334 | 3290 | ATCAGAGCACCAAAGGGAAT | 12 | 345 |
| 301060 | intron: | 334 | 3381 | CCAGCTCAACCTGAGAATTC | 17 | 346 |
| | exon | | | | | |
| | junction | | | | | |
| 301061 | exon: | 334 | 3527 | CATGACTTACCTGGACATGG | 52 | 347 |
| | intron | | | | | |
| | junction | | | | | |
| 301062 | intron | 334 | 3566 | CCTCAGCGGACACACACACA | 21 | 348 |
| 301063 | intron | 334 | 3603 | GTCACATCCGTGCCTGGTGC | 41 | 349 |
| 301064 | intron | 334 | 3864 | CAGTGCCTCTGGGACCCCAC | 60 | 350 |
| 301065 | intron | 334 | 3990 | AGCTGCAGTGGCCGATCAGC | 50 | 351 |
| 301066 | intron | 334 | 4251 | GACCTCCCCAGCCACGTGGA | 61 | 352 |
| 301067 | intron | 334 | 4853 | TCTGATCACCATACATTACA | 45 | 353 |
| 301068 | intron | 334 | 5023 | ATTTCCCACTGGGTACTCTC | 44 | 354 |
| 301069 | intron | 334 | 5055 | GGCTGAAGCCCATGCTGACT | 44 | 355 |
| 301070 | intron | 334 | 5091 | GTTGGACAGTCATTCTTTTG | 38 | 356 |
| 301071 | intron | 334 | 5096 | CACTTGTTGGACAGTCATTC | 48 | 357 |
| 301072 | intron | 334 | 5301 | ATTTTAAATTACAGTAGATA | 43 | 358 |
| 301073 | intron | 334 | 5780 | CTGTTCTCCACCCATATCAG | 37 | 359 |
| 301074 | intron: | 334 | 6353 | GAGCTCATACCTGTCCCAGA | 75 | 360 |
| | exon | | | | | |
| | junction | | | | | |
| 301075 | intron | 334 | 6534 | TTCAAGGGCCACTGCTATCA | 52 | 361 |
| 301076 | intron | 334 | 6641 | CCAGTATTTCACGCCAATCC | 36 | 362 |
| 301077 | intron | 334 | 6661 | GGCAGGAGGAACCTCGGGCA | 55 | 363 |
| 301078 | intron | 334 | 6721 | TTTTAAAATTAGACCCAACC | 22 | 364 |
| 301079 | intron | 334 | 6727 | TGACTGTTTTAAAATTAGAC | 20 | 365 |
| 301080 | intron | 334 | 6788 | CCCAGCAAACACAGGTGAAG | 25 | 366 |
| 301081 | intron | 334 | 7059 | GAGTGTGGTCTTGCTAGTGC | 46 | 367 |
| 301082 | intron | 334 | 7066 | CTATGCAGAGTGTGGTCTTG | 41 | 368 |
| 301083 | intron | 334 | 7189 | AGAAGATGCAACCACATGTA | 29 | 369 |
| 301084 | intron: | 334 | 7209 | ACACGGTATCCTATGGAGGA | 49 | 370 |
| | exon | | | | | |
| | junction | | | | | |
| 301085 | exon: | 334 | 7365 | TGGGACTTACCATGCCTTTG | 11 | 371 |
| | intron | | | | | |
| | junction | | | | | |
| 301086 | intron | 334 | 7702 | GGTTTTGCTGCCCTACATCC | 30 | 372 |
| 301087 | intron | 334 | 7736 | ACAAGGAGTCCTTGTGCAGA | 40 | 373 |
| 301088 | intron | 334 | 8006 | ATGTTCACTGAGACAGGCTG | 41 | 374 |
| 301089 | intron | 334 | 8215 | GAAGGTCCATGGTTCATCTG | 0 | 375 |
| 301090 | intron | 334 | 8239 | ATTAGACTGGAAGCATCCTG | 39 | 376 |
| 301091 | intron | 334 | 8738 | GAGATTGGAGACGAGCATTT | 35 | 377 |
| 301092 | exon: | 334 | 8881 | CATGACCTACTTGTAGGAGA | 22 | 378 |
| | intron | | | | | |
| | junction | | | | | |
| 301093 | intron | 334 | 9208 | TGGATTTGGATACACAAGTT | 42 | 379 |
| 301094 | intron | 334 | 9244 | ACTCAATATATATTCATTGA | 22 | 380 |
| 301095 | intron | 334 | 9545 | CAAGGAAGCACACCATGTCA | 38 | 381 |
| 301096 | intron: | 334 | 9563 | ATACTTATTCCTGGTAACCA | 24 | 382 |
| | exon | | | | | |
| | junction | | | | | |
| 301097 | intron | 334 | 9770 | GGTAGCCAGAACACCAGTGT | 50 | 383 |
| 301098 | intron | 334 | 9776 | ACTAGAGGTAGCCAGAACAC | 34 | 384 |
| 301099 | intron | 334 | 10149 | ACCACCTGACATCACAGGTT | 24 | 385 |
| 301100 | intron | 334 | 10341 | TACTGTGACCTATGCCAGGA | 55 | 386 |
| 301101 | intron | 334 | 10467 | GGAGGTGCTACTGTTGACAT | 42 | 387 |
| 301102 | intron | 334 | 10522 | TCCAGACTTGTCTGAGTCTA | 47 | 388 |
| 301103 | intron | 334 | 10547 | TCTAAGAGGTAGAGCTAAAG | 7 | 389 |
| 301104 | intron | 334 | 10587 | CCAGAGATGAGCAACTTAGG | 38 | 390 |
| 301105 | intron | 334 | 10675 | GGCCATGTAAATTGCTCATC | 7 | 391 |
| 301106 | intron | 334 | 10831 | AAAGAAACTATCCTGTATTC | 12 | 392 |
| 301107 | intron: | 334 | 10946 | TTCTTAGTACCTGGAAGATG | 23 | 393 |
| | exon | | | | | |
| | junction | | | | | |
| 301108 | exon: | 334 | 11166 | CATTAGATACCTGGACACCT | 29 | 394 |
| | intron | | | | | |
| | junction | | | | | |
| 301109 | intron | 334 | 11337 | GTTTCATGGAACTCAGCGCA | 44 | 395 |
| 301110 | intron | 334 | 11457 | CTGGAGAGCACCTGCAATAG | 35 | 396 |
| 301111 | intron | 334 | 11521 | TGAAGGGTAGAGAAATCATA | 9 | 397 |
| 301112 | exon: | 334 | 12111 | GGAAACTCACTTGTTGACCG | 25 | 398 |
| | intron | | | | | |
| | junction | | | | | |
| 301113 | intron | 334 | 12155 | AGGTGCAAGATGTTCCTCTG | 46 | 399 |
| 301114 | intron | 334 | 12162 | TGCACAGAGGTGCAAGATGT | 16 | 400 |
| 301115 | intron | 334 | 12221 | CACAAGAGTAAGGAGCAGAG | 39 | 401 |
| 301116 | intron | 334 | 12987 | GATGGATGGTGAGAAATTAC | 33 | 402 |
| 301117 | intron | 334 | 13025 | TAGACAATTGAGACTCAGAA | 39 | 403 |
| 301118 | intron | 334 | 13057 | ATGTGCACACAAGGACATAG | 33 | 404 |
| 301119 | intron | 334 | 13634 | ACATACAAATGGCAATAGGC | 33 | 405 |
| 301120 | intron | 334 | 13673 | TAGGCAAAGGACATGAATAG | 30 | 406 |
| 301121 | coding | 334 | 14448 | TTATGATAGCTACAGAATAA | 29 | 407 |
| 301122 | exon: | 334 | 14567 | CTGAGATTACCCGCAGAATC | 32 | 408 |
| | intron | | | | | |
| | junction | | | | | |
| 301123 | intron | 334 | 14587 | GATGTATGTCATATAAAAGA | 26 | 409 |
| 301124 | intron: | 334 | 14680 | TTTCCAATGACCTGCATTGA | 48 | 410 |
| | exon | | | | | |
| | junction | | | | | |
| 301125 | intron | 334 | 15444 | AGGGATGGTCAATCTGGTAG | 57 | 411 |
| 301126 | intron | 334 | 15562 | GGCTAATAAATAGGGTAGTT | 22 | 412 |
| 301127 | intron | 334 | 15757 | TCCTAGAGCACTATCAAGTA | 41 | 413 |
| 301128 | intron: | 334 | 15926 | CCTCCTGGTCCTGCAGTCAA | 56 | 414 |
| | exon | | | | | |
| | junction | | | | | |
| 301129 | intron | 334 | 16245 | CATTTGCACAAGTGTTTGTT | 35 | 415 |
| 301130 | intron | 334 | 16363 | CTGACACACCATGTTATTAT | 10 | 416 |
| 301131 | intron: | 334 | 16399 | CTTTTTCAGACTAGATAAGA | 0 | 417 |
| | exon | | | | | |
| | junction | | | | | |
| 301132 | exon: | 334 | 16637 | TCACACTTACCTCGATGAGG | 29 | 418 |
| | intron | | | | | |
| | junction | | | | | |
| 301133 | intron | 334 | 17471 | AAGAAAATGGCATCAGGTTT | 13 | 419 |
| 301134 | intron: | 334 | 17500 | CCAAGCCAATCTGAGAAAGA | 25 | 420 |
| | exon | | | | | |
| | junction | | | | | |
| 301135 | exon: | 334 | 17677 | AAATACACACCTGCTCATGT | 20 | 421 |
| | intron | | | | | |
| | junction | | | | | |
| 301136 | exon: | 334 | 17683 | CTTCACAAATACACACCTGC | 20 | 422 |
| | intron | | | | | |
| | junction | | | | | |
| 301137 | intron | 334 | 18519 | AGTGGAAGTTTGGTCTCATT | 41 | 423 |
| 301138 | intron | 334 | 18532 | TTGCTAGCTTCAAAGTGGAA | 44 | 424 |
| 301139 | intron | 334 | 18586 | TCAAGAATAAGCTCCAGATC | 41 | 425 |
| 301140 | intron | 334 | 18697 | GCATACAAGTCACATGAGGT | 34 | 426 |
| 301141 | intron | 334 | 18969 | TACAAGGTGTTTCTTAAGAA | 38 | 427 |
| 301142 | intron | 334 | 19250 | ATGCAGCCAGGATGGGCCTA | 54 | 428 |
| 301143 | intron: | 334 | 19340 | TTACCATATCCTGAGAGTTT | 55 | 429 |
| | exon | | | | | |
| | junction | | | | | |
| 301144 | intron | 334 | 19802 | GCAAAGGTAGAGGAAGGTAT | 32 | 430 |
| 301145 | intron | 334 | 19813 | AAGGACCTTCAGCAAAGGTA | 36 | 431 |
| 301146 | intron | 334 | 20253 | CATAGGAGTACATTTATATA | 23 | 432 |
| 301147 | intron | 334 | 20398 | ATTATGATAAAATCAATTTT | 19 | 433 |
| 301148 | intron | 334 | 20567 | AGAAATTTCACTAGATAGAT | 31 | 434 |
| 301149 | intron | 334 | 20647 | AGCATATTTTGATGAGCTGA | 44 | 435 |
| 301150 | intron | 334 | 20660 | GAAAGGAAGGACTAGCATAT | 39 | 436 |
| 301151 | intron: | 334 | 20772 | CCTCTCCAATCTGTAGACCC | 28 | 437 |
| | exon | | | | | |
| | junction | | | | | |
| 301152 | intron | 334 | 21316 | CTGGATAACTCAGACCTTTG | 40 | 438 |
| 301153 | intron | 334 | 21407 | AGTCAGAAAACAACCTATTC | 11 | 439 |
| 301154 | intron: | 334 | 21422 | CAGCCTGCATCTATAAGTCA | 31 | 440 |
| | exon | | | | | |
| | junction | | | | | |
| 301155 | exon: | 334 | 21634 | AAAGAATTACCCTCCACTGA | 33 | 441 |
| | intron | | | | | |
| | junction | | | | | |
| 301156 | intron | 334 | 21664 | TCTTTCAAACTGGCTAGGCA | 39 | 442 |
| 301157 | intron | 334 | 21700 | GCCTGGCAAAATTCTGCAGG | 37 | 443 |
| 301158 | intron | 334 | 22032 | CTACCTCAAATCAATATGTT | 28 | 444 |
| 301159 | intron | 334 | 22048 | TGCTTTACCTACCTAGCTAC | 36 | 445 |
| 301160 | intron | 334 | 22551 | ACCTTGTGTGTCTCACTCAA | 49 | 446 |
| 301161 | intron | 334 | 22694 | ATGCATTCCCTGACTAGCAC | 34 | 447 |
| 301162 | intron | 334 | 22866 | CATCTCTGAGCCCCTTACCA | 24 | 448 |
| 301163 | intron | 334 | 22903 | GCTGGGCATGCTCTCTCCCC | 51 | 449 |
| 301164 | intron | 334 | 22912 | GCTTTCGCAGCTGGGCATGC | 55 | 450 |
| 301165 | intron | 334 | 23137 | ACTCCTTTCTATACCTGGCT | 47 | 451 |
| 301166 | intron | 334 | 23170 | ATTCTGCCTCTTAGAAAGTT | 38 | 452 |
| 301167 | intron | 334 | 23402 | CCAAGCCTCTTTACTGGGCT | 29 | 453 |
| 301168 | intron | 334 | 23882 | CACTCATGACCAGACTAAGA | 35 | 454 |
| 301169 | intron | 334 | 23911 | ACCTCCCAGAAGCCTTCCAT | 22 | 455 |
| 301170 | intron | 334 | 24184 | TTCATATGAAATCTCCTACT | 40 | 456 |
| 301171 | intron | 334 | 24425 | TATTTAATTTACTGAGAAAC | 7 | 457 |
| 301172 | intron: | 334 | 24559 | TAATGTGTTGCTGGTGAAGA | 35 | 458 |
| | exon | | | | | |
| | junction | | | | | |
| 301173 | exon: | 334 | 24742 | CATCTCTAACCTGGTGTCCC | 21 | 459 |
| | intron | | | | | |
| | junction | | | | | |
| 301174 | intron | 334 | 24800 | GTGCCATGCTAGGTGGCCAT | 37 | 460 |
| 301175 | intron | 334 | 24957 | AGCAAATTGGGATCTGTGCT | 29 | 461 |
| 301176 | intron | 334 | 24991 | TCTGGAGGCTCAGAAACATG | 57 | 462 |
| 301177 | intron | 334 | 25067 | TGAAGACAGGGAGCCACCTA | 40 | 463 |
| 301178 | intron | 334 | 25152 | AGGATTCCCAAGACTTTGGA | 38 | 464 |
| 301179 | intron: | 334 | 25351 | CAGCTCTAATCTAAAGACAT | 22 | 465 |
| | exon | | | | | |
| | junction | | | | | |
| 301180 | exon: | 334 | 25473 | GAATACTCACCTTCTGCTTG | 6 | 466 |
| | intron | | | | | |
| | junction | | | | | |
| 301181 | intron | 334 | 26047 | ATCTCTCTGTCCTCATCTTC | 28 | 467 |
| 301182 | intron | 334 | 26749 | CCAACTCCCCCTTTCTTTGT | 37 | 468 |
| 301183 | intron | 334 | 26841 | TCTGGGCCAGGAAGACACGA | 68 | 469 |
| 301184 | intron | 334 | 27210 | TATTGTGTGCTGGGCACTGC | 52 | 470 |
| 301185 | intron: | 334 | 27815 | TGCTTCGCACCTGGACGAGT | 51 | 471 |
| | exon | | | | | |
| | junction | | | | | |
| 301186 | exon: | 334 | 28026 | CCTTCTTTACCTTAGGTGGC | 37 | 472 |
| | intron | | | | | |
| | junction | | | | | |
| 301187 | intron | 334 | 28145 | GCTCTCTCTGCCACTCTGAT | 47 | 473 |
| 301188 | intron | 334 | 28769 | AACTTCTAAAGCCAACATTC | 27 | 474 |
| 301189 | intron: | 334 | 28919 | TGTGTCACAACTATGGTAAA | 63 | 475 |
| | exon | | | | | |
| | junction | | | | | |
| 301190 | exon: | 334 | 29095 | AGACACATACCATAATGCCA | 22 | 476 |
| | intron | | | | | |
| | junction | | | | | |
| 301191 | intron: | 334 | 29204 | TTCTCTTCATCTGAAAATAC | 21 | 477 |
| | exon | | | | | |
| | junction | | | | | |
| 301192 | intron | 334 | 29440 | TGAGGATGTAATTAGCACTT | 27 | 478 |
| 301193 | intron: | 334 | 29871 | AGCTCATTGCCTACAAAATG | 31 | 479 |
| | exon | | | | | |
| | junction | | | | | |
| 301194 | intron | 334 | 30181 | GTTCTCATGTTTACTAATGC | 40 | 480 |
| 301195 | intron | 334 | 30465 | GAATTGAGACAACTTGATTT | 26 | 481 |
| 301196 | intron: | 334 | 30931 | CCGGCCATCGCTGAAATGAA | 54 | 482 |
| | exon | | | | | |
| | junction | | | | | |
| 301197 | exon: | 334 | 31305 | CATAGCTCACCTTGCACATT | 28 | 483 |
| | intron | | | | | |
| | junction | | | | | |
| 301198 | intron | 334 | 31325 | CGGTGCACCCTTTACCTGAG | 28 | 484 |
| 301199 | intron: | 334 | 31813 | TCTCCAGATCCTAACATAAA | 19 | 485 |
| | exon | | | | | |
| | junction | | | | | |
| 301200 | intron | 334 | 39562 | TTGAATGACACTAGATTTTC | 37 | 486 |
| 301201 | intron | 334 | 39591 | AAAATCCATTTTCTTTAAAG | 12 | 487 |
| 301202 | intron | 334 | 39654 | CAGCTCACACTTATTTTAAA | 7 | 488 |
| 301203 | intron: | 334 | 39789 | GTTCCCAAAACTGTATAGGA | 36 | 489 |
| | exon | | | | | |
| | junction | | | | | |
| 301204 | exon: | 334 | 39904 | AGCTCCATACTGAAGTCCTT | 37 | 490 |
| | intron | | | | | |
| | junction | | | | | |
| 301205 | intron | 334 | 39916 | CAATTCAATAAAAGCTCCAT | 31 | 491 |
| 301206 | intron | 334 | 39938 | GTTTTCAAAAGGTATAAGGT | 28 | 492 |
| 301207 | intron: | 334 | 40012 | TTCCCATTCCCTGAAAGCAG | 13 | 493 |
| | exon | | | | | |
| | junction | | | | | |
| 301208 | exon: | 334 | 40196 | TGGTATTTACCTGAGGGCTG | 21 | 494 |
| | intron | | | | | |
| | junction | | | | | |
| 301209 | intron | 334 | 40412 | ATAAATAATAGTGCTGATGG | 39 | 495 |
| 301210 | intron | 334 | 40483 | CTATGGCTGAGCTTGCCTAT | 33 | 496 |
| 301211 | intron | 334 | 40505 | CTCTCTGAAAAATATACCCT | 17 | 497 |
| 301212 | intron | 334 | 40576 | TTGATGTATCTCATCTAGCA | 41 | 498 |
| 301213 | intron | 334 | 40658 | TAGAACCATGTTTGGTCTTC | 35 | 499 |
| 301214 | intron | 334 | 40935 | TTTCTCTTTATCACATGCCC | 29 | 500 |
| 301215 | intron | 334 | 41066 | TATAGTACACTAAAACTTCA | 1 | 501 |
| 301216 | intron: | 334 | 41130 | CTGGAGAGGACTAAACAGAG | 49 | 502 |
| | exon | | | | | |
| | junction | | | | | |

As shown in Table 14, SEQ ID Nos 335, 339, 341, 342, 343, 347, 349, 350, 351, 352, 353, 354, 355, 356, 357, 358, 359, 360, 361, 362, 363, 367, 368, 370, 372, 373, 374, 376, 377, 379, 381, 383, 384, 386, 387, 388, 390, 395, 396, 399, 401, 402, 403, 404, 405, 406, 408, 410, 411, 413, 414, 415, 423, 424, 425, 426, 427, 428, 429, 430, 431, 434, 435, 436, 438, 440, 441, 442, 443, 445, 446, 447, 449, 450, 451, 452, 454, 456, 458, 460, 462, 463, 464, 468, 469, 470, 471, 472, 473, 475, 479, 480, 482, 486, 489, 490, 491, 495, 496, 498, 499, and 502 demonstrated at least 30% inhibition of human apolipoprotein B expression in this assay and are therefore preferred. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Table 14. “Target site” indicates the first (5′-most) nucleotide number on the particular target nucleic acid to which the oligonucleotide binds. Also shown in Table 18 is the species in which each of the preferred target segments was found.

### Example 34

Antisense Inhibition of Human Apolipoprotein B Expression by Chimeric Phosphorothioate Oligonucleotides Having 2′-MOE Wings and a Deoxy Gap—Targeting GenBank Accession Number AI249040.1

In accordance with the present invention, another series of oligonucleotides was designed to target different regions of the human apolipoprotein B RNA, using published sequence (the complement of the sequence with GenBank accession number AI249040.1, incorporated herein as SEQ ID NO: 503). The oligonucleotides are shown in Table 15. “Target site” indicates the first (5′-most) nucleotide number on the particular target sequence to which the oligonucleotide binds. All compounds in Table 15 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described in other examples herein. Data are averages from two experiments in which HepG2 cells were treated with 150 nM of the oligonucleotides in Table 15. If present, “N.D.” indicates “no data”.

TABLE 15
Inhibition of human apolipoprotein B mRNA levels by chimeric phosphorothioate
oligonucleotides having 2′-MOE wings and a deoxy gap

| | | TARGET | | | | |
|---|---|---|---|---|---|---|
| | | SEQ ID | TARGET | | % | |
| ISIS # | REGION | NO | SITE | SEQUENCE | INHIB | SEQ ID NO |
| 301218 | 3′UTR | 503 | 484 | ACATTTTATCAATGCCCTCG | 23 | 504 |
| 301219 | 3′UTR | 503 | 490 | GCCAGAACATTTTATCAATG | 35 | 505 |
| 301220 | 3′UTR | 503 | 504 | AGAGGTTTTGCTGTGCCAGA | 51 | 506 |
| 301221 | 3′UTR | 503 | 506 | CTAGAGGTTTTGCTGTGCCA | 61 | 507 |
| 301222 | 3′UTR | 503 | 507 | TCTAGAGGTTTTGCTGTGCC | 14 | 508 |
| 301223 | 3′UTR | 503 | 522 | AATCACACTATGTGTTCTAG | 26 | 509 |
| 301224 | 3′UTR | 503 | 523 | AAATCACACTATGTGTTCTA | 33 | 510 |
| 301225 | 3′UTR | 503 | 524 | TAAATCACACTATGTGTTCT | 3 | 511 |
| 301226 | 3′UTR | 503 | 526 | CTTAAATCACACTATGTGTT | 39 | 512 |
| 301227 | 3′UTR | 503 | 536 | TATTCTGTTACTTAAATCAC | 23 | 513 |

As shown in Table 15, SEQ ID Nos 505, 506, 507, 510, and 512 demonstrated at least 30% inhibition of human apolipoprotein B expression in this assay and are therefore preferred. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Table 15. “Target site” indicates the first (5′-most) nucleotide number on the particular target nucleic acid to which the oligonucleotide binds. Also shown in Table 18 is the species in which each of the preferred target segments was found.

### Example 35

Antisense Inhibition of Human Apolipoprotein B Expression by Chimeric Phosphorothioate Oligonucleotides Having 2′-MOE Wings and a Deoxy Gap—Variation in Position of the Gap

In accordance with the present invention, a series of antisense compounds was designed to target different regions of the human apolipoprotein B RNA, using published sequences (GenBank accession number NM_000384.1, incorporated herein as SEQ ID NO: 3). The compounds are shown in Table 16. “Target site” indicates the first (5′-most) nucleotide number on the particular target sequence to which the compound binds. All compounds in Table 16 are chimeric oligonucleotides (“gapmers”) 20 nucleotides in length. The “gap” region consists of 2′-deoxynucleotides, which is flanked on one or both sides (5′ and 3′ directions) by “wings” composed of 2′-methoxyethyl (2′-MOE)nucleotides. The number of 2′-MOE nucleotides on either side of the gap varies such that the total number of 2′-MOE nucleotides always equals 10 and the total length of the chimeric oligonucleotide is 20 nucleotides. The exact structure of each oligonucleotide is designated in Table 16 as the “gap structure” and the 2′-deoxynucleotides are in bold type. A designation of 8˜10˜2, for instance, indicates that the first (5′-most) 8 nucleotides and the last (3′-most) 2 nucleotides are 2′-MOE nucleotides and the 10 nucleotides in the gap are 2′-deoxynucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels by quantitative real-time PCR as described in other examples herein. Data, shown in Table 16, are averages from three experiments in which HepG2 cells were treated with the antisense oligonucleotides of the present invention at doses of 50 nM and 150 nM. If present, “N.D.” indicates “no data”.

TABLE 16
Inhibition of human apolipoprotein B mRNA levels by chimeric
phosphorothioate oligonucleotides having 2′-MOE wings
and a variable deoxy gap

| | TARGET | | | % | % | | |
|---|---|---|---|---|---|---|---|
| | SEQ ID | TARGET | | INHIB | INHIB | gap | SEQ |
| ISIS # | NO | SITE | SEQUENCE | 150 nM | 50 nM | structure | ID NO |
| 308631 | 3 | 5589 | AGGTTACCAGCCACATGCAG | 94 | 74 | 0~10~10 | 224 |
| 308632 | 3 | 3249 | GCCTCAGTCTGCTTCGCACC | 97 | 41 | 0~10~10 | 247 |
| 308634 | 3 | 5589 | AGGTTACCAGCCACATGCAG | 67 | 45 | 10~10~0 | 224 |
| 308635 | 3 | 3249 | GCCTCAGTCTGCTTCGCACC | 93 | 69 | 10~10~0 | 247 |
| 308637 | 3 | 5589 | AGGTTACCAGCCACATGCAG | 95 | 79 | 1~10~9 | 224 |
| 308638 | 3 | 3249 | GCCTCAGTCTGCTTCGCACC | 94 | 91 | 1~10~9 | 247 |
| 308640 | 3 | 5589 | AGGTTACCAGCCACATGCAG | 96 | 76 | 2~10~8 | 224 |
| 308641 | 3 | 3249 | GCCTCAGTCTGCTTCGCACC | 89 | 77 | 2~10~8 | 247 |
| 308643 | 3 | 5589 | AGGTTACCAGCCACATGCAG | 96 | 56 | 3~10~7 | 224 |
| 308644 | 3 | 3249 | GCCTCAGTCTGCTTCGCACC | 93 | 71 | 3~10~7 | 247 |
| 308646 | 3 | 5589 | AGGTTACCAGCCACATGCAG | 76 | 50 | 4~10~6 | 224 |
| 308647 | 3 | 3249 | GCCTCAGTCTGCTTCGCACC | 86 | 53 | 4~10~6 | 247 |
| 308649 | 3 | 5589 | AGGTTACCAGCCACATGCAG | 91 | 68 | 6~10~4 | 224 |
| 308650 | 3 | 3249 | GCCTCAGTCTGCTTCGCACC | 94 | 74 | 6~10~4 | 247 |
| 308652 | 3 | 5589 | AGGTTACCAGCCACATGCAG | 95 | 73 | 7~10~3 | 224 |
| 308653 | 3 | 3249 | GCCTCAGTCTGCTTCGCACC | 89 | 73 | 7~10~3 | 247 |
| 308655 | 3 | 5589 | AGGTTACCAGCCACATGCAG | 83 | 84 | 8~10~2 | 224 |
| 308656 | 3 | 3249 | GCCTCAGTCTGCTTCGCACC | 97 | 37 | 8~10~2 | 247 |
| 308658 | 3 | 5589 | AGGTTACCAGCCACATGCAG | 78 | 86 | 9~10~1 | 224 |
| 308659 | 3 | 3249 | GCCTCAGTCTGCTTCGCACC | 93 | 70 | 9~10~1 | 247 |
| 308660 | 3 | 3254 | TGGTAGCCTCAGTCTGCTTC | 92 | 72 | 2~10~8 | 514 |
| 308662 | 3 | 3254 | TGGTAGCCTCAGTCTGCTTC | 83 | 76 | 8~10~2 | 514 |

As shown in Table 16, SEQ ID Nos 224, 247, and 514 demonstrated at least 30% inhibition of human apolipoprotein B expression in this assay at both doses. These data suggest that the oligonucleotides are effective with a number of variations in the gap placement. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Table 16. “Target site” indicates the first (5′-most) nucleotide number on the particular target nucleic acid to which the oligonucleotide binds. Also shown in Table 18 is the species in which each of the preferred target segments was found.

### Example 36

Antisense Inhibition of Human Apolipoprotein B Expression by Chimeric Phosphorothioate Oligonucleotides Having 2′-MOE Wings and a Deoxy Gap—Variation in Position of the Gap of SEQ ID Nos: 319 and 515

In accordance with the present invention, a series of antisense compounds was designed based on SEQ ID Nos 319 and 515, with variations in the gap structure. The compounds are shown in Table 17. “Target site” indicates the first (5′-most) nucleotide number on the particular target sequence to which the compound binds. All compounds in Table 17 are chimeric oligonucleotides (“gapmers”) 20 nucleotides in length. The “gap” region consists of 2′-deoxynucleotides, which is flanked on one or both sides (5′ and 3′ directions) by “wings” composed of 2′-methoxyethyl (2′-MOE)nucleotides. The number of 2′-MOE nucleotides on either side of the gap varies such that the total number of 2′-MOE nucleotides always equals 10 and the total length of the chimeric oligonucleotide is 20 nucleotides. The exact structure of each oligonucleotide is designated in Table 17 as the “gap structure” and the 2′-deoxynucleotides are in bold type. A designation of 8˜10˜2, for instance, indicates that the first (5′-most) 8 nucleotides and the last (3′-most) 2 nucleotides are 2′-MOE nucleotides and the 10 nucleotides in the gap are 2′-deoxynucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels by quantitative real-time PCR as described in other examples herein. Data, shown in Table 17, are averages from three experiments in which HepG2 cells were treated with the antisense oligonucleotides of the present invention at doses of 50 nM and 150 nM. If present, “N.D.” indicates “no data”.

TABLE 17
Inhibition of human apolipoprotein B mRNA levels by chimeric
phosphorothioate oligonucleotides having 2′-MOE wings
and a variable deoxy gap

| | TARGET | | | % | % | | |
|---|---|---|---|---|---|---|---|
| | SEQ ID | TARGET | | INHIB | INHIB | gap | SEQ ID |
| ISIS # | NO | SITE | SEQUENCE | 150 nM | 50 nM | structure | NO |
| 308630 | 318 | 3121 | GCCTCAGTCTGCTTCGCGCC | 89 | 69 | 0~10~10 | 319 |
| 308633 | 318 | 3121 | GCCTCAGTCTGCTTCGCGCC | 83 | 66 | 10~10~0 | 319 |
| 308636 | 318 | 3121 | GCCTCAGTCTGCTTCGCGCC | 91 | 81 | 1~10~9 | 319 |
| 308639 | 318 | 3121 | GCCTCAGTCTGCTTCGCGCC | 94 | 86 | 2~10~8 | 319 |
| 308642 | 318 | 3121 | GCCTCAGTCTGCTTCGCGCC | 95 | 85 | 3~10~7 | 319 |
| 308645 | 318 | 3121 | GCCTCAGTCTGCTTCGCGCC | 98 | 57 | 4~10~6 | 319 |
| 308648 | 318 | 3121 | GCCTCAGTCTGCTTCGCGCC | 89 | 78 | 6~10~4 | 319 |
| 308651 | 318 | 3121 | GCCTCAGTCTGCTTCGCGCC | 88 | 87 | 7~10~3 | 319 |
| 308654 | 318 | 3121 | GCCTCAGTCTGCTTCGCGCC | 90 | 81 | 8~10~2 | 319 |
| 308657 | 318 | 3121 | GCCTCAGTCTGCTTCGCGCC | 78 | 61 | 9~10~1 | 319 |
| 308661 | 318 | 3116 | AGTCTGCTTCGCGCCTTCTG | 91 | 70 | 2~10~8 | 515 |
| 308663 | 318 | 3116 | AGTCTGCTTCGCGCCTTCTG | 84 | 44 | 8~10~2 | 515 |

As shown in Table 17, SEQ ID Nos 319 and 515 demonstrated at least 44% inhibition of human apolipoprotein B expression in this assay for either dose. These data suggest that the compounds are effective with a number of variations in gap placement. The target regions to which these preferred sequences are complementary are herein referred to as “preferred target segments” and are therefore preferred for targeting by compounds of the present invention. These preferred target segments are shown in Table 18. The sequences represent the reverse complement of the preferred antisense compounds shown in Table 17. “Target site” indicates the first (5′-most) nucleotide number on the particular target nucleic acid to which the oligonucleotide binds. Also shown in Table 18 is the species in which each of the preferred target segments was found.

TABLE 18
Sequence and position of preferred target segments
identified in apolipoprotein B.

| | TARGET | | | REV COMP | | |
|---|---|---|---|---|---|---|
| | SEQ ID | TARGET | | OF SEQ | | SEQ ID |
| SITE ID | NO | SITE | SEQUENCE | ID NO | ACTIVE IN | NO |
| 187342 | 3 | 199 | GCGCCAGGGCCGAAGAGGAA | 124 | H. sapiens | 516 |
| 187346 | 3 | 509 | CAGGTATGAGCTCAAGCTGG | 128 | H. sapiens | 517 |
| 187347 | 3 | 584 | CATCCTGAACATCAAGAGGG | 129 | H. sapiens | 518 |
| 187350 | 3 | 756 | GGGCAGTGTGATCGCTTCAA | 132 | H. sapiens | 519 |
| 187351 | 3 | 799 | CACTTGCTCTCATCAAAGGC | 133 | H. sapiens | 520 |
| 187352 | 3 | 869 | CACACTGGACGCTAAGAGGA | 134 | H. sapiens | 521 |
| 187356 | 3 | 1459 | CGCTGAGCCACGCGGTCAAC | 138 | H. sapiens | 522 |
| 187358 | 3 | 1859 | TGTCCAAATTCTACCATGGG | 140 | H. sapiens | 523 |
| 187359 | 3 | 2179 | CAGCTGACCTCATCGAGATT | 141 | H. sapiens | 524 |
| 187360 | 3 | 2299 | GTCAAGTTCCTGATGGTGTC | 142 | H. sapiens | 525 |
| 187362 | 3 | 2518 | AGCTGCTTCTGATGGGTGCC | 144 | H. sapiens | 526 |
| 187363 | 3 | 2789 | GGGCATCATCATTCCGGACT | 145 | H. sapiens | 527 |
| 187365 | 3 | 3100 | CCTACTATCCGCTGACCGGG | 147 | H. sapiens | 528 |
| 187367 | 3 | 3449 | GGGCCACCTAAGTTGTGACA | 148 | H. sapiens | 529 |
| 187368 | 3 | 3919 | AGAACATGGGATTGCCAGAC | 149 | H. sapiens | 530 |
| 187369 | 3 | 4089 | CTCCACTTCAAGTCTGTGGG | 150 | H. sapiens | 531 |
| 187371 | 3 | 5146 | CAGAGCTTGGCCTCTCTGGG | 152 | H. sapiens | 532 |
| 187372 | 3 | 5189 | TGGCCGCTTCAGGGAACACA | 153 | H. sapiens | 533 |
| 187374 | 3 | 6049 | CAGCTGAGCAGACAGGCACC | 155 | H. sapiens | 534 |
| 187375 | 3 | 6520 | GGGAGAGACAAGTTTCACAT | 156 | H. sapiens | 535 |
| 187377 | 3 | 6859 | GTACTGCATCCTGGATTCAA | 158 | H. sapiens | 536 |
| 187378 | 3 | 7459 | GTGAGGTGACTCAGAGACTC | 159 | H. sapiens | 537 |
| 187379 | 3 | 7819 | TTGCAGAGCAATATTCTATC | 160 | H. sapiens | 538 |
| 187380 | 3 | 7861 | AAGCATTGGTAGAGCAAGGG | 161 | H. sapiens | 539 |
| 187383 | 3 | 8589 | CCGCTGGCTCTGAAGGAGTC | 163 | H. sapiens | 540 |
| 187385 | 3 | 8829 | TCTAGTCAGGCTGACCTGCG | 165 | H. sapiens | 541 |
| 187387 | 3 | 9119 | GGGCCACAGTGTTCTAACTG | 166 | H. sapiens | 542 |
| 187388 | 3 | 10159 | AATCAAGTGTCATCACACTG | 167 | H. sapiens | 543 |
| 187390 | 3 | 10349 | GGGTAGTCATAACAGTACTG | 169 | H. sapiens | 544 |
| 187391 | 3 | 10699 | AGAGCACACGGTCTTCAGTG | 170 | H. sapiens | 545 |
| 187393 | 3 | 10839 | TTACAGCTAGAGGGCCTCTT | 172 | H. sapiens | 546 |
| 187398 | 3 | 12149 | CACCGTGGGCATGGATATGG | 176 | H. sapiens | 547 |
| 187399 | 3 | 12265 | GGGAATCTGATGAGGAAACT | 177 | H. sapiens | 548 |
| 187400 | 3 | 12380 | TGTCAACAAGTACCACTGGG | 178 | H. sapiens | 549 |
| 187403 | 3 | 12749 | ACCTGGGATATACACTAGGG | 181 | H. sapiens | 550 |
| 187406 | 3 | 13299 | CCAAGTATAGTTGGCTGGAC | 183 | H. sapiens | 551 |
| 187407 | 3 | 13779 | TACATGAAGCTTGCTCCAGG | 184 | H. sapiens | 552 |
| 197724 | 3 | 229 | ATGTCAGCCTGGTCTGTCCA | 185 | H. sapiens | 553 |
| 197725 | 3 | 269 | GCACCTCCGGAAGTACACAT | 186 | H. sapiens | 554 |
| 197726 | 3 | 389 | CTGCAGCTTCATCCTGAAGA | 187 | H. sapiens | 555 |
| 197727 | 3 | 449 | TGAGGGCAAAGCCTTGCTGA | 188 | H. sapiens | 556 |
| 197728 | 3 | 529 | CCATTCCAGAAGGGAAGCAG | 189 | H. sapiens | 557 |
| 197729 | 3 | 709 | CGAGGAAGGGCAATGTGGCA | 190 | H. sapiens | 558 |
| 197730 | 3 | 829 | CCTTGTCAACTCTGATCAGC | 191 | H. sapiens | 559 |
| 197731 | 3 | 849 | AGCAGCCAGTCCTGTCAGTA | 192 | H. sapiens | 560 |
| 197732 | 3 | 889 | AGCATGTGGCAGAAGCCATC | 193 | H. sapiens | 561 |
| 197733 | 3 | 1059 | GAGAGCACCAAATCCACATC | 194 | H. sapiens | 562 |
| 197734 | 3 | 1199 | CCTCAGTGATGAAGCAGTCA | 195 | H. sapiens | 563 |
| 197735 | 3 | 1349 | GATAGATGTGGTCACCTACC | 196 | H. sapiens | 564 |
| 197736 | 3 | 1390 | CCTCAGCACAGCAGCTGCGA | 197 | H. sapiens | 565 |
| 197737 | 3 | 1589 | GATTCTGCGGGTCATTGGAA | 198 | H. sapiens | 566 |
| 197738 | 3 | 1678 | CAAAGCCATCACTGATGATC | 199 | H. sapiens | 567 |
| 197739 | 3 | 1699 | AGAAAGCTGCCATCCAGGCT | 200 | H. sapiens | 568 |
| 197740 | 3 | 1749 | CAGGAGGTTCTTCTTCAGAC | 201 | H. sapiens | 569 |
| 197741 | 3 | 1829 | GAGTCCTTCACAGGCAGATA | 202 | H. sapiens | 570 |
| 197742 | 3 | 1919 | TGCCAATATCTTGAACTCAG | 203 | H. sapiens | 571 |
| 197743 | 3 | 2189 | CATCGAGATTGGCTTGGAAG | 204 | H. sapiens | 572 |
| 197744 | 3 | 2649 | GGAGCTGGATTACAGTTGCA | 205 | H. sapiens | 573 |
| 197745 | 3 | 2729 | CAACATGCAGGCTGAACTGG | 206 | H. sapiens | 574 |
| 197746 | 3 | 2949 | ACATTACATTTGGTCTCTAC | 207 | H. sapiens | 575 |
| 197747 | 3 | 3059 | CTCAGGCGCTTACTCCAACG | 208 | H. sapiens | 576 |
| 197748 | 3 | 3118 | GGGACACCAGATTAGAGCTG | 209 | H. sapiens | 577 |
| 197749 | 3 | 3189 | GAGCTCCAGAGAGAGGACAG | 210 | H. sapiens | 578 |
| 197750 | 3 | 3289 | ATCGGCAGAGTATGACCTTG | 211 | H. sapiens | 579 |
| 197751 | 3 | 3488 | CAAGGGTGTTATTTCCATAC | 212 | H. sapiens | 580 |
| 197752 | 3 | 3579 | GACTCATCTGCTACAGCTTA | 213 | H. sapiens | 581 |
| 197753 | 3 | 4039 | GCAAATCCTCCAGAGATCTA | 214 | H. sapiens | 582 |
| 197754 | 3 | 4180 | CTCTCCTGGGTGTTCTAGAC | 215 | H. sapiens | 583 |
| 197755 | 3 | 4299 | ATGAAGGCTGACTCTGTGGT | 216 | H. sapiens | 584 |
| 197756 | 3 | 4511 | GGGACCACAGATGTCTGCTT | 217 | H. sapiens | 585 |
| 197757 | 3 | 4660 | CTGGCCGGCTCAATGGAGAG | 218 | H. sapiens | 586 |
| 197758 | 3 | 4919 | GCTGCGTTCTGAATATCAGG | 219 | H. sapiens | 587 |
| 197759 | 3 | 5009 | TGCTGACATCTTAGGCACTG | 220 | H. sapiens | 588 |
| 197760 | 3 | 5109 | AAGTGTAGTCTCCTGGTGCT | 221 | H. sapiens | 589 |
| 197761 | 3 | 5212 | CAAAATTCAGTCTGGATGGG | 222 | H. sapiens | 590 |
| 197762 | 3 | 5562 | GGGAAACTACGGCTAGAACC | 223 | H. sapiens | 591 |
| 197763 | 3 | 5589 | CTGCATGTGGCTGGTAACCT | 224 | H. sapiens | 592 |
| 197764 | 3 | 5839 | CCATGACCATCGATGCACAT | 225 | H. sapiens | 593 |
| 197765 | 3 | 5869 | ATGGGAAACTCGCTCTCTGG | 226 | H. sapiens | 594 |
| 197766 | 3 | 5979 | AGTCATCATCTCGTGTCTAG | 227 | H. sapiens | 595 |
| 197767 | 3 | 6099 | GAATACAGCCAGGACTTGGA | 228 | H. sapiens | 596 |
| 197768 | 3 | 6144 | GGCGTGGAGCTTACTGGACG | 229 | H. sapiens | 597 |
| 197769 | 3 | 6249 | GAGATGAGAGATGCCGTTGA | 230 | H. sapiens | 598 |
| 197770 | 3 | 6759 | AGTCTTGATGAGCACTATCA | 231 | H. sapiens | 599 |
| 197771 | 3 | 6889 | CTAAGTACCAAATCAGAATC | 232 | H. sapiens | 600 |
| 197772 | 3 | 7149 | GTCCATGAGTTAATCGAGAG | 233 | H. sapiens | 601 |
| 197773 | 3 | 7549 | AGGCCACAGTTGCAGTGTAT | 234 | H. sapiens | 602 |
| 197774 | 3 | 7779 | TCTGATTGGTGGACTCTTGC | 235 | H. sapiens | 603 |
| 197775 | 3 | 7929 | GAAGTCAGTCTTCAGGCTCT | 236 | H. sapiens | 604 |
| 197776 | 3 | 8929 | CCAGATTCTCAGATGAGGGA | 237 | H. sapiens | 605 |
| 197778 | 3 | 10240 | CATCTGTCATTGATGCACTG | 238 | H. sapiens | 606 |
| 197779 | 3 | 10619 | AGGAGATGTCAAGGGTTCGG | 239 | H. sapiens | 607 |
| 197780 | 3 | 10659 | GGAACTATTGCTAGTGAGGC | 240 | H. sapiens | 608 |
| 197781 | 3 | 10899 | CTCTCTCCATGGCAAATGTC | 241 | H. sapiens | 609 |
| 197783 | 3 | 11979 | CACCGTGACTTCAGTGCAGA | 243 | H. sapiens | 610 |
| 197784 | 3 | 12249 | ACTGAGTTGAGGGTCCGGGA | 244 | H. sapiens | 611 |
| 197786 | 3 | 13958 | CACATATGAACTGGACCTGC | 245 | H. sapiens | 612 |
| 197787 | 3 | 14008 | TCTGAACTCAGAAGGATGGC | 246 | H. sapiens | 613 |
| 216825 | 3 | 3249 | GGTGCGAAGCAGACTGAGGC | 247 | H. sapiens | 614 |
| 216826 | 3 | 3 | TCCCACCGGGACCTGCGGGG | 248 | H. sapiens | 615 |
| 216827 | 3 | 6 | CACCGGGACCTGCGGGGCTG | 249 | H. sapiens | 616 |
| 216828 | 3 | 23 | CTGAGTGCCCTTCTCGGTTG | 250 | H. sapiens | 617 |
| 216829 | 3 | 35 | CTCGGTTGCTGCCGCTGAGG | 251 | H. sapiens | 618 |
| 216830 | 3 | 36 | TCGGTTGCTGCCGCTGAGGA | 252 | H. sapiens | 619 |
| 216831 | 3 | 37 | CGGTTGCTGCCGCTGAGGAG | 253 | H. sapiens | 620 |
| 216832 | 3 | 39 | GTTGCTGCCGCTGAGGAGCC | 254 | H. sapiens | 621 |
| 216833 | 3 | 43 | CTGCCGCTGAGGAGCCCGCC | 255 | H. sapiens | 622 |
| 216834 | 3 | 116 | ACCGCAGCTGGCGATGGACC | 256 | H. sapiens | 623 |
| 216835 | 3 | 120 | CAGCTGGCGATGGACCCGCC | 257 | H. sapiens | 624 |
| 216836 | 3 | 13800 | GAACTTACTATCATCCTCTA | 258 | H. sapiens | 625 |
| 216838 | 3 | 13854 | TCCAATTGAACTTTCACATA | 260 | H. sapiens | 626 |
| 216839 | 3 | 13882 | AAAATTCAAACTGCCTATAT | 261 | H. sapiens | 627 |
| 216840 | 3 | 13903 | GATAAAACCATACAGTGAGC | 262 | H. sapiens | 628 |
| 216841 | 3 | 13904 | ATAAAACCATACAGTGAGCC | 263 | H. sapiens | 629 |
| 216842 | 3 | 13908 | AACCATACAGTGAGCCAGCC | 264 | H. sapiens | 630 |
| 216843 | 3 | 13909 | ACCATACAGTGAGCCAGCCT | 265 | H. sapiens | 631 |
| 216844 | 3 | 13910 | CCATACAGTGAGCCAGCCTT | 266 | H. sapiens | 632 |
| 216845 | 3 | 13917 | GTGAGCCAGCCTTGCAGTAG | 267 | H. sapiens | 633 |
| 216846 | 3 | 13922 | CCAGCCTTGCAGTAGGCAGT | 268 | H. sapiens | 634 |
| 216847 | 3 | 13934 | TAGGCAGTAGACTATAAGCA | 269 | H. sapiens | 635 |
| 216848 | 3 | 13937 | GCAGTAGACTATAAGCAGAA | 270 | H. sapiens | 636 |
| 216849 | 3 | 13964 | TGAACTGGACCTGCACCAAA | 271 | H. sapiens | 637 |
| 216850 | 3 | 13968 | CTGGACCTGCACCAAAGCTG | 272 | H. sapiens | 638 |
| 216851 | 3 | 13970 | GGACCTGCACCAAAGCTGGC | 273 | H. sapiens | 639 |
| 216852 | 3 | 13974 | CTGCACCAAAGCTGGCACCA | 274 | H. sapiens | 640 |
| 216853 | 3 | 13978 | ACCAAAGCTGGCACCAGGGC | 275 | H. sapiens | 641 |
| 216854 | 3 | 13997 | CTCGGAAGGTCTCTGAACTC | 276 | H. sapiens | 642 |
| 216855 | 3 | 14012 | AACTCAGAAGGATGGCATTT | 277 | H. sapiens | 643 |
| 216856 | 3 | 14014 | CTCAGAAGGATGGCATTTTT | 278 | H. sapiens | 644 |
| 216857 | 3 | 14049 | ATCAGGATCTGAGTTATTTT | 279 | H. sapiens | 645 |
| 216858 | 3 | 14052 | AGGATCTGAGTTATTTTGCT | 280 | H. sapiens | 646 |
| 216859 | 3 | 14057 | CTGAGTTATTTTGCTAAACT | 281 | H. sapiens | 647 |
| 216860 | 3 | 14064 | ATTTTGCTAAACTTGGGGGA | 282 | H. sapiens | 648 |
| 216861 | 3 | 14071 | TAAACTTGGGGGAGGAGGAA | 283 | H. sapiens | 649 |
| 217030 | 3 | 14087 | GGAACAAATAAATGGAGTCT | 284 | H. sapiens | 650 |
| 224316 | 3 | 3230 | GTTTGTAACTCAAGCAGAAG | 285 | H. sapiens | 651 |
| 224317 | 3 | 3232 | TTGTAACTCAAGCAGAAGGT | 286 | H. sapiens | 652 |
| 224318 | 3 | 3234 | GTAACTCAAGCAGAAGGTGC | 287 | H. sapiens | 653 |
| 224319 | 3 | 3236 | AACTCAAGCAGAAGGTGCGA | 288 | H. sapiens | 654 |
| 224320 | 3 | 3238 | CTCAAGCAGAAGGTGCGAAG | 289 | H. sapiens | 655 |
| 224321 | 3 | 3240 | CAAGCAGAAGGTGCGAAGCA | 290 | H. sapiens | 656 |
| 224322 | 3 | 3242 | AGCAGAAGGTGCGAAGCAGA | 291 | H. sapiens | 657 |
| 224323 | 3 | 3244 | CAGAAGGTGCGAAGCAGACT | 292 | H. sapiens | 658 |
| 224324 | 3 | 3246 | GAAGGTGCGAAGCAGACTGA | 293 | H. sapiens | 659 |
| 224325 | 3 | 3248 | AGGTGCGAAGCAGACTGAGG | 294 | H. sapiens | 660 |
| 224326 | 3 | 3250 | GTGCGAAGCAGACTGAGGCT | 295 | H. sapiens | 661 |
| 224327 | 3 | 3252 | GCGAAGCAGACTGAGGCTAC | 296 | H. sapiens | 662 |
| 224328 | 3 | 3254 | GAAGCAGACTGAGGCTACCA | 297 | H. sapiens | 663 |
| 224329 | 3 | 3256 | AGCAGACTGAGGCTACCATG | 298 | H. sapiens | 664 |
| 224330 | 3 | 3258 | CAGACTGAGGCTACCATGAC | 299 | H. sapiens | 665 |
| 224331 | 3 | 3260 | GACTGAGGCTACCATGACAT | 300 | H. sapiens | 666 |
| 224332 | 3 | 3262 | CTGAGGCTACCATGACATTC | 301 | H. sapiens | 667 |
| 224333 | 3 | 3264 | GAGGCTACCATGACATTCAA | 302 | H. sapiens | 668 |
| 224334 | 3 | 3266 | GGCTACCATGACATTCAAAT | 303 | H. sapiens | 669 |
| 224335 | 3 | 3268 | CTACCATGACATTCAAATAT | 304 | H. sapiens | 670 |
| 224336 | 3 | 5582 | CCTGAAGCTGCATGTGGCTG | 305 | H. sapiens | 671 |
| 224337 | 3 | 5584 | TGAAGCTGCATGTGGCTGGT | 306 | H. sapiens | 672 |
| 224338 | 3 | 5586 | AAGCTGCATGTGGCTGGTAA | 307 | H. sapiens | 673 |
| 224339 | 3 | 5588 | GCTGCATGTGGCTGGTAACC | 308 | H. sapiens | 674 |
| 224340 | 3 | 5590 | TGCATGTGGCTGGTAACCTA | 309 | H. sapiens | 675 |
| 224341 | 3 | 5592 | CATGTGGCTGGTAACCTAAA | 310 | H. sapiens | 676 |
| 224342 | 3 | 5594 | TGTGGCTGGTAACCTAAAAG | 311 | H. sapiens | 677 |
| 224343 | 3 | 5596 | TGGCTGGTAACCTAAAAGGA | 312 | H. sapiens | 678 |
| 224344 | 3 | 5598 | GCTGGTAACCTAAAAGGAGC | 313 | H. sapiens | 679 |
| 224345 | 3 | 5600 | TGGTAACCTAAAAGGAGCCT | 314 | H. sapiens | 680 |
| 224346 | 3 | 5602 | GTAACCTAAAAGGAGCCTAC | 315 | H. sapiens | 681 |
| 224347 | 3 | 5604 | AACCTAAAAGGAGCCTACCA | 316 | H. sapiens | 682 |
| 224348 | 3 | 5606 | CCTAAAAGGAGCCTACCAAA | 317 | H. sapiens | 683 |
| 187366 | 318 | 3121 | GGCGCGAAGCAGACTGAGGC | 319 | H. sapiens | 684 |
| 187404 | 318 | 12651 | CACTATGTTCATGAGGGAGG | 323 | H. sapiens | 685 |
| 197777 | 318 | 9851 | CCATCATAGGTTCTGACGTC | 324 | H. sapiens | 686 |
| 197785 | 318 | 12561 | GAAGCTGATTGACTCACTCA | 325 | H. sapiens | 687 |
| 224349 | 318 | 3104 | TTGTAACTCAAGCAGAAGGC | 326 | H. sapiens | 688 |
| 224350 | 318 | 3106 | GTAACTCAAGCAGAAGGCGC | 327 | H. sapiens | 689 |
| 224351 | 318 | 3108 | AACTCAAGCAGAAGGCGCGA | 328 | H. sapiens | 690 |
| 224352 | 318 | 3110 | CTCAAGCAGAAGGCGCGAAG | 329 | H. sapiens | 691 |
| 224353 | 318 | 3116 | CAGAAGGCGCGAAGCAGACT | 330 | H. sapiens | 692 |
| 224354 | 318 | 3118 | GAAGGCGCGAAGCAGACTGA | 331 | H. sapiens | 693 |
| 224355 | 318 | 3120 | AGGCGCGAAGCAGACTGAGG | 332 | H. sapiens | 694 |
| 224356 | 318 | 3122 | GCGCGAAGCAGACTGAGGCT | 333 | H. sapiens | 695 |
| 224328 | 3 | 3254 | GAAGCAGACTGAGGCTACCA | 514 | H. sapiens | 696 |
| 224353 | 318 | 3116 | CAGAAGGCGCGAAGCAGACT | 515 | H. sapiens | 697 |
| 216862 | 334 | 904 | TCTTTCTCCTGTCTTACAGA | 335 | H. sapiens | 698 |
| 216866 | 334 | 1988 | CCCACGTTAGAAGATGCGAC | 339 | H. sapiens | 699 |
| 216868 | 334 | 2722 | AATATGGTAGACATGAGCCA | 341 | H. sapiens | 700 |
| 216869 | 334 | 2791 | CATTAGCTGCATTTCAACTG | 342 | H. sapiens | 701 |
| 216870 | 334 | 3045 | ACTTTCACTCCTAGTCTGCA | 343 | H. sapiens | 702 |
| 216874 | 334 | 3527 | CCATGTCCAGGTAAGTCATG | 347 | H. sapiens | 703 |
| 216876 | 334 | 3603 | GCACCAGGCACGGATGTGAC | 349 | H. sapiens | 704 |
| 216877 | 334 | 3864 | GTGGGGTCCCAGAGGCACTG | 350 | H. sapiens | 705 |
| 216878 | 334 | 3990 | GCTGATCGGCCACTGCAGCT | 351 | H. sapiens | 706 |
| 216879 | 334 | 4251 | TCCACGTGGCTGGGGAGGTC | 352 | H. sapiens | 707 |
| 216880 | 334 | 4853 | TGTAATGTATGGTGATCAGA | 353 | H. sapiens | 708 |
| 216881 | 334 | 5023 | GAGAGTACCCAGTGGGAAAT | 354 | H. sapiens | 709 |
| 216882 | 334 | 5055 | AGTCAGCATGGGCTTCAGCC | 355 | H. sapiens | 710 |
| 216883 | 334 | 5091 | CAAAAGAATGACTGTCCAAC | 356 | H. sapiens | 711 |
| 216884 | 334 | 5096 | GAATGACTGTCCAACAAGTG | 357 | H. sapiens | 712 |
| 216885 | 334 | 5301 | TATCTACTGTAATTTAAAAT | 358 | H. sapiens | 713 |
| 216886 | 334 | 5780 | CTGATATGGGTGGAGAACAG | 359 | H. sapiens | 714 |
| 216887 | 334 | 6353 | TCTGGGACAGGTATGAGCTC | 360 | H. sapiens | 715 |
| 216888 | 334 | 6534 | TGATAGCAGTGGCCCTTGAA | 361 | H. sapiens | 716 |
| 216889 | 334 | 6641 | GGATTGGCGTGAAATACTGG | 362 | H. sapiens | 717 |
| 216890 | 334 | 6661 | TGCCCGAGGTTCCTCCTGCC | 363 | H. sapiens | 718 |
| 216894 | 334 | 7059 | GCACTAGCAAGACCACACTC | 367 | H. sapiens | 719 |
| 216895 | 334 | 7066 | CAAGACCACACTCTGCATAG | 368 | H. sapiens | 720 |
| 216897 | 334 | 7209 | TCCTCCATAGGATACCGTGT | 370 | H. sapiens | 721 |
| 216899 | 334 | 7702 | GGATGTAGGGCAGCAAAACC | 372 | H. sapiens | 722 |
| 216900 | 334 | 7736 | TCTGCACAAGGACTCCTTGT | 373 | H. sapiens | 723 |
| 216901 | 334 | 8006 | CAGCCTGTCTCAGTGAACAT | 374 | H. sapiens | 724 |
| 216903 | 334 | 8239 | CAGGATGCTTCCAGTCTAAT | 376 | H. sapiens | 725 |
| 216904 | 334 | 8738 | AAATGCTCGTCTCCAATCTC | 377 | H. sapiens | 726 |
| 216906 | 334 | 9208 | AACTTGTGTATCCAAATCCA | 379 | H. sapiens | 727 |
| 216908 | 334 | 9545 | TGACATGGTGTGCTTCCTTG | 381 | H. sapiens | 728 |
| 216910 | 334 | 9770 | ACACTGGTGTTCTGGCTACC | 383 | H. sapiens | 729 |
| 216911 | 334 | 9776 | GTGTTCTGGCTACCTCTAGT | 384 | H. sapiens | 730 |
| 216913 | 334 | 10341 | TCCTGGCATAGGTCACAGTA | 386 | H. sapiens | 731 |
| 216914 | 334 | 10467 | ATGTCAACAGTAGCACCTCC | 387 | H. sapiens | 732 |
| 216915 | 334 | 10522 | TAGACTCAGACAAGTCTGGA | 388 | H. sapiens | 733 |
| 216917 | 334 | 10587 | CCTAAGTTGCTCATCTCTGG | 390 | H. sapiens | 734 |
| 216922 | 334 | 11337 | TGCGCTGAGTTCCATGAAAC | 395 | H. sapiens | 735 |
| 216923 | 334 | 11457 | CTATTGCAGGTGCTCTCCAG | 396 | H. sapiens | 736 |
| 216926 | 334 | 12155 | CAGAGGAACATCTTGCACCT | 399 | H. sapiens | 737 |
| 216928 | 334 | 12221 | CTCTGCTCCTTACTCTTGTG | 401 | H. sapiens | 738 |
| 216929 | 334 | 12987 | GTAATTTCTCACCATCCATC | 402 | H. sapiens | 739 |
| 216930 | 334 | 13025 | TTCTGAGTCTCAATTGTCTA | 403 | H. sapiens | 740 |
| 216931 | 334 | 13057 | CTATGTCCTTGTGTGCACAT | 404 | H. sapiens | 741 |
| 216932 | 334 | 13634 | GCCTATTGCCATTTGTATGT | 405 | H. sapiens | 742 |
| 216933 | 334 | 13673 | CTATTCATGTCCTTTGCCTA | 406 | H. sapiens | 743 |
| 216935 | 334 | 14567 | GATTCTGCGGGTAATCTCAG | 408 | H. sapiens | 744 |
| 216937 | 334 | 14680 | TCAATGCAGGTCATTGGAAA | 410 | H. sapiens | 745 |
| 216938 | 334 | 15444 | CTACCAGATTGACCATCCCT | 411 | H. sapiens | 746 |
| 216940 | 334 | 15757 | TACTTGATAGTGCTCTAGGA | 413 | H. sapiens | 747 |
| 216941 | 334 | 15926 | TTGACTGCAGGACCAGGAGG | 414 | H. sapiens | 748 |
| 216942 | 334 | 16245 | AACAAACACTTGTGCAAATG | 415 | H. sapiens | 749 |
| 216950 | 334 | 18519 | AATGAGACCAAACTTCCACT | 423 | H. sapiens | 750 |
| 216951 | 334 | 18532 | TTCCACTTTGAAGCTAGCAA | 424 | H. sapiens | 751 |
| 216952 | 334 | 18586 | GATCTGGAGCTTATTCTTGA | 425 | H. sapiens | 752 |
| 216953 | 334 | 18697 | ACCTCATGTGACTTGTATGC | 426 | H. sapiens | 753 |
| 216954 | 334 | 18969 | TTCTTAAGAAACACCTTGTA | 427 | H. sapiens | 754 |
| 216955 | 334 | 19250 | TAGGCCCATCCTGGCTGCAT | 428 | H. sapiens | 755 |
| 216956 | 334 | 19340 | AAACTCTCAGGATATGGTAA | 429 | H. sapiens | 756 |
| 216957 | 334 | 19802 | ATACCTTCCTCTACCTTTGC | 430 | H. sapiens | 757 |
| 216958 | 334 | 19813 | TACCTTTGCTGAAGGTCCTT | 431 | H. sapiens | 758 |
| 216961 | 334 | 20567 | ATCTATCTAGTGAAATTTCT | 434 | H. sapiens | 759 |
| 216962 | 334 | 20647 | TCAGCTCATCAAAATATGCT | 435 | H. sapiens | 760 |
| 216963 | 334 | 20660 | ATATGCTAGTCCTTCCTTTC | 436 | H. sapiens | 761 |
| 216965 | 334 | 21316 | CAAAGGTCTGAGTTATCCAG | 438 | H. sapiens | 762 |
| 216967 | 334 | 21422 | TGACTTATAGATGCAGGCTG | 440 | H. sapiens | 763 |
| 216968 | 334 | 21634 | TCAGTGGAGGGTAATTCTTT | 441 | H. sapiens | 764 |
| 216969 | 334 | 21664 | TGCCTAGCCAGTTTGAAAGA | 442 | H. sapiens | 765 |
| 216970 | 334 | 21700 | CCTGCAGAATTTTGCCAGGC | 443 | H. sapiens | 766 |
| 216972 | 334 | 22048 | GTAGCTAGGTAGGTAAAGCA | 445 | H. sapiens | 767 |
| 216973 | 334 | 22551 | TTGAGTGAGACACACAAGGT | 446 | H. sapiens | 768 |
| 216974 | 334 | 22694 | GTGCTAGTCAGGGAATGCAT | 447 | H. sapiens | 769 |
| 216976 | 334 | 22903 | GGGGAGAGAGCATGCCCAGC | 449 | H. sapiens | 770 |
| 216977 | 334 | 22912 | GCATGCCCAGCTGCGAAAGC | 450 | H. sapiens | 771 |
| 216978 | 334 | 23137 | AGCCAGGTATAGAAAGGAGT | 451 | H. sapiens | 772 |
| 216979 | 334 | 23170 | AACTTTCTAAGAGGCAGAAT | 452 | H. sapiens | 773 |
| 216981 | 334 | 23882 | TCTTAGTCTGGTCATGAGTG | 454 | H. sapiens | 774 |
| 216983 | 334 | 24184 | AGTAGGAGATTTCATATGAA | 456 | H. sapiens | 775 |
| 216985 | 334 | 24559 | TCTTCACCAGCAACACATTA | 458 | H. sapiens | 776 |
| 216987 | 334 | 24800 | ATGGCCACCTAGCATGGCAC | 460 | H. sapiens | 777 |
| 216989 | 334 | 24991 | CATGTTTCTGAGCCTCCAGA | 462 | H. sapiens | 778 |
| 216990 | 334 | 25067 | TAGGTGGCTCCCTGTCTTCA | 463 | H. sapiens | 779 |
| 216991 | 334 | 25152 | TCCAAAGTCTTGGGAATCCT | 464 | H. sapiens | 780 |
| 216995 | 334 | 26749 | ACAAAGAAAGGGGGAGTTGG | 468 | H. sapiens | 781 |
| 216996 | 334 | 26841 | TCGTGTCTTCCTGGCCCAGA | 469 | H. sapiens | 782 |
| 216997 | 334 | 27210 | GCAGTGCCCAGCACACAATA | 470 | H. sapiens | 783 |
| 216998 | 334 | 27815 | ACTCGTCCAGGTGCGAAGCA | 471 | H. sapiens | 784 |
| 216999 | 334 | 28026 | GCCACCTAAGGTAAAGAAGG | 472 | H. sapiens | 785 |
| 217000 | 334 | 28145 | ATCAGAGTGGCAGAGAGAGC | 473 | H. sapiens | 786 |
| 217002 | 334 | 28919 | TTTACCATAGTTGTGACACA | 475 | H. sapiens | 787 |
| 217006 | 334 | 29871 | CATTTTGTAGGCAATGAGCT | 479 | H. sapiens | 788 |
| 217007 | 334 | 30181 | GCATTAGTAAACATGAGAAC | 480 | H. sapiens | 789 |
| 217009 | 334 | 30931 | TTCATTTCAGCGATGGCCGG | 482 | H. sapiens | 790 |
| 217013 | 334 | 39562 | GAAAATCTAGTGTCATTCAA | 486 | H. sapiens | 791 |
| 217016 | 334 | 39789 | TCCTATACAGTTTTGGGAAC | 489 | H. sapiens | 792 |
| 217017 | 334 | 39904 | AAGGACTTCAGTATGGAGCT | 490 | H. sapiens | 793 |
| 217018 | 334 | 39916 | ATGGAGCTTTTATTGAATTG | 491 | H. sapiens | 794 |
| 217022 | 334 | 40412 | CCATCAGCACTATTATTTAT | 495 | H. sapiens | 795 |
| 217023 | 334 | 40483 | ATAGGCAAGCTCAGCCATAG | 496 | H. sapiens | 796 |
| 217025 | 334 | 40576 | TGCTAGATGAGATACATCAA | 498 | H. sapiens | 797 |
| 217026 | 334 | 40658 | GAAGACCAAACATGGTTCTA | 499 | H. sapiens | 798 |
| 217029 | 334 | 41130 | CTCTGTTTAGTCCTCTCCAG | 502 | H. sapiens | 799 |
| 217032 | 503 | 490 | CATTGATAAAATGTTCTGGC | 505 | H. sapiens | 800 |
| 217033 | 503 | 504 | TCTGGCACAGCAAAACCTCT | 506 | H. sapiens | 801 |
| 217034 | 503 | 506 | TGGCACAGCAAAACCTCTAG | 507 | H. sapiens | 802 |
| 217037 | 503 | 523 | TAGAACACATAGTGTGATTT | 510 | H. sapiens | 803 |
| 217039 | 503 | 526 | AACACATAGTGTGATTTAAG | 512 | H. sapiens | 804 |

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of apolipoprotein B.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 37

Antisense Inhibition of Human Apolipoprotein B Expression—Dose Response of Oligonucleotides

In accordance with the present invention, 12 oligonucleotides described in Examples 29 and 31 were further investigated in a dose response study. The control oligonucleotides used in this study were ISIS 18076 (SEQ ID NO: 805) and ISIS 13650 (SEQ ID NO: 806).

All compounds in this study, including the controls, were chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotides. All cytidine residues are 5-methylcytidines.

In the dose-response experiment, with mRNA levels as the endpoint, HepG2 cells were treated with the antisense oligonucleotides or the control oligonucleotides at doses of 37, 75, 150, and 300 nM oligonucleotide. Data were obtained by real-time quantitative PCR as described in other examples herein and are averaged from two experiments with mRNA levels in the treatment groups being normalized to an untreated control group. The data are shown in Table 19.

TABLE 19
Inhibition of apolipoprotein B mRNA levels by chimeric
phosphorothioate oligonucleotides having 2′-
MOE wings and a deoxy gap - Dose Response

| | Dose | | | | |
|---|---|---|---|---|---|
| | 37 nM | 75 nM | 150 nM | 300 nM | |
| ISIS # | % inhibition | | | | SEQ ID NO |
| 271009 | 82 | 91 | 94 | 96 | 319 |
| 281625 | 62 | 76 | 84 | 94 | 224 |
| 301014 | 75 | 90 | 96 | 98 | 249 |
| 301027 | 80 | 90 | 95 | 96 | 262 |
| 301028 | 70 | 79 | 85 | 92 | 263 |
| 301029 | 54 | 67 | 79 | 85 | 264 |
| 301030 | 64 | 75 | 87 | 92 | 265 |
| 301031 | 61 | 82 | 92 | 96 | 266 |
| 301034 | 73 | 87 | 93 | 97 | 269 |
| 301036 | 67 | 83 | 92 | 95 | 271 |
| 301037 | 73 | 85 | 89 | 96 | 272 |
| 301045 | 77 | 86 | 94 | 98 | 280 |

### Example 38

Antisense Inhibition of Human Apolipoprotein B Expression—Dose Response—Lower Dose Range

In accordance with the present invention, seven oligonucleotides described in Examples 29, 31, 35, and 36 were further investigated in a dose response study. The control oligonucleotides used in this study were ISIS 18076 (SEQ ID NO: 805), ISIS 13650 (SEQ ID NO: 806), and ISIS 129695 (SEQ ID NO: 807).

All compounds in this study, including the controls, were chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotides. All cytidine residues are 5-methylcytidines.

In the dose-response experiment, with mRNA levels as the endpoint, HepG2 cells were treated with the antisense oligonucleotides or the control oligonucleotides at doses of 12.5, 37, 75, 150, and 300 nM oligonucleotide. Data were obtained by real-time quantitative PCR as described in other examples herein and are averaged from two experiments with mRNA levels in the treatment groups being normalized to an untreated control group. The data are shown in Table 20.

TABLE 20
Inhibition of apolipoprotein B mRNA levels by chimeric
phosphorothioate oligonucleotides having 2′-
MOE wings and a deoxy gap - Dose Response

| | Dose | | | | | |
|---|---|---|---|---|---|---|
| | 12.5 nM | 37 nM | 75 nM | 150 nM | 300 nM | |
| ISIS # | % inhibition | | | | | SEQ ID # |
| 271009 | 67 | 86 | 92 | 94 | 95 | 319 |
| 281625 | 44 | 66 | 83 | 85 | 94 | 224 |
| 301012 | 63 | 79 | 90 | 92 | 95 | 247 |
| 308638 | 42 | 73 | 91 | 96 | 97 | 247 |
| 308642 | 59 | 84 | 91 | 97 | 98 | 319 |
| 308651 | 57 | 76 | 84 | 90 | 88 | 319 |
| 308658 | 29 | 61 | 73 | 78 | 90 | 224 |

### Example 39

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5″-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5″-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be stably annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 40

Design and Screening of Duplexed Antisense Compounds Targeting Apolipoprotein B

In accordance with the present invention, a series of nucleic acid duplexes comprising the antisense compounds of the present invention and their complements are designed to target apolipoprotein B. The nucleobase sequence of the antisense strand of the duplex comprises at least a portion of an oligonucleotide described herein. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini. The antisense and sense strands of the duplex comprise from about 17 to 25 nucleotides, or from about 19 to 23 nucleotides. Alternatively, the antisense and sense strands comprise 20, 21 or 22 nucleotides.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

In another embodiment, a duplex comprising an antisense strand having the same sequence CGAGAGGCGGACGGGACCG may be prepared with blunt ends (no single stranded overhang) as shown:

| | |
|---|---|
| cgagaggcggacgggaccg | Antisense Strand |
| \|\|\|\|\|\|\|\|\|\|\|\|\|\|\|\|\|\|\| | |
| gctctccgcctgccctggc | Complement |

RNA strands of the duplex can be synthesized by methods disclosed herein or purchased from Dharmacon Research Inc., (Lafayette, Colo.). Once synthesized, the complementary strands are stably annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate apolipoprotein B expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 41

Design of Phenotypic Assays and In Vivo Studies for the Use of Apolipoprotein B Inhibitors

Phenotypic Assays

Once apolipoprotein B inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of apolipoprotein B in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with apolipoprotein B inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the genotype of the cell (measurement of the expression of one or more of the genes of the cell) after treatment is also used as an indicator of the efficacy or potency of the apolipoprotein B inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or apolipoprotein B inhibitor. Furthermore, to prevent the doctors from being biased in treatments, they are not informed as to whether the medication they are administering is a apolipoprotein B inhibitor or a placebo. Using this randomization approach, each volunteer has the same chance of being given either the new treatment or the placebo.

Volunteers receive either the apolipoprotein B inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding apolipoprotein B or apolipoprotein B protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and apolipoprotein B inhibitor treatment. In general, the volunteers treated with placebo have little or no response to treatment, whereas the volunteers treated with the apolipoprotein B inhibitor show positive trends in their disease state or condition index at the conclusion of the study.

### Example 42

Antisense Inhibition of Rabbit Apolipoprotein B Expression by Chimeric Phosphorothioate Oligonucleotides Having 2′-MOE Wings and a Deoxy Gap

In accordance with the present invention, a series of oligonucleotides was designed to target different regions of rabbit apolipoprotein B, using published sequences (GenBank accession number X07480.1, incorporated herein as SEQ ID NO: 808, GenBank accession number M17780.1, incorporated herein as SEQ ID NO: 809, and a sequence was derived using previously described primers (Tanaka, Journ. Biol. Chem., 1993, 268, 12713-12718) representing an mRNA of the rabbit apolipoprotein B, incorporated herein as SEQ ID NO: 810). The oligonucleotides are shown in Table 21. “Target site” indicates the first (5′-most) nucleotide number on the particular target sequence to which the oligonucleotide binds. All compounds in Table 21 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on rabbit apolipoprotein B mRNA levels in primary rabbit hepatocytes by quantitative real-time PCR as described in other examples herein. Primary rabbit hepatocytes were treated with 150 nM of the compounds in Table 21. For rabbit apolipoprotein B the PCR primers were:
forward primer: AAGCACCCCCAATGTCACC (SEQ ID NO: 811)
reverse primer: GGGATGGCAGAGCCAATGTA (SEQ ID NO: 812) and the PCR probe was: FAM-TCCTGGATTCAAGCTTCTATGTGCCTTCA-TAMRA (SEQ ID NO: 813) where FAM (PE-Applied Biosystems, Foster City, Calif.) is the fluorescent reporter dye) and TAMRA (PE-Applied Biosystems, Foster City, Calif.) is the quencher dye. Data are averages from two experiments. If present, “N.D.” indicates “no data”.

TABLE 21
Inhibition of rabbit apolipoprotein B mRNA
levels by chimeric phosphorothioate
oligonucleotides having 2′-MOE wings
and a deoxy gap

| | TARGET | | | | SEQ |
|---|---|---|---|---|---|
| ISIS | SEQ | TARGET | | % | ID |
| # | ID NO | SITE | SEQUENCE | INHIB | NO |
| 233149 | 808 | 1 | TGCTTGGAGAAGGTAAGATC | 0 | 814 |
| 233150 | 810 | 1 | GCGTTGTCTCCGATGTTCTG | 20 | 815 |
| 233151 | 809 | 13 | TAATCATTAACTTGCTGTGG | 20 | 816 |
| 233152 | 808 | 22 | TCAGCACGTAGCAATGCATT | 0 | 817 |
| 233153 | 808 | 31 | GCCTGATACTCAGCACGTAG | 0 | 818 |
| 233154 | 809 | 31 | CAATTGAATGTACTCAGATA | 18 | 819 |
| 233155 | 808 | 51 | ACCTCAGTGACTTGTAATCA | 47 | 820 |
| 233156 | 809 | 51 | CACTGGAAACTTGTCTCTCC | 23 | 821 |
| 233157 | 809 | 71 | AGTAGTTAGTTTCTCCTTGG | 0 | 822 |
| 233159 | 808 | 121 | TCAGTGCCCAAGATGTCAGC | 0 | 823 |
| 233160 | 810 | 121 | ATTGGAATAATGTATCCAGG | 81 | 824 |
| 233161 | 809 | 130 | TTGGCATTATCCAATGCAGT | 28 | 825 |
| 233162 | 808 | 151 | GTTGCCTTGTGAGCAGCAGT | 0 | 826 |
| 233163 | 810 | 151 | ATTGTGAGTGGAGATACTTC | 80 | 827 |
| 233164 | 809 | 171 | CATATGTCTGAAGTTGAGAC | 8 | 828 |
| 233165 | 808 | 181 | GTAGATACTCCATTTTGGCC | 0 | 829 |
| 233166 | 810 | 181 | GGATCACATGACTGAATGCT | 82 | 830 |
| 233167 | 808 | 201 | TCAAGCTGGTTGTTGCACTG | 28 | 831 |
| 233168 | 808 | 211 | GGACTGTACCTCAAGCTGGT | 0 | 832 |
| 233169 | 808 | 231 | GCTCATTCTCCAGCATCAGG | 14 | 833 |
| 233170 | 809 | 251 | TTGATCTATAATACTAGCTA | 23 | 834 |
| 233172 | 810 | 282 | ATGGAAGACTGGCAGCTCTA | 86 | 835 |
| 233173 | 808 | 301 | TTGTGTTCCTTGAAGCGGCC | 3 | 836 |
| 233174 | 809 | 301 | TGTGCACGGATATGATAACG | 21 | 837 |
| 233175 | 810 | 306 | GACCTTGAGTAGATTCCTGG | 90 | 838 |
| 233176 | 810 | 321 | GAAATCTGGAAGAGAGACCT | 62 | 839 |
| 233177 | 808 | 331 | GTAGCTTTCCCATCTAGGCT | 0 | 840 |
| 233178 | 808 | 346 | GATAACTCTGTGAGGGTAGC | 0 | 841 |
| 233179 | 810 | 371 | ATGTTGCCCATGGCTGGAAT | 65 | 842 |
| 233180 | 809 | 381 | AAGATGCAGTACTACTTCCA | 13 | 843 |
| 233181 | 808 | 382 | GCACCCAGAATCATGGCCTG | 0 | 844 |
| 233182 | 809 | 411 | CTTGATACTTGGTATCCACA | 59 | 845 |
| 233183 | 810 | 411 | CAGTGTAATGATCGTTGATT | 88 | 846 |
| 233184 | 810 | 431 | TAAAGTCCAGCATTGGTATT | 69 | 847 |
| 233185 | 810 | 451 | CAACAATGTCTGATTGGTTA | 73 | 848 |
| 233186 | 810 | 473 | GAAGAGGAAGAAAGGATATG | 60 | 849 |
| 233187 | 810 | 481 | TGACAGATGAAGAGGAAGAA | 66 | 850 |
| 233188 | 810 | 500 | TTGTACTGTAGTGCATCAAT | 74 | 851 |
| 233189 | 809 | 511 | GCCTCAATCTGTTGTTTCAG | 46 | 852 |
| 233190 | 810 | 520 | ACTTGAGCGTGCCCTCTAAT | 69 | 853 |
| 233191 | 809 | 561 | GAAATGGAATTGTAGTTCTC | 31 | 854 |

### Example 43

Antisense Inhibition of Rabbit Apolipoprotein B Expression by Chimeric Phosphorothioate Oligonucleotides Having 2′-MOE Wings and a Deoxy Gap-Dose Response Study

In accordance with the present invention, a subset of the antisense oligonuclotides in Example 42 was further investigated in dose-response studies. Treatment doses were 10, 50, 150 and 300 nM. ISIS 233160 (SEQ ID NO: 824), ISIS 233166 (SEQ ID NO: 830), ISIS 233172 (SEQ ID NO: 835), ISIS 233175 (SEQ ID NO: 838), and ISIS 233183 (SEQ ID NO: 846) were analyzed for their effect on rabbit apolipoprotein B mRNA levels in primary rabbit hepatocytes by quantitative real-time PCR as described in other examples herein. Data are averages from two experiments and are shown in Table 22.

TABLE 22
Inhibition of rabbit apolipoprotein B mRNA
levels by chimeric phosphorothioate oligonucleotides
having 2′-MOE wings and a deoxy gap

Percent Inhibition

| ISIS # | 300 nM | 150 nM | 50 nM | 10 nM |
|---|---|---|---|---|
| 233160 | 80 | 74 | 67 | 33 |
| 233166 | 73 | 79 | 81 | 66 |
| 233172 | 84 | 81 | 76 | 60 |
| 233175 | 93 | 90 | 85 | 67 |
| 233183 | 80 | 81 | 71 | 30 |

### Example 44

Effects of Antisense Inhibition of Apolipoprotein B in LDLr−/− Mice—Dose Response

LDL receptor-deficient mice (LDLr(−/−)mice), a strain that cannot edit the apolipoprotein B mRNA and therefore synthesize exclusively apolipoprotein B-100, have markedly elevated LDL cholesterol and apolipoprotein B-100 levels and develop extensive atherosclerosis.

LDLr(−/−) mice, purchased from Taconic (Germantown, N.Y.) were used to evaluate antisense oligonucleotides for their potential to lower apolipoprotein B mRNA or protein levels, as well as phenotypic endpoints associated with apolipoprotein B. LDLr(−/−) mice were separated into groups of males and females. LDLr(−/−) mice were dosed intraperitoneally twice a week for six weeks with either 10, 25, or 50 mg/kg of ISIS 147764 (SEQ ID NO: 109) or ISIS 270906 (SEQ ID NO: 856) which is a 4 base mismatch of ISIS 147764, or with saline, or 20 mg/kg of Atorvastatin. At study termination animals were sacrificed and evaluated for several phenotypic markers.

ISIS 147764 was able to lower cholesterol, triglycerides, and mRNA levels in a dose-dependent manner in both male and female mice while the 4-base mismatch ISIS 270906 was not able to do this. The results of the study are summarized in Table 23.

TABLE 23
Effects of ISIS 147764 treatment in male and female LDLr−/− mice
on apolipoprotein B mRNA, liver enzyme, cholesterol, and triglyceride levels.

| | Dose | Liver Enzymes IU/L | | Lipoproteins mg/dL | | | | mRNA % |
|---|---|---|---|---|---|---|---|---|
| ISIS No. | mg/kg | AST | ALT | CHOL | HDL | LDL | TRIG | control |

Males

| Saline | | 68.4 | 26.6 | 279.2 | 125.4 | 134.7 | 170.6 | 100.0 |
|---|---|---|---|---|---|---|---|---|
| 147764 | 10 | 57.6 | 29.8 | 314.2 | 150.0 | 134.7 | 198.6 | 61.7 |
| | 25 | 112.6 | 78.8 | 185.0 | 110.6 | 66.2 | 104.2 | 30.7 |
| | 50 | 163.6 | 156.8 | 165.6 | 107.8 | 51.2 | 113.4 | 16.6 |
| 270906 | 50 | 167.4 | 348.0 | 941.0 | 244.2 | 541.9 | 844.8 | N.D. |
| Atorvastatin | 20 | N.D. | N.D. | N.D. | N.D. | N.D. | N.D. | 110.9 |

Females

| Saline | | 65.0 | 23.4 | 265.8 | 105.8 | 154.9 | 121.4 | 100.0 |
|---|---|---|---|---|---|---|---|---|
| 147764 | 10 | 82.0 | 27.2 | 269.6 | 121.0 | 127.8 | 140.8 | 64.2 |
| | 25 | 61.4 | 32.2 | 175.8 | 99.5 | 68.9 | 100.4 | 41.3 |
| | 50 | 134.6 | 120.4 | 138.2 | 92.2 | 45.9 | 98.0 | 18.5 |
| 270906 | 50 | 96.0 | 88.6 | 564.6 | 200.0 | 310.0 | 240.4 | N.D. |
| Atorvastatin | 20 | N.D. | N.D. | N.D. | N.D. | N.D. | N.D. | 109.0 |

### Example 45

Effects of Antisense Inhibition of Apolipoprotein B in Cynomolgus Monkeys

Cynomolgus monkeys fed an atherogenic diet develop atherosclerosis with many similarities to atherosclerosis of human beings. Female Cynomolgus macaques share several similarities in lipoproteins and the cardiovascular system with humans. In addition to these characteristics, there are similarities in reproductive biology. The Cynomolgus female has a 28-day menstrual cycle like that of women. Plasma hormone concentrations have been measured throughout the Cynomolgus menstrual cycle, and the duration of the follicular and luteal phases, as well as plasma estradiol and progesterone concentrations across the cycle, are also remarkably similar to those in women.

Cynomolgus monkeys (male or female) can be used to evaluate antisense oligonucleotides for their potential to lower apolipoprotein B mRNA or protein levels, as well as phenotypic endpoints associated with apolipoprotein B including, but not limited to cardiovascular indicators, atherosclerosis, lipid diseases, obesity, and plaque formation. One study could include normal and induced hypercholesterolemic monkeys fed diets that are normal or high in lipid and cholesterol. Cynomolgus monkeys can be dosed in a variety of regimens, one being subcutaneously with 10-20 mg/kg of the oligomeric compound for 1-2 months. Parameters that may observed during the test period could include: total plasma cholesterol, LDL-cholesterol, HDL-cholesterol, triglyceride, arterial wall cholesterol content, and coronary intimal thickening.

### Example 46

Sequencing of Cynomolgus Monkey (Macaca fascicularis) Apolipoprotein B Preferred Target Segment

In accordance with the present invention, a portion of the cynomolgus monkey apolipoprotein B mRNA not available in the art, was amplified. Positions 2920 to 3420 of the human apolipoprotein B mRNA sequence (GenBank accession number NM_000384.1, incorporated herein as SEQ ID NO: 3) contain the preferred target segment to which ISIS 301012 hybridizes and the corresponding segment of cynomolgus monkey apolipoprotein B mRNA was amplified and sequenced. The site to which ISIS 301012 hybridizes in the human apolipoprotein B was amplified by placing primers at 5′ position 2920 and 3′ position 3420. The cynomolgus monkey hepatocytes were purchased from In Vitro Technologies (Gaithersburg, Md.). The 500 bp fragments were produced using human and cynomolgus monkey 1° hepatocyte cDNA and were produced by reverse transcription of purified total RNA followed by 40 rounds of PCR amplification. Following gel purification of the human and cynomolgus amplicons, the forward and reverse sequencing reactions of each product were performed by Retrogen (Invitrogen kit was used to create the single-stranded cDNA and provided reagents for Amplitaq PCR reaction). This cynomolgus monkey sequence is incorporated herein as SEQ ID NO: 855 and is 96% identical to positions 2920 to 3420 of the human apolipoprotein B mRNA.

### Example 47

Effects of Antisense Inhibition of Human Apolipoprotein B Gene (ISIS 281625 and 301012) in C57BL/6NTac-TgN(APOB100) Transgenic Mice

C57BL/6NTac-TgN(APOB100) transgenic mice have the human apolipoprotein B gene “knocked-in”. These mice express high levels of human apolipoprotein B100 resulting in mice with elevated serum levels of LDL cholesterol. These mice are useful in identifying and evaluating compounds to reduce elevated levels of LDL cholesterol and the risk of atherosclerosis. When fed a high fat cholesterol diet, these mice develop significant foam cell accumulation underlying the endothelium and within the media, and have significantly more complex atherosclerotic lesions than control animals.

C57BL/6NTac-TgN(APOB100) mice were divided into two groups—one group receiving oligonucleotide treatment and control animals receiving saline treatment. After overnight fasting, mice were dosed intraperitoneally twice a week with saline or 25 mg/kg ISIS 281625 (SEQ ID No: 224) or ISIS 301012 (SEQ ID No: 247) for eight weeks. At study termination and forty eight hours after the final injections, animals were sacrificed and evaluated for target mRNA levels in liver, cholesterol and triglyceride levels, and liver enzyme levels. In addition, the endogenous mouse apolipoprotein B levels in liver were measured to evaluate any effects of these antisense oligonucleotides targeted to the human apolipoprotein B.

Upon treatment with either ISIS 281625 or ISIS 301012, the AST and ALT levels were increased, yet did not exceed normal levels (˜300 IU/L). Cholesterol levels were slightly increased relative to saline treatment, while triglyceride levels were slightly decreased. Treatment with either of these oligonucleotides targeted to the human apolipoprotein B which is expressed in these mice markedly decreased the mRNA levels of the human apolipoprotein, while the levels of the endogenous mouse apolipoprotein B were unaffected, indicating that these oligonucleotides exhibit specificity for the human apolipoprotein B. The results of the comparative studies are shown in Table 24.

TABLE 24
Effects of ISIS 281625 and 301012 treatment in mice on apolipoprotein
B mRNA, liver enzyme, cholesterol, and triglyceride levels.

ISIS No.

| | SALINE | 281625 | 301012 |
|---|---|---|---|
| Liver Enzymes IU/L | | | |
| AST | 70.3 | 265.8 | 208.4 |
| ALT | 32.8 | 363.8 | 137.4 |
| Lipoproteins mg/dL | | | |
| CHOL | 109.5 | 152.0 | 145.1 |
| HDL | 67.3 | 84.6 | 98.6 |
| LDL | 30.2 | 49.8 | 36.6 |
| TRIG | 194.5 | 171.1 | 157.8 |
| mRNA % control | | | |
| human mRNA | 100.0 | 45.2 | 23.7 |
| mouse mRNA | 100.0 | 111.0 | 94.6 |

Following 2 and 4 weeks of ISIS 301012 treatment, LDL-cholesterol levels were significantly reduced to 22 mg/dL and 17 mg/dL, respectively.

Apolipoprotein B protein levels in liver were also evaluated at the end of the 8 week treatment period. Liver protein was isolated and subjected to immunoblot analysis using antibodies specific for human or mouse apolipoprotein B protein (US Biologicals, Swampscott, Mass. and Santa Cruz Biotechnology, Inc., Santa Cruz, Calif., respectively). Immunoblot analysis of liver protein samples reveals a reduction in the expression of both forms of human apolipoprotein B, apolipoprotein B-100 and apolipoprotein B-48. Mouse apolipoprotein B levels in liver were not significantly changed, as judged by immunoblot analysis.

Serum samples were also collected at 2, 4, 6 and 8 weeks and were evaluated for human apolipoprotein B expression by using a human apolipoprotein B specific ELISA kit (ALerCHEK Inc., Portland, Me.). Quantitation of serum human apolipoprotein B protein by ELISA revealed that treatment with ISIS 281625 reduced serum human apolipoprotein B protein by 31, 26, 11 and 26% at 2, 4, 6 and 8 weeks, respectively, relative to saline-treated animals. Treatment with ISIS 301012 reduced serum human apolipoprotein B protein by 70, 87, 81 and 41% at 2, 4, 6 and 8 weeks, respectively, relative to saline-treated control animals. Serum from transgenic mice was also subjected to immunoblot analysis using both human and mouse specific apolipoprotein B antibodies (US Biologicals, Swampscott, Mass. and Santa Cruz Biotechnology, Inc., Santa Cruz, Calif., respectively). Immunoblot analysis of serum samples taken from animals shows a similar pattern of human apolipoprotein B expression, with a significant reduction in serum apolipoprotein B protein after 2, 4 and 6 weeks of treatment and a slight reduction at 8 weeks. Mouse apolipoprotein B in serum was not significantly changed, as judged by immunoblot analysis.

### Example 48

Effects of Antisense Inhibition of Apolipoprotein B (ISIS 233172, 233175, 281625, 301012, and 301027) in C57BL/6 mice

C57BL/6 mice, a strain reported to be susceptible to hyperlipidemia-induced atherosclerotic plaque formation were used in the following studies to evaluate the toxicity in mice of several antisense oligonucleotides targeted to human or rabbit apolipoprotein B.

C57BL/6 mice were divided into two groups—one group receiving oligonucleotide treatment and control animals receiving saline treatment. After overnight fasting, mice were dosed intraperitoneally twice a week with saline or 25 mg/kg of one of several oligonucleotides for two weeks. The antisense oligonucleotides used in the present study were ISIS 233172 (SEQ ID NO: 835) and ISIS 233175 (SEQ ID NO: 838), both targeted to rabbit apolipoprotein B, and ISIS 281625 (SEQ ID NO: 224), ISIS 301012 (SEQ ID NO: 247), and ISIS 301027 (SEQ ID NO: 262), targeted to human apolipoprotein B. At study termination and forty eight hours after the final injections, animals were sacrificed and evaluated for liver enzyme levels, body weight, liver weight, and spleen weight.

The levels of liver enzymes in mice were decreased relative to saline treatment for three of the antisense oligonucleotide. However, the rabbit oligonucleotide ISIS 233175 and the human oligonucleotide ISIS 301027 both elicited drastically increased levels of these liver enzymes, indicating toxicity. For all of the oligonucleotides tested, the change in weight of body, liver, and spleen were minor. The results of the comparative studies are shown in Table 25.

TABLE 25
Effects of antisense oligonucleotides targeted to human or rabbit
apolipoprotein B on mouse apolipoprotein B mRNA, liver enzyme,
cholesterol, and triglyceride levels.

| | | ISIS No. | | | | |
|---|---|---|---|---|---|---|
| | SALINE | 233172 | 233175 | 281625 | 301012 | 301027 |
| Liver Enzymes | | | | | | |
| AST IU/L | 104.5 | 94.3 | 346.7 | 89.5 | 50.6 | 455.3 |
| ALT IU/L | 39.5 | 43.3 | 230.2 | 36.2 | 21.2 | 221.3 |
| Weight | | | | | | |
| BODY | 21.2 | 21.3 | 21.5 | 20.9 | 21.3 | 21.2 |
| LIVER | 1.1 | 1.3 | 1.4 | 1.2 | 1.1 | 1.3 |
| SPLEEN | 0.1 | 0.1 | 0.1 | 0.1 | 0.1 | 0.1 |

### Example 49

Time Course Evaluation of Oligonucleotide at Two Different Doses

C57BL/6 mice, a strain reported to be susceptible to hyperlipidemia-induced atherosclerotic plaque formation were used in the following studies to evaluate the toxicity in mice of several antisense oligonucleotides targeted to human apolipoprotein B.

Female C57BL/6 mice were divided into two groups—one group receiving oligonucleotide treatment and control animals receiving saline treatment. After overnight fasting, mice were dosed intraperitoneally twice a week with saline or 25 mg/kg or 50 mg/kg of ISIS 281625 (SEQ ID NO: 224), ISIS 301012 (SEQ ID NO: 247), or ISIS 301027 (SEQ ID NO: 262). After 2 weeks, a blood sample was taken from the tail of the mice and evaluated for liver enzyme. After 4 weeks, and study termination, animals were sacrificed and evaluated for liver enzyme levels.

For ISIS 281625 and ISIS 301012, AST and ALT levels remained close to those of saline at either dose after 2 weeks. After 4 weeks, AST and ALT levels showed a moderate increase over saline treated animals for the lower dose, but a large increase at the higher dose. ISIS 301027, administered at either dose, showed a small increase in AST and ALT levels after 2 weeks and a huge increase in AST and ALT levels after 4 weeks. The results of the studies are summarized in Table 26.

TABLE 26
AST and ALT levels in mice treated with ISIS
281625, 301012, or 301027 after 2 and 4 weeks

| | | AST (IU/L) | | ALT (IU/L) | |
|---|---|---|---|---|---|
| | | 2 weeks | 4 weeks | 2 weeks | 4 weeks |
| SALINE | | 49.6 | 63.2 | 22.4 | 25.2 |
| | Dose | | | | |
| ISIS No. | (mg/kg) | | | | |
| 281625 | 25 | 40.8 | 75 | 21.2 | 31.8 |
| | 50 | 44.4 | 152.4 | 30.8 | 210.4 |
| 301012 | 25 | 37.2 | 89.8 | 22.4 | 24.8 |
| | 50 | 38.4 | 107.4 | 23.2 | 29.2 |
| 301027 | 25 | 55.4 | 537.6 | 27.2 | 311.2 |
| | 50 | 64 | 1884 | 34.8 | 1194 |

### Example 50

Effects of Antisense Inhibition of Apolipoprotein B (ISIS 147483 and 147764) in Ob/Ob Mice

Leptin is a hormone produced by fat that regulates appetite. Deficiencies in this hormone in both humans and non-human animals leads to obesity. ob/ob mice have a mutation in the leptin gene which results in obesity and hyperglycemia. As such, these mice are a useful model for the investigation of obesity and diabetes and treatments designed to treat these conditions.

Ob/ob mice receiving a high fat, high cholesterol diet (60% kcal fat supplemented with 0.15% cholesterol) were treated with one of several oligonucleotides to evaluate their effect on apolipoprotein B-related phenotypic endpoints in ob/ob mice. After overnight fasting, mice from each group were dosed intraperitoneally twice a week with 50 mg/kg of ISIS 147483 (SEQ ID NO: 79), or 147764 (SEQ ID NO: 109), or the controls ISIS 116847 (SEQ ID NO: 857), or 141923 (SEQ ID NO: 858), or saline for six weeks. At study termination and forty eight hours after the final injections, animals were sacrificed and evaluated for target mRNA levels in liver, cholesterol and triglyceride levels, liver enzyme levels, serum glucose levels, and PTEN levels.

ISIS 147483 and 147764 were both able to lower apolipoprotein B mRNA levels, as well as glucose, cholesterol, and triglyceride levels. The results of the comparative studies are shown in Table 27.

TABLE 27
Effects of ISIS 147483 and 147764 treatment in ob/ob
mice on apolipoprotein B mRNA, cholesterol, lipid, triglyceride,
liver enzyme, glucose, and PTEN levels.

| | | ISIS No. | | | |
|---|---|---|---|---|---|
| | SALINE | 116847 | 141923 | 147483 | 147764 |
| Glucose mg/dL | 269.6 | 135.5 | 328.5 | 213.2 | 209.2 |
| Liver Enzymes IU/L | | | | | |
| AST | 422.3 | 343.2 | 329.3 | 790.2 | 406.5 |
| ALT | 884.3 | 607.5 | 701.7 | 941.7 | 835.0 |
| Lipoproteins mg/dL | | | | | |
| CHOL | 431.9 | 287.5 | 646.3 | 250.0 | 286.3 |
| TRIG | 128.6 | 196.5 | 196.5 | 99.8 | 101.2 |
| mRNA % control | | | | | |
| ApoB | 100.0 | 77.0 | 100.0 | 25.2 | 43.1 |
| PTEN | 100.0 | 20.0 | 113.6 | 143.2 | 115.3 |

### Example 51

Antisense Inhibition of Apolipoprotein B in High Fat Fed Mice: Time-Dependent Effects

In a further embodiment of the invention, the inhibition of apolipoprotein B mRNA in mice was compared to liver oligonucleotide concentration, total cholesterol, LDL-cholesterol and HDL-cholesterol. Male C57Bl/6 mice receiving a high fat diet (60% fat) were evaluated over the course of 6 weeks for the effects of treatment with twice weekly intraperitoneal injections of 50 mg/kg ISIS 147764 (SEQ ID NO: 109) or 50 mg/kg of the control oligonucleotide ISIS 141923 (SEQ ID NO: 858). Control animals received saline treatment. Animals were sacrificed after 2 days, 1, 2, 4 and 6 weeks of treatment. Each treatment group at each time point consisted of 8 mice.

Target expression in liver was measured by real-time PCR as described by other examples herein and is expressed as percent inhibition relative to saline treated mice. Total, LDL- and HDL-cholesterol levels were measured by routine clinical analysis using an Olympus Clinical Analyzer (Olympus America Inc., Melville, N.Y.) and are presented in mg/dL. Results from saline-treated animals are shown for comparison. Intact oligonucleotide in liver tissue was measured by capillary gel electrophoresis and is presented as micrograms of oligonucleotide per gram of tissue. All results are the average of 8 animals and are shown in Table 28.

TABLE 28
Correlation between liver drug concentration, apolipoprotein B mRNA
expression and serum lipids during ISIS 147764 treatment

| | | Treatment period | | | | |
|---|---|---|---|---|---|---|
| | | 2 | 1 | 2 | 4 | 6 |
| | ISIS # | days | week | weeks | weeks | weeks |
| % Inhibition | 141923 | 9 | 4 | 7 | 0 | 0 |
| apolipoprotein B mRNA | 147764 | 50 | 57 | 73 | 82 | 88 |
| Intact oligonucleotide | 141923 | 58 | 61 | 152 | 261 | 631 |
| ug/g | 147764 | 85 | 121 | 194 | 340 | 586 |
| Total cholesterol | saline | 105 | 152 | 144 | 180 | 191 |
| mg/dL | 141923 | 99 | 146 | 152 | 169 | 225 |
| | 147764 | 101 | 128 | 121 | 75 | 73 |
| LDL-cholesterol | saline | 8 | 32 | 28 | 50 | 46 |
| mg/dL | 141923 | 8 | 27 | 27 | 38 | 56 |
| | 147764 | 7 | 19 | 14 | 7 | 7 |
| HDL-cholesterol | saline | 74 | 117 | 114 | 127 | 141 |
| mg/dL | 141923 | 70 | 116 | 122 | 128 | 166 |
| | 147764 | 76 | 107 | 105 | 66 | 64 |

These results illustrate that inhibition of apolipoprotein B mRNA by ISIS 147764 occurred within 2 days of treatment, increased with successive treatments and persisted for 6 weeks of treatment. Quantitation of liver oligonucleotide levels reveals a strong correlation between the extent of target inhibition and liver drug concentration. Furthermore, at 1, 2, 3 and 4 weeks of treatment, a inverse correlation between inhibition of target mRNA and cholesterol levels (total, HDL and LDL) is observed, with cholesterol levels lowering as percent inhibition of apolipoprotein B mRNA becomes greater. Serum samples were subjected to immunoblot analysis using an antibody to detect mouse apolipoprotein B protein (Gladstone Institute, San Francisco, Calif.). The expression of protein follows the same pattern as that of the mRNA, with apolipoprotein B protein in serum markedly reduced within 48 hours and lowered throughout the 6 week treatment period.

The oligonucleotide treatments described in this example were duplicated to investigate the extent to which effects of ISIS 147764 persist following cessation of treatment. Mice were treated as described, and sacrificed 1, 2, 4, 6 and 8 weeks following the cessation of oligonucleotide treatment. The same parameters were analyzed and the results are shown in Table 29.

TABLE 29
Correlation between liver drug concentration, apolipoprotein B mRNA
expression, and serum lipids after cessation of dosing

| | | Treatment period | | | | |
|---|---|---|---|---|---|---|
| | | 1 | 2 | 4 | 6 | 8 |
| | ISIS # | week | weeks | weeks | weeks | weeks |
| % Inhibition | 141923 | 15 | 2 | 7 | 11 | 7 |
| apolipoprotein B mRNA | 147764 | 82 | 78 | 49 | 37 | 19 |
| Intact oligonucleotide | 141923 | 297 | 250 | 207 | 212 | 128 |
| ug/g | 147764 | 215 | 168 | 124 | 70 | 43 |
| Total cholesterol | saline | 114 | 144 | 195 | 221 | 160 |
| mg/dL | 141923 | 158 | 139 | 185 | 186 | 151 |
| | 147764 | 69 | 67 | 111 | 138 | 135 |
| LDL-cholesterol | saline | 21 | 24 | 34 | 37 | 22 |
| mg/dL | 141923 | 24 | 24 | 32 | 32 | 24 |
| | 147764 | 14 | 14 | 18 | 24 | 21 |
| HDL-cholesterol | saline | 86 | 109 | 134 | 158 | 117 |
| mg/dL | 141923 | 121 | 105 | 135 | 136 | 108 |
| | 147764 | 51 | 49 | 79 | 100 | 94 |

These data demonstrate that after termination of oligonucleotide treatment, the effects of ISIS 147764, including apolipoprotein B mRNA inhibition, and cholesterol lowering, persist for up to 8 weeks. Immunoblot analysis demonstrates that apolipoprotein B protein levels follow a pattern similar that observed for mRNA expression levels.

### Example 52

Effects of Antisense Inhibition of Human Apolipoprotein B Gene by 301012 in C57BL/6NTac-TgN(APOB100) Transgenic Mice: Dosing Study

C57BL/6NTac-TgN(APOB100) transgenic mice have the human apolipoprotein B gene “knocked-in”. These mice express high levels of human apolipoprotein B resulting in mice with elevated serum levels of LDL cholesterol. These mice are useful in identifying and evaluating compounds to reduce elevated levels of LDL cholesterol and the risk of atherosclerosis. When fed a high fat cholesterol diet, these mice develop significant foam cell accumulation underlying the endothelium and within the media, and have significantly more complex atherosclerotic plaque lesions than control animals.

A long-term study of inhibition of human apolipoprotein B by ISIS 301012 in C57BL/6NTac-TgN(APOB100) mice (Taconic, Germantown, N.Y.) was conducted for a 3 month period. Mice were dosed intraperitoneally twice a week with 10 or 25 mg/kg ISIS 301012 (SEQ ID No: 247) for 12 weeks. Saline-injected animals served as controls. Each treatment group comprised 4 animals.

After 2, 4, 6, 8 and 12 weeks of treatment, serum samples were collected for the purpose of measuring human apolipoprotein B protein. Serum protein was quantitated using an ELISA kit specific for human apolipoprotein B (ALerCHEK Inc., Portland, Me.). The data are shown in Table 30 and each result represents the average of 4 animals. Data are normalized to saline-treated control animals.

TABLE 30
Reduction of human apolipoprotein B protein in transgenic
mouse serum following ISIS 301012 treatment

| | % Reduction in human apolipoprotein B | | | | |
|---|---|---|---|---|---|
| Dose of | protein in serum | | | | |
| oligonucleotide | 2 | 4 | 6 | 8 | 12 |
| mg/kg | weeks | weeks | weeks | weeks | weeks |
| 10 | 76 | 78 | 73 | 42 | 85 |
| 25 | 80 | 87 | 86 | 47 | 79 |

These data illustrate that following 2, 4, 6 or 12 weeks of treatment with ISIS 301012, the level of human apolipoprotein B protein in serum from transgenic mice is lowered by approximately 80%, demonstrating that in addition to inhibiting mRNA expression, ISIS 301012 effectively inhibits human apolipoprotein B protein expression in mice carrying the human apolipoprotein B transgene. Apolipoprotein B protein in serum was also assessed by immunoblot analysis using an antibody directed to human apolipoprotein B protein (US Biologicals, Swampscott, Mass.). This analysis shows that the levels human apolipoprotein B protein, both the apolipoprotein B-100 and apolipoprotein B-48 forms, are lowered at 2, 4, 6 and 12 weeks of treatment. Immunoblot analysis using a mouse apolipoprotein B specific antibody (Santa Cruz Biotechnology, Inc., Santa Cruz, Calif.) reveals no significant change in the expression of the mouse protein in serum.

At the beginning of the treatment (start) and after 2, 4, 6 and 8 weeks of treatment, serum samples were collected and total, LDL- and HDL-cholesterol levels were measured by routine clinical analysis using an Olympus Clinical Analyzer (Olympus America Inc., Melville, N.Y.), and these data are presented in Table 31. Results are presented as mg/dL in serum and represent the average of 4 animals. Results from the saline control animals are also shown.

TABLE 31
Effects of ISIS 301012 on serum lipids in human
apolipoprotein B transgenic mice

| | | Treatment period | | | | |
|---|---|---|---|---|---|---|
| | | | 2 | 4 | 6 | 8 |
| | Treatment | Start | weeks | weeks | weeks | weeks |
| Total cholesterol | Saline | 120 | 110 | 129 | 121 | 126 |
| mg/dL | 10 | 115 | 97 | 111 | 120 | 122 |
| | 25 | 107 | 101 | 107 | 124 | 147 |
| HDL-cholesterol | Saline | 67 | 61 | 69 | 62 | 64 |
| mg/dL | 10 | 70 | 69 | 78 | 72 | 79 |
| | 25 | 64 | 73 | 76 | 80 | 91 |
| LDL-cholesterol | Saline | 39 | 41 | 50 | 45 | 47 |
| mg/dL | 10 | 35 | 20 | 23 | 37 | 33 |
| | 25 | 33 | 19 | 19 | 37 | 44 |

These data demonstrate that LDL-cholesterol is lowered by treatment with 10 or 25 mg/kg of ISIS 147764 during the first 4 weeks of treatment.

The study was terminated forty eight hours after the final injections in the eighth week of treatment, when animals were sacrificed and evaluated for target mRNA levels in liver, apolipoprotein B protein levels in liver and serum cholesterol and liver enzyme levels. In addition, the expression of endogenous mouse apolipoprotein B levels in liver was measured to evaluate any effects of ISIS 301012 on mouse apolipoprotein B mRNA expression.

Human and mouse apolipoprotein B mRNA levels in livers of animals treated for 12 weeks were measured by real-time PCR as described herein. Each result represents the average of data from 4 animals. The data were normalized to saline controls and are shown in Table 32.

TABLE 32
Effects of ISIS 301012 on human and mouse apolipoprotein
B mRNA levels in transgenic mice

| | % Inhibition | |
|---|---|---|
| | Dose of | |
| | ISIS 301012 | |
| | 10 | 25 |
| mRNA species measured | mg/kg | mg/kg |
| human apolipoprotein B | 65 | 75 |
| mouse apolipoprotein B | 6 | 6 |

These data demonstrate that following 12 weeks of treatment with ISIS 301012, human apolipoprotein B mRNA is reduced by as much as 75% in the livers of transgenic mice, whereas mouse liver apolipoprotein B mRNA was unaffected. Furthermore, ELISA analysis of apolipoprotein B protein in livers of transgenic mice reveals an 80% and 82% reduction in the human protein following 10 and 20 mg/kg ISIS 301012, respectively. Immunoblot analysis using an antibody directed to human apolipoprotein B also demonstrates a reduction in the expression of human apolipoprotein B, both the apolipoprotein B-100 and apolipoprotein B-48 forms, in the livers of transgenic mice. Immunoblot analysis using an antibody directed to mouse apolipoprotein B protein (Santa Cruz Biotechnology, Inc., Santa Cruz, Calif.) reveals that expression of the mouse protein in liver does not change significantly.

ALT and AST levels in serum were also measured using the Olympus Clinical Analyzer (Olympus America Inc., Melville, N.Y.) and showed that following treatment with ISIS 301012, the AST and ALT levels were increased, yet did not exceed normal levels (˜300 IU/L), indicating a lack of toxicity due to ISIS 301012 treatment.

### Example 53

Assessment of In Vitro Immunostimulatory Effects of ISIS 301012

Immunostimulatory activity is defined by the production of cytokines upon exposure to a proinflammatory agent. In a further embodiment of the invention, ISIS 301012 was tested for immunostimulatory, or proinflammatory, activity. These studies were performed by MDS Pharma Services (Saint Germain sur l'Arbresle, France). Whole blood was collected from naive B6C3F1 mice, which had not been knowingly exposed to viral, chemical or radiation treatment. Cultured blood cells were exposed to 0.5, 5 or 50 μM of ISIS 301012 for a period of 14 to 16 hours. Antisense oligonucleotides known to possess proinflammatory activity served as positive controls. Each treatment was performed in triplicate. At the end of the treatment period, supernatants were collected and cytokine analysis was performed using a flow cytometry method with the mouse Inflammation CBA kit (Becton Dickinson, Franklin Lakes, N.J.). The results revealed that ISIS 301012 does not stimulate the release of any of the tested cytokines, which were interleukin-12p70 (IL-12p70), tumor necrosis factor-alpha (TNF-alpha), interferon-gamma (IFN-gamma), interleukin-6 (IL-6), macrophage chemoattractant protein-1 (MCP-1) and interleukin-10 (IL-10). Thus, ISIS 301012 does not possess immunostimulatory activity, as determined by the in vitro immunostimulatory assay.

### Example 54

Comparative Genomic Analysis of Apolipoprotein B

In accordance with the present invention, a comparative genomic analysis of apolipoprotein B sequences from human, mouse and monkey was performed and illustrated that apolipoprotein B sequences are conserved across species. The organization of human and mouse apolipoprotein B genes is also highly conserved. The human and mouse genes are comprised of 29 and 26 exons, respectively. The mouse mRNA is approximately 81% homologous to the human sequence. The complete sequence and gene structure of the apolipoprotein B gene in non-human primates have not been identified. However, as illustrated in Example 46, a 500 base pair fragment which contains the ISIS 301012 target sequence exhibits approximately 96% identity to the human sequence.

The binding site for ISIS 301012 lies within the coding region, within exon 22 of the human apolipoprotein B mRNA. When the ISIS 301012 binding sites from human, mouse and monkey were compared, significant sequence diversity was observed. Although the overall sequence conservation between human and monkey over a 500 nucleotide region was approximately 96%, the ISIS 301012 binding site of the monkey sequence contains 2 mismatches relative to the human sequence. Likewise, though the mouse apolipoprotein B mRNA sequence is approximately 81% homologous to human, within the ISIS 301012 binding site, 5 nucleotides are divergent. The sequence comparisons for the ISIS 301012 binding site for human, mouse and monkey apolipoprotein B sequences are shown in Table 33. Mismatched nucleotides relative to the ISIS 301012 target sequence are underlined.

TABLE 33
Comparison of ISIS 301012 binding site among
human, monkey and mouse apolipoprotein B
sequences

| | # | ISIS 301012 target |
|---|---|---|
| Species | Mismatches | sequence |
| Human | 0 | aggtgcgaagcagactgagg |
| Monkey | 2 | aggtgtaaagcagactgagg |
| Mouse | 5 | aggagtgcagcagtctgaag |

The target sequence to which the mouse antisense oligonucleotide ISIS 147764 hybridizes lies within exon 24 of the mouse apolipoprotein B gene. The sequence comparisons for the ISIS 147764 binding site in mouse and human apolipoprotein B sequences are shown in Table 34. Mismatched nucleotides relative to the ISIS 147764 target sequence are underlined.

TABLE 34
Comparison of ISIS 147764 binding site between
mouse and human apolipoprotein B sequences

| | # | ISIS 147764 binding |
|---|---|---|
| Species | Mismatches | site |
| Human | 5 | gcattgacatcttcagggac |
| Mouse | 0 | gcatggacttcttctggaaa |

### Example 55

BLAST Analysis of ISIS 301012

In accordance with the present invention, the number of regions in the human genome to which ISIS 301012 will hybridize with perfect complementarity was determined. Percent complementarity of an antisense compound with a region of a target nucleic acid was determined using BLAST programs (basic local alignment search tools) and PowerBLAST programs known in the art (Altschul et al., J. Mol. Biol., 1990, 215, 403-410; Zhang and Madden, Genome Res., 1997, 7, 649-656). This analysis assessed sequence complementarity in genomic or pre-mRNA regions and in coding sequences.

In genomic regions, ISIS 301012 shows perfect sequence complementarity to the apolipoprotein B gene only. No target sequences with one mismatch relative to ISIS 301012 were found. Two mismatches are found between the ISIS 301012 target sequence and the heparanase gene, and 3 mismatches are found between the ISIS 301012 target sequence and 28 unique genomic sites.

In RNA sequences, perfect sequence complementarity is found between ISIS 301012 and the apolipoprotein B mRNA and three expressed sequence tags that bear moderate similarity to a human apolipoprotein B precursor. A single mismatch is found between ISIS 301012 and an expressed sequence tag similar to the smooth muscle form of myosin light chain.

### Example 56

Antisense Inhibition of Apolipoprotein B in Primary Human Hepatocytes: Dose Response Studies

In accordance with the present invention, antisense oligonucleotides targeted to human apolipoprotein B were tested in dose response studies in primary human hepatocytes. Pre-plated primary human hepatocytes were purchased from InVitro Technologies (Baltimore, Md.). Cells were cultured in high-glucose DMEM (Invitrogen Corporation, Carlsbad, Calif.) supplemented with 10% fetal bovine serum (Invitrogen Corporation, Carlsbad, Calif.), 100 units/mL and 100 μg/mL streptomycin (Invitrogen Corporation, Carlsbad, Calif.).

Human primary hepatocytes were treated with ISIS 301012 (SEQ ID NO: 247) at 10, 50, 150 or 300 nM. Untreated cells and cells treated with the scrambled control oligonucleotide ISIS 113529 (CTCTTACTGTGCTGTGGACA, SEQ ID NO: 859) served as two groups of control cells. ISIS 113529 is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidines are 5-methylcytidines.

Oligonucleotides were introduced into cells through LIPOFECTIN-mediated transfection as described by other examples herein. Cells were harvested both 24 and 48 hours after treatment with oligonucleotide, and both RNA and protein were isolated. Additionally, the culture media from treated cells was collected for ELISA analysis of apolipoprotein B protein secretion.

Apolipoprotein B mRNA expression was determined by real-time PCR of RNA samples as described by other examples herein. Each result represents 6 experiments. The data are normalized to untreated control cells and are shown in Table 35.

TABLE 35
Inhibition of apolipoprotein B mRNA by antisense oligonucleotides
in human primary hepatocytes

| | | % Inhibition of | |
|---|---|---|---|
| | | apolipoprotein B mRNA | |
| Dose of | Treatment | ISIS # | |
| oligonucleotide | (hours) | 301012 | 113529 |
| 10 nM | 24 | 65 | N.D. |
| | 48 | 33 | N.D. |
| 50 nM | 24 | 75 | N.D. |
| | 48 | 48 | N.D. |
| 150 nM | 24 | 90 | 16 |
| | 48 | 78 | 5 |
| 300 nM | 24 | 89 | 10 |
| | 48 | 72 | 18 |

These data demonstrate that ISIS 301012 inhibits apolipoprotein B expression in a dose-dependent manner in human primary hepatocytes.

Apolipoprotein B protein secreted from into the cultured cell media was measured in the samples treated with 50 and 150 nM of oligonucleotide, using a target protein specific ELISA kit (ALerCHEK Inc., Portland, Me.). Each result represents 3 experiments. The data are normalized to untreated control cells and are shown in Table 36.

TABLE 36
Inhibition of apolipoprotein B protein secretion from
human primary hepatocytes by ISIS 301012

| | | % Change in | |
|---|---|---|---|
| | | apolipoprotein B | |
| | | protein secretion | |
| | Treatment | ISIS # | |
| Dose | (hours) | 301012 | 113529 |
| 150 nM | 24 | −57 | +6 |
| | 48 | −75 | +4 |
| 300 nM | 24 | −41 | −2 |
| | 48 | −48 | −5 |

Protein samples from 50, 150 and 300 nM doses after 24 hours and 150 and 300 nM doses after 48 hours were subjected to immunoblot analysis as described by other examples herein, using a human apolipoprotein B protein specific antibody purchased from US Biological (Swampscott, Mass.). Immunoblot analysis further demonstrates that apolipoprotein B protein in human hepatocytes is reduced in a dose-dependent manner following antisense oligonucleotide treatment with ISIS 301012.

An additional experiment was performed to test the effects of ISIS 271009 (SEQ ID NO: 319), ISIS 281625 (SEQ ID NO: 224) and ISIS 301027 (SEQ ID NO: 262) on human apolipoprotein B mRNA in human primary hepatocytes. Cells were cultured as described herein and treated with 5, 10, 50 or 150 nM of ISIS 271009, ISIS 281625 or ISIS 301027 for a period of 24 hours. The control oligonucleotides ISIS 13650 (SEQ ID NO: 806) and ISIS 113529 (SEQ ID NO: 859) were used at 50 or 150 nM. Human apolipoprotein B mRNA expression was evaluated by real-time PCR as described by other examples herein. Apolipoprotein B protein secreted into the cultured cell media was measured in the samples treated with 50 and 150 nM of oligonucleotide, using a target protein specific ELISA kit (ALerCHEK Inc., Portland, Me.).

The data, shown in Table 37, represent the average 2 experiments and are normalized to untreated control cells. Where present, a “+” indicates that gene expression was increased.

TABLE 37
Antisense inhibition of human apolipoprotein B mRNA by ISIS 271009, ISIS 281625
and ISIS 301027

| | Oligonucleotide | ISIS | ISIS | ISIS | ISIS | ISIS |
|---|---|---|---|---|---|---|
| | dose | 271009 | 281625 | 301027 | 13650 | 113529 |
| % Inhibition of | 5 nM | +4 | 8 | 11 | N.D. | N.D. |
| apolipoprotein B | 10 nM | 5 | 22 | 37 | N.D. | N.D. |
| mRNA expression | 50 nM | 52 | 49 | 50 | 38 | 0 |
| | 150 nM | 81 | 52 | 70 | 26 | 14 |
| % Inhibition of | 50 nM | 17 | 18 | 21 | N.D. | N.D. |
| apolipoprotein B | 150 nM | 32 | 18 | 32 | +18 | +1 |
| protein secretion | | | | | | |

These data demonstrate that ISIS 271009, ISIS 281625 and ISIS 301027 inhibit apolipoprotein B mRNA expression in a dose-dependent manner in human primary hepatocytes. ISIS 271009 and ISIS 301027 inhibit the secretion of apolipoprotein B protein from cells in a dose-dependent manner.

### Example 57

Effects of apolipoproteinB-100 Antisense Oligonucleotides on Apolipoprotein(a) Expression

Lipoprotein(a) [Lp(a)] contains two disulfide-linked distinct proteins, apolipoprotein(a) and apolipoprotein B (Rainwater and Kammerer, J. Exp. Zool., 1998, 282, 54-61). In accordance with the present invention, antisense oligonucleotides targeted to apolipoprotein B were tested for effects on the expression of the apolipoprotein(a) component of the lipoprotein(a) particle in primary human hepatocytes.

Primary human hepatocytes (InVitro Technologies, Baltimore, Md.), cultured and transfected as described herein, were treated with 5, 10, 50 or 150 nM of ISIS 271009 (SEQ ID NO: 319), 281625 (SEQ ID NO: 224), 301012 (SEQ ID NO: 247) or 301027 (SEQ ID NO: 262). Cells were also treated with 50 or 150 nM of the control oligonucleotides ISIS 113529 (SEQ ID NO: 859) or ISIS 13650 (SEQ ID NO: 806). Untreated cells served as a control. Following 24 hours of oligonucleotide treatment, apolipoprotein(a) mRNA expression was measured by quantitative real-time PCR as described in other examples herein.

Probes and primers to human apolipoprotein(a) were designed to hybridize to a human apolipoprotein(a) sequence, using published sequence information (GenBank accession number NM_005577.1, incorporated herein as SEQ ID NO: 860). For human apolipoprotein(a) the PCR primers were:
forward primer: CAGCTCCTTATTGTTATACGAGGGA (SEQ ID NO: 861)
reverse primer: TGCGTCTGAGCATTGCGT (SEQ ID NO: 862) and the PCR probe was: FAM-CCCGGTGTCAGGTGGGAGTACTGC-TAMRA
   (SEQ ID NO: 863) where FAM is the fluorescent dye and TAMRA is the quencher dye. Data are the average of three experiments and are expressed as percent inhibition relative to untreated controls. The results are shown in Table 38. A “+” or “−” preceding the number indicates that apolipoprotein(a) expression was increased or decreased, respectively, following treatment with antisense oligonucleotides.

TABLE 38
Effects of apolipoprotein B antisense oligonucleotides
on apolipoprotein (a) expression

| | % Change in apolipoprotein (a) mRNA expression | | | | | |
|---|---|---|---|---|---|---|
| | following antisense inhibition of apolipoprotein B | | | | | |
| Oligonucleotide | ISIS # | | | | | |
| Dose | 271009 | 281625 | 301012 | 301027 | 13650 | 113529 |
| 5 nM | +70 | −9 | +34 | −16 | N.D. | N.D. |
| 10 nM | +31 | −23 | +86 | −45 | N.D. | N.D. |
| 50 nM | +25 | −34 | +30 | −39 | −68 | +14 |
| 150 nM | −47 | +32 | +38 | −43 | −37 | −9 |

These results illustrate that ISIS 301012 did not inhibit the expression of apolipoprotein(a) in human primary hepatocytes. ISIS 271009 inhibited apolipoprotein(a) expression at the highest dose. ISIS 281625 and ISIS 301027 decreased the levels of apolipoprotein(a) mRNA.

### Example 58

Inhibition of Lipoprotein(a) Particle Secretion with Antisense Oligonucleotides Targeted to apolipoproteinB-100

In accordance with the present invention, the secretion of lipoprotein(a) particles, which are comprised of one apolipoprotein(a) molecule covalently linked to one apolipoprotein B molecule, was evaluated in primary human hepatocytes treated with antisense oligonucleotides targeted to the apolipoprotein B component of lipoprotein(a).

Primary human hepatocytes (InVitro Technologies, Baltimore, Md.), cultured and transfected as described herein, were treated for 24 hours with 50 or 150 nM of ISIS 271009 (SEQ ID NO: 319), 281625 (SEQ ID NO: 224), 301012 (SEQ ID NO: 247) or 301027 (SEQ ID NO: 262). Cells were also treated with 150 nM of the control oligonucleotides ISIS 113529 (SEQ ID NO: 859) or ISIS 13650 (SEQ ID NO: 806). Untreated cells served as a control. Following 24 hours of oligonucleotide treatment, the amount of lipoprotein(a) in the culture medium collected from the treated cells was measured using a commercially available ELISA kit (ALerCHEK Inc., Portland, Me.). The results are the average of three experiments and are expressed as percent change in lipoprotein(a) secretion relative to untreated controls. The data are shown in Table 39. A “+” or “−” preceding the number indicates that lipoprotein(a) particle secretion was increased or decreased, respectively, following treatment with antisense oligonucleotides targeted to apolipoprotein B.

TABLE 39
Inhibition of lipoprotein (a) particle secretion with antisense
oligonucleotides targeted to apolipoprotein B

| | % Change in lipoprotein (a) secretion | | | | | |
|---|---|---|---|---|---|---|
| Oligonucleotide | ISIS # | | | | | |
| Dose | 271009 | 281625 | 301012 | 301027 | 13650 | 113529 |
| 50 nM | −25 | −26 | −27 | −33 | N.D. | N.D. |
| 150 nM | −42 | −24 | −37 | −44 | +14 | +14 |

These data demonstrate that antisense inhibition of apolipoprotein B, a component of the lipoprotein(a) particle, can reduce the secretion of lipoprotein(a) from human primary hepatocytes. In addition, this reduction in lipoprotein(a) secretion is not necessarily concomitant with a decrease in apolipoprotein(a) mRNA expression, as shown in Example 57.

### Example 59

Mismatched and Truncated Derivatives of ISIS 301012

As demonstrated herein, ISIS 301012 (SEQ ID NO: 247) reduces apolipoprotein B mRNA levels in cultured human cell lines as well as in human primary hepatocytes. In a further embodiment of the invention, a study was performed using nucleotide sequence derivatives of ISIS 301012. A series of oligonucleotides containing from 1 to 7 base mismatches, starting in the center of the ISIS 301012 sequence, was designed. This series was designed to introduce the consecutive loss of Watson-Crick base pairing between ISIS 301012 and its target mRNA sequence. These compounds are shown in Table 40. The antisense compounds with mismatched nucleotides relative to ISIS 301012 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide.

An additional derivative of ISIS 301012 was designed, comprising the ISIS 301012 sequence with 2′MOE nucleotides throughout the oligonucleotide (uniform 2′-MOE). This compound is 20 nucleotides in length, with phosphorothioate linkages throughout the oligonucleotide. This compound is also shown in Table 40.

HepG2 cells were treated with 50 or 150 nM of the compounds in Table 40 for a 24 hour period, after which RNA was isolated and target expression was measured by real-time PCR as described herein. Untreated cells served as controls. The results are shown in Tables 40 and are normalized to untreated control samples.

TABLE 40
Effects of ISIS 301012 mismatched oligonucleotides and
a uniform 2′MOE oligonucleotide on apolipoprotein B
expression in HepG2 cells

| | | | % Change in | | |
|---|---|---|---|---|---|
| | | | apolipoprotein | | |
| | | | B mRNA | | |
| | | | expression | | |
| | | | Dose of | | SEQ |
| | | # | oligonucleotide | | ID |
| ISIS # | SEQUENCE | Mismatches | 50 | 150 | NO |

| | | | | | |
|---|---|---|---|---|---|
| 301012 | GCCTCAGTCTGCTTCGCACC | 0 | −44 | −75 | 247 |

Mismatch Series, chimeric oligonucleotides

| | | | | | |
|---|---|---|---|---|---|
| 332770 | GCCTCAGTCTTCTTCGCACC | 1 | +7 | −22 | 864 |
| 332771 | GCCTCAGTCTTATTCGCACC | 2 | +37 | +37 | 865 |
| 332772 | GCCTCAGTATTATTCGCACC | 3 | +99 | +84 | 866 |
| 332773 | GCCTCATTATTATTCGCACC | 4 | +75 | +80 | 867 |
| 332774 | GCCTCATTATTATTAGCACC | 5 | +62 | +66 | 868 |
| 332775 | GCCTCATTATTATTATCACC | 6 | −1 | +10 | 869 |
| 332776 | GCCTAATTATTATTATCACC | 7 | +10 | +20 | 870 |

Uniform 2′-MOE oligonucleotide

| | | | | | |
|---|---|---|---|---|---|
| 332769 | GCCTCAGTCTGCTTCGCACC | 0 | −11 | −14 | 247 |

The results of treatment of HepG2 cells with the compounds in Table 40 reveals that none of the compounds displays the dose-dependent inhibition observed following treatment with the parent ISIS 301012 sequence. ISIS 332770, which has only a single thymidine to cytosine substitution in the center of the oligonucleotide, was 3-fold less potent than ISIS 301012. Further nucleotide substitutions abrogated antisense inhibition of apolipoprotein B expression.

Phosphorothioate chimeric oligonucleotides are metabolized in vivo predominantly by endonucleolytic cleavage. In accordance with the present invention, a series of oligonucleotides was designed by truncating the ISIS 301012 sequence in 1 or 2 base increments from the 5′ and/or 3′ end. The truncated oligonucleotides represent the possible products that result from endonucleolytic cleavage. These compounds are shown in Table 41. The compounds in Table 41 are chimeric oligonucleotides (“gapmers”) of varying lengths, composed of a central “gap” region consisting of 2′-deoxynucleotides, which is flanked on both ends by 2′-methoxyethyl (2′-MOE)nucleotides. The exact structure of each chimeric oligonucleotide is designated in Table 41 as the “chimera structure”. For example, a designation of 4˜10˜4 indicates that the first 4 (5′ most) and last 4 (3′ most) nucleotides are 2′-MOE nucleotides, and the 10 nucleotides in the gap are 2′-deoxynucleotides. 2′-MOE nucleotides are indicated by bold type. The internucleoside (backbone) linkages are phosphodiester (P═O) between underscored nucleotides; all other internucleoside linkages are phosphorothioate (P═S).

These compounds were tested for their ability to reduce the expression of apolipoprotein B mRNA. HepG2 cells were treated with 10, 50 or 150 nM of each antisense compound in Table 41 for a 24 hour period, after which RNA was isolated and target expression was measured by real-time PCR as described herein. Untreated cells served as controls. The results are shown in Tables 41 and are normalized to untreated control samples.

TABLE 41
Effect of ISIS 301012 truncation mutants on apolipoprotein B
expression in HepG2 cells

| | | | | | % Change in | | | |
|---|---|---|---|---|---|---|---|---|
| | | | | | apolipoprotein | | | |
| | | | | | B mRNA | | | |
| | | | | | expression | | | |
| | Target | | | | Dose of | | | SEQ |
| | SEQ ID | Target | | Chimeric | oligonucleotide | | | ID |
| ISIS # | NO | Site | SEQUENCE | structure | 10 | 50 | 150 | NO |
| 301012 | 3 | 3249 | GCCTCAGTCTGCTTCGCACC | 5~10~5 | −51 | −72 | −92 | 247 |
| 331022 | 3 | 3249 | GCCTCAGTCTGCTTCGCAC | 5~10~4 | −33 | −49 | −87 | 871 |
| 332777 | 3 | 3249 | GCCTCAGTCTGCTTCGCA | 5~10~3 | −27 | −53 | −80 | 872 |
| 332778 | 3 | 3249 | GCCTCAGTCTGCTTC | 5~10~0 | −11 | −20 | −58 | 873 |
| 332780 | 3 | 3248 | CCTCAGTCTGCTTCGCAC | 4~10~4 | −3 | −43 | −74 | 874 |
| 332781 | 3 | 3247 | CTCAGTCTGCTTCGCA | 3~10~3 | −9 | −35 | −60 | 875 |
| 332782 | 3 | 3246 | TCAGTCTGCTTCGC | 2~10~2 | −16 | −16 | −69 | 876 |
| 332784 | 3 | 3249 | GCCTCAGTCT | 5~5~0 | +12 | −1 | +7 | 877 |
| 332785 | 3 | 3238 | GCTTCGCACC | 0~5~5 | +5 | −2 | −4 | 878 |

The results in Table 41 illustrate that inhibition of apolipoprotein B is dependent upon sequence length, as well as upon sequence complementarity and dose, as demonstrated in Table 41, but truncated versions of ISIS 301012 are to a certain degree capable of inhibiting apolipoprotein B mRNA expression.

### Example 60

Design and Screening of dsRNAs Targeting Human Apolipoprotein B

In accordance with the present invention, a series of nucleic acid duplexes comprising the antisense compounds of the present invention and their complements were designed to target apolipoprotein B and are shown in Table 42. All compounds in Table 42 are oligoribonucleotides 20 nucleotides in length with phosphodiester internucleoside linkages (backbones) throughout the compound. The compounds were prepared with blunt ends. Table 41 shows the antisense strand of the dsRNA, and the sense strand is synthesized as the complement of the antisense strand. These sequences are shown to contain uracil (U) but one of skill in the art will appreciate that uracil (U) is generally replaced by thymine (T) in DNA sequences. “Target site” indicates the first (5′-most) nucleotide number on the particular target sequence to which the compound binds. A subset of the compounds in Table 42 are the RNA equivalents of DNA antisense oligonucleotides described herein, and, where applicable, this is noted by the ISIS # of the DNA oligonucleotide in the column “RNA equivalent of ISIS #”.

TABLE 42
dsRNAs targeted to human apolipoprotein B

| | | Target | | | SEQ | RNA |
|---|---|---|---|---|---|---|
| | | SEQ ID | Target | | ID | equivalent |
| ISIS # | Region | NO | Site | Sequence | NO | of ISIS # |
| 342855 | coding | 3 | 3249 | GCCUCAGUCUGCUUCGCACC | 247 | 301012 |
| 342856 | 3′ UTR | 3 | 13903 | GCUCACUGUAUGGUUUUAUC | 262 | 301027 |
| 342857 | coding | 3 | 5589 | AGGUUACCAGCCACAUGCAG | 224 | 308361 |
| 342858 | coding | 3 | 669 | GAGCAGUUUCCAUACACGGU | 130 | 270991 |
| 342859 | coding | 3 | 1179 | CCUCUCAGCUCAGUAACCAG | 135 | 270996 |
| 342860 | coding | 3 | 2331 | GUAUAGCCAAAGUGGUCCAC | 34 | 147797 |
| 342861 | coding | 3 | 3579 | UAAGCUGUAGCAGAUGAGUC | 213 | 281614 |
| 342862 | 5′ UTR | 3 | 6 | CAGCCCCGCAGGUCCCGGUG | 249 | 301014 |
| 342863 | 5′ UTR | 3 | 116 | GGUCCAUCGCCAGCUGCGGU | 256 | 301021 |
| 342864 | 3′ UTR | 3 | 13910 | AAGGCUGGCUCACUGUAUGG | 266 | 301031 |
| 342865 | 3′ UTR | 3 | 13970 | GCCAGCUUUGGUGCAGGUCC | 273 | 301038 |
| 342866 | coding | 3 | 426 | UUGAAGCCAUACACCUCUUU | 879 | none |
| 342867 | coding | 3 | 3001 | UGACCAGGACUGCCUGUUCU | 880 | none |
| 342868 | coding | 3 | 5484 | GAAUAGGGCUGUAGCUGUAA | 881 | none |
| 342869 | coding | 3 | 6662 | UAUACUGAUCAAAUUGUAUC | 882 | none |
| 342870 | coding | 3 | 8334 | UGGAAUUCUGGUAUGUGAAG | 883 | none |
| 342871 | coding | 3 | 9621 | AAAUCAAAUGAUUGCUUUGU | 883 | none |
| 342872 | coding | 3 | 10155 | GUGAUGACACUUGAUUUAAA | 885 | none |
| 342873 | coding | 3 | 12300 | GAAGCUGCCUCUUCUUCCCA | 886 | none |
| 342874 | coding | 3 | 13629 | GAGAGUUGGUCUGAAAAAUC | 887 | none |

The dsRNA compounds in Table 42 were tested for their effects on human apolipoprotein mRNA in HepG2 cells. HepG2 cells were treated with 100 nM of dsRNA compounds mixed with 5 μg/mL LIPOFECTIN (Invitrogen Corporation, Carlsbad, Calif.) for a period of 16 hours. In the same experiment, HepG2 cells were also treated with 150 nM of subset of the antisense oligonucleotides described herein mixed with 3.75 μg/mL LIPOFECTIN; these compounds are listed in Table 43. Control oligonucleotides included ISIS 18078 (GTGCGCGCGAGCCCGAAATC, SEQ ID NO: 888). ISIS 18078 is a chimeric oligonucleotide (“gapmer”) 20 nucleotides in length, composed of a central “gap” region consisting of 9 2′-deoxynucleotides, which is flanked on the 5′ and 3′ ends by a five-nucleotide “wing” and a six-nucleotide “wing”, respectively. The wings are composed of 2′-methoxyethyl (2′-MOE)nucleotides. The internucleoside (backbone) linkages are phosphorothioate (P═S) throughout the oligonucleotide. All cytidines are 5-methylcytidines.

The duplex of ISIS 263188 (CUUCUGGCAUCCGGUUUAGTT, SEQ ID NO: 889) and its complement was also used as a control. ISIS 263188 is an oligoribonucleotide 21 nucleotides in length with the 2 nucleotides on the 3′ end being oligodeoxyribonucleotides (TT) and with phosphodiester internucleoside linkages (backbones) throughout the compound.

Cells were treated for 4 hours, after which human apolipoprotein B mRNA expression was measured as described by examples herein. Results were normalized to untreated control cells, which were not treated with LIPOFECTIN or oligonucleotide. Data are the average of 4 experiments and are presented in Table 43.

TABLE 43
Inhibition of apolipoprotein B mRNA by dsRNAs in
HepG2 cells

| ISIS | | % | |
|---|---|---|---|
| # | Dose | Inhibition | SEQ ID # |
| 342855 | 100 nM | 53 | 247 |
| 342856 | 100 nM | 34 | 262 |
| 342857 | 100 nM | 55 | 224 |
| 342858 | 100 nM | 44 | 130 |
| 342859 | 100 nM | 23 | 135 |
| 342860 | 100 nM | 34 | 34 |
| 342861 | 100 nM | 42 | 213 |
| 342862 | 100 nM | 16 | 249 |
| 342863 | 100 nM | 34 | 256 |
| 342864 | 100 nM | 53 | 266 |
| 342865 | 100 nM | 50 | 273 |
| 342866 | 100 nM | 12 | 879 |
| 342867 | 100 nM | 26 | 880 |
| 342868 | 100 nM | 36 | 881 |
| 342869 | 100 nM | 78 | 882 |
| 342870 | 100 nM | 71 | 883 |
| 342871 | 100 nM | 9 | 883 |
| 342872 | 100 nM | 2 | 885 |
| 342873 | 100 nM | 53 | 886 |
| 342874 | 100 nM | 73 | 887 |
| 281625 | 150 nM | 79 | 224 |
| 301012 | 150 nM | 77 | 247 |
| 301014 | 150 nM | 88 | 249 |
| 301021 | 150 nM | 67 | 256 |
| 301027 | 150 nM | 79 | 262 |
| 301028 | 150 nM | 85 | 263 |
| 301029 | 150 nM | 77 | 264 |
| 301030 | 150 nM | 70 | 265 |
| 301031 | 150 nM | 73 | 266 |
| 301037 | 150 nM | 80 | 272 |
| 301038 | 150 nM | 84 | 273 |
| 301045 | 150 nM | 77 | 280 |
| 263188 | 150 nM | 26 | 888 |
| 18078 | 150 nM | 13 | 889 |

### Example 61

Antisense Inhibition of Apolipoprotein B in Cynomolgous Monkey Primary Hepatocytes

As demonstrated in Example 46, the region containing the target site to which ISIS 301012 hybridizes shares 96% identity with the corresponding region of Cynomolgus monkey apolipoprotein B mRNA sequence. ISIS 301012 contains two mismatched nucleotides relative to the Cynomolgous monkey apolipoprotein B mRNA sequence to which it hybridizes. In a further embodiment of the invention, oligonucleotides were designed to target regions of the monkey apolipoprotein B mRNA, using the partial Cynomolgous monkey apolipoprotein B sequence described herein (SEQ ID NO: 855) and an additional portion of Cynomolgous monkey apolipoprotein B RNA sequence, incorporated herein as SEQ ID NO: 890. The target site indicates the first (5′-most) nucleotide number on the particular target sequence to which the oligonucleotide binds. For ISIS 326358 (GCCTCAGTCTGCTTTACACC, SEQ ID NO: 891) the target site is nucleotide 168 of SEQ ID NO: 855 and for ISIS 315089 (AGATTACCAGCCATATGCAG, SEQ ID NO: 892) the target site is nucleotide 19 of SEQ ID NO: 890. ISIS 326358 and ISIS 315089 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. ISIS 326358 and ISIS 315089 are the Cynomolgous monkey equivalents of the human apolipoprotein B antisense oligonucleotides ISIS 301012 (SEQ ID NO: 247) and ISIS 281625 (SEQ ID NO: 224), respectively.

Antisense inhibition by ISIS 301012 was compared to that of ISIS 326358, which is a perfect match to the Cynomolgous monkey apolipoprotein B sequence to which ISIS 301012 hybridizes. The compounds were analyzed for their effect on Cynomolgous monkey apolipoprotein B mRNA levels in primary Cynomolgous monkey hepatocytes purchased from In Vitro Technologies (Gaithersburg, Md.). Pre-plated primary Cynonomolgous monkey hepatocytes were purchased from InVitro Technologies (Baltimore, Md.). Cells were cultured in high-glucose DMEM (Invitrogen Corporation, Carlsbad, Calif.) supplemented with 10% fetal bovine serum (Invitrogen Corporation, Carlsbad, Calif.), 100 units/mL and 100 μg/mL streptomycin (Invitrogen Corporation, Carlsbad, Calif.).

Primary Cynomolgous monkey hepatocytes were treated with 10, 50, 150 or 300 nM of antisense oligonucleotides for 48 hours. ISIS 113529 (SEQ ID NO: 859) was used as a control oligonucleotide. Untreated cells also served as a control. Cynomolgous monkey apolipoprotein B mRNA levels were quantitated by real-time PCR using the human apolipoprotein B and GAPDH primers and probes described by other examples herein. The results, shown in Table 44, are the average of 6 experiments and are expressed as percent inhibition of apolipoprotein B mRNA normalized to untreated control cells.

TABLE 44
Inhibition of Cynomolgous monkey apolipoprotein B
mRNA by ISIS 301012 and ISIS 326358

| | | % Inhibition of | | |
|---|---|---|---|---|
| | Time of | apolipoprotein B mRNA | | |
| Dose of | treatment | ISIS # | | |
| oligonucleotide | (hours) | 326358 | 301012 | 113529 |
| 10 nM | 24 | 35 | 24 | N.D. |
| | 48 | 85 | 76 | N.D. |
| 50 nM | 24 | 66 | 60 | N.D. |
| | 48 | 88 | 77 | N.D. |
| 150 nM | 24 | 61 | 56 | 5 |
| | 48 | 82 | 88 | 42 |
| 300 nM | 24 | 64 | 61 | 19 |
| | 48 | 87 | 86 | 13 |

These data demonstrate that both ISIS 326359 and ISIS 301012 (despite two mismatches with the Cynomolgous monkey apolipoprotein B sequence) can inhibit the expression of apolipoprotein B mRNA in cynomolgous monkey primary hepatocytes, in a dose- and time-dependent manner.

Apolipoprotein B protein secreted from primary Cynomolgous hepatocytes treated with 150 and 300 nM of oligonucleotide was measured by ELISA using an apolipoprotein B protein specific kit (ALerCHEK Inc., Portland, Me.). Each result represents the average of 3 experiments. The data are normalized to untreated control cells and are shown in Table 45.

TABLE 45
Reduction in apolipoprotein B protein secreted from Cynomolgous
monkey hepatocytes following antisense oligonucleotide treatment

| | | % Reduction in secreted | | |
|---|---|---|---|---|
| | Time of | apolipoprotein B protein | | |
| Dose of | treatment | ISIS # | | |
| oligonucleotide | (hours) | 326358 | 301012 | 113529 |
| 150 nM | 24 | 21 | 31 | 11 |
| | 48 | 29 | 25 | 18 |
| 300 nM | 24 | 17 | 10 | 12 |
| | 48 | 35 | 17 | 8 |

These results demonstrate that antisense inhibition by ISIS 301012 and ISIS 326358 leads to a decrease in the secretion of apolipoprotein B protein from cultured primary Cynomolgous hepatocytes.

Additionally, protein was isolated from oligonucleotide-treated primary Cynomolgous monkey hepatocytes and subjected to immunoblot analysis to further assess apolipoprotein B protein expression. Immunoblotting was performed as described herein, using an antibody to human apolipoprotein B protein (US Biologicals, Swampscott, Mass.). Immunoblot analysis of apolipoprotein B expression following antisense oligonucleotide treatment with ISIS 326358 and ISIS 301012 reveals a substantial reduction in apolipoprotein B expression.

In a further embodiment of the invention, antisense inhibition by ISIS 281625 was compared to that by ISIS 315089, which is a perfect match to the Cynomolgous monkey apolipoprotein B sequence to which ISIS 281625 hybridizes. Primary Cynomolgous monkey hepatocytes, cultured as described herein, were treated with 10, 50, 150 or 300 nM of ISIS 315089 or ISIS 281625 for 24 hours. Cells were treated with the control oligonucleotide ISIS 13650 (SEQ ID NO: 806) at 150 and 300 nM or ISIS 113529 (SEQ ID NO: 859) at 300 nM. Untreated cells also served as a control. Cynomolgous monkey apolipoprotein B mRNA levels in primary Cynomolgous monkey hepatocytes was quantitated using real-time PCR with human primers and probe as described by other examples herein. The results, shown in Table 46, are the average of 3 experiments and are expressed as percent inhibition of apolipoprotein B mRNA normalized to untreated control cells. Where present, a “+” preceding the value indicates that mRNA expression was increased.

TABLE 46
Antisense inhibition of apolipoprotein B mRNA expression
in Cynomolgous monkey hepatocytes

| | % Inhibition of apolipoprotein B | | | |
|---|---|---|---|---|
| | mRNA | | | |
| Dose of | ISIS # | | | |
| oligonucleotide | 315089 | 281625 | 13650 | 113529 |
| 10 nM | 70 | +5 | N.D. | N.D. |
| 50 nM | 83 | 41 | N.D. | N.D. |
| 150 nM | 81 | 35 | +50 | N.D. |
| 300 nM | 82 | 69 | 33 | 28 |

These data demonstrate that both ISIS 315089 and ISIS 281625 can inhibit the expression of apolipoprotein B mRNA in Cynomolgous monkey primary hepatocytes, in a dose-dependent manner.

Apolipoprotein B protein secreted primary Cynomolgous hepatocytes treated with 50 and 150 nM of ISIS 315089 and ISIS 281625 was measured by ELISA using an apolipoprotein B protein specific kit (ALerCHEK Inc., Portland, Me.). Each result represents the average of 3 experiments. The data are normalized to untreated control cells and are shown in Table 47.

TABLE 47
Reduction in apolipoprotein B protein secreted from Cynomolgous
monkey hepatocytes following antisense oligonucleotide treatment

| | % Reduction of monkey | | | |
|---|---|---|---|---|
| | apolipoprotein B protein | | | |
| | secretion | | | |
| Dose of | ISIS # | | | |
| oligonucleotide | 315089 | 281625 | 13650 | 113529 |
| 50 nM | 11 | 6 | 16 | N.D. |
| 150 nM | 25 | 13 | 13 | 12 |

These results demonstrate that antisense inhibition by 150 nM of ISIS 315089 leads to a decrease in the secretion of apolipoprotein B protein from cultured primary Cynomolgous hepatocytes.

ISIS 271009 (SEQ ID NO: 319) and ISIS 301027 (SEQ ID NO: 262) were also tested for their effects on apolipoprotein B mRNA and protein expression in Cynomolgous primary hepatocytes. Cells, cultured as described herein, were treated with 10, 50 and 150 nM of ISIS 271009 or ISIS 301027 for 24 hours. Cells were treated with the control oligonucleotide ISIS 113529 (SEQ ID NO: 859) at 150 nM. Untreated cells also served as a control. Cynomolgous monkey apolipoprotein B mRNA levels in primary Cynomolgous monkey hepatocytes was quantitated using real-time PCR with human primers and probe as described by other examples herein. The results, shown in Table 48, are the average of 2 experiments and are expressed as percent inhibition of apolipoprotein B mRNA normalized to untreated control cells.

TABLE 48
Antisense inhibition of apolipoprotein B mRNA
expression in Cynomolgous monkey hepatocytes

| | % Inhibition of | | |
|---|---|---|---|
| | apolipoprotein B mRNA | | |
| Dose of | ISIS # | | |
| oligonucleotide | 271009 | 301027 | 113529 |
| 10 nM | 42 | 40 | N.D. |
| 50 nM | 66 | 54 | N.D. |
| 150 nM | 69 | 67 | 11 |

These data demonstrate that both ISIS 271009 and ISIS 301027 can inhibit the expression of apolipoprotein B mRNA in Cynomolgous monkey primary hepatocytes, in a dose-dependent manner.

Apolipoprotein B protein secreted from primary Cynomolgous hepatocytes treated with 50 and 150 nM of ISIS 271009 and ISIS 301027 was measured by ELISA using an apolipoprotein B protein specific kit (ALerCHEK Inc., Portland, Me.).

Each result represents the average of 3 experiments. The data are shown as percent reduction in secreted protein, normalized to untreated control cells, and are shown in Table 49. Where present, a “+” indicates that protein secretion was increased.

TABLE 49
Reduction in apolipoprotein B protein secreted from
Cynomolgous monkey hepatocytes
following antisense oligonucleotide treatment

| | % Reduction of monkey | | | |
|---|---|---|---|---|
| | apolipoprotein B protein | | | |
| | secretion | | | |
| Dose of | ISIS # | | | |
| oligonucleotide | 271009 | 301027 | 13650 | 113529 |
| 50 nM | +30 | 25 | N.D. | N.D. |
| 150 nM | 26 | 31 | +1 | 15 |

These results demonstrate that antisense inhibition by ISIS 315089 and ISIS 281625 leads to a decrease in the secretion of apolipoprotein B protein from cultured primary Cynomolgous hepatocytes.

### Example 62

Methods for Evaluating Hepatic Steatosis

Hepatic steatosis refers to the accumulation of lipids in the liver, or “fatty liver”, which is frequently caused by alcohol consumption, diabetes and hyperlipidemia. Livers of animals treated with antisense oligonucleotides targeted to apolipoprotein B were evaluated for the presence of steatosis. Steatosis is assessed by histological analysis of liver tissue and measurement of liver triglyceride levels.

Tissue resected from liver is immediately immersed in Tissue Tek OCT embedding compound (Ted Pella, Inc., Redding, Calif.) and frozen in a 2-methyl-butane dry ice slurry. Tissue sections are cut at a thickness of 4-5 μm and then fixed in 5% neutral-buffered formalin. Tissue sections are stained with hematoxylin and eosin following standard histological procedures to visualize nuclei and cytoplasm, respectively, and oil red O according to the manufacturer's instructions (Newcomers Supply, Middleton, Wis.) to visualize lipids.

Alternatively, tissues are fixed in 10% neutral-buffered formalin, embedded in paraffin, sectioned at a thickness of 4-5 μm, deparaffinized and stained with hematoxylin and eosin, all according to standard histological procedures.

Quantitation of liver triglyceride content is also used to assess steatosis. Tissue triglyceride levels are measured using a Triglyceride GPO Assay (Sigma-Aldrich, St. Louis, Mo.).

### Example 63

Effects of Antisense Inhibition by ISIS 301012 in Lean Mice: Long-Term Study

In accordance with the present invention, the toxicity of ISIS 301012 (SEQ ID NO: 247) is investigated in a long-term, 3 month study in mice. Two-month old male and female CD-1 mice (Charles River Laboratories, Wilmington, Mass.) are dosed with 2, 5, 12.5, 25 or 50 mg/kg of ISIS 301012 twice per week for first week, and every 4 days thereafter. The mice are maintained on a standard rodent diet. Saline and control oligonucleotide animals serve as controls and are injected on the same schedule. Each treatment group contains 6 to 10 mice of each sex, and each treatment group is duplicated, one group for a 1 month study termination, the other for a 3 month study termination. After the 1 or 3 month treatment periods, the mice are sacrificed and evaluated for target expression in liver, lipid levels in serum and indicators of toxicity. Liver samples are procured, RNA is isolated and apolipoprotein B mRNA expression is measured by real-time PCR as described in other examples herein. Serum lipids, including total cholesterol, LDL-cholesterol, HDL-cholesterol and triglycerides, are evaluated by routine clinical analysis using an Olympus Clinical Analyzer (Olympus America Inc., Melville, N.Y.). Ratios of LDL-cholesterol to HDL-cholesterol and total cholesterol to HDL-cholesterol are also calculated. Analyses of serum ALT and AST, inflammatory infiltrates in tissue and basophilic granules in tissue provide an assessment of toxicities related to the treatment. Hepatic steatosis, or accumulation of lipids in the liver, is assessed by routine histological analysis with oil red 0 stain and measurement of liver tissue triglycerides using a Triglyceride GPO Assay (Sigma-Aldrich, St. Louis, Mo.).

The toxicity study also includes groups of animals allowed to recover following cessation of oligonucleotide treatment. Both male and female CD-1 mice (Charles River Laboratories, Wilmington, Mass.) are treated with 5, 10, 50 mg/kg of ISIS 301012 twice per week for the first week and every 4 days thereafter. Saline and control oligonucleotide injected animals serve as controls. Each treatment group includes 6 animals per sex. After 3 months of treatment, animals remain untreated for an additional 3 months, after which they are sacrificed. The same parameters are evaluated as in the mice sacrificed immediately after 3 months of treatment.

After one month of treatment, real-time PCR quantitation reveals that mouse apolipoprotein B mRNA levels in liver are reduced by 53%. Additionally, the expected dose-response toxicities were observed. ALT and AST levels, measured by routine clinical procedures on an Olympus Clinical Analyzer (Olympus America Inc., Melville, N.Y.), are increased in mice treated with 25 or 50 mg/kg of ISIS 301012. Tissues were prepared for analysis by routine histological procedures. Basophilic granules in liver and kidney tissue were observed at doses of ISIS 301012 above 12.5 mg/kg. Mild lymphohistiocytic infiltrates were observed in various tissues at doses greater than 12.5 mg/kg of ISIS 301012. Staining of tissue sections with oil red 0 reveals no steatosis present following the oligonucleotide treatments.

### Example 64

Effects of Antisense Inhibition by ISIS 301012 in Lean Cynomolgous Monkeys: Long-Term Study

As discussed in Example 45, Cynomolgus monkeys (male or female) are used to evaluate antisense oligonucleotides for their potential to lower apolipoprotein B mRNA or protein levels, as well as phenotypic endpoints associated with apolipoprotein B including, but not limited to cardiovascular indicators, atherosclerosis, lipid diseases, obesity, and plaque formation. Accordingly, in a further embodiment of the invention, ISIS 301012 (SEQ ID NO: 247) is investigated in a long-term study for its effects on apolipoprotein B expression and serum lipids in Cynomolgous monkeys. Such a long-term study is also used to evaluate the toxicity of antisense compounds.

Male and female Cynomolgous monkeys are treated with 2, 4 or 12 mg/kg of ISIS 301012 intravenously or 2 or 20 mg/kg subcutaneously at a frequency of every two days for the first week, and every 4 days thereafter, for 1 and 3 month treatment periods. Saline-treated animals serve as controls. Each treatment group includes 2 to 3 animals of each sex.

At a one month interval and at the 3 month study termination, the animals are sacrificed and evaluated for target expression in liver, lipid levels in serum and indicators of toxicity. Liver samples are procured, RNA is isolated and apolipoprotein B mRNA expression is measured by real-time PCR as described in other examples herein. Serum lipids, including total cholesterol, LDL-cholesterol, HDL-cholesterol and triglycerides, are evaluated by routine clinical analysis using an Olympus Clinical Analyzer (Olympus America Inc., Melville, N.Y.). Ratios of LDL-cholesterol to HDL-cholesterol and total cholesterol to HDL-cholesterol are also calculated. Analyses of serum ALT and AST, inflammatory infiltrates in tissue and basophilic granules in tissue provide an assessment of toxicities related to the treatment. Hepatic steatosis, or accumulation of lipids in the liver, is assessed by routine histological analysis with oil red 0 stain and measurement of liver tissue triglycerides using a Triglyceride GPO Assay (Sigma-Aldrich, St. Louis, Mo.).

Additional treatment groups consisting of 2 animals per sex are treated with saline (0 mg/kg), 12 or 20 mg/kg ISIS 301012 at a frequency of every two days for the first week, and every 4 days thereafter, for a 3 month period. Following the treatment period, the animals receive no treatment for an additional three months. These treatment groups are for the purpose of studying the effects of apolipoprotein B inhibition 3 months after cessation of treatment. At the end of the 3 month recovery period, animals are sacrificed and evaluated for the same parameters as the animals sacrificed immediately after 1 and 3 months of treatment.

The results from the one month interval of the long term treatment are shown in Table 50 and are normalized to saline-treated animals for mRNA and to untreated baseline values for lipid levels. Total cholesterol, LDL-cholesterol, HDL-cholesterol, LDL particle concentration and triglyceride levels in serum were measured by nuclear magnetic resonance spectroscopy by Liposcience (Raleigh, N.C.). Additionally, the concentration of intact oligonucleotide in liver was measured by capillary gel electrophoresis and is presented as micrograms of oligonucleotide per gram of liver tissue. Each result represents the average of data from 4 animals (2 males and 2 females).

TABLE 50
Effects of antisense inhibition by ISIS 301012 in lean Cynomolgous monkeys

| | | Intravenous | | | Subcutaneous | |
|---|---|---|---|---|---|---|
| | | delivery | | | injection | |
| | | 2 mg/kg | 4 mg/kg | 12 mg/kg | 3.5 mg/kg | 20 mg/kg |
| apolipoprotein B expression | | −45 | −76 | −96 | N.D. | −94 |
| % change normalized to saline | | | | | | |
| antisense oligonucleotide | | 92 | 179 | 550 | N.D. | 855 |
| concentration μg/g | | | | | | |
| Lipid parameters, | | | | | | |
| % change normalized to | | 2 | 4 | 12 | 3.5 | 20 |
| untreated baseline value | Saline | mg/kg | mg/kg | mg/kg | mg/kg | mg/kg |
| Total cholesterol | +1 | −6 | −2 | −2 | +5 | −5 |
| LDL-cholesterol | +17 | +15 | +9 | +3 | −4 | −16 |
| HDL-cholesterol | −11 | −23 | −15 | −8 | +13 | +5 |
| LDL/HDL | +62 | +94 | +38 | +44 | −15 | −19 |
| Total cholesterol/HDL | +30 | +44 | +22 | +21 | −7 | −10 |
| Triglyceride | +37 | +26 | +32 | +15 | +1 | −3 |
| LDL Particle concentration | +15 | +8 | +8 | −11 | −14 | −21 |

These data show that ISIS 301012 inhibits apolipoprotein B expression in a dose-dependent manner in a primate species and concomitantly lowers lipid levels at higher doses of ISIS 301012. Furthermore, these results demonstrate that antisense oligonucleotide accumulates in the liver in a dose-dependent manner.

Hepatic steatosis, or accumulation of lipids in the liver, was not observed following 4 weeks of treatment with the doses indicated. Expected dose-related toxicities were observed at the higher doses of 12 and 20 mg/kg, including a transient 1.2-1.3 fold increase in activated partial thromboplastin time (APTT) during the first 4 hours and basophilic granules in the liver and kidney (as assessed by routine histological examination of tissue samples). No functional changes in kidney were observed.

In a similar experiment, male and female Cynomolgous monkeys received an intravenous dose of ISIS 301012 at 4 mg/kg, every two days for the first week and every 4 days thereafter. Groups of animals were sacrificed after the first dose and the fourth dose, as well as 11, 15 and 23 days following the fourth and final dose. Liver RNA was isolated and apolipoprotein B mRNA levels were evaluated by real-time PCR as described herein. The results of this experiment demonstrate a 40% reduction in apolipoprotein B mRNA expression after a single intravenous dose of 4 mg/kg ISIS 301012. Furthermore, after 4 doses of ISIS 301012 at 4 mg/kg, target mRNA was reduced by approximately 85% and a 50% reduction in target mRNA was sustained for up to 16 days following the cessation of antisense oligonucleotide treatment.

### Example 65

Microarray Analysis: Gene Expression Patterns in Lean Versus High-Fat Fed Mice

Male C57Bl/6 mice were divided into the following groups, consisting of 5 animals each: (1) mice on a lean diet, injected with saline (lean control); (2) mice on a high fat diet; (3) mice on a high fat diet injected with 50 mg/kg of the control oligonucleotide 141923 (SEQ ID NO: 858); (4) mice on a high fat diet given 20 mg/kg atorvastatin calcium (Lipitor®, Pfizer Inc.); (5) mice on a high fat diet injected with 10, 25 or 50 mg/kg ISIS 147764 (SEQ ID NO: 109). Saline and oligonucleotide treatments were administered intraperitoneally twice weekly for 6 weeks. Atorvastatin was administered daily for 6 weeks. At study termination, liver samples were isolated from each animal and RNA was isolated for Northern blot qualitative assessment, DNA microarray and quantitative real-time PCR. Northern blot assessment and quantitative real-time PCR were performed as described by other examples herein.

For DNA microarray analysis, hybridization samples were prepared from 10 μg of total RNA isolated from each mouse liver according to the Affymetrix Expression Analysis Technical Manual (Affymetrix, Inc., Santa Clara, Calif.). Samples were hybridized to a mouse gene chip containing approximately 22,000 genes, which was subsequently washed and double-stained using the Fluidics Station 400 (Affymetrix, Inc., Santa Clara, Calif.) as defined by the manufacturer's protocol. Stained gene chips were scanned for probe cell intensity with the GeneArray scanner (Affymetrix, Inc., Santa Clara, Calif.). Signal values for each probe set were calculated using the Affymetrix Microarray Suite v5.0 software (Affymetrix, Inc., Santa Clara, Calif.). Each condition was profiled from 5 biological samples per group, one chip per sample. Fold change in expression was computed using the geometric mean of signal values as generated by Microarray Suite v5.0. Statistical analysis utilized one-way ANOVA followed by 9 pair-wise comparisons. All groups were compared to the high fat group to determine gene expression changes resulting from ISIS 147764 treatment. Microarray data was interpreted using hierarchical clustering to visualize global gene expression patterns.

The results of the microarray analysis reveal that treatment with ISIS 147764 drives the gene expression profile in high fat fed mice to the profile observed in lean mice. Real-time PCR analysis confirmed the reduction in mRNA expression for the following genes involved in the lipid metabolism: hepatic lipase, fatty acid synthase ATP-binding cassette, sub-family D (ALD) member 2, intestinal fatty acid binding protein 2, stearol CoA desaturase-1 and HMG CoA reductase.

Mouse apolipoprotein B mRNA and serum cholesterol levels, measured as described herein, were evaluated to confirm antisense inhibition by ISIS 147764 and ISIS 147483. Both mRNA and cholesterol levels were lowered in a dose-dependent manner following treatment with ISIS 147764 or ISIS 147483, as demonstrated in other examples herein. The 50 mg/kg dose of ISIS 147483 increased ALT and AST levels. The 10, 25 and 50 mg/kg doses of ISIS 147764 and the 10 and 25 mg/kg doses of ISIS 147483 did not significantly elevate ALT or AST levels.

### Example 66

Evaluation of Hepatic Steatosis in Animals Treated with Apolipoprotein B Antisense Oligonucleotides

Livers of animals treated with antisense oligonucleotides targeted to apolipoprotein B were evaluated for the presence of steatosis. Steatosis is assessed by histological analysis of liver tissue and measurement of liver triglyceride levels.

Evaluation of Steatosis in High Fat Fed Animals Treated with ISIS 147764 for 6 Weeks

Liver tissue from ISIS 147764 (SEQ ID NO: 109) and control-treated animals described in Example 21 was evaluated for steatosis at study termination following 6 weeks of treatment. Tissue sections were stained with oil red 0 and hematoxylin to visualize lipids and nuclei, respectively. Tissue sections were also stained with hematoxylin and eosin to visualize nuclei and cytoplasm, respectively. Histological analysis of tissue sections stained by either method reveal no difference in steatosis between saline treated and ISIS 147764 treated animals, demonstrating that a 6 week treatment with ISIS 147764 does not lead to accumulation of lipids in the liver.

Evaluation of Steatosis Following Long-Term Treatment with Apolipoprotein B Inhibitor in High-Fat Fed Animals

Male C57Bl/6 mice were treated with twice weekly intraperitoneal injections of 25 mg/kg ISIS 147764 (SEQ ID NO: 109) or 25 mg/kg ISIS 141923 (SEQ ID NO: 858) for 6, 12 and 20 weeks. Saline treated animals served as controls. Each treatment group contained 4 animals. Animals were sacrificed at 6, 12 and 20 weeks and liver tissue was procured for histological analysis and measurement of tissue triglyeride content. The results reveal no significant differences in liver tissue triglyceride content when ISIS 147764 treated animals are compared to saline treated animals. Furthermore, histological analysis of liver tissue section demonstrates that steatosis is reduced at 12 and 20 weeks following treatment of high fat fed mice with ISIS 147764, in comparison to saline control animals that received a high fat diet.

Evaluation of Steatosis in Lean Mice

The accumulation of lipids in liver tissue was also evaluated in lean mice. Male C67Bl/6 mice (Charles River Laboratories (Wilmington, Mass.) at 6 to 7 weeks of age were maintained on a standard rodent diet and were treated twice weekly with intraperitoneal injections of 25 or 50 mg/kg 147764 (SEQ ID NO: 109) or 147483 (SEQ ID NO: 79) for 6 weeks. Saline treated animals served as controls. Each treatment group was comprised of 4 animals. Animals were sacrificed after the 6 week treatment period, at which point liver tissue and serum were collected.

Apolipoprotein B mRNA levels were measured by real-time PCR as described by other examples herein. The data, shown in Table 51, represent the average of 4 animals and are presented as inhibition relative to saline treated controls. The results demonstrate that both ISIS 147483 and ISIS 147764 inhibit apolipoprotein B mRNA expression in lean mice in a dose-dependent manner.

TABLE 51
Antisense inhibition of apolipoprotein B mRNA in lean mice

| | Treatment and dose | | | |
|---|---|---|---|---|
| | ISIS | | ISIS | |
| | 147483 | | 147764 | |
| | 25 | 50 | 25 | 50 |
| | mg/kg | mg/kg | mg/kg | mg/kg |
| % inhibition | 79 | 91 | 48 | 77 |
| apolipoprotein B | | | | |
| mRNA | | | | |

Total cholesterol, LDL-cholesterol, HDL-cholesterol and triglycerides in serum were measured by routine clinical analysis using an Olympus Clinical Analyzer (Olympus America Inc., Melville, N.Y.). The liver enzymes ALT and ALT in serum were also measured using the Olympus Clinical Analyzer. These results demonstrate that ISIS 147764 lowers serum lipids relative to saline-treated control animals. ALT and AST levels do not exceed the normal range for mice (300 IU/L), indicating a lack of treatment-associated toxicity. The results are the average of data from 4 animals and are shown in Table 52.

TABLE 52
Serum lipids and liver enzyme levels in lean mice
treated with ISIS 147764 and ISIS 147483

| | Treatment and dose | | | | |
|---|---|---|---|---|---|
| | | ISIS 147483 | | ISIS 147764 | |
| | | 25 | 50 | 25 | 50 |
| | Saline | mg/kg | mg/kg | mg/kg | mg/kg |
| Serum lipids | | | | | |
| Total | 164 | 153 | 183 | 114 | 57 |
| cholesterol | | | | | |
| mg/dL | | | | | |
| LDL- | 25 | 26 | 39 | 29 | 18 |
| cholesterol | | | | | |
| mg/dL | | | | | |
| HDL- | 127 | 117 | 131 | 79 | 38 |
| cholesterol | | | | | |
| mg/dL | | | | | |
| Triglycerides | 121 | 138 | 127 | 80 | 30 |
| mg/dL | | | | | |
| Liver enzymes | | | | | |
| ALT | 105 | 73 | 57 | 47 | 48 |
| IU/L | | | | | |
| AST | 109 | 78 | 72 | 81 | 101 |
| IU/L | | | | | |

Liver tissue was prepared by routine histological methods to evaluate steatosis, as described herein. Examination of tissue samples stained with oil red 0 or hematoxylin and eosin reveals that treatment of lean mice with apolipoprotein B antisense oligonucleotides does not result in steatosis.

Six Month Study to Further Evaluate Steatosis in Mice Treated with Apolipoprotein B Antisense Oligonucleotides

A long-term treatment of mice with antisense oligonucleotides targeted to apolipoprotein B is used to evaluate the toxicological and pharmacological effects of extended treatment with antisense compounds. Both male and female C57Bl/6 mice at 2 months of age are treated with 2, 5, 25 or 50 mg/kg of apolipoprotein B antisense oligonucleotide. Treatments are administered intraperitoneally every 2 days for the first week and every 4 days thereafter. Mice treated with saline alone or control oligonucleotide serve as control groups. Each treatment group contains 25 to 30 mice. After 6 months of treatment, a subset of the mice in each treatment group is sacrificed. The remaining mice are allowed a 3 month recovery period without treatment, after which they are sacrificed. Apolipoprotein B mRNA expression in liver is measured by real-time PCR as described by other methods herein. Liver tissue is also prepared for measurement of triglyceride content using a Triglyceride GPO Assay (Sigma-Aldrich, St. Louis, Mo.). Serum is collected and evaluated for lipid content, including total cholesterol, LDL-cholesterol, HDL-cholesterol and triglyceride, using an Olympus Clinical Analyzer (Olympus America Inc., Melville, N.Y.). The liver enzymes ALT and AST are also measured in serum, also using the clinical analyzer. Serum samples are subjected to immunoblot analysis using an antibody directed to apolipoprotein B (Santa Cruz Biotechnology, Inc., Santa Cruz, Calif.). Liver, kidney and other tissues are prepared by routine procedures for histological analyses. Tissues are evaluated for the presence of basophilic granules and inflammatory infiltrates. Steatosis is evaluated by oil red 0 stain of liver tissue sections.

### Example 67

A Mouse Model for Atherosclerotic Plaque Formation: Human Apolipoprotein B Transgenic Mice Lacking the LDL Receptor Gene

The LDL receptor is responsible for clearing apolipoprotein B-containing LDL particles. Without the LDL receptor, animals cannot effectively clear apolipoprotein B-containing LDL particles from the plasma. Thus the serum levels of apolipoprotein B and LDL cholesterol are markedly elevated. Mice expressing the human apolipoprotein B transgene (TgN-hApoB +/+) and mice deficient for the LDL receptor (LDLr −/−) are both used as animal models of atherosclerotic plaque development. When the LDL receptor deficiency genotype is combined with a human apolipoprotein B transgenic genotype (TgN-hApoB +/+; LDLr −/−), atherosclerotic plaques develop rapidly. In accordance with the present invention, mice of this genetic background are used to investigate the ability of compounds to prevent atherosclerosis and plaque formation.

Male TgN-hApoB+/+; LDLr −/− mice are treated twice weekly with 10 or 20 mg/kg of human apolipoprotein B antisense oligonucleotides for 12 weeks. Control groups are treated with saline or control oligonucleotide. Serum total cholesterol, HDL-cholesterol, LDL-cholesterol and triglycerides are measured at 2, 4, 6, 8 and 12 weeks by routine clinical analysis using an Olympus Clinical Analyzer (Olympus America Inc., Melville, N.Y.). Serum human apolipoprotein B protein is measured at 2, 4, 6, 8 and 12 weeks using an ELISA kit (ALerCHEK Inc., Portland, Me.). Human and mouse apolipoprotein mRNA in liver is measured at 12 weeks. The results of the 12 week study serve to evaluate the pharmacological behavior of ISIS 301012 in a doubly transgenic model.

Additionally, a four month study is performed in TgN-hApoB+/+; LDLr−/− mice, with treatment conditions used in the 12 week study. Mice are treated for 4 months with antisense oligonucleotides targeted to human apolipoprotein B to evaluate the ability of such compounds to prevent atherosclerotic plaque formation. At the end of the 4 month treatment period, mice are anesthetized and perfused with 10% formalin. The perfused arterial tree is isolated and examined for the presence of atherosclerotic plaques. Sections of the arterial tree are embedded in paraffin and prepared for histological analysis using routine methods. Serum total cholesterol, HDL-cholesterol, LDL-cholesterol and triglycerides are measured at 2, 4, 6, 8, 12 and 16 weeks by routine clinical analysis using an Olympus Clinical Analyzer (Olympus America Inc., Melville, N.Y.). Serum human apolipoprotein B protein is measured at 2, 4, 6, 8, 12 and 16 weeks using an ELISA kit (ALerCHEK Inc., Portland, Me.). Human and mouse apolipoprotein mRNA in liver at 16 weeks is measured by real-time PCR.

### Example 68

Rabbit Models for Study of Atherosclerotic Plaque Formation

The Watanabe heritable hyperlipidemic (WHHL) strain of rabbit is used as a model for atherosclerotic plaque formation. New Zealand white rabbits on a high-fat diet are also used as a model of atherosclerotic plaque formation. Treatment of WHHL or high fat fed New Zealand white rabbits with apolipoprotein B antisense compounds is used to test their potential as therapeutic or prophylactic treatments for atherosclerotic plaque disease. Rabbits are injected with 5, 10, 25 or 50 mg/kg of antisense oligonucleotides targeted to apolipoprotein B. Animals treated with saline alone or a control oligonucleotide serve as controls. Throughout the treatment, serum samples are collected and evaluated for apolipoprotein B protein levels by ELISA (kit from ALerCHEK Inc., Portland, Me.) and serum lipids (cholesterol, LDL-cholesterol, VLDL-cholesterol, HDL-cholesterol, triglycerides) by routine clinical analysis. Liver tissue triglyceride content is measured using a Triglyceride GPO Assay (Sigma-Aldrich, St. Louis, Mo.). Liver, kidney, heart, aorta and other tissues are procured and processed for histological analysis using routine procedures. Liver and kidney tissues are examined for evidence of basophilic granules and inflammatory infiltrates. Liver tissue is evaluated for steatosis using oil red 0 stain. Additionally, aortic sections stained with oil red 0 stain and hematoxylin are examined to evaluate the formation of atherosclerotic lesions.

### Example 69

Oral Delivery of Apolipoprotein B Inhibitors

Oligonucleotides may be formulated for delivery in vivo in an acceptable dosage form, e.g. as parenteral or non-parenteral formulations. Parenteral formulations include intravenous (IV), subcutaneous (SC), intraperitoneal (IP), intravitreal and intramuscular (IM) formulations, as well as formulations for delivery via pulmonary inhalation, intranasal administration, topical administration, etc. Non-parenteral formulations include formulations for delivery via the alimentary canal, e.g. oral administration, rectal administration, intrajejunal instillation, etc. Rectal administration includes administration as an enema or a suppository. Oral administration includes administration as a capsule, a gel capsule, a pill, an elixir, etc.

In some embodiments, an oligonucleotide may be administered to a subject via an oral route of administration. The subject may be an animal or a human (man). An animal subject may be a mammal, such as a mouse, rat, mouse, a rat, a dog, a guinea pig, a monkey, a non-human primate, a cat or a pig. Non-human primates include monkeys and chimpanzees. A suitable animal subject may be an experimental animal, such as a mouse, rat, mouse, a rat, a dog, a monkey, a non-human primate, a cat or a pig.

In some embodiments, the subject may be a human. In certain embodiments, the subject may be a human patient in need of therapeutic treatment as discussed in more detail herein. In certain embodiments, the subject may be in need of modulation of expression of one or more genes as discussed in more detail herein. In some particular embodiments, the subject may be in need of inhibition of expression of one or more genes as discussed in more detail herein. In particular embodiments, the subject may be in need of modulation, i.e. inhibition or enhancement, of apolipoprotein B in order to obtain therapeutic indications discussed in more detail herein.

In some embodiments, non-parenteral (e.g. oral) oligonucleotide formulations according to the present invention result in enhanced bioavailability of the oligonucleotide. In this context, the term “bioavailability” refers to a measurement of that portion of an administered drug which reaches the circulatory system (e.g. blood, especially blood plasma) when a particular mode of administration is used to deliver the drug. Enhanced bioavailability refers to a particular mode of administration's ability to deliver oligonucleotide to the peripheral blood plasma of a subject relative to another mode of administration. For example, when a non-parenteral mode of administration (e.g. an oral mode) is used to introduce the drug into a subject, the bioavailability for that mode of administration may be compared to a different mode of administration, e.g. an IV mode of administration. In some embodiments, the area under a compound's blood plasma concentration curve (AUC₀) after non-parenteral (e.g. oral, rectal, intrajejunal) administration may be divided by the area under the drug's plasma concentration curve after intravenous (i.v.) administration (AUCᵢᵥ) to provide a dimensionless quotient (relative bioavailability, RB) that represents fraction of compound absorbed via the non-parenteral route as compared to the IV route. A composition's bioavailability is said to be enhanced in comparison to another composition's bioavailability when the first composition's relative bioavailability (RB₁) is greater than the second composition's relative bioavailability (RB₂).

In general, bioavailability correlates with therapeutic efficacy when a compound's therapeutic efficacy is related to the blood concentration achieved, even if the drug's ultimate site of action is intracellular (van Berge-Henegouwen et al., Gastroenterol., 1977, 73, 300). Bioavailability studies have been used to determine the degree of intestinal absorption of a drug by measuring the change in peripheral blood levels of the drug after an oral dose (DiSanto, Chapter 76 In: Remington=s Pharmaceutical Sciences, 18th Ed., Gennaro, ed., Mack Publishing Co., Easton, Pa., 1990, pages 1451-1458).

In general, an oral composition's bioavailability is said to be “enhanced” when its relative bioavailability is greater than the bioavailability of a composition substantially consisting of pure oligonucleotide, i.e. oligonucleotide in the absence of a penetration enhancer.

Organ bioavailability refers to the concentration of compound in an organ. Organ bioavailability may be measured in test subjects by a number of means, such as by whole-body radiography. Organ bioavailability may be modified, e.g. enhanced, by one or more modifications to the oligonucleotide, by use of one or more carrier compounds or excipients, etc. as discussed in more detail herein. In general, an increase in bioavailability will result in an increase in organ bioavailability.

Oral oligonucleotide compositions according to the present invention may comprise one or more “mucosal penetration enhancers,” also known as “absorption enhancers” or simply as “penetration enhancers.” Accordingly, some embodiments of the invention comprise at least one oligonucleotide in combination with at least one penetration enhancer. In general, a penetration enhancer is a substance that facilitates the transport of a drug across mucous membrane(s) associated with the desired mode of administration, e.g. intestinal epithelial membranes. Accordingly it is desirable to select one or more penetration enhancers that facilitate the uptake of an oligonucleotide, without interfering with the activity of the oligonucleotide, and in a such a manner the oligonucleotide can be introduced into the body of an animal without unacceptable side-effects such as toxicity, irritation or allergic response.

Embodiments of the present invention provide compositions comprising one or more pharmaceutically acceptable penetration enhancers, and methods of using such compositions, which result in the improved bioavailability of oligonucleotides administered via non-parenteral modes of administration. Heretofore, certain penetration enhancers have been used to improve the bioavailability of certain drugs. See Muranishi, Crit. Rev. Ther. Drug Carrier Systems, 1990, 7, 1 and Lee et al., Crit. Rev. Ther. Drug Carrier Systems, 1991, 8, 91. It has been found that the uptake and delivery of oligonucleotides, relatively complex molecules which are known to be difficult to administer to animals and man, can be greatly improved even when administered by non-parenteral means through the use of a number of different classes of penetration enhancers.

In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property. Modifications may be made to the base, the linker, or the sugar, in general, as discussed in more detail herein with regards to oligonucleotide chemistry. In some embodiments of the invention, compositions for administration to a subject, and in particular oral compositions for administration to an animal or human subject, will comprise modified oligonucleotides having one or more modifications for enhancing affinity, stability, tissue distribution, or other biological property.

Suitable modified linkers include phosphorothioate linkers. In some embodiments according to the invention, the oligonucleotide has at least one phosphorothioate linker. Phosphorothioate linkers provide nuclease stability as well as plasma protein binding characteristics to the oligonucleotide. Nuclease stability is useful for increasing the in vivo lifetime of oligonucleotides, while plasma protein binding decreases the rate of first pass clearance of oligonucleotide via renal excretion. In some embodiments according to the present invention, the oligonucleotide has at least two phosphorothioate linkers. In some embodiments, wherein the oligonucleotide has exactly n nucleosides, the oligonucleotide has from one to n−1 phosphorothioate linkages. In some embodiments, wherein the oligonucleotide has exactly n nucleosides, the oligonucleotide has n−1 phosphorothioate linkages. In other embodiments wherein the oligonucleotide has exactly n nucleoside, and n is even, the oligonucleotide has from 1 to n/2 phosphorothioate linkages, or, when n is odd, from 1 to (n−1)/2 phosphorothioate linkages. In some embodiments, the oligonucleotide has alternating phosphodiester (PO) and phosphorothioate (PS) linkages. In other embodiments, the oligonucleotide has at least one stretch of two or more consecutive PO linkages and at least one stretch of two or more PS linkages. In other embodiments, the oligonucleotide has at least two stretches of PO linkages interrupted by at least on PS linkage.

In some embodiments, at least one of the nucleosides is modified on the ribosyl sugar unit by a modification that imparts nuclease stability, binding affinity or some other beneficial biological property to the sugar. In some cases, the sugar modification includes a 2′-modification, e.g. the 2′-OH of the ribosyl sugar is replaced or substituted. Suitable replacements for 2′-OH include 2′-F and 2′-arabino-F. Suitable substitutions for OH include 2′-O-alkyl, e.g. 2-O-methyl, and 2′-O-substituted alkyl, e.g. 2′-O-methoxyethyl, 2′-O-aminopropyl, etc. In some embodiments, the oligonucleotide contains at least one 2′-modification. In some embodiments, the oligonucleotide contains at least 2 2′-modifications. In some embodiments, the oligonucleotide has at least one 2′-modification at each of the termini (i.e. the 3′- and 5′-terminal nucleosides each have the same or different 2′-modifications). In some embodiments, the oligonucleotide has at least two sequential 2′-modifications at each end of the oligonucleotide. In some embodiments, oligonucleotides further comprise at least one deoxynucleoside. In particular embodiments, oligonucleotides comprise a stretch of deoxynucleosides such that the stretch is capable of activating RNase (e.g. RNase H) cleavage of an RNA to which the oligonucleotide is capable of hybridizing. In some embodiments, a stretch of deoxynucleosides capable of activating RNase-mediated cleavage of RNA comprises about 6 to about 16, e.g. about 8 to about 16 consecutive deoxynucleosides.

Oral compositions for administration of non-parenteral oligonucleotide compositions of the present invention may be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. A common requirement for these modes of administration is absorption over some portion or all of the alimentary tract and a need for efficient mucosal penetration of the nucleic acid(s) so administered.

Delivery of a drug via the oral mucosa, as in the case of buccal and sublingual administration, has several desirable features, including, in many instances, a more rapid rise in plasma concentration of the drug than via oral delivery (Harvey, Chapter 35 In: Remington=s Pharmaceutical Sciences, 18th Ed., Gennaro, ed., Mack Publishing Co., Easton, Pa., 1990, page 711).

Endoscopy may be used for drug delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., Gan To Kagaku Ryoho, 1992, 19(10 Suppl), 1591). Pharmaceutical compositions, including liposomal formulations, can be delivered directly into portions of the alimentary canal, such as, e.g., the duodenum (Somogyi et al., Pharm. Res., 1995, 12, 149) or the gastric submucosa (Akamo et al., Japanese J. Cancer Res., 1994, 85, 652) via endoscopic means. Gastric lavage devices (Inoue et al., Artif. Organs, 1997, 21, 28) and percutaneous endoscopic feeding devices (Pennington et al., Ailment Pharmacol. Ther., 1995, 9, 471) can also be used for direct alimentary delivery of pharmaceutical compositions.

In some embodiments, oligonucleotide formulations may be administered through the anus into the rectum or lower intestine. Rectal suppositories, retention enemas or rectal catheters can be used for this purpose and may be preferred when patient compliance might otherwise be difficult to achieve (e.g., in pediatric and geriatric applications, or when the patient is vomiting or unconscious). Rectal administration can result in more prompt and higher blood levels than the oral route. (Harvey, Chapter 35 In: Remington=s Pharmaceutical Sciences, 18th Ed., Gennaro, ed., Mack Publishing Co., Easton, Pa., 1990, page 711). Because about 50% of the drug that is absorbed from the rectum will bypass the liver, administration by this route significantly reduces the potential for first-pass metabolism (Benet et al., Chapter 1 In: Goodman & Gilman=s The Pharmacological Basis of Therapeutics, 9th Ed., Hardman et al., eds., McGraw-Hill, New York, N.Y., 1996).

One advantageous method of non-parenteral administration oligonucleotide compositions is oral delivery. Some embodiments employ various penetration enhancers in order to effect transport of oligonucleotides and other nucleic acids across mucosal and epithelial membranes. Penetration enhancers may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Accordingly, some embodiments comprise oral oligonucleotide compositions comprising at least one member of the group consisting of surfactants, fatty acids, bile salts, chelating agents, and non-chelating surfactants. Further embodiments comprise oral oligonucleotide comprising at least one fatty acid, e.g. capric or lauric acid, or combinations or salts thereof. Other embodiments comprise methods of enhancing the oral bioavailability of an oligonucleotide, the method comprising co-administering the oligonucleotide and at least one penetration enhancer.

Other excipients that may be added to oral oligonucleotide compositions include surfactants (or “surface-active agents”), which are chemical entities which, when dissolved in an aqueous solution, reduce the surface tension of the solution or the interfacial tension between the aqueous solution and another liquid, with the result that absorption of oligonucleotides through the alimentary mucosa and other epithelial membranes is enhanced. In addition to bile salts and fatty acids, surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorohemical emulsions, such as FC-43 (Takahashi et al., J. Pharm. Pharmacol., 1988, 40, 252).

Fatty acids and their derivatives which act as penetration enhancers and may be used in compositions of the present invention include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines and mono- and di-glycerides thereof and/or physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 1990, 7, 1; El-Hariri et al., J. Pharm. Pharmacol., 1992, 44, 651).

In some embodiments, oligonucleotide compositions for oral delivery comprise at least two discrete phases, which phases may comprise particles, capsules, gel-capsules, microspheres, etc. Each phase may contain one or more oligonucleotides, penetration enhancers, surfactants, bioadhesives, effervescent agents, or other adjuvant, excipient or diluent. In some embodiments, one phase comprises at least one oligonucleotide and at lease one penetration enhancer. In some embodiments, a first phase comprises at least one oligonucleotide and at least one penetration enhancer, while a second phase comprises at least one penetration enhancer. In some embodiments, a first phase comprises at least one oligonucleotide and at least one penetration enhancer, while a second phase comprises at least one penetration enhancer and substantially no oligonucleotide. In some embodiments, at least one phase is compounded with at least one degradation retardant, such as a coating or a matrix, which delays release of the contents of that phase. In some embodiments, at least one phase. In some embodiments, a first phase comprises at least one oligonucleotide, at least one penetration enhancer, while a second phase comprises at least one penetration enhancer and a release-retardant. In particular embodiments, an oral oligonucleotide comprises a first phase comprising particles containing an oligonucleotide and a penetration enhancer, and a second phase comprising particles coated with a release-retarding agent and containing penetration enhancer.

A variety of bile salts also function as penetration enhancers to facilitate the uptake and bioavailability of drugs. The physiological roles of bile include the facilitation of dispersion and absorption of lipids and fat-soluble vitamins (Brunton, Chapter 38 In: Goodman & Gilman=s The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington=s Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1; Yamamoto et al., J. Pharm. Exp. Ther., 1992, 263, 25; Yamashita et al., J. Pharm. Sci., 1990, 79, 579).

In some embodiments, penetration enhancers useful in some embodiments of present invention are mixtures of penetration enhancing compounds. One such penetration enhancer is a mixture of UDCA (and/or CDCA) with capric and/or lauric acids or salts thereof e.g. sodium. Such mixtures are useful for enhancing the delivery of biologically active substances across mucosal membranes, in particular intestinal mucosa. Other penetration enhancer mixtures comprise about 5-95% of bile acid or salt(s) UDCA and/or CDCA with 5-95% capric and/or lauric acid. Particular penetration enhancers are mixtures of the sodium salts of UDCA, capric acid and lauric acid in a ratio of about 1:2:2 respectively. Anther such penetration enhancer is a mixture of capric and lauric acid (or salts thereof) in a 0.01:1 to 1:0.01 ratio (mole basis). In particular embodiments capric acid and lauric acid are present in molar ratios of e.g. about 0.1:1 to about 1:0.1, in particular about 0.5:1 to about 1:0.5.

Other excipients include chelating agents, i.e. compounds that remove metallic ions from solution by forming complexes therewith, with the result that absorption of oligonucleotides through the alimentary and other mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1; Buur et al., J. Control Rel., 1990, 14, 43).

As used herein, non-chelating non-surfactant penetration enhancers may be defined as compounds that demonstrate insignificant activity as chelating agents or as surfactants but that nonetheless enhance absorption of oligonucleotides through the alimentary and other mucosal membranes (Muranishi, Critical Reviews in Therapeutic Drug Carrier Systems, 1990, 7, 1). This class of penetration enhancers includes, but is not limited to, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), can be used.

Some oral oligonucleotide compositions also incorporate carrier compounds in the formulation. As used herein, “carrier compound” or “carrier” can refer to a nucleic acid, or analog thereof, which may be inert (i.e., does not possess biological activity per se) or may be necessary for transport, recognition or pathway activation or mediation, or is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

A “pharmaceutical carrier” or “excipient” may be a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.). Oral oligonucleotide compositions may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the present invention.

### Example 70

Microarray Analysis: Evaluation of Dose-Dependent Gene Expression Patterns in Lean Versus High-Fat Fed Mice

DNA array analysis of gene expression patterns is a useful tool for investigating global mRNA changes following antisense inhibition of a target gene. To this end, gene expression patterns in mouse liver were evaluated following antisense inhibition of apolipoprotein B. ISIS 147764 and ISIS 147483 are targeted to mouse apolipoprotein B and were the antisense compounds used in this study. ISIS 147764 (GTCCCTGAAGATGTCAATGC, SEQ ID NO: 17) and ISIS 147483 (ATGTCAATGCCACATGTCCA, SEQ ID NO: 18) were designed using published mouse apolipoprotein B sequence (SEQ ID NO: 10). ISIS 141923 (CCTTCCCTGAAGGTTCCTCC, SEQ ID NO: 19) does not target apolipoprotein B and was used as a control antisense oligonucleotide. These compounds are chimeric oligomeric compounds 20 nucleotides in length, composed of a central gap region consisting of 10 2′-deoxynucleotides, which is flanked on both sides (5′ and 3′ directions) by 5-nucleotide “wing” segments. The wings are composed of 2′-O-methoxyethyl nucleotides, or 2′-MOE nucleotides. The internucleoside (backbone) linkages are phosphorothioate throughout, and all cytidine residues are 5-methylcytidines.

Liver gene expression patterns were evaluated as a function of apolipoprotein B antisense oligonucleotide dose. Male C57Bl/6 mice were divided into the following groups: (1) mice on a lean diet, injected with saline (lean control); (2) mice on a high fat diet, injected with saline (high-fat fed); (3) mice on a high fat diet injected with 50 mg/kg of the control oligonucleotide 141923 (SEQ ID NO: 19); (4) mice on a high fat diet given 20 mg/kg atorvastatin calcium (Lipitor®, Pfizer Inc.); (5) mice on a high fat diet injected with 10, 25 or 50 mg/kg ISIS 147764 (SEQ ID NO: 17) (6) mice on a high fat diet injected with 10, 25 or 50 mg/kg ISIS 147483 (SEQ ID NO: 18). Each dose of apolipoprotein B antisense oligonucleotide was administered to a total of 5 mice, thus groups (5) and (6) consisted of 15 animals each. All other groups consisted of 5 animals each. Mice in the high-fat diet groups were maintained on a diet of 60% lard for 4 weeks prior to treatment. Saline and oligonucleotide treatments were administered intraperitoneally twice weekly for 6 weeks. Atorvastatin was administered daily for 6 weeks. At study termination, liver samples were isolated from each animal and RNA was isolated for Northern blot qualitative assessment, DNA microarray and quantitative real-time PCR. Northern blot assessment and quantitative real-time PCR were performed as described herein.

Mouse apolipoprotein B mRNA expression, measured by real-time PCR, was evaluated to confirm antisense inhibition by ISIS 147764 and ISIS 147483. Serum cholesterol levels, measured by routine clinical analysis (for example, using an Olympus AU640e Chemistry Immuno Analyzer, Olympus, Melville, N.Y.) were also determined. Both apolipoprotein B mRNA and serum cholesterol levels were lowered in a dose-dependent manner following treatment with ISIS 147764 or ISIS 147483. The 50 mg/kg dose of ISIS 147483 increased ALT and AST levels. The 10, 25 and 50 mg/kg doses of ISIS 147764 and the 10 and 25 mg/kg doses of ISIS 147483 did not significantly elevate ALT or AST levels, indicating that the treatment did not result in toxicity.

DNA microarray analysis was performed using Affymetrix® gene expression analysis arrays, instruments and software tools, according to the manufacturer's instructions. Hybridization samples were prepared from 10 μg of total RNA isolated from each mouse liver according to the Affymetrix® Expression Analysis Technical Manual (Affymetrix, Inc., Santa Clara, Calif.). Samples were hybridized to a mouse gene chip containing approximately 22,000 genes (GENECHIP® Mouse Genome 430A 2.0 Array), which was subsequently washed and double-stained using the Fluidics Station 400 (Affymetrix, Inc., Santa Clara, Calif.) as defined by the manufacturer's protocol. Stained gene chips were scanned for probe cell intensity with the GENECHIP® Scanner (Affymetrix, Inc., Santa Clara, Calif.). Signal values for each probe set were calculated using the Affymetrix® Microarray Suite v5.0 software (Affymetrix, Inc., Santa Clara, Calif.). Each condition was profiled from 5 biological samples per group, one chip per sample. Fold change in expression was computed using the geometric mean of signal values as generated by Affymetrix® Microarray Suite v5.0. Statistical analysis utilized one-way ANOVA followed by 9 pair-wise comparisons. All groups were compared to the high fat group to determine gene expression changes resulting from ISIS 147764 and ISIS 147483 treatment. Fold changes in gene expression for genes on the chip are described in the tables provided in U.S. Provisional Application Ser. No. 60/568,825, which are herein incorporated by reference in their entirety: modified_GeneList_APOBOnly.xls, modified_GeneList_AtorOnly.xls, modified_AtorAPOB.xls, and modified_GeneList_NonSpecific.xls.

Microarray data was interpreted using hierarchical clustering and principal component analysis to visualize global gene expression patterns. Principal component analysis (PCA) involves a mathematical procedure that transforms a number of (possibly) correlated variables into a (smaller) number of uncorrelated variables called principal components. The first principal component accounts for as much of the variability in the data as possible, and each succeeding component accounts for as much of the remaining variability as possible. Hierarchical clustering is a multivariate technique useful in identifying distinct groups in the data, in such a way that objects belonging to the same cluster resemble each other, whereas objects in different clusters are dissimilar. Statistical analyses of the microarray data in the dose-dependence study are further described in U.S. Provisional Application Ser. No. 60/568,825, (“MicroArrayReport 7.pdf”), which is herein incorporated by reference in its entirety.

Both hierarchical clustering and PCA revealed that treatment with ISIS 147764 shifts the gene expression profile in high fat fed mice to the profile observed in lean mice. Thus, antisense inhibition of apolipoprotein B shifts a gene expression profile of an obese animal to that of a lean animal in a dose-dependent fashion.

### Example 71

Microarray Analysis: Evaluation of Time-Dependent Gene Expression Patterns in Lean Versus High-Fat Fed Mice

In a further embodiment, the effects of antisense inhibition of apolipoprotein B as a function of time were investigated using DNA microarray analysis. In this study, microarray analyses of liver gene expression patterns were performed following 48 hours, 1 week, 2 weeks and 4 weeks of treatment. Male C57Bl/6 mice were divided into the following groups: (1) mice on a lean diet, injected with saline (lean control); (2) mice on a high fat diet (high-fat fed); (3) mice on a high fat diet injected with 50 mg/kg of the control oligonucleotide 141923 (SEQ ID NO: 19); (4) mice on a high fat diet given 20 mg/kg atorvastatin calcium (Lipitor®, Pfizer Inc.); (5) mice on a high fat diet injected with 10, 25 or 50 mg/kg ISIS 147764 (SEQ ID NO: 17). Mice in the high-fat diet groups were maintained on a diet of 60% lard for 4 weeks prior to treatment. Saline and oligonucleotide treatments were administered intraperitoneally twice weekly throughout the treatment period. Atorvastatin was administered daily throughout the treatment period. Each individual dose, time and treatment group consisted of 8 animals. Animals were sacrificed and liver samples were procured after 48 hours, 1 week, 2 weeks or 4 weeks of treatment. RNA was isolated from liver tissue for Northern blot qualitative assessment, DNA microarray and quantitative real-time PCR. Northern blot assessment and quantitative real-time PCR were performed as described herein. DNA microarray analysis was performed as described for the 6 week dose-dependence study. All groups were compared to the high fat group to determine gene expression changes resulting from ISIS 147764 and ISIS 147883 treatment.

For the time-dependence study, fold changes in gene expression for genes on the chips are described in the table provided in U.S. Provisional Application Ser. No. 60/568,825, which is herein incorporated by reference in its entirety: modified_MGraham_TimeCourse.xls.

Statistical analyses were carried out as described for the dose-dependence study, and are further described in U.S. Provisional Application Ser. No. 60/568,825, (MicroArray Report 11.doc) which is herein incorporated by reference in its entirety.

Analysis of the microarray data from the time dependence study revealed that, as was observed in the dose-dependence study, the gene expression profile following treatment with ISIS 147764 shifts from that of high-fat fed mice to that of a lean mouse. Thus, antisense inhibition of apolipoprotein B shifts a gene expression profile of an obese animal to that of a lean animal in a time-dependent manner.

### Example 72

Gene Expression Changes Induced by Antisense Inhibition of Apolipoprotein B

Differentially expressed genes were classified according to gene family assignments in the Gene Ontology database. Comparison of the ISIS 147764-treated samples from the dose-dependence study with the ISIS 147764-treated samples from the time-dependence study revealed that many genes involved in metabolic processes were concurrently down-regulated as a function of both antisense oligonucleotide dose and length of treatment. Gene families with members down-regulated in a dose- and time-dependent manner are those of lipid metabolism, lipid biosynthesis, fatty acid biosynthesis, fatty acid binding proteins, phosphatidylcholine biosynthesis, steroid biosynthesis, lipid transport, glycogen synthesis, gluconeogenesis, complement activation, acute phase response, inflammatory response, pro-apoptosis and anti-apoptosis. Gene families with members upregulated in a dose and time-dependent manner following apolipoprotein B antisense inhibition included lipid metabolism, fatty acid biosynthesis, steroid biosynthesis, cholesterol metabolism, complement activation, acute phase response, inflammatory response, matrix metalloproteinases and pro-apoptosis. Some gene families, for example, lipid metabolism, contained both up- and down-regulated genes.

Gene expression changes for a subset of genes analyzed by DNA microarray in both the dose- and time-dependence studies are presented by gene family in Tables 53, 54, 55, 56, and 57. Gene names used are the official symbols from the National Center for Biotechnology Information (NCBI). GENBANK® accession numbers corresponding to gene symbols are provided in the tables in U.S. Provisional Application Ser. No. 60/568,825, which is herein incorporated by reference in its entirety. “Lean” indicates data from mice on a lean diet receiving saline treatment. “141923” indicates data from animals treated with the control oligonucleotide ISIS 141923. “ISIS 147764” indicates data from the high-fat fed mice treated with ISIS 147764 in the dose dependence study. “ISIS 147764 50 mg/kg” indicates data from the high-fat fed mice treated with ISIS 147765 in the time-dependence study. The data shown in this table represent the fold change of the indicated sample relative to samples from high-fat fed mice receiving saline treatment. For example, in high-fat fed mice receiving a 50 mg/kg dose of ISIS 147764 in the dose-dependence study, Lcat gene expression experienced a fold change of −1.29 relative to gene expression levels in high-fat fed mice receiving saline treatment in the same study, i.e. ISIS 147764 treatment reduce liver Lcat gene expression by 1.29 fold.

Fold changes less than or equal to −1.1 or greater than or equal to 1.1 (decrease or increase in gene expression level, respectively) that have a P-value of less than or equal to 0.05 are underscored. Fold changes with a P-value less than or equal to 0.05 are considered have the highest statistical significance. For example, the −1.29 fold reduction in Lcat gene expression is highly statistically significant. Fold changes less than or equal to −1.1 or greater than or equal to 1.1 that have a P-value of greater than 0.05 are presented in plain type. P-values for fold changes between −1.1 and 1.1 are not indicated.

The Mouse Genome 430A 2.0 Array used for these studies contains multiple probe sets for some genes. For these genes, results from each individual probe set are shown in Tables 53, 54, 55, 56, and 57. For example, in Table 53, Lip1 expression was measured by 2 probe sets, and the results from each probe set are shown in separate rows in the table.

TABLE 53
Lipid Biosynthesis and Metabolism Gene Changes

| | | 141923 | ISIS 147764 | | | ISIS 147764 (50 mg/kg) | | | |
|---|---|---|---|---|---|---|---|---|---|
| Gene | Lean | 50 mg/kg | 10 mg/kg | 25 mg/kg | 50 mg/kg | 48 hr | 1 week | 2 week | 4 week |
| Lcat | 1.04 | −1.12 | −1.21 | −1.33 | −1.29 | 1.11 | 1.12 | −1.21 | −1.15 |
| Lip1 | −1.01 | −1.06 | −1.25 | −1.41 | −1.19 | −1.36 | −1.03 | −1.25 | −2.04 |
| Lip1 | 1.55 | −1.15 | −1.63 | −1.49 | −1.25 | −1.09 | −1.28 | −1.3 | −1.6 |
| Lipc | 1.33 | −1.12 | −2.15 | −4.17 | −6.59 | −1.15 | −1.83 | −2.36 | −5.96 |
| Ppara | −2.04 | −1.08 | −1.09 | −1.09 | −1.42 | 1.01 | 1.02 | 1.12 | −1.37 |
| Pparg | −2.35 | −2.35 | −2.15 | −6.99 | −5.46 | −1.85 | 1.36 | 1.44 | −4.95 |
| Pcx | −1.14 | −1.23 | −1.24 | −1.44 | −1.5 | −1.04 | −1.22 | −1.17 | −1.77 |

TABLE 54
Cholesterol/Lipid Transport Gene Changes

| | | 141923 | ISIS 147764 | | | ISIS 147764 (50 mg/kg) | | | |
|---|---|---|---|---|---|---|---|---|---|
| Gene | Lean | 50 mg/kg | 10 mg/kg | 25 mg/kg | 50 mg/kg | 48 hr | 1 week | 2 week | 4 week |
| Apoa4 | −3.39 | −1.27 | −1.37 | −3.86 | −3.83 | −1.19 | −1.17 | −1.59 | −3.35 |
| Apoa4 | −2.48 | 1.03 | −1.04 | −2.99 | −2.68 | −1.26 | −1.17 | −1.38 | −3.13 |
| Apoc1 | −1.11 | −1.08 | −1.07 | −1.14 | −1.19 | 1.04 | 1.03 | −1.01 | −1.04 |
| Apoc2 | −1.49 | −1.17 | −1.18 | −1.39 | −1.35 | −1.19 | −1.16 | −1.15 | −1.28 |
| Apoc4 | −1.19 | 1.01 | 1.01 | −1.14 | −1.4 | 1 | −1.02 | −1 | −1.12 |
| Mttp | −1.34 | −1.33 | −1.12 | −1.12 | −1.03 | −1.13 | −1.01 | −1.11 | −1.05 |
| Mttp | −1.08 | −1.04 | −1.01 | −1.1 | −1.18 | −1.12 | 1.01 | 1.01 | −1.05 |

TABLE 55
Fatty Acid Biosynthesis/Binding Proteins Gene Changes

| | | | ISIS 147764 | | | ISIS 147764 (50 mg/kg) | | | |
|---|---|---|---|---|---|---|---|---|---|
| | | 141923 | 10 | 25 | 50 | 48 | 1 | 2 | 4 |
| Gene | Lean | 50 mg/kg | mg/kg | mg/kg | mg/kg | hr | week | week | week |
| Prkab1 | 1.29 | −1.13 | 1.04 | 1.25 | 1.29 | 1.13 | −1.04 | 1.03 | 1.08 |
| Prkag1 | −1.12 | 1.03 | −1.14 | 1.09 | 1.06 | −1.18 | −1.26 | 1.01 | 1.29 |
| Srebp-1 | −1.35 | −1.35 | −1.47 | −1.7 | −1.8 | −1.09 | −1.37 | −1.49 | −2.95 |
| Scd2 | 1.24 | 1.43 | 1.5 | 1.66 | 1.93 | 1.11 | 1.02 | −1.12 | 1.38 |
| Scd2 | 1.07 | −1.22 | 1.06 | 1.37 | 1.15 | −1.09 | 1.03 | 1.04 | 1.16 |
| Scd1 | 1.12 | −1.48 | −2.04 | −6.49 | −3.81 | −1.22 | −1.36 | −1.59 | −11.66 |
| Scd1 | −1.03 | −4.19 | −4.87 | −13.89 | −11.65 | −2.11 | −2.53 | −3.25 | −35.33 |
| Acadl | −1.05 | −1.1 | 1 | −1.11 | −1.28 | −1.01 | −1.02 | 1.14 | −1.23 |
| Acadm | −1.11 | −1.14 | 1.02 | −1.2 | −1.3 | 1.01 | 1.04 | 1.06 | −1.24 |
| Acads | −1.16 | 1.08 | −1.01 | −1.09 | −1.29 | −1.09 | 1.06 | 1.13 | −1.06 |
| Acox1 | −1.12 | −1.45 | −1.12 | −1.23 | −1.43 | 1.01 | −1.01 | 1.01 | −1.19 |
| Acox1 | −1.39 | −2.03 | −1.3 | −1.36 | −1.64 | 1 | −1.06 | 1.02 | −1.49 |
| Cpt1a | 1.37 | 1.43 | 1.31 | 1.06 | −1.74 | 1.07 | −1.33 | 1.06 | −1.11 |
| Cpt1a | −1.31 | −1.25 | −1.24 | −1.34 | −1.74 | −1.07 | −1.13 | −1.14 | −1.68 |
| Cpt1a | 1.03 | 1.22 | 1.11 | −1.08 | −1.59 | 1 | −1.24 | 1.02 | −1.9 |
| Cpt2 | −1.18 | −1.1 | −1.16 | −1.08 | −1.2 | 1.1 | 1.1 | 1.08 | −1.04 |
| Crat | −1.07 | −1.36 | −1.35 | −1.77 | −2.68 | −1.22 | −1.08 | −1.21 | −2.39 |
| Elovl2 | −1.2 | 1.01 | −1.34 | −1.38 | −2.5 | −1.12 | −1.34 | −1.38 | −2.53 |
| Elovl3 | −9.91 | 1.74 | −1.18 | −1.43 | −2.27 | −1.35 | −1.08 | −1.27 | −1.91 |
| Acadsb | −1.18 | 1.08 | −1.25 | −1.88 | −2.43 | −1.12 | −1.19 | −1.44 | −1.79 |
| Fads2 | −1.83 | −2.16 | −2.93 | −4.44 | −5.64 | −1.38 | −1.68 | −2.51 | −6.83 |
| Fasn | 1.17 | −1.2 | −1.05 | −1.96 | −1.35 | −1.18 | −1.11 | −1.37 | −3.14 |
| Facl2 | −1.3 | −1.41 | −1.31 | −1.4 | −1.65 | −1.16 | −1.26 | −1.36 | −1.44 |
| Facl2 | −1.3 | −1.23 | −1.19 | −1.27 | −1.69 | −1.08 | −1.25 | −1.16 | −1.2 |
| Facl4 | −1.5 | −1.07 | 1.23 | 1.59 | 1.71 | −1.05 | 1.19 | 1.26 | 1.9 |
| Abcd2 | 1.56 | −10.58 | −11.39 | −28.14 | −39.3 | −2.4 | −6.01 | −3.83 | −35.7 |
| Dbi | −1.15 | −1.11 | −1.15 | −1.26 | −1.5 | −1.09 | −1.12 | 1.09 | −1.2 |
| Dbi | −1.05 | −1.2 | −1.19 | −1.56 | −1.56 | −1.08 | −1.02 | −1.06 | −1.27 |
| Dbi | 1.04 | −1.16 | −1.14 | −1.29 | −1.48 | −1.21 | 1.02 | 1.08 | −1.15 |
| Dbi | −1.09 | −1.05 | −1.15 | −1.36 | −1.32 | −1.15 | −1.09 | 1.01 | −1.18 |
| Fabp1 | −1.16 | −1.12 | −1.11 | −1.11 | −1.46 | 1.26 | 1.11 | −1.02 | −1.04 |
| Fabp1 | −1.27 | −1.18 | −1.09 | −1.14 | −1.29 | 1.07 | 1.12 | 1.07 | 1.01 |
| Fabp2 | −3.46 | −1.2 | −1.82 | −3.88 | −4.87 | −1.48 | −1.76 | −1.4 | −4.74 |
| Fabp7 | −1.68 | 1.26 | 1.07 | −1.18 | 1.54 | 1.3 | 1.58 | 1.25 | 1.76 |

TABLE 56
Cholesterol Metabolism Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Acat-1 | −1.63 | −1.63 | −145 | −1.94 | −3.17 | −1.18 | −1.43 | −1.13 | −2.49 |
| Acat-1 | −1.39 | −1.29 | −1.22 | −1.49 | −4.49 | −1.12 | −1.15 | −1.08 | −1.75 |
| Acat-1 | −1.31 | −1.31 | −1.3 | −1.64 | −2.73 | −1.27 | −1.15 | −1.06 | −1.94 |
| Acca-1 | −1.12 | −1.2 | −1.11 | −1.16 | −1.31 | −1.11 | 1.06 | −1.09 | −1.46 |
| Cyp7a1 | 1.02 | −1.53 | −1.39 | −1.09 | −1.87 | 1.28 | 1.2 | −1.92 | −1.73 |
| Cyp7b1 | −4.68 | 2.52 | 1.57 | 2.02 | 1.4 | 1.06 | 1.42 | −1.01 | 2 |
| Cyp7b1 | −5.47 | 1.88 | 1.34 | 1.77 | 1.24 | −1.1 | 1.4 | −1.07 | 1.81 |
| Soat2 | 1.01 | −1.52 | 1.02 | 1.33 | 1.32 | 1.12 | 1.18 | 1.45 | 1.15 |
| Ldlr | 1.07 | 1.07 | −1.34 | −1.71 | −1.4 | −1.12 | −1.11 | −1.38 | −1.9 |
| Hmgcs1 | −1.01 | −1.01 | −1.29 | −2.06 | −1.66 | −1.01 | 1.31 | −1.06 | −2.21 |
| Hmgcs1 | 1.02 | 1.02 | −1.44 | −1.72 | −1.7 | −1.1 | 1.28 | −1.2 | −2.07 |
| Hmgcs1 | 1.05 | 1.05 | −1.39 | −1.78 | −1.56 | −1.13 | 1.24 | −1.16 | −1.84 |
| Hmgcs1 | −1.05 | −1.05 | −1.47 | −1.85 | −1.74 | −1.11 | 1.16 | −1.23 | −2.26 |
| Hmgcs2 | −1.31 | −1.31 | −1.07 | −1.23 | −1.61 | 1.03 | 1.17 | 1.13 | −1.39 |

TABLE 57
Glucose/Glycogen Synthesis Gene Changes

| | | 141923 | ISIS 147764 | | | ISIS 147764 (50 mg/kg) | | | |
|---|---|---|---|---|---|---|---|---|---|
| Gene | Lean | 50 mg/kg | 10 mg/kg | 25 mg/kg | 50 mg/kg | 48 hr | 1 week | 2 week | 4 week |
| Car5a | 1.03 | 1.01 | −1.15 | −1.18 | −1.47 | 1.04 | 1.01 | −1.06 | −1.4 |
| Gck | −2.74 | −2.74 | −2.01 | −3.39 | −11.23 | −1.28 | −1.4 | −1.78 | −7.34 |
| Gck | −1.65 | −1.65 | −1.45 | −1.93 | −3.64 | −1.12 | −1.03 | −1.53 | −3.16 |
| G6pc | −1.17 | −1 | −1.11 | −3.69 | −3.09 | −1.11 | 1.53 | −1.33 | −3.09 |

Real-time PCR analysis confirmed the reduction in mRNA expression for the following genes involved in lipid metabolism: ATP-binding cassette, sub-family D (ALD) member 2 (ABCD2), intestinal fatty acid binding protein 2 (FABP2), stearol CoA desaturase-1 (SCD1) and HMG CoA reductase (HMGCR). Probes and primers were designed to hybridize to these genes, using publicly available sequences. Probes and primers for real-time PCR can be designed using commercially available tools, for example, Primer Express® software (Applied Biosystems, Foster City, Calif.). Real-time PCR was performed as described herein, and results were normalized to GAPDH real-time PCR results. Results are presented in Table 58 and are normalized to mRNA levels from high-fat fed mice.

TABLE 58
Real-time PCR confirmation of gene expression changes
following antisene inhibition of apolipoprotein B in mice

| | | % Expression, normalized to | | | |
|---|---|---|---|---|---|
| | | high-fat diet, saline treated mice | | | |
| Diet | Treatment | ABCD2 | SCD1 | HMGCR | FABP2 |
| Lean | Saline | 193 | 64 | 117 | 28 |
| High Fat | 141923 | 32 | 43 | 131 | 109 |
| High Fat | 147764, 10 mg/kg | 52 | 25 | 109 | 66 |
| High Fat | 147764, 25 mg/kg | 5 | 4 | 102 | 32 |
| High Fat | 147764, 50 mg/kg | 7 | 3 | 207 | 22 |
| High Fat | 147483, 10 mg/kg | 42 | 27 | 91 | 71 |
| High Fat | 147483, 25 mg/kg | 70 | 19 | 135 | 74 |
| High Fat | 147483, 50 mg/kg | 71 | 29 | 163 | 96 |
| High Fat | Atorvastatin | 69 | 25 | 358 | 63 |

These results confirm the reduction in ABCD2, SCD1 and FABP2 gene expression as a result of inhibition of apolipoprotein B following treatment with ISIS 147764.

Real-time PCR analysis confirmed the reduction in mRNA expression for the following additional genes involved in lipid metabolism: hepatic lipase, fatty acid synthase, HMG-CoA synthase 2 (HMGCS2), diazepam binding inhibitor (DBI), fatty acid Coenzyme A ligase, long chain 2 (FACL2), fatty acid-Coenzyme A ligase, long chain 4 (FACL4), fatty acid synthase (FASN), glucose-6-phosphatase, catalytic subunit (G6PC), hydroxysteroid (17-beta) dehydrogenase 12 (HSD17b12), low density lipoprotein receptor (LDLr), microsomal triglyceride transfer protein (MTP or MTTP), pyruvate carboxylase (PCX), peroxisome proliferator activated receptor-gamma (PPAR-gamma), matrix metalloproteinase-12 (MMP-12), activating transcription factor 5 (ATF5) and Bcl2-associated X protein (BAX).

Together, these gene expression studies reveal that antisense inhibition of apolipoprotein B can modulate a number of downstream events in several different gene pathways. Treatment of high-fat fed mice with an antisense inhibitor of apolipoprotein B shifted the gene expression profile to resemble that of a mouse on a lean diet. Thus, antisense inhibitors of apolipoprotein B are candidate therapeutic agents for the treatment of conditions characterized by abnormal lipid metabolism, such as hyperlipidemia, or conditions that increase cardiovascular disease risk, such as obesity.

### Example 73

AMPK Activation Following Antisense Inhibition of Apolipoprotein B

Additional analyses of gene expression profiles from mice treated with antisense oligonucleotide targeted to apolipoprotein B revealed an increase in AMP-activated protein kinase (AMPK). AMPK is the downstream component of a kinase cascade that acts as a sensor for glucose and lipid metabolism. AMPK is a ubiquitous serine/threonine kinase activated in response to environmental or nutritional stress factors which deplete intracellular ATP levels, including heat shock, hypoxia, hypoglycemia and prolonged exercise. The result of AMPK activation is the inhibition of energy-consuming biosynthetic pathways, such as fatty acid and sterol synthesis, and activation of ATP-producing catabolic pathways, such as fatty acid oxidation. AMPK exists as a heterotrimer, comprising a catalytic alpha subunit and regulatory beta and gamma subunits. In mammals, each subunit is encoded by multiple genes: alpha 1, alpha 2, beta 1, beta 2, gamma 1, gamma 2 and gamma 3 (reviewed in Kahn, et al., Cell Metabolism, 2005, 1, 15-25).

The microarray analyses described herein revealed that AMPK beta 1 (gene symbol Prkab1) and gamma 1 (gene symbol Prkag1) regulatory subunits were increased following treatment with ISIS 147764. Real-time PCR analysis of liver samples from both the dose-dependence and time-dependence studies revealed that AMPK alpha 2 (gene symbol Prkaa2) expression was elevated as well. Relative to expression in high-fat fed mice treated with saline, AMPK alpha 2 expression was increased by 41%, 49%, and 87% in animals treated twice weekly with 10, 25 and 50 mg/kg ISIS 147754, respectively, whereas AMPK alpha 2 expression was elevated by 25% and 8% in lean, saline-treated and ISIS 141923-treated animals, respectively. AMPK alpha 2 was similarly increased at the end of the time-dependence study, at which time AMPK alpha 2 levels were 31% greater in mice treated with 50 mg/kg ISIS 147764 twice weekly, relative to high fat fed mice treated with saline. In an additional study, in which mice were treated with ISIS 147764 at a dose of 50 mg/kg per week, twice weekly, for a period of 3 months, AMPK alpha 1 liver protein levels were increased by 2.4 fold relative to saline-treated animals (as determined by routine western blotting). These data illustrate that the levels of AMPK subunits, including the catalytic alpha subunits, are increased as a result of antisense inhibition of apolipoprotein B.

The increase in AMPK subunits is gene expression profile change characteristic of a lean animal; this gene profile change provides an additional marker for assessing shifts in gene expression profile following antisense inhibition of apolipoprotein B. Activation of AMPK is known to inhibit energy-consuming biosynthetic pathways, such as fatty acid and sterol synthesis, and activate ATP-producing catabolic pathways, such as fatty acid oxidation. Metformin, a drug widely used for the treatment of type 2 diabetes that also has beneficial effects on circulating lipids linked to cardiovascular risk, activates AMPK activity in cultured hepatocytes and also increases AMPK alpha 2 activity in the skeletal muscle of subjects treated with metformin, (Zhou et al., J. Clin. Invest., 2001, 108, 1167-1173; Musi, et al., Diabetes, 2002, 51, 2074-2081). Therefore, antisense oligonucleotides targeted to apolipoprotein B are candidate therapeutic agents with application in the treatment of cardiovascular disease, such as hyperlipidemia, and metabolic disorders, such as type 2 diabetes.

### Example 74

Antisense Inhibition of Apolipoprotein B in Functional Assays

Functional assays are used to evaluate how gene expression affects cellular pathways and metabolic processes. In a further embodiment, a variety of functional assays were performed to investigate how apolipoprotein B participates in cell proliferation and survival, angiogenesis, adipocytes differentiation and the inflammatory response. Such assays can be used, by way of example, to determine the function of apolipoprotein B in different cellular pathways and metabolic processes and to identify new therapeutic areas where inhibition of apolipoprotein B can be beneficial.

The effects of antisense inhibition of apolipoprotein B on cellular pathways and metabolic processes were evaluated using ISIS 147788 (TTTCTGTTGCCACATTGCCC, SEQ ID NO: 20), which targets human apolipoprotein B and was designed using publicly available sequence (SEQ ID NO: 3). ISIS 147788 is a chimeric oligomeric compounds 20 nucleotides in length, composed of a central gap region consisting of 10 2′-deoxynucleotides, which is flanked on both sides (5′ and 3′ directions) by 5-nucleotide “wing” segments. The wings are composed of 2′-O-methoxylethyl nucleotides, or 2′-MOE nucleotides. The internucleoside (backbone) linkages are phosphorothioate throughout, and all cytidine residues are 5-methylcytidines.

Cell Proliferation and Survival

Cell cycle regulation is the basis for various cancer therapeutics. Unregulated cell proliferation is a characteristic of cancer cells, thus most current chemotherapy agents target dividing cells, for example, by blocking the synthesis of new DNA required for cell division. However, cells in healthy tissues are also affected by agents that modulate cell proliferation.

In some cases, a cell cycle inhibitor will cause apoptosis in cancer cells, but allow normal cells to undergo growth arrest and therefore remain unaffected (Blagosklonny, Bioessays, 1999, 21, 704-709; Chen et al., Cancer Res., 1997, 57, 2013-2019; Evan and Littlewood, Science, 1998, 281, 1317-1322; Lees and Weinberg, Proc. Natl. Acad. Sci. USA, 1999, 96, 4221-4223). An example of sensitization to anti-cancer agents is observed in cells that have reduced or absent expression of the tumor suppressor genes p53 (Bunz et al., Science, 1998, 282, 1497-1501; Bunz et al., J. Clin. Invest., 1999, 104, 263-269; Stewart et al., Cancer Res., 1999, 59, 3831-3837; Wahl et al., Nat. Med., 1996, 2, 72-79). However, cancer cells often escape apoptosis (Lowe and Lin, Carcinogenesis, 2000, 21, 485-495; Reed, Cancer J. Sci. Am., 1998, 4 Suppl 1, S8-14). Further disruption of cell cycle checkpoints in cancer cells can increase sensitivity to chemotherapy while allowing normal cells to take refuge in G1 and remain unaffected. Cell cycle assays are employed to identify genes, such as p53, whose inhibition will sensitize cells to anti-cancer agents.

Cell Cycle Assay

The effects of antisense inhibition of apolipoprotein B were examined in normal human mammary epithelial cells (HMECs) as well as two breast carcinoma cell lines, MCF7 and T47D. All of the cell lines are obtained from the American Type Culture Collection (Manassas, Va.). The latter two cell lines express similar genes but MCF7 cells express the tumor suppressor p53, while T47D cells are deficient in p53. MCF-7 and HMECs cells are routinely cultured in DMEM low glucose (Invitrogen Life Technologies, Carlsbad, Calif.) supplemented with 10% fetal bovine serum (Invitrogen Life Technologies, Carlsbad, Calif.). T47D cells were cultured in DMEM High glucose media (Invitrogen Life Technologies, Carlsbad, Calif.) supplemented with 10% fetal bovine serum. Cells were routinely passaged by trypsinization and dilution when they reached approximately 90% confluence. Cells were plated in 24-well plates at approximately 50,000-60,000 cells per well for HMEC cells, approximately 140,000 cells per well for MCF-7 and approximately 170,000 cells per well for T47D cells, and allowed to attach to wells overnight.

ISIS 147788 (SEQ ID NO: 20) was used to inhibit apolipoprotein B mRNA expression. An oligonucleotide with a randomized sequence, ISIS 29848 (NNNNNNNNNNNNNNNNNNNN; where N is A, T, C or G; herein incorporated as SEQ ID NO: 21) was used a negative control, a compound that does not modulate cell cycle progression. In addition, a positive control for the inhibition of cell proliferation was assayed. The positive control was ISIS 183881 (ATCCAAGTGCTACTGTAGTA; herein incorporated as SEQ ID NO: 22) targets kinesin-like 1 and served as a positive control for the inhibition of cell cycle progression. ISIS 29248 and ISIS 183881 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 200 nM of oligonucleotide and 6 μg/mL LIPOFECTIN®. Before adding to cells, the oligonucleotide, LIPOFECTIN® and OPTI-MEM® 1 were mixed thoroughly and incubated for 0.5 hrs. The medium was removed from the plates and the plates were tapped on sterile gauze. Each well containing T47D or MCF7 cells was washed with 150 μl of phosphate-buffered saline. Each well containing HMECs was washed with 150 μL of Hank's balanced salt solution. The wash buffer in each well was replaced with 100 μL of the oligonucleotide/OPTI-MEM® 1/LIPOFECTIN® cocktail. Control cells received LIPOFECTIN® only. The plates were incubated for approximately 4 hours at 37° C., after which the medium was removed and the plate was tapped on sterile gauze. 100 μl of full growth medium was added to each well. After approximately 72 hours, routine procedures were used to prepare cells for flow cytometry analysis and cells were stained with propidium iodide to generate a cell cycle profile using a flow cytometer. The cell cycle profile was analyzed with the MODFIT™ program (Verity Software House, Inc., Topsham Me.).

Fragmentation of nuclear DNA is a hallmark of apoptosis and produces an increase in cells with a hypodiploid DNA content, which are categorized as “subG1”. An increase in cells in G1 phase is indicative of a cell cycle arrest prior to entry into S phase; an increase in cells in S phase is indicative of cell cycle arrest during DNA synthesis; and an increase in cells in the G2/M phase is indicative of cell cycle arrest just prior to or during mitosis. Data are expressed as percentage of cells in each phase relative to the cell cycle profile of untreated control cells and are shown in Table 59. Values above or below 100% indicate an increase or decrease, respectively, in each cell cycle population. For example, following treatment of MCF7 cells with ISIS 147788, 109% of the cells were in G1 phase, relative to the untreated cells, demonstrating an increase of 9% in the G1 phase population and indicative of a cell cycle arrest prior to entry into S phase.

TABLE 59
Cell cycle profile of cells treated with oligomeric compounds
targeted to apolipoprotein B

| Cell | | | Sub G1 | G1 | S | |
|---|---|---|---|---|---|---|
| Type | Treatment | Target | Phase | Phase | Phase | G2/M |
| MCF7 | ISIS 147788 | apolipoprotein B | 158 | 109 | 88 | 98 |
| | ISIS 29848 | negative control | 130 | 104 | 94 | 98 |
| | ISIS 183881 | positive control | 57 | 126 | 108 | 51 |
| T47D | ISIS 147788 | apolipoprotein B | 140 | 107 | 92 | 90 |
| | ISIS 29848 | negative control | 111 | 105 | 113 | 74 |
| | ISIS 183881 | positive control | 39 | 120 | 133 | 52 |
| HMEC | ISIS 147788 | apolipoprotein B | 584 | 95 | 108 | 107 |
| | ISIS 29848 | negative control | 376 | 92 | 120 | 105 |
| | ISIS 183881 | positive control | 289 | 110 | 106 | 72 |

Treatment of MCF7 and T47D cells and HMECs with ISIS 147788 did not result in a significant arrest in cell cycle progression. SubG1 populations were increased by antisense inhibition of apolipoprotein B, indicating an increase in apopoptotic cells.

Caspase Assay

Programmed cell death, or apoptosis, is an important aspect of various biological processes, including normal cell turnover, as well as immune system and embryonic development. Apoptosis involves the activation of caspases, a family of intracellular proteases through which a cascade of events leads to the cleavage of a select set of proteins. The caspase family can be divided into two groups: the initiator caspases, such as caspase-8 and -9, and the executioner caspases, such as caspase-3, -6 and -7, which are activated by the initiator caspases. The caspase family contains at least 14 members, with differing substrate preferences (Thornberry and Lazebnik, Science, 1998, 281, 1312-1316). A caspase assay is utilized to identify genes whose inhibition selectively causes apoptosis in breast carcinoma cell lines, without affecting normal cells, and to identify genes whose inhibition results in cell death in the p53-deficient T47D cells, and not in the MCF7 cells which express p53 (Ross et al., Nat. Genet., 2000, 24, 227-235; Scherf et al., Nat. Genet., 2000, 24, 236-244). The chemotherapeutic drugs taxol, cisplatin, etoposide, gemcitabine, camptothecin, aphidicolin and 5-fluorouracil all have been shown to induce apoptosis in a caspase-dependent manner.

In a further embodiment, antisense inhibition of apolipoprotein B was examined in normal human mammary epithelial cells (HMECs) as well as two breast carcinoma cell lines, MCF7 and T47D. All cells were cultured as described for the cell cycle assay in 96-well plates with black sides and flat, transparent bottoms (Corning Incorporated, Corning, N.Y.). DMEM media, with and without phenol red, were obtained from Invitrogen Life Technologies (Carlsbad, Calif.). MEGM media, with and without phenol red, were obtained from Cambrex Bioscience (Walkersville, Md.).

ISIS 147788 (SEQ ID NO: 20) was used to inhibit apolipoprotein B mRNA expression. An oligonucleotide with a randomized sequence, ISIS 29848 (NNNNNNNNNNNNNNNNNNNN; where N is A, T, C or G; incorporated herein as SEQ ID NO: 21) was used as a negative control, a compound that does not effect caspase activity. As a positive control for caspase activation, an oligonucleotide targeted to human Jagged2 ISIS 148715 (TTGTCCCAGTCCCAGGCCTC; herein incorporated as SEQ ID NO: 23) or human Notch1 ISIS 226844 (GCCCTCCATGCTGGCACAGG; herein incorporated as SEQ ID NO: 24) was also assayed. Both of these genes are known to induce caspase activity, and subsequently apoptosis, when inhibited. ISIS 29248, ISIS 148715 and ISIS 226844 are all chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Cells were treated as described for the cell cycle assay with 200 nM oligonucleotide in 6 μg/mL LIPOFECTIN®. Caspase-3 activity was evaluated with a fluorometric HTS Caspase-3 assay (Catalog # HTS02; EMD Biosciences, San Diego, Calif.) that detects cleavage after aspartate residues in the peptide sequence (DEVD). The DEVD substrate is labeled with a fluorescent molecule, which exhibits a blue to green shift in fluorescence upon cleavage by caspase-3. Active caspase-3 in the oligonucleotide treated cells is measured by this assay according to the manufacturer's instructions. Approximately 48 hours following oligonucleotide treatment, 50 uL of assay buffer containing 10 μM dithiothreitol was added to each well, followed by addition 20 uL of the caspase-3 fluorescent substrate conjugate. Fluorescence in wells was immediately detected (excitation/emission 400/505 nm) using a fluorescent plate reader (SPECTRAMAX® GEMINI XS, Molecular Devices, Sunnyvale, Calif.). The plate was covered and incubated at 37° C. for and additional three hours, after which the fluorescence was again measured (excitation/emission 400/505 nm). The value at time zero was subtracted from the measurement obtained at 3 hours. The measurement obtained from the untreated control cells was designated as 100% activity.

The experiment was replicated in each of the 3 cell types, HMECs, T47D and MCF7 and the results are shown in Table 60. From these data, values for caspase activity above or below 100% are considered to indicate that the compound has the ability to stimulate or inhibit caspase activity, respectively. The data are shown as percent increase in fluorescence relative to untreated control values.

TABLE 60
Effects of antisense inhibition of apolipoprotein
B on apoptosis in the caspase assay

| | | | % Caspase activity relative to |
|---|---|---|---|
| Cell Type | Treatment | Target | untreated control |
| MCF7 | ISIS 147788 | apolipoprotein B | 253 |
| | ISIS 148715 | positive control | 463 |
| | ISIS 29848 | negative control | 118 |
| T47D | ISIS 147788 | apolipoprotein B | 91 |
| | ISIS 148715 | positive control | 950 |
| | ISIS 29848 | negative control | 81 |
| HMEC | ISIS 147788 | apolipoprotein B | 97 |
| | ISIS 148715 | positive control | 1418 |
| | ISIS 29848 | negative control | 69 |

These results demonstrate that ISIS 147788 causes a significant increase in apoptosis in MCF7 cells.

In a further embodiment, a similar caspase assay was performed to compare caspase-3 activity in T47D cells, which lack functional p53, to that in T47D cells engineered to harbor a functional p53 gene. T47D+p53 cells are T47D cells that have been transfected with and selected for maintenance of a plasmid that expresses a wildtype copy of the p53 gene (for example, pCMV-p53; Clontech, Palo Alto, Calif.), using standard laboratory procedures. The cells were treated with oligonucleotide as described for T47D cells and caspase-3 activity was measured after approximately 24 and 48 hours of treatment, as described herein. Untreated control cells served as the control to which data were normalized. The results are presented in Table 61. From these data, values for caspase activity above or below 100% are considered to indicate that the compound has the ability to stimulate or inhibit caspase activity, respectively. The data are shown as percent increase in fluorescence relative to untreated control values.

TABLE 61
Caspase activity in the presence and absence of p53, following
antisense inhibition of apolipoprotein B

| | | | | % Caspase |
|---|---|---|---|---|
| | | | | activity |
| | Time | | | relative to |
| | following | | | untreated |
| Cell Type | treatment | Treatment | Target | control |
| T47D | 24 hours | ISIS 147788 | apolipoprotein B | 94 |
| | | ISIS 148715 | positive control | 147 |
| | | ISIS 29848 | negative control | 106 |
| T47D + p53 | 24 hours | ISIS 147788 | apolipoprotein B | 101 |
| | | ISIS 148715 | positive control | 172 |
| | | ISIS 29848 | negative control | 120 |
| T47D | 48 hours | ISIS 147788 | apolipoprotein B | 167 |
| | | ISIS 148715 | positive control | 143 |
| | | ISIS 29848 | negative control | 74 |
| T47D + p53 | 48 hours | ISIS 147788 | apolipoprotein B | 110 |
| | | ISIS 148715 | positive control | 218 |
| | | ISIS 29848 | negative control | 111 |

From these data it is evident that inhibition of apolipoprotein B expression by ISIS 147788 for 48 hours resulted in a significant induction of apoptosis T47D cells without p53, compared to untreated control cells controls, whereas apoptosis was neither induced nor inhibited in cells with functional p53.

These data demonstrate that, in the absence of a wild-type p53 gene, antisense inhibition of apolipoprotein B in T47D cells leads to a greater apoptotic cell fraction than in the presence of functional p53. Thus, the reintroduction of p53 into T47D cells resulted in decreased sensitivity of the cells to antisense inhibition of apolipoprotein B. Therefore, the inhibition of apolipoprotein B expression can be used to selectively modulate the growth of p53-deficient cells, such as cancer cells.

Angiogenesis Assays

Angiogenesis is the growth of new blood vessels (veins and arteries) by endothelial cells. This process is important in the development of a number of human diseases, and is believed to be particularly important in regulating the growth of solid tumors. Without new vessel formation it is believed that tumors will not grow beyond a few millimeters in size. In addition to their use as anti-cancer agents, inhibitors of angiogenesis have potential for the treatment of diabetic retinopathy, cardiovascular disease, rheumatoid arthritis and psoriasis (Carmeliet and Jain, Nature, 2000, 407, 249-257; Freedman and Isner, J. Mol. Cell. Cardiol., 2001, 33, 379-393; Jackson et al., Faseb J., 1997, 11, 457-465; Saaristo et al., Oncogene, 2000, 19, 6122-6129; Weber and De Bandt, Joint Bone Spine, 2000, 67, 366-383; Yoshida et al., Histol. Histopathol., 1999, 14, 1287-1294).

Endothelial Tube Formation Assay as a Measure of Angiogenesis

Angiogenesis is stimulated by numerous factors that promote interaction of endothelial cells with each other and with extracellular matrix molecules, resulting in the formation of capillary tubes. This morphogenic process is necessary for the delivery of oxygen to nearby tissues and plays an essential role in embryonic development, wound healing, and tumor growth (Carmeliet and Jain, Nature, 2000, 407, 249-257). Moreover, this process can be reproduced in a tissue culture assay that evaluated the formation of tube-like structures by endothelial cells. There are several different variations of the assay that use different matrices, such as collagen I (Kanayasu et al., Lipids, 1991, 26, 271-276), Matrigel (Yamagishi et al., J. Biol. Chem., 1997, 272, 8723-8730) and fibrin (Bach et al., Exp. Cell Res., 1998, 238, 324-334), as growth substrates for the cells. In this assay, HUVECs are plated on a matrix derived from the Engelbreth-Holm-Swarm mouse tumor, which is very similar to Matrigel (Kleinman et al., Biochemistry, 1986, 25, 312-318; Madri and Pratt, J. Histochem. Cytochem., 1986, 34, 85-91). Untreated HUVECs form tube-like structures when grown on this substrate. Loss of tube formation in vitro has been correlated with the inhibition of angiogenesis in vivo (Carmeliet and Jain, Nature, 2000, 407, 249-257; Zhang et al., Cancer Res., 2002, 62, 2034-2042), which supports the use of in vitro tube formation as an endpoint for angiogenesis.

In a further embodiment, primary human umbilical vein endothelial cells (HuVECs) were used to measure the effects of antisense inhibition of apolipoprotein B on tube formation activity. HuVECs were routinely cultured in EGM® (Clonetics Corporation, Walkersville, Md.) supplemented with SINGLEQUOTS® supplements (Clonetics Corporation, Walkersville, Md.). Cells were routinely passaged by trypsinization and dilution when they reached approximately 90% confluence and were maintained for up to 15 passages. HuVECs are plated at approximately 3000 cells/well in 96-well plates. One day later, cells are transfected with antisense oligonucleotides. The tube formation assay is performed using an in vitro Angiogenesis Assay Kit (Chemicon International, Temecula, Calif.).

HUVECs were treated with ISIS 147788 (SEQ ID NO: 20) to inhibit apolipoprotein B expression. An oligonucleotide with a randomized sequence, ISIS 29848 (NNNNNNNNNNNNNNNNNNNN; where N is A, T, C or G; herein incorporated as SEQ ID NO: 21) served as a negative control, a compound that does not affect tube formation. ISIS 25237 (GCCCATTGCTGGACATGC, SEQ ID NO: 25), an oligomeric compound targeted to integrin P3 (ISIS 25237) known to inhibit angiogenesis, was used as a positive control. ISIS 25237 is a chimeric oligonucleotide (“gapmers”) 18 nucleotides in length, composed of a central “gap” region consisting of ten 2′-deoxynucleotides, which is flanked on both sides (5′ and 3′ directions) by four-nucleotide “wings”. The wings are composed of 2′-O-methoxyethyl (2′-MOE) nucleotides. The internucleoside (backbone) linkages are phosphorothioate (P═S) throughout the oligonucleotides. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 75 nM of oligonucleotide and 2.25 μg/mL LIPOFECTIN®. Before adding to cells, the oligonucleotide, LIPOFECTIN® and OPTI-MEM® 1 were mixed thoroughly and incubated for 0.5 hrs. Untreated control cells received LIPOFECTIN® only. The medium was removed from the plates and the plates were tapped on sterile gauze. Each well was washed in 150 μl of phosphate-buffered saline. The wash buffer in each well was replaced with 100 μL of the oligonucleotide/OPTI-MEM® 1/LIPOFECTIN® cocktail. ISIS 147788 was tested in triplicate, and the ISIS 29848 was tested in up to six replicates. The plates were incubated for approximately 4 hours at 37° C., after which the medium was removed and the plate was tapped on sterile gauze. 100 μl of full growth medium was added to each well. Approximately 50 hours after transfection, cells are transferred to 96-well plates coated with ECMATRIX® (Chemicon Inter-national). Under these conditions, untreated HUVECs form tube-like structures. After an overnight incubation at 37° C., treated and untreated cells are inspected by light microscopy. Individual wells are assigned discrete scores from 1 to 5 depending on the extent of tube formation. A score of 1 refers to a well with no tube formation while a score of 5 is given to wells where all cells are forming an extensive tubular network. Results are expressed relative to untreated control samples. Following treatment with ISIS 147788, ISIS 25237 and ISIS 29848, tube formation was 100%, 40% and 100% relative to tube formation in untreated control samples. ISIS 147788 did not significantly inhibit tube formation by HUVECs.

Matrix Metalloproteinase Activity

In a further embodiment, the antisense inhibition of apolipoprotein B was evaluated for effects on MMP activity in the media above human umbilical-vein endothelial cells (HUVECs). MMP activity was measured using the ENZCHEK® Gelatinase/Collagenase Assay Kit (Molecular Probes, Eugene, Oreg.). HUVECs are cultured as described for the tube formation assay. HUVECs are plated at approximately 4000 cells per well in 96-well plates and transfected one day later.

HUVECs were treated with ISIS 147788 (SEQ ID NO: 20) to inhibit apolipoprotein B mRNA expression. An oligonucleotide with a randomized sequence, ISIS 29848 (NNNNNNNNNNNNNNNNNNNN; where N is A, T, C or G; herein incorporated as SEQ ID NO: 21) served as a negative control, or a treatment not expected to affect MMP activity. ISIS 25237 (GCCCATTGCTGGACATGC, SEQ ID NO: 25) targets integrin beta 3 and was used as a positive control for the inhibition of MMP activity.

Cells were treated as described for the tube formation assay, with 75 nM of oligonucleotide and 2.25 μg/mL LIPOFECTIN®. ISIS 147788 and ISIS 25237 were tested in triplicate, and the ISIS 29848 was tested in up to six replicates. The plates were incubated for approximately 4 hours at 37° C., after which the medium was removed and the plate was tapped on sterile gauze. 100 μl of full growth medium was added to each well. Approximately 50 hours after transfection, a p-aminophenylmercuric acetate (APMA, Sigma-Aldrich, St. Louis, Mo.) solution is added to each well of a Corning-Costar 96-well clear bottom plate (VWR International, Brisbane, Calif.). The APMA solution is used to promote cleavage of inactive MMP precursor proteins. Media above the HUVECs is then transferred to the wells in the 96-well plate. After 30 minutes, the quenched, fluorogenic MMP cleavage substrate is added, and baseline fluorescence is read immediately at 485 nm excitation/530 nm emission. Following an overnight incubation at 37° C. in the dark, plates are read again to determine the amount of fluorescence, which corresponds to MMP activity. Total protein from HUVEC lysates is used to normalize the readings, and MMP activities are expressed as a percent relative to MMP activity from untreated control cells that did not receive oligonucleotide treatment. MMP activities were 39%, 49% and 84% in the culture media from cells treated with ISIS 147788, ISIS 25237 and ISIS 29848. These data reveal that ISIS 147788, like the positive control 25237, can inhibit MMP activity and is a candidate therapeutic agent for the inhibition of angiogenesis where such activity is desired, for example, in the treatment of cancer, diabetic retinopathy, cardiovascular disease, rheumatoid arthritis and psoriasis.

Metabolism Assays

Insulin is an essential signaling molecule throughout the body, but its major target organs are the liver, skeletal muscle and adipose tissue. Insulin is the primary modulator of glucose homeostasis and helps maintain a balance of peripheral glucose utilization and hepatic glucose production. The reduced ability of normal circulating concentrations of insulin to maintain glucose homeostasis manifests in insulin resistance which is often associated with diabetes, central obesity, hypertension, polycystic ovarian syndrome, dyslipidemia and atherosclerosis (Saltiel, Cell, 2001, 104, 517-529; Saltiel and Kahn, Nature, 2001, 414, 799-806).

Response of Undifferentiated Adipocytes to Insulin

Insulin promotes the differentiation of preadipocytes into adipocytes. The condition of obesity, which results in increases in fat cell number, occurs even in insulin-resistant states in which glucose transport is impaired due to the antilipolytic effect of insulin. Inhibition of triglyceride breakdown requires much lower insulin concentrations than stimulation of glucose transport, resulting in maintenance or expansion of adipose stores (Kitamura et al., Mol. Cell. Biol., 1999, 19, 6286-6296; Kitamura et al., Mol. Cell. Biol., 1998, 18, 3708-3717).

One of the hallmarks of cellular differentiation is the upregulation of gene expression. During adipocyte differentiation, the gene expression patterns in adipocytes change considerably. Some genes known to be upregulated during adipocyte differentiation include hormone-sensitive lipase (HSL), adipocyte lipid binding protein (aP2), glucose transporter 4 (Glut4), and peroxisome proliferator-activated receptor gamma (PPAR-γ). Insulin signaling is improved by compounds that bind and inactivate PPAR-γ, a key regulator of adipocyte differentiation (Olefsky, J. Clin. Invest., 2000, 106, 467-472). Insulin induces the translocation of GLUT4 to the adipocyte cell surface, where it transports glucose into the cell, an activity necessary for triglyceride synthesis. In all forms of obesity and diabetes, a major factor contributing to the impaired insulin-stimulated glucose transport in adipocytes is the downregulation of GLUT4. Insulin also induces hormone sensitive lipase (HSL), which is the predominant lipase in adipocytes that functions to promote fatty acid synthesis and lipogenesis (Fredrikson et al., J. Biol. Chem., 1981, 256, 6311-6320). Adipocyte fatty acid binding protein (aP2) belongs to a multi-gene family of fatty acid and retinoid transport proteins. aP2 is postulated to serve as a lipid shuttle, solubilizing hydrophobic fatty acids and delivering them to the appropriate metabolic system for utilization (Fu et al., J. Lipid Res., 2000, 41, 2017-2023; Pelton et al., Biochem. Biophys. Res. Commun., 1999, 261, 456-458). Together, these genes play important roles in the uptake of glucose and the metabolism and utilization of fats.

Leptin secretion and an increase in triglyceride content are also well-established markers of adipocyte differentiation. While it serves as a marker for differentiated adipocytes, leptin also regulates glucose homeostasis through mechanisms (autocrine, paracrine, endocrine and neural) independent of the adipocyte's role in energy storage and release. As adipocytes differentiate, insulin increases triglyceride accumulation by both promoting triglyceride synthesis and inhibiting triglyceride breakdown (Spiegelman and Flier, Cell, 2001, 104, 531-543). As triglyceride accumulation correlates tightly with cell size and cell number, it is an excellent indicator of differentiated adipocytes.

The effects of antisense inhibition of apolipoprotein B on the expression of markers of cellular differentiation were examined in preadipocytes. Human white preadipocytes (Zen-Bio Inc., Research Triangle Park, N.C.) were grown in preadipocyte media (ZenBio Inc., Research Triangle Park, N.C.). One day before transfection, 96-well plates were seeded with approximately 3000 cells/well.

Cells were treated with ISIS 147788 (SEQ ID NO: 20) to inhibit apolipoprotein B expression. An oligonucleotide with a randomized sequence, ISIS 29848 (NNNNNNNNNNNNNNNNNNNN; where N is A, T, C or G; herein incorporated as SEQ ID NO: 25) was used a negative control, a compound that does not modulate adipocyte differentiation. Tumor necrosis factor alpha (TNF-α), which inhibits adipocyte differentiation, was used as a positive control for the inhibition of adipocyte differentiation as evaluated by leptin secretion. For all other parameters measured, ISIS 105990 (AGCAAAAGATCAATCCGTTA, incorporated herein as SEQ ID NO: 26), an inhibitor of PPAR-γ, served as a positive control for the inhibition of adipocyte differentiation. ISIS 29848 and ISIS 105990 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 250 nM of oligonucleotide and 6.5 μg/mL LIPOFECTIN®. Before adding to cells, the oligonucleotide, LIPOFECTIN® and OPTI-MEM® 1 were mixed thoroughly and incubated for 0.5 hrs. Untreated control cells received LIPOFECTIN® only. The medium was removed from the plates and the plates were tapped on sterile gauze. Each well was washed in 150 μl of phosphate-buffered saline. The wash buffer in each well was replaced with 100 μL of the oligonucleotide/OPTI-MEM®/LIPOFECTIN® cocktail. Compounds of the invention and ISIS 105990 were tested in triplicate, ISIS 29848 was tested in up to six replicate wells. The plates were incubated for approximately 4 hours at 37° C., after which the medium was removed and the plate was tapped on sterile gauze. 100 μl of full growth medium was added to each well. After the cells have reached confluence (approximately three days), they were exposed for three days to differentiation media (Zen-Bio, Inc.) containing a PPAR-γ agonist, IBMX, dexamethasone, and insulin. Cells were then fed adipocyte media (Zen-Bio, Inc.), which was replaced at 2 to 3 day intervals.

Leptin secretion into the media in which adipocytes are cultured was measured by protein ELISA. On day nine post-transfection, 96-well plates were coated with a monoclonal antibody to human leptin (R&D Systems, Minneapolis, Minn.) and left at 4° C. overnight. The plates were blocked with bovine serum albumin (BSA), and a dilution of the treated adipocyte media was incubated in the plate at room temperature for approximately 2 hours. After washing to remove unbound components, a second monoclonal antibody to human leptin (conjugated with biotin) was added. The plate was then incubated with strepavidin-conjugated horse radish peroxidase (HRP) and enzyme levels were determined by incubation with 3,3′,5,5′-tetramethylbenzidine, which turns blue when cleaved by HRP. The OD₄₅₀ was read for each well, where the dye absorbance is proportional to the leptin concentration in the cell lysate. Results, shown in Table 58, are expressed as a percent control relative to untreated control samples. With respect to leptin secretion, values above or below 100% are considered to indicate that the compound has the ability to stimulate or inhibit leptin secretion, respectively.

The triglyceride accumulation assay measures the synthesis of triglyceride by adipocytes. Triglyceride accumulation is measured using the INFINITY® Triglyceride reagent kit (Sigma-Aldrich, St. Louis, Mo.). On day nine post-transfection, cells are washed and lysed at room temperature, and the triglyceride assay reagent is added. Triglyceride accumulation is measured based on the amount of glycerol liberated from triglycerides by the enzyme lipoprotein lipase. Liberated glycerol is phosphorylated by glycerol kinase, and hydrogen peroxide is generated during the oxidation of glycerol-1-phosphate to dihydroxyacetone phosphate by glycerol phosphate oxidase. Horseradish peroxidase (HRP) uses H₂O₂ to oxidize 4-aminoantipyrine and 3,5 dichloro-2-hydroxybenzene sulfonate to produce a red-colored dye. Dye absorbance, which is proportional to the concentration of glycerol, is measured at 515 nm using an UV spectrophotometer. Glycerol concentration is calculated from a standard curve for each assay, and data are normalized to total cellular protein as determined by a Bradford assay (Bio-Rad Laboratories, Hercules, Calif.).

Expression of the four hallmark genes, HSL, aP2, Glut4, and PPARγ, was also measured in adipocytes transfected with compounds of the invention. Cells were lysed on day nine post-transfection, in a guanidinium-containing buffer and total RNA is harvested. The amount of total RNA in each sample was determined using a RIBOGREEN® Assay (Invitrogen Life Technologies, Carlsbad, Calif.). Real-time PCR was performed on the total RNA using primer/probe sets for the adipocyte differentiation hallmark genes Glut4, HSL, aP2, and PPAR-γ. mRNA levels, shown in Table 62, are expressed as percent control relative to the untreated control values. With respect to the four adipocyte differentiation hallmark genes, values above or below 100% are considered to indicate that the compound has the ability to stimulate or inhibit adipocyte differentiation, respectively.

TABLE 62
Effects of antisense inhibition of Apolipoprotein B on
adipocyte differentiation

| Treatment | Target | Leptin | aP2 | Glut4 | HSL | PPARγ |
|---|---|---|---|---|---|---|
| ISIS 147788 | apolipoprotein B | 137 | 99 | 101 | 74 | 149 |
| ISIS 29848 | negative control | 106 | 95 | 85 | 75 | 96 |
| ISIS 105990 | positive control | N.D. | 55 | 58 | 49 | 38 |
| TNF-alpha | positive control | 30 | N.D. | N.D. | N.D. | N.D. |

ISIS 147788 resulted in an increase in leptin secretion, indicating that this compound is potentially useful for the treatment of obesity. PPAR-γ mRNA expression was also increased.

Inflammation Assays

Inflammation assays are designed to identify genes that regulate the activation and effector phases of the adaptive immune response. During the activation phase, T lymphocytes (also known as T-cells) receiving signals from the appropriate antigens undergo clonal expansion, secrete cytokines, and upregulate their receptors for soluble growth factors, cytokines and co-stimulatory molecules (Cantrell, Annu. Rev. Immunol., 1996, 14, 259-274). These changes drive T-cell differentiation and effector function. In the effector phase, response to cytokines by non-immune effector cells controls the production of inflammatory mediators that can do extensive damage to host tissues. The cells of the adaptive immune systems, their products, as well as their interactions with various enzyme cascades involved in inflammation (e.g., the complement, clotting, fibrinolytic and kinin cascades) all represent potential points for intervention in inflammatory disease. The inflammation assay presented here measures hallmarks of the activation phase of the immune response.

Dendritic cells treated with antisense compounds are used to identify regulators of dendritic cell-mediated T-cell costimulation. The level of interleukin-2 (IL-2) production by T-cells, a critical consequence of T-cell activation (DeSilva et al., J. Immunol., 1991, 147, 3261-3267; Salomon and Bluestone, Annu. Rev. Immunol., 2001, 19, 225-252), is used as an endpoint for T-cell activation. T lymphocytes are important immunoregulatory cells that mediate pathological inflammatory responses. Optimal activation of T lymphocytes requires both primary antigen recognition events as well as secondary or costimulatory signals from antigen presenting cells (APC). Dendritic cells are the most efficient APCs known and are principally responsible for antigen presentation to T-cells, expression of high levels of costimulatory molecules during infection and disease, and the induction and maintenance of immunological memory (Banchereau and Steinman, Nature, 1998, 392, 245-252). While a number of costimulatory ligand-receptor pairs have been shown to influence T-cell activation, a principal signal is delivered by engagement of CD28 on T-cells by CD80 (B7-1) and CD86 (B7-2) on APCs (Boussiotis et al., Curr. Opin. Immunol., 1994, 6, 797-807; Lenschow et al., Annu. Rev. Immunol., 1996, 14, 233-258). Inhibition of T-cell co-stimulation by APCs holds promise for novel and more specific strategies of immune suppression. In addition, blocking costimulatory signals may lead to the development of long-term immunological anergy (unresponsiveness or tolerance) that would offer utility for promoting transplantation or dampening autoimmunity. T-cell anergy is the direct consequence of failure of T-cells to produce the growth factor IL-2 (DeSilva et al., J. Immunol., 1991, 147, 3261-3267; Salomon and Bluestone, Annu. Rev. Immunol., 2001, 19, 225-252).

Dendritic Cell Cytokine Production as a Measure of the Activation Phase of the Immune Response

In a further embodiment of the present invention, the effect of ISIS 147788 (SEQ ID NO: 20) was examined on the dendritic cell-mediated costimulation of T-cells. Dendritic cells (DCs, Clonetics Corp., San Diego, Calif.) were plated at approximately 6500 cells/well on anti-CD3 (UCHT1, Pharmingen-BD, San Diego, Calif.) coated 96-well plates in 500 U/mL granulocyte macrophase-colony stimulation factor (GM-CSF) and interleukin-4 (IL-4). DCs were treated with antisense compounds approximately 24 hours after plating.

Cells were treated with ISIS 147788 (SEQ ID NO: 20) to inhibit apolipoprotein B expression. An oligonucleotide with a randomized sequence, ISIS 29848 (NNNNNNNNNNNNNNNNNNNN; where N is A, T, C or G; herein incorporated as SEQ ID NO: 21) served as a negative control, a compound that does not affect dendritic cell-mediated T-cell costimulation. ISIS 113131 (CGTGTGTCTGTGCTAGTCCC, incorporated herein as SEQ ID NO: 27), an inhibitor of CD86, served as a positive control for the inhibition of dendritic cell-mediated T-cell costimulation. ISIS 29848 and ISIS 113131 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 200 nM of oligonucleotide and 6 μg/mL LIPOFECTIN®. Before adding to cells, the oligonucleotide, LIPOFECTIN® and OPTI-MEM® 1 were mixed thoroughly and incubated for 0.5 hrs. The medium was removed from the cells and the plates were tapped on sterile gauze. Each well was washed in 150 μl of phosphate-buffered saline. The wash buffer in each well was replaced with 100 μL of the oligonucleotide/OPTI-MEM® 1/LIPOFECTIN® cocktail. Untreated control cells received LIPOFECTIN® only. ISIS 147788 and ISIS 113131 were tested in triplicate, and the negative control oligonucleotide was tested in up to six replicates. The plates were incubated with oligonucleotide for approximately 4 hours at 37° C., after which the medium was removed and the plate was tapped on sterile gauze. Fresh growth media plus cytokines was added and DC culture was continued for an additional 48 hours. DCs are then co-cultured with Jurkat T-cells in RPMI medium (Invitrogen Life Technologies, Carlsbad, Calif.) supplemented with 10% heat-inactivated fetal bovine serum (Sigma Chemical Company, St. Louis, Mo.). Culture supernatants are collected approximately 24 hours later and assayed for IL-2 levels (IL-2 DuoSet, R&D Systems, Minneapolis, Minn.), which are expressed as a percent relative to untreated control samples. A value greater than 100% indicates an induction of the inflammatory response, whereas a value less than 100% demonstrates a reduction in the inflammatory response.

The culture supernatant of cells treated with ISIS 147788, ISIS 113131 and ISIS 29848 contained IL-2 at 51%, 50% and 91% of the IL-2 concentration found in culture supernatant from untreated control cells, respectively. These results indicate that ISIS 147788 inhibited T-cell co-stimulation and reduced the inflammatory response. As such, antisense oligonucleotides targeting apolipoprotein B are candidate therapeutic compounds with applications in the prevention, treatment or attenuation of conditions associated with hyperstimulation of the immune system, including rheumatoid arthritis, irritable bowel disease, athsma, lupus and multiple sclerosis.

### Example 75

Compounds Useful for the Improvement of Cardiovascular Risk Profiles

Research from experimental animals, laboratory investigations, epidemiology, and genetic forms of hypercholesterolemia indicate that elevated LDL cholesterol (LDL-C) is a major cause of coronary heart disease (CHD). In addition, recent clinical trials robustly show that LDL-lowering therapy reduces risk for CHD. For these reasons, the NCEP Adult Treatment Panel III (ATP III) guidelines identify elevated LDL-cholesterol as the primary target of cholesterol-lowering therapy. Despite the availability of lipid-lowering therapeutic agents, only approximately 20% of high-risk patients with coronary heart disease attain the aggressive LDL-cholesterol levels recommended by the United States National Cholesterol Education Program (NCEP) Guidelines (Adult Treatment Panel III, Circulation, 2002, 106, 3143-3421). Thus, there exists a need for additional safe and effective lipid-lowering agents.

Antisense inhibition of apolipoprotein B reduces liver and serum apolipoprotein B and lowers serum LDL-cholesterol, as evidenced by studies in multiple animal models (as described in U.S. patent application Ser. No. 10/712,795, which is herein incorporated by reference in its entirety). Thus, antisense inhibition of apolipoprotein B accomplishes the cholesterol-lowering effects suggested by the NCEP. Furthermore, as described herein, antisense inhibition of apolipoprotein B shifts the gene expression profile of a high-fat fed mouse from that of an obese animal to that of a lean animal. This shift in gene expression profile provides a means for the identification of antisense compounds, including those targeted to apolipoprotein B, that are candidate lipid-lowering agents. Compounds that shift gene expression patterns from high-fat fed profiles to lean profiles are candidate therapeutic agents for the treatment of conditions such as cardiovascular disease and hyperlipidemia.

### Example 76

Design and Screening of Duplexed Oligomeric Compounds Targeting Apolipoprotein B

In a further embodiment, a series of duplexes, including dsRNA (or siRNAs) and mimetics thereof, comprising oligomeric compounds targeted to apolipoprotein B and their complements can be designed. The nucleobase sequence of the antisense strand of the duplex comprises at least a portion of an oligonucleotide targeted to apolipoprotein B. The ends of the strands may be modified by the addition of one or more natural or modified nucleobases to form an overhang. The sense strand of the nucleic acid duplex is then designed and synthesized as the complement of the antisense strand and may also contain modifications or additions to either terminus. The antisense and sense strands of the duplex comprise from about 17 to 25 nucleotides, or from about 19 to 23 nucleotides. Alternatively, the antisense and sense strands comprise 20, 21 or 22 nucleotides.

In one embodiment, a duplex comprising an antisense strand having the sequence CGAGAGGCGGACGGGACCG (SEQ ID NO: 28), can be prepared with blunt ends (no single stranded overhang) as shown:

| | |
|---|---|
| cgagaggcggacgggaccg | Antisense Strand (SEQ ID NO: 28) |
| \|\|\|\|\|\|\|\|\|\|\|\|\|\|\|\|\|\|\| | |
| gctctccgcctgccctggc | Complement (SEQ ID NO: 29) |

In another embodiment, both strands of the dsRNA duplex would be complementary over the central nucleobases, each having overhangs at one or both termini. For example, a duplex comprising an antisense strand having the sequence CGAGAGGCGGACGGGACCG (SEQ ID NO: 28) and having a two-nucleobase overhang of deoxythymidine(dT) would have the following structure:

| | |
|---|---|
| cgagaggcggacgggaccgTT | Antisense Strand (SEQ ID NO: 30) |
| \|\|\|\|\|\|\|\|\|\|\|\|\|\|\|\|\|\|\| | |
| TTgctctccgcctgccctggc | Complement (SEQ ID NO: 31) |

Overhangs can range from 2 to 6 nucleobases and these nucleobases may or may not be complementary to the target nucleic acid. In another embodiment, the duplexes can have an overhang on only one terminus.

The RNA duplex can be unimolecular or bimolecular; i.e., the two strands can be part of a single molecule or may be separate molecules.

RNA strands of the duplex can be synthesized by methods routine to the skilled artisan or purchased from Dharmacon Research Inc. (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM.

Once prepared, the duplexed compounds are evaluated for their ability to modulate apolipoprotein B. When cells reach approximately 80% confluency, they are treated with duplexed compounds of the invention. For cells grown in 96-well plates, wells are washed once with 200 μL OPTI-MEM® 1 reduced-serum medium (Invitrogen Life Technologies, Carlsbad, Calif.) and then treated with 130 μL of OPTI-MEM® 1 containing 12 μg/mL LIPOFECTIN® (Invitrogen Life Technologies, Carlsbad, Calif.) and the desired duplex antisense compound (e.g. 200 nM) at a ratio of 6 μg/mL LIPOFECTIN® per 100 nM duplex antisense compound. After approximately 5 hours of treatment, the medium is replaced with fresh medium. Cells are harvested approximately 16 hours after treatment, at which time RNA is isolated and target reduction measured by real-time PCR.

What is claimed is:

1. A compound 8 to 50 nucleobases in length targeted to a nucleic acid molecule encoding apolipoprotein B, wherein said compound specifically hybridizes with and inhibits the expression of a nucleic acid molecule encoding apolipoprotein B.

2. A method of treating an animal suspected of having or being prone to a disease or condition associated with cardiovascular disease by administering a therapeutically or prophylactically effective amount of the compound of claim 1.
